(19) United States
(12) Patent Application Publication
(10) Pub. No.: US 2010/0093696 A1
(43) Pub. Date: Apr. 15, 2010

(54) 2-AMINO PYRIMIDINE COMPOUNDS

(75) Inventors: Michael J. Bennett, San Diego, CA (US); Luke R. Zehnder, San Diego, CA (US); Sacha Ninkovic, Carlsbad, CA (US); Pei-Pei Kung, San Diego, CA (US); Jerry Jialun Meng, San Diego, CA (US); Huang Buwen, San Diego, CA (US)

Correspondence Address: PFIZER INC, 10555 SCIENCE CENTER DRIVE, SAN DIEGO, CA, 92121, US

(73) Assignee: Pfizer Inc.

(21) Appl. No.: 12/524,892

(22) PCT Filed: Jan. 25, 2008
(86) PCT No.: PCT/IB2008/000200

§ 371 Date: Dec. 7, 2009

Related U.S. Application Data
(60) Provisional application No. 60/888,433, filed on Feb. 6, 2007.
(60) Provisional application No. 61/020,661, filed on Jan. 11, 2008.

(51) Int. Cl. A61K 31/397; C07D 487/02; A61K 31/519; C12N 5/00; C07D 471/02; A61P 35/00; C07D 413/14; A61K 31/5377

(52) U.S. Cl. 514/210.21; 544/280; 514/265.1; 435/375; 544/279; 514/264.11; 544/117; 514/234.2

(57) ABSTRACT

The present invention is directed to compounds of formula (I), and pharmaceutically acceptable salts thereof, their synthesis, and their use as HSP-90 inhibitors.

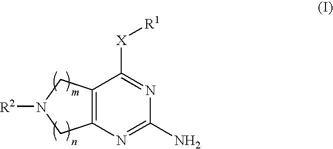

This application claims the benefit of U.S. Provisional Patent Application Nos. 60/888,433 filed Feb. 6, 2007 and 61/020,661 filed on Jan. 11, 2008, the disclosure of which is hereby incorporated by reference in its entirety.

### FIELD OF THE INVENTION

The present invention is directed to compounds, and pharmaceutically acceptable salts and solvates thereof, their synthesis, and their use as modulators or inhibitors of HSP-90. The compounds of the present invention are useful for modulating (e.g. inhibiting) HSP-90 activity and for treating diseases or conditions mediated by HSP-90, such as for example, disease states associated with abnormal cell growth such as cancer.

### BACKGROUND

Molecular chaperones play important roles in cellular function by ensuring proper folding of proteins upon synthesis as well as their refolding under conditions of denaturing stress. By regulating the balance between protein synthesis and degradation, molecular chaperones are a significant part of the cellular response to stress. In addition, by regulating the proper folding of various cellular proteins, chaperones play an important role in regulating cellular functions such as cell proliferation and apoptosis. (See, e.g. Jolly, et al., J. Natl. Cancer Inst. 92: 1564-1572 (2000)). Heat shock proteins (HSPs) are a class of chaperones that accumulate in the cell in response to various environmental stresses, such as heat shock, oxidative stress, or the presence of alcohols or heavy metals. In addition to their role in protecting the cell from such environmental stresses, HSPs may also play a significant role as chaperones for a variety of cellular proteins under stress-free conditions. Members of the HSP family are classified according to their molecular weight (e.g. HSP-27, HSP-70, and HSP-90). Evidence of differential expression of HSPs in various stages of tumor progression suggests HSPs play a role in cancer. (See, e.g. Martin, et al., Cancer Res. 60:2232-2238 (2000)).

HSP-90 is a homodimer with ATPase activity and functions in a series of complex interactions with a variety of substrate proteins (Young, et al., J. Cell Biol. 154: 267-273 (2001)). HSP-90 is unique with regard to other chaperones, however, since most of its known substrate proteins are signal transduction proteins. Thus, HSP-90 plays an essential role in regulating cellular signal transduction networks. (See, e.g. Xu, et al., Proc. Natl. Acad. Sci 90:7074-7078 (1993)). In particular, substrate proteins of HSP-90 include many mutated or over-expressed proteins implicated in cancer such as p53, Bcr-Ab1 kinase, Raf-1 kinase, Akt kinase, Npm-Alk kinase p185^{ErbB2} transmembrane kinase, Cdk4, Cdk6, Wee1 (a cell cycle-dependent kinase), HER2/Neu (ErbB2), and hypoxia inducible factor-1α (HIF-1α). Thus inhibition of HSP-90 results in selective degradation of these important signaling proteins involved in apoptosis, cell proliferation, and cell cycle regulation (Holstein, et al., Cancer Res. 61:4003-4009 (2001)). Accordingly, HSP-90 is an attractive therapeutic target because of the important roles played by these signaling proteins in disease states involving abnormal cell growth, such as cancer. It is thus desirable to discover and develop new inhibitors of HSP-90 activity that can provide a therapeutic benefit to, patients suffering from disease states related to abnormal cell growth such as cancer.

### SUMMARY

In one embodiment, the present invention provides a compound of formula (I)

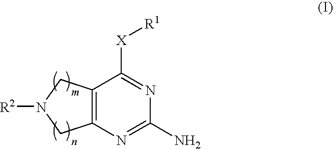

wherein:

m is 1 or 2, n is 1 or 2, when m is 2, n is 1;

X is a bond or a diradical selected from the group consisting of —O—, —S—, —(C₁-C₃ alkylene)-, —O—(C₁-C₃ alkylene)-, —NH—(C₁-C₃ alkylene)-, —S—(C₁-C₃ alkylene)-, —C(O)—, —C(O)—O—, —C(O)—NH—, —OC(O)—NH—, —NH—C(O)—NH—, —S(O)—, —S(O)₂—, —S(O)₂—O— and —S(O)₂—NH—, wherein each end of the diradical may be connected to R¹ or to the aminopyrimidine ring of formula I;

where permissible, each nitrogen or carbon atom of X is optionally further substituted by one group selected from —(C₁-C₃ alkylene)ₜ-CN, —(C₁-C₃ alkylene)ₜ-F, —(C₁-C₃ alkylene)ₜ-(C₁-C₃ perfluoroalkyl), —(C₁-C₃ alkylene)ₜ-O—(C₁-C₆ alkyl), —(C₁-C₃ alkylene)ₜ-OH, —(C₁-C₃ alkylene)ₜ-NH₂, —(C₁-C₃ alkylene)ₜ-NH(C₁-C₃ alkyl) and —(C₁-C₃ alkylene)ₜ-N(C₁-C₃ alkyl)(C₁-C₃ alkyl), and t is 0 or 1;

R¹ is selected from the group consisting of C₆-C₁₂ aryl, 5 to 12 member heteroaryl, C₃-C₁₂ cycloalkyl, 3-12 member heterocyclyl and C₅-C₁₂ unsaturated nonaromatic carbocyclyl, and each R¹ is optionally further substituted with 1-5 R, provided that when R¹ is phenyl, then R¹ is further substituted with at least two R and at least one of the R is not a halogen;

R is selected from the group consisting of R^{x}, —(C₁-C₆ alkylene)ₚ-O—(C₁-C₆ alkylene)ₚ-(C₆-C₁₀ aryl), —(C₁-C₆ alkylene)ₚ-O—(C₁-C₆ alkylene)ₚ-(C₇-C₁₀ cycloalkyl), —(C₁-C₆ alkylene)ₚ-O—(C₁-C₆ alkylene)ₚ-(7-10 member heteroaryl), —(C₁-C₆ alkylene)ₚ-O—(C₁-C₆ alkylene)ₚ-(7-10 member heterocyclyl), —(C₁-C₆ alkylene)ₚ-O—(C₂-C₆ alkenyl), —(C₁-C₆ alkylene)ₚ-O—(C₂-C₆ alkenylene)ₚ-(C₆-C₁₀ aryl), —(C₁-C₆ alkylene)ₚ-O—(C₂-C₆ alkenylene)ₚ-(C₃-C₁₀ cycloalkyl), —(C₁-C₆ alkylene)ₚ-O—(C₂-C₆ alkenylene)ₚ-(5-10 member heteroaryl), —(C₁-C₆ alkylene)ₚ-O—(C₂-C₆ alkenylene)ₚ-(3-10 member heterocyclyl), —(C₁-C₆ alkylene)ₚ-O—(C₂-C₆ alkynyl), —(C₁-C₆ alkylene)ₚ-O—(C₂-C₆ alkynylene)ₚ-(C₆-C₁₀ aryl), —(C₁-C₆ alkylene)ₚ-O—(C₂-C₆ alkynylene)ₚ-(C₃-C₁₀ cycloalkyl), —(C₁-C₆ alkylene)ₚ-O—(C₂-C₆ alkynylene)ₚ-(5-10 member heteroaryl) and —(C₁-C₆ alkylene)ₚ-O—(C₂-C₆ alkynylene)ₚ-(3-10 member heterocyclyl);

R² is selected from the group consisting of —(C₁-C₆ alkylene)ₚ-C(O)—R^{b}, alkylene)ₚ-C(O)—O—R^{a}, —(C₁-C₆ alkylene)ₚ-C(O)—N(R^{a})₂, —(C₁-C₆ alkylene)ₚ-S(O)—R^{a}, —(C₁-C₆ alkylene)ₚ-S(O)₂—R^{a}, —(C₁-C₆ alkylene)ₚ-S(O)₂—N(R^{a})₂, —(C₁-C₆ alkylene)ₚ-S(O)₂—O—R^{a}, and R³, wherein R³ is selected from —(C₁-C₆ alkylene)-(C₁-C₃ perfluoroalkyl), C₂-C₈ alkenyl, C₂-C₈ alkynyl, —(C₁-C₆ alkylene)ₚ-(C₃-C₁₂ cycloalkyl), —(C₁-C₆ alkylene)ₚ-(3-12 member heterocyclyl), —(C₁-C₆ alkylene)ₚ-(5-12 member heteroaryl) and —(C₁-C₆ alkylene)ₚ-(C₅-C₁₂ unsaturated nonaromatic carbocyclyl);

each R^{a} is independently selected from the group consisting of H, C₁-C₈ alkyl, C₂-C₈ alkenyl, C₂-C₈ alkynyl, C₁-C₈ perfluoroalkyl, —(C₁-C₆ alkylene)ₚ-(C₆-C₁₂ aryl), —(C₁-C₆ alkylene)ₚ-(5 to 12 member heteroaryl), —(C₁-C₆ alkylene)ₚ-(C₃-C₁₂ cycloalkyl), —(C₁-C₆ alkylene)ₚ-(3-12 member heterocyclyl), —(C₁-C₆ alkylene)ₚ-(C₅-C₁₂ unsaturated nonaromatic carbocyclyl);

two R^{a} attached to the same nitrogen atom, together with the nitrogen atom, may optionally form a 3-12 member heterocyclyl or a 5-12 member heteroaryl; the said 3-12 member heterocyclyl and the said 5-12 member heteroaryl is optionally further substituted by 1-5 R^{x};

R^{b} is selected from the group consisting of —NR^{a}N(R^{a})₂, —NR^{a}OR^{a}, C₂-C₈ alkenyl, C₂-C₈ alkynyl, C₁-C₈ perfluoroalkyl, —(C₃-C₆ alkylene)-(C₁-C₃ perfluoroalkyl), —(C₁-C₆ alkylene)ₚ-(C₆-C₁₂ aryl), —(C₁-C₆ alkylene)ₚ-(C₃-C₁₂ cycloalkyl), —(C₁-C₆ alkylene)ₚ-(3-12 member heterocyclyl), —(C₁-C₆ alkylene)ₚ-(5-12 member heteroaryl), —(C₁-C₆ alkylene)ₚ-(C₅-C₁₂ unsaturated nonaromatic carbocyclyl);

p is 0 or 1;

each R, R^{a}, R^{b} and R³ is optionally further substituted with 1-5 R^{x};

each R^{x} is independently selected from the group consisting of -oxo-, —(C₁-C₄ alkylene)-, halogen, —CN, —OH, C₁-C₆ alkyl, C₂-C₆ alkenyl, C₂-C₆ alkynyl, C₁-C₆ perfluoroalkyl, —(C₁-C₆ alkylene)-halogen, —(C₁-C₆ alkylene)-OH, —(C₁-C₆ alkylene)-CN, —(C₁-C₆ alkylene)_{q}-(C₃-C₆ cycloalkyl), —(C₁-C₆ alkylene)_{q}-(3-6 member heterocyclyl), —(C₁-C₆ alkylene)_{q}-(5-6 member heteroaryl), —(C₁-C₆ alkylene)_{q}-C(O)—(C₁-C₆ alkyl), —(C₁-C₆ alkylene)_{q}-C(O)—(C₃-C₆ cycloalkyl), —(C₁-C₆ alkylene)_{q}-C(O)—(C₁-C₆ alkylene)-(C₃-C₆ cycloalkyl), —(C₁-C₆ alkylene)_{q}-C(O)—O—(C₁-C₆ alkyl), —(C₁-C₆ alkylene)_{q}-C(O)—NH—(C₁-C₆ alkyl), —(C₁-C₆ alkylene)_{q}-C(O)—N(C₁-C₆ alkyl)(C₁-C₆ alkyl), —(C₁-C₆ alkylene)_{q}-O—(C₁-C₆ alkyl), —(C₁-C₆ alkylene)_{q}-O—(C₁-C₆ alkylene)-halogen, —(C₁-C₆ alkylene)_{q}-O—(C₁-C₆ alkylene)-(C₁-C₃ perfluoroalkyl), —(C₁-C₆ alkylene)_{q}-O—(C₁-C₆ alkylene)_{q}-(C₃-C₆ cycloalkyl), alkylene)_{q}-O—(C₁-C₆ alkylene)_{q}-(3-6 member heterocyclyl), —(C₁-C₆ alkylene)_{q}-O—(C₁-C₆ alkylene)_{q}-(5-6 member heteroaryl), —(C₁-C₆ alkylene)_{q}-O—(C₁-C₆ alkylene)-NH₂, —(C₁-C₆ alkylene)_{q}-O—(C₁-C₆ alkylene)-NH—(C₁-C₆ alkyl), —(C₁-C₆ alkylene)_{q}-O—(C₁-C₆ alkylene)-NH—(C₃-C₆ cycloalkyl), —(C₁-C₆ alkylene)_{q}-O—(C₁-C₆ alkylene)-N(C₁-C₆ alkyl)₂, —(C₁-C₆ alkylene)_{q}-NH₂, —(C₁-C₆ alkylene)_{q}-NH—(C₁-C₆ alkyl), —(C₁-C₆ alkylene)_{q}-NH—(C₃-C₆ cycloalkyl), —(C₁-C₆ alkylene)_{q}-N(C₁-C₆ alkyl)(C₁-C₆ alkyl), —(C₁-C₆ alkylene)_{q}-NHC(O)—(C₁-C₆ alkyl), —(C₁-C₆ alkylene)_{q}-NH—SO₂—(C₁-C₆ alkyl), —(C₁-C₆ alkylene)_{q}-SO₂—(C₁-C₆ alkyl), —(C₁-C₆ alkylene)_{q}-SO₂—(C₁-C₃ alkylene)_{q}-(C₃-C₆ cycloalkyl), —(C₁-C₆ alkylene)_{q}-SO₂—NH₂, —(C₁-C₆ alkylene)_{q}-SO₂—NH(C₁-C₃ alkyl), —(C₁-C₆ alkylene)_{q}-SO₂—NH—(C₁-C₃ alkylene)_{q}-(C₃-C₆ cycloalkyl) and —(C₁-C₆ alkylene)_{q}-SO₂—N(C₁-C₃ alkyl)₂; each q is independently 0 or 1; where permissible, each carbon atom of R^{x} is optionally further substituted by 1-3 fluorine;

or a pharmaceutically acceptable salt thereof.

In a preferred aspect of this embodiment, and in combination of any other aspects not inconsistent, m is 1, n is 1, and the compound is of formula II,

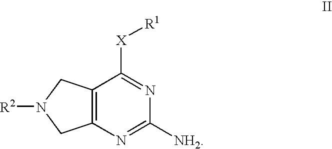

In another preferred aspect of this embodiment, and in combination of any other aspects not inconsistent, X is a bond or —O—, R¹ is C₆-C₁₂ aryl, 5 to 12 member heteroaryl, or 3-12 member heterocyclyl, and R¹ is further substituted with 2-5 R. More preferably, X is a bond and R¹ is a C₆-C₁₂ aryl further substituted with 2-5 R. Even more preferably, R¹ is phenyl further substituted with 2-5 R and at least one of the R is not a halogen.

In another preferred aspect of this embodiment, and in combination of any other aspects not inconsistent, R is selected from the group consisting of F, Cl, Br, —OH, —CN, C₁-C₃ alkyl, C₁-C₃ perfluoroalkyl, —(C₁-C₆ alkylene)-OH, —O—(C₁-C₆ alkyl), —(C₁-C₆ alkylene)-O—(C₁-C₆—(C₁-C₆ alkylene)ₚ-O—(C₁-C₆ alkylene)ₚ-(C₆-C₁₀ aryl), —(C₁-C₆ alkylene)ₚ-O—(C₁-C₆ alkylene)ₚ-(C₃-C₁₀ cycloalkyl), —(C₁-C₆ alkylene)ₚ-O—(C₁-C₆ alkylene)ₚ-(5-10 member heteroaryl), —(C₁-C₆ alkylene)ₚ-O—(C₁-C₆ alkylene)ₚ-(3-10 member heterocyclyl), —(C₁-C₆ alkylene)ₚ-O—(C₂-C₆ alkenyl), —(C₁-C₆ alkylene)ₚ-O—(C₂-C₆ alkenylene)ₚ-(C₆-C₁₀ aryl), —(C₁-C₆ alkylene)ₚ-O—(C₂-C₆ alkenylene)ₚ-(C₃-C₁₀ cycloalkyl), —(C₁-C₆ alkylene)ₚ-O—(C₂-C₆ alkenylene)ₚ-(5-10 member heteroaryl), —(C₁-C₆ alkylene)ₚ-O—(C₂-C₆ alkenylene)ₚ-(3-10 member heterocyclyl), —(C₁-C₆ alkylene)ₚ-O—(C₂-C₆ alkynyl), —(C₁-C₆ alkylene)ₚ-O—(C₂-C₆ alkynylene)ₚ-(C₆-C₁₀ aryl), —(C₁-C₆ alkylene)ₚ-β-(C₂-C₆ alkynylene)ₚ-(C₃-C₁₀ cycloalkyl), —(C₁-C₆ alkylene)ₚ-O—(C₂-C₆ alkynylene)ₚ-(5-10 member heteroaryl) and —(C₁-C₆ alkylene)ₚ-O—(C₂-C₆ alkynylene)ₚ-(3-10 member heterocyclyl); wherein each R is optionally further substituted by 1-5 R^{x}.

In another preferred aspect of this embodiment, and in combination with any other aspects not inconsistent, m is 1, n is 1 and the compound is of formula V,

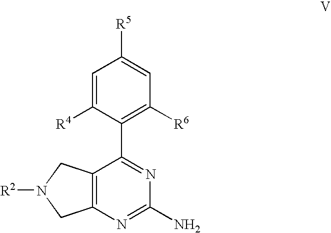

wherein

R⁴ and R⁵ are independently F, Cl, Br, —OH, —CN, unsubstituted C₁-C₃ alkyl, C₁-C₃ perfluoroalkyl, unsubstituted —(C₁-C₆ alkylene)-OH or unsubstituted —O—(C₁-C₆ alkyl);

R⁶ is selected from the group consisting of —(C₁-C₆ alkylene)-OH, —O—(C₁-C₆ alkyl), —(C₁-C₆ alkylene)-O—(C₁-C₆ alkyl), —(C₁-C₆ alkylene)ₚ-O—(C₁-C₆ alkylene)ₚ-(C₆-C₁₀ aryl), —(C₁-C₆ alkylene)ₚ-O—(C₁-C₆ alkylene)ₚ-(C₃-C₁₀ cycloalkyl), —(C₁-C₆ alkylene)ₚ-O—(C₁-C₆ alkylene)ₚ-(5-10 member heteroaryl), —(C₁-C₆ alkylene)ₚ-O—(C₁-C₆ alkylene)ₚ-(3-10 member heterocyclyl), —(C₁-C₆ alkylene)ₚ-O—(C₂-C₆ alkenyl), —(C₁-C₆ alkylene)ₚ-β-(C₂-C₆ alkenylene)ₚ-(C₆-C₁₀ aryl), —(C₁-C₆ alkylene)ₚ-O—(C₂-C₆ alkenylene)ₚ-(C₃-C₁₀ cycloalkyl), —(C₁-C₆ alkylene)ₚ-O—(C₂-C₆ alkenylene)ₚ-(5-10 member heteroaryl), —(C₁-C₆ alkylene)ₚ-O—(C₂-C₆ alkenylene)ₚ-(3-10 member heterocyclyl), —(C₁-C₆ alkylene)ₚ-O—(C₂-C₆ alkynyl), —(C₁-C₆ alkylene)ₚ-O—(C₂-C₆ alkynylene)ₚ-(C₆-C₁₀ aryl), —(C₁-C₆ alkylene)ₚ-O—(C₂-C₆ alkynylene)ₚ-(C₃-C₁₀ cycloalkyl), —(C₁-C₆ alkylene)ₚ-O—(C₂-C₆ alkynylene)ₚ-(5-10 member heteroaryl) and —(C₁-C₆ alkylene)ₚ-O—(C₂-C₆ alkynylene)ₚ-(3-10 member heterocyclyl); and R⁶ is optionally further substituted with 1-5 R^{x}.

In another preferred aspect of this embodiment, and in combination with any other aspects not inconsistent, m is 1, n is 1 and the compound is of formula VI,

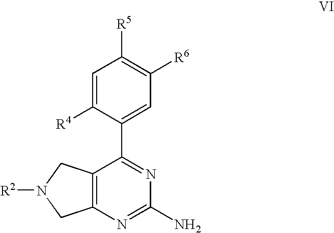

wherein

R⁴ and R⁵ are independently F, Cl, Br, —OH, —CN, unsubstituted C₁-C₃ alkyl, C₁-C₃ perfluoroalkyl, unsubstituted —(C₁-C₆ alkylene)-OH or unsubstituted —O—(C₁-C₆ alkyl);

R⁶ is selected from the group consisting of —(C₁-C₆ alkylene)-OH, —O—(C₁-C₆ alkyl), —(C₁-C₆ alkylene)-O—(C₁-C₆ alkyl), —(C₁-C₆ alkylene)ₚ-O—(C₁-C₆ alkylene)ₚ-(C₆-C₁₀ aryl), —(C₁-C₆ alkylene)ₚ-O—(C₁-C₆ alkylene)ₚ-(C₃-C₁₀ cycloalkyl), —(C₁-C₆ alkylene)ₚ-β-(C₁-C₆ alkylene)ₚ-(5-10 member heteroaryl), —(C₁-C₆ alkylene)ₚ-O—(C₁-C₆ alkylene)ₚ-(3-10 member heterocyclyl), —(C₁-C₆ alkylene)ₚ-O—(C₂-C₆ alkenyl), —(C₁-C₆ alkylene)ₚ-β-(C₂-C₆ alkenylene)ₚ-(C₆-C₁₀ aryl), —(C₁-C₆ alkylene)ₚ-O—(C₂-C₆ alkenylene)ₚ-(C₃-C₁₀ cycloalkyl), —(C₁-C₆ alkylene)ₚ-O—(C₂-C₆ alkenylene)ₚ-(5-10 member heteroaryl), —(C₁-C₆ alkylene)ₚ-O—(C₂-C₆ alkenylene)ₚ-(3-10 member heterocyclyl), —(C₁-C₆ alkylene)ₚ-O—(C₂-C₆ alkynyl), —(C₁-C₆ alkylene)ₚ-O—(C₂-C₆ alkynylene)ₚ-(C₆-C₁₀ aryl), —(C₁-C₆ alkylene)ₚ-O—(C₂-C₆ alkynylene)ₚ-(C₃-C₁₀ cycloalkyl), —(C₁-C₆ alkylene)ₚ-O—(C₂-C₆ alkynylene)ₚ-(5-10 member heteroaryl) and —(C₁-C₆ alkylene)ₚ-O—(C₂-C₆ alkynylene)ₚ-(3-10 member heterocyclyl); and R⁶ is optionally further substituted with 1-5 R^{x}.

In formula V and formula VI, preferably, R⁶ is —O—(C₁-C₆ alkylene)-(5-10 member heteroaryl), and R⁶ is optionally further substituted with 1-5 R^{x}; more preferably, R⁶ is —O—(C₁-C₆ alkylene)-(5 member heteroaryl), and R⁶ is optionally further substituted with 1-5 R^{x}; also preferably, R⁶ is —O—(C₁-C₆ alkylene)-(3-10 member heterocyclyl), and R⁶ is optionally further substituted with 1-5 R^{x}; also preferably, R⁶ is —O—(C₁-C₆ alkylene)-phenyl, and R⁶ is optionally further substituted with 1-5 R^{x}; also preferably, R⁶ is —O—(C₁-C₆ alkyl) or —O—(C₂-C₆ alkenyl), and R⁶ is optionally further substituted with 1-5 R^{x}. Preferred substituent of R⁶ is F, Cl, C₁-C₃ alkyl, —O—(C₁-C₃ alkyl), —OH, —CN and —(C₁-C₃ alkylene)-CN. Most preferred R⁶ includes the following group, wherein each group is unsubstituted or substituted with 1-5 R^{x}; preferably, the following group is unsubstituted or substituted with 1-5 groups of F, Cl, C₁-C₃ alkyl, —O—(C₁-C₃ alkyl), —OH, —CN or —(C₁-C₃ alkylene)-CN:

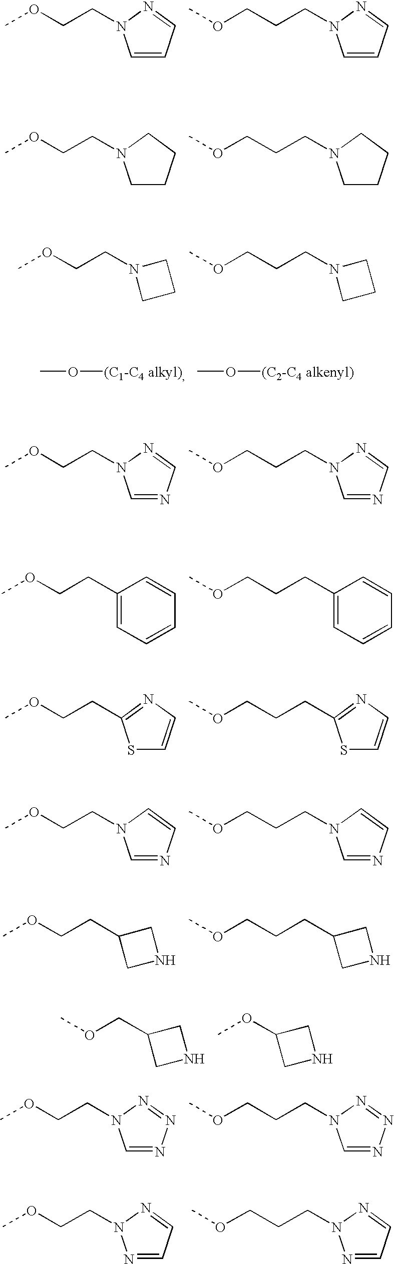

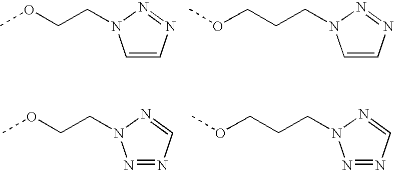

In another preferred aspect of this embodiment, and in combination with any other aspects not inconsistent, particularly in combination with the aspect wherein the compound is of formula V, also particularly in combination with the aspect wherein the compound is of formula VI, R² is —C(O)—N(R^{a})₂. Preferably, a first R^{a} is H or unsubstituted C₁-C₃ alkyl; and a second R^{a} is selected from C₁-C₆ alkyl, cyclopropyl, cyclobutyl, bicyclo[1.1.1]pent-1-yl, (1S,5R)-3-aza-bicyclo[3.1.0]hex-6-yl, 5-6 membered heteroaryl, 3-7 member heterocyclyl and C₂-C₆ alkenyl, and the second R^{a} is optionally further substituted by 1-5 groups selected from F, Cl, C₁-C₃ alkyl, —O—(C₁-C₃ alkyl), —OH, —CN and —(C₁-C₃ alkylene)-CN. More preferably, the first R^{a} is H.

In another preferred aspect of this embodiment, and in combination with any other aspects not inconsistent, particularly in combination with the aspect wherein the compound is of formula V, also particularly in combination with the aspect wherein the compound is of formula VI, R² is —C(O)—OR^{a}. Preferably, R^{a} is selected from C₁-C₆ alkyl, cyclopropyl, cyclobutyl, bicyclo[1.1.1]pent-1-yl, 5-6 membered heteroaryl, 3-7 member heterocyclyl and C₂-C₆ alkenyl, and R^{a} is optionally further substituted by 1-5 groups selected from F, Cl, C₁-C₃ alkyl, —O—(C₁-C₃ alkyl), —OH, —CN and —(C₁-C₃ alkylene)-CN.

In another embodiment, the present invention provides a compound of formula (I)

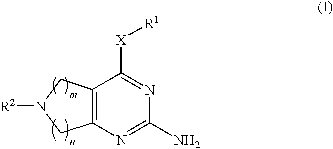

wherein:

m is 1 or 2, n is 1 or 2, when m is 2, n is 1;

X is a bond or a diradical selected from the group consisting of —O—, —S—, —(C₁-C₃ alkylene)-, —O—(C₁-C₃ alkylene)-, —NH—(C₁-C₃ alkylene)-, —S—(C₁-C₃ alkylene)-, —C(O)—, —C(O)—O—, —C(O)—NH—, —OC(O)—NH—, —NH—C(O)—NH—, —S(O)—, —S(O)₂—, —S(O)₂—O— and —S(O)₂—NH—, wherein each end of the diradical may be connected to R¹ or to the aminopyrimidine ring of formula I;

where permissible, each nitrogen or carbon atom of X is optionally further substituted by one group selected from —(C₁-C₃ alkylene)ₜ-CN, —(C₁-C₃ alkylene)ₜ-F, —(C₁-C₃ alkylene)ₜ-(C₁-C₃ perfluoroalkyl), —(C₁-C₃ alkylene)ₜ-O—(C₁-C₆ alkyl), —(C₁-C₃ alkylene)ₜ-OH, —(C₁-C₃ alkylene)ₜ-NH₂, —(C₁-C₃ alkylene)ₜ-NH(C₁-C₃ alkyl) and —(C₁-C₃ alkylene)ₜ-N(C₁-C₃ alkyl)(C₁-C₃ alkyl), and t is 0 or 1;

R¹ is selected from the group consisting of C₆-C₁₂ aryl, 5 to 12 member heteroaryl, C₃-C₁₂ cycloalkyl, 3-12 member heterocyclyl and C₅-C₁₂ unsaturated nonaromatic carbocyclyl, and each R¹ is optionally further substituted with 1-5 R^{x}, provided that when R¹ is phenyl, R¹ is further substituted with at least two R^{x} and one of the R^{x} is not a halogen;

preferred substituents of R¹ are selected from F, Cl, Br, —OH, —CN, C₁-C₃ alkyl, C₁-C₃ perfluoroalkyl, —(C₁-C₆ alkylene)-OH, —O—(C₁-C₆ alkyl), —O—(C₁-C₆ alkylene)-halogen, —O—(C₁-C₆ alkylene)-(C₁-C₃ perfluoroalkyl), —O—(C₁-C₆ alkylene)-N(C₁-C₆ alkyl)₂, —O—(C₁-C₆ alkylene)-NH₂ and —O—(C₁-C₆ alkylene)-NH(C₁-C₆ alkyl);

R² is selected from the group consisting of —(C₁-C₆ alkylene)ₚ-C(O)—R^{b}, —(C₁-C₆ alkylene)ₚ-C(O)—O—R^{a}, —(C₁-C₆ alkylene)ₚ-C(O)—N(R^{a})₂, —(C₁-C₆ alkylene)ₚ-S(O)—R^{a}, —(C₁-C₆ alkylene)ₚ-S(O)₂—R^{a}, —(C₁-C₆ alkylene)ₚ-S(O)₂—N(R^{a})₂, —(C₁-C₆ alkylene)ₚ-S(O)₂—O—R^{a}, and R³, wherein R³ is selected from —(C₁-C₆ alkylene)-(C₁-C₃ perfluoroalkyl), C₂-C₈ alkynyl, alkylene)ₚ-(C₃-C₁₂ cycloalkyl), —(C₁-C₆ alkylene)ₚ-(3-12 member heterocyclyl), —(C₁-C₆ alkylene)ₚ-(5-12 member heteroaryl) and —(C₁-C₆ alkylene)ₚ-(C₅-C₁₂ unsaturated nonaromatic carbocyclyl);

each R^{a} is independently selected from the group consisting of H, C₁-C₈ alkyl, C₂-C₈ alkenyl, C₂-C₈ alkynyl, C₁-C₈ perfluoroalkyl, —(C₁-C₆ alkylene)ₚ-(C₆-C₁₂ aryl), —(C₁-C₆ alkylene)ₚ-(5 to 12 member heteroaryl), —(C₁-C₆ alkylene)ₚ-(C₃-C₁₂ cycloalkyl), —(C₁-C₆ alkylene)ₚ-(3-12 member heterocyclyl), —(C₁-C₆ alkylene)ₚ-(C₅-C₁₂ unsaturated nonaromatic carbocyclyl);

two R^{a} attached to the same nitrogen atom, together with the nitrogen atom, may optionally form a 3-12 member heterocyclyl or a 5-12 member heteroaryl; the said 3-12 member heterocyclyl and the said 5-12 member heteroaryl is optionally further substituted by 1-5 R^{x};

R^{b} is selected from the group consisting of C₂-C₈ alkenyl, C₂-C₈ alkynyl, C₁-C₈ perfluoroalkyl, —(C₃-C₆ alkylene)-(C₁-C₃ perfluoroalkyl), —(C₁-C₆ alkylene)ₚ-(C₆-C₁₂ aryl), —(C₁-C₆ alkylene)ₚ-(C₃-C₁₂ cycloalkyl), —(C₁-C₆ alkylene)ₚ-(3-12 member heterocyclyl), —(C₁-C₆ alkylene)ₚ-(5-12 member heteroaryl), alkylene)ₚ-(C₅-C₁₂ unsaturated nonaromatic carbocyclyl);

p is 0 or 1; each R^{a}, R^{b} and R³ is optionally further substituted with 1-5 R^{x};

each R^{x} is independently selected from the group consisting of -oxo-, —(C₁-C₄ alkylene)-, halogen, —CN, —OH, C₁-C₆ alkyl, C₂-C₆ alkenyl, C₂-C₆ alkynyl, C₁-C₆ perfluoroalkyl, —(C₁-C₆ alkylene)-halogen, —(C₁-C₆ alkylene)-OH, —(C₁-C₆ alkylene)-CN, —(C₁-C₆ alkylene)_{q}-(C₃-C₆ cycloalkyl), —(C₁-C₆ alkylene)_{q}-(3-6 member heterocyclyl), —(C₁-C₆ alkylene)_{q}-(5-6 member heteroaryl), —(C₁-C₆ alkylene)_{q}-C(O)—(C₁-C₆ alkyl), —(C₁-C₆ alkylene)_{q}-C(O)—(C₃-C₆ cycloalkyl), —(C₁-C₆ alkylene)_{q}-C(O)—(C₁-C₆ alkylene)-(C₃-C₆ cycloalkyl), —(C₁-C₆ alkylene)_{q}-C(O)—O—(C₁-C₆ alkyl), —(C₁-C₆ alkylene)_{q}-C(O)—NH—(C₁-C₆ alkyl), —(C₁-C₆ alkylene)_{q}-C(O)—N(C₁-C₆ alkyl)(C₁-C₆ alkyl), —(C₁-C₆ alkylene)_{q}-O—(C₁-C₆ alkyl), —(C₁-C₆ alkylene)_{q}-O—(C₁-C₆ alkylene)-halogen, —(C₁-C₆ alkylene)_{q}-O—(C₁-C₆ alkylene)-(C₁-C₃ perfluoroalkyl), —(C₁-C₆ alkylene)_{q}-O—(C₁-C₆ alkylene)_{q} (C₃-C₆ cycloalkyl), —(C₁-C₆ alkylene)_{q}-O—(C₁-C₆ alkylene)_{q}-(3-6 member heterocyclyl), —(C₁-C₆ alkylene)_{q}-O—(C₁-C₆ alkylene)_{q}-(5-6 member heteroaryl), —(C₁-C₆ alkylene)_{q}-O—(C₁-C₆ alkylene)-NH₂, —(C₁-C₆ alkylene)_{q}-O—(C₁-C₆ alkylene)-NH—(C₁-C₆ alkyl), —(C₁-C₆ alkylene)_{q}-O—(C₁-C₆ alkylene)-NH—(C₃-C₆ cycloalkyl), —(C₁-C₆ alkylene)_{q}-O—(C₁-C₆ alkylene)-N(C₁-C₆ alkyl)₂, —(C₁-C₆ alkylene)_{q}-NH₂, —(C₁-C₆ alkylene)_{q}-NH—(C₁-C₆ alkyl), —(C₁-C₆ alkylene)_{q}-NH—(C₃-C₆ cycloalkyl), —(C₁-C₆ alkylene)_{q}-N(C₁-C₆ alkyl)(C₁-C₆ alkyl), —(C₁-C₆ alkylene)_{q}-NHC(O)—(C₁-C₆ alkyl), —(C₁-C₆ alkylene)_{q}-NH—SO₂—(C₁-C₆ alkyl), —(C₁-C₆ alkylene)_{q}-SO₂—(C₁-C₆ alkyl), —(C₁-C₆ alkylene)_{q}-SO₂—(C₁-C₃ alkylene)_{q}-(C₃-C₆ cycloalkyl), —(C₁-C₆ alkylene)_{q}-SO₂—NH₂, —(C₁-C₆ alkylene)_{q}-SO₂—NH(C₁-C₃ alkyl), —(C₁-C₆ alkylene)_{q}-SO₂—NH—(C₁-C₃ alkylene)_{q}-(C₃-C₆ cycloalkyl) and —(C₁-C₆ alkylene)_{q}-SO₂—N(C₁-C₃ alkyl)₂; each q is independently 0 or 1; where permissible, each carbon atom of R^{x} is optionally further substituted by 1-3 fluorine;

or a pharmaceutically acceptable salt thereof.

In the first particular aspect of this embodiment, and in combination with any other particular aspects not inconsistent, m is 1 and n is 1, and the compound is of formula II,

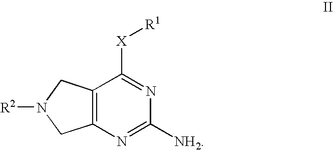

In the second particular aspect of this embodiment, and in combination with any other particular aspects not inconsistent, m is 1 and n is 2, and the compound is of formula III,

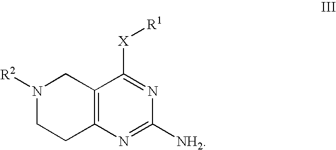

In the third particular aspect of this embodiment, and in combination with any other particular aspects not inconsistent, m is 2 and n is 1, and the compound is of formula IV,

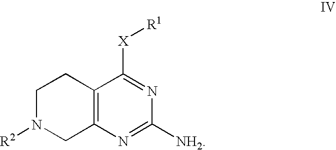

In the fourth particular aspect of this embodiment, and in combination with any other particular aspects not inconsistent, especially in combination with particular aspect one, two or three, X is a bond and R¹ is a C₆-C₁₂ aryl optionally substituted with 1-5 R^{x}. Preferably, R¹ is phenyl substituted with 2-5 R^{x} and at least one of the R^{x} is not a halogen. More preferably, R¹ is phenyl substituted with 2-3 groups selected from F, Cl, Br, —OH, —CN, C₁-C₃ alkyl, C₁-C₃ perfluoroalkyl, —(C₁-C₆ alkylene)-OH, —O—(C₁-C₆ alkyl), —O—(C₁-C₆ alkylene)-halogen, —O—(C₁-C₆ alkylene)-(C₁-C₃ perfluoroalkyl), —O—(C₁-C₆ alkylene)-N(C₁-C₆ alkyl)₂, —O—(C₁-C₆ alkylene)-NH₂ and —O—(C₁-C₆ alkylene)-NH(C₁-C₆ alkyl), provided that at least one of the said 2-3 groups is not a halogen.

In the fifth particular aspect of this embodiment, and in combination with any other particular aspects not inconsistent, especially in combination with particular aspect one, two or three, X is a bond, R¹ is 5-12 member heteroaryl, and R¹ is optionally further substituted with 1-5 R^{x}.

In the sixth particular aspect of this embodiment, and in combination with any other particular aspects not inconsistent, especially in combination with particular aspect one, two or three, X is a bond, R¹ is 3-12 member heterocyclyl, and R¹ is optionally further substituted with 1-5 R^{x}.

In the seventh particular aspect of this embodiment, and in combination with any other particular aspects not inconsistent, especially in combination with particular aspect one, two or three, X is a bond, R¹ is C₃-C₁₂ cycloalkyl, and R¹ is optionally further substituted with 1-5 R^{x}.

In the eighth particular aspect of this embodiment, and in combination with any other particular aspects not inconsistent, especially in combination with particular aspect one, two or three, X is —O— or unsubstituted —O—(C₁-C₃ alkylene)-, R¹ is C₆-C₁₂ aryl, and R¹ is optionally further substituted with 1-5 R^{x}.

In the ninth particular aspect of this embodiment, and in combination with any other particular aspects not inconsistent, especially in combination with particular aspect one, two or three, X is —O— or unsubstituted —O—(C₁-C₃ alkylene)-, R¹ is 5-12 member heteroaryl, and R¹ is optionally further substituted with 1-5 R^{x}.

In the tenth particular aspect of this embodiment, and in combination with any other particular aspects not inconsistent, especially in combination with particular aspect one, two or three, X is —O— or unsubstituted —O—(C₁-C₃ alkylene)-, R¹ is 3-12 member heterocyclyl, and R¹ is optionally further substituted with 1-5 R^{x}.

In the eleventh particular aspect of this embodiment, and in combination with any other particular aspects not inconsistent, especially in combination with particular aspect one, two or three, X is —O— or unsubstituted —O—(C₁-C₃ alkylene)-, R¹ is C₃-C₁₂ cycloalkyl, and R¹ is optionally further substituted with 1-5 R^{x}.

In the twelfth particular aspect of this embodiment, and in combination with any other particular aspects not inconsistent, especially in combination with particular aspect one, two or three, X is unsubstituted —NH—(C₁-C₃ alkylene)-, R¹ is C₆-C₁₂ aryl, and R¹ is optionally further substituted with 1-5 R^{x}.

In the thirteenth particular aspect of this embodiment, and in combination with any other particular aspects not inconsistent, especially in combination with particular aspect one, two or three, X is unsubstituted —NH—(C₁-C₃ alkylene)-, R¹ is 5-12 member heteroaryl, and R¹ is optionally further substituted with 1-5 R^{x}.

In the fourteenth particular aspect of this embodiment, and in combination with any other particular aspects not inconsistent, especially in combination with particular aspect one, two or three, X is unsubstituted —NH—(C₁-C₃ alkylene)-, R¹ is 3-12 member heterocyclyl, and R¹ is optionally further substituted with 1-5 R^{x}.

In the fifteenth particular aspect of this embodiment, and in combination with any other particular aspects not inconsistent, especially in combination with particular aspect one, two or three, X is unsubstituted —NH—(C₁-C₃ alkylene)-, R¹ is C₃-C₁₂ cycloalkyl, and R¹ is optionally further substituted with 1-5 R^{x}.

In the sixteenth particular aspect of this embodiment, and in combination with any other particular aspects not inconsistent, especially in combination with particular aspect one, two or three, wherein X is unsubstituted —(C₁-C₃ alkylene)-, R¹ is C₆-C₁₂ aryl, and R¹ is optionally further substituted with 1-5 R^{x}.

In the nineteenth particular aspect of this embodiment, and in combination with any other particular aspects not inconsistent, especially in combination with particular aspect one, two or three, X is unsubstituted —(C₁-C₃ alkylene)-, R¹ is 5-12 member heteroaryl, and R¹ is optionally further substituted with 1-5 R^{x}.

In the twentieth particular aspect of this embodiment, and in combination with any other particular aspects not inconsistent, especially in combination with particular aspect one, two or three, X is unsubstituted —(C₁-C₃ alkylene)-, R¹ is 3-12 member heterocyclyl, and R¹ is optionally further substituted with 1-5 R^{x}.

In the twenty-first particular aspect of this embodiment, and in combination with any other particular aspects not inconsistent, especially in combination with particular aspect one, two or three, X is unsubstituted —(C₁-C₃ alkylene)-, R¹ is C₃-C₁₂ cycloalkyl, and R¹ is optionally further substituted with 1-5 R^{x}.

In the twenty-second particular aspect of this embodiment, and in combination with any other particular aspects not inconsistent, including any of the particular aspect one to twenty-one, R² is —C(O)—OR⁸ or —(C₁-C₆ alkylene)-C(O)—O—R^{a}.

In the twenty-third particular aspect of this embodiment, and in combination with any other particular aspects not inconsistent, including any of the particular aspect one to twenty-one, R² is —C(O)—N(R^{a})₂ or —(C₁-C₆ alkylene)-C(O)—N(R^{a})₂.

In the twenty-fourth particular aspect of this embodiment, and in combination with any other particular aspects not inconsistent, including any of the particular aspect one to twenty-one, R² is selected from —S(O)₂—R^{a}, —S(O)₂—N(R^{a})₂, —S(O)₂—O—R^{a}, —(C₁-C₆ alkylene)-S(O)₂—R^{a}, —(C₁-C₆ alkylene)-S(O)₂—O—R^{a} and —(C₁-C₆ alkylene)-S(O)₂—N(R)₂.

In the twenty-fifth particular aspect of this embodiment, and in combination with any other particular aspects not inconsistent, including any of the particular aspect one to twenty-one, R² is —C(O)—R^{b} or —(C₁-C₆ alkylene)-C(O)—R^{b}.

In another embodiment, the present invention provides a compound of formula I,

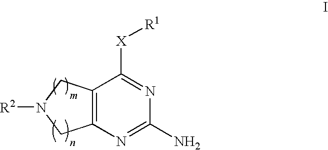

wherein:

m is 1 or 2, n is 1 or 2, when m is 2, n is 1;

X is a bond or a diradical selected from the group consisting of —O— and unsubstituted —O—(C₁-C₃ alkylene)-, wherein each end of the diradical may be connected to R¹ or to the aminopyrimidine ring of formula I;

R¹ is phenyl, 5-12 member heteroaryl or 3-12 member heterocyclyl;

when R¹ is phenyl, R¹ is further substituted with 2-5 groups selected from F, Cl, Br, —OH, —CN, C₁-C₃ alkyl, C₁-C₃ perfluoroalkyl, —(C₁-C₆ alkylene)-OH, —O—(C₁-C₆ alkyl), —O—(C₁-C₆ alkylene)-halogen, —O—(C₁-C₆ alkylene)-(C₁-C₃ perfluoroalkyl), —O—(C₁-C₆ alkylene)-N(C₁-C₆ alkyl)₂, —O—(C₁-C₆ alkylene)-NH₂ and —O—(C₁-C₆ alkylene)-NH(C₁-C₆ alkyl); and at least one of the substituent of R¹ is not a halogen;

when R¹ is 5-12 member heteroaryl or 3-12 member heterocyclyl, R¹ is optionally further substituted with 1-5 groups selected from F, Cl, Br, —OH, —CN, C₁-C₃ alkyl, C₁-C₃ perfluoroalkyl, —(C₁-C₆ alkylene)-OH, —O—(C₁-C₆ alkyl), —O—(C₁-C₆ alkylene)-halogen, —O—(C₁-C₆ alkylene)-(C₁-C₃ perfluoroalkyl), —O—(C₁-C₆ alkylene)-N(C₁-C₆ alkyl)₂, —O—(C₁-C₆ alkylene)-NH₂ and —O—(C₁-C₆ alkylene)-NH(C₁-C₆ alkyl);

R² is selected from the group consisting of —(C₁-C₆ alkylene)ₚ-C(O)—O—R^{a}, —(C₁-C₆ alkylene)ₚ-C(O)—N(R^{a})₂, —(C₁-C₆ alkylene)ₚ-C(O)—R^{b} and —(C₁-C₆ alkylene)ₚ-SO₂—R^{a};

R^{a} is selected from the group consisting of H, C₁-C₈ alkyl, C₂-C₈ alkenyl, C₂-C₈ alkynyl, C₁-C₈ perfluoroalkyl, —(C₁-C₆ alkylene)ₚ-(C₆-C₁₂ aryl), —(C₁-C₆ alkylene)ₚ-(5 to 12 member heteroaryl), —(C₁-C₆ alkylene)ₚ-(C₃-C₁₂ cycloalkyl), —(C₁-C₆ alkylene)ₚ-(3-12 member heterocyclyl) and —(C₁-C₆ alkylene)ₚ-(C₅-C₁₂ unsaturated nonaromatic carbocyclyl);

two R^{a} attached to the same nitrogen atom, together with the nitrogen atom; may optionally form a 3-12 member heterocyclyl or a 5-12 member heteroaryl; the said 3-12 member heterocyclyl and the said 5-12 member heteroaryl is optionally further substituted by 1-5 groups selected from C₁-C₃ alkyl, —CN, —F, —Cl, —Br, —O—(C₁-C₃ alkyl) and C₁-C₃ perfluoroalkyl;

R^{b} is selected from the group consisting of C₂-C₈ alkenyl, C₂-C₈ alkynyl, C₁-C₈ perfluoroalkyl, —(C₃-C₆ alkylene)-(C₁-C₃ perfluoroalkyl), —(C₁-C₆ alkylene)ₚ-(C₆-C₁₂ aryl), —(C₁-C₆ alkylene)ₚ-(C₃-C₁₂ cycloalkyl), —(C₁-C₆ alkylene)ₚ-(3-12 member heterocyclyl), —(C₁-C₆ alkylene)ₚ-(5-12 member heteroaryl), —(C₁-C₆ alkylene)ₚ-(C₈-C₁₂ unsaturated nonaromatic carbocyclyl);

each R^{a} and R^{b} is independently optionally further substituted by 1-5 groups selected from C₁-C₃ alkyl, —CN, —F, —Cl, —Br, —O—(C₁-C₃ alkyl), C₁-C₃ perfluoroalkyl, —NH₂, —NH—(C₁-C₃ alkyl), —N(C₁-C₃ alkyl)₂ and OH;

p is 0 or 1;

or a pharmaceutically acceptable salt thereof.

In the first particular aspect of this embodiment, and in combination with any other particular aspects not inconsistent, m is 1 and n is 1.

In the second particular aspect of this embodiment, and in combination with any other particular aspects not inconsistent, m is 1 and n is 2.

In the third particular aspect of this embodiment, and in combination with any other particular aspects not inconsistent, m is 2 and n is 1.

In the fourth particular aspect of this embodiment, and in combination with any other particular aspects not inconsistent, X is a bond.

In the fifth particular aspect of this embodiment, and in combination with any other particular aspects not inconsistent, X is diradical of —O— or unsubstituted —O—(C₁-C₃ alkylene)-.

In another embodiment, the present invention provides a compound of formula II,

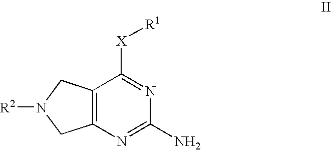

wherein:

X is a bond or a diradical selected from the group consisting of —O—, —NH—, —S—, —(C₁-C₃ alkylene)-, —O—(C₁-C₃ alkylene)-, —NH—(C₁-C₃ alkylene)-, —S—(C₁-C₃ alkylene)-, —C(O)—, —C(O)—O—, —C(O)—NH—, —OC(O)—NH—, —NH—C(O)—NH—, —S(O)—, —S(O)₂—, —S(O)₂—O— and —S(O)₂—NH—, wherein each end of the diradical may be connected to R¹ or the aminopyrimidine ring of formula II;

where permissible, each nitrogen or carbon atom of X is optionally further substituted by a group selected from —(C₁-C₃ alkylene)ₜ-CN, —(C₁-C₃ alkylene)ₜ-F, —(C₁-C₃ alkylene)ₜ-(C₁-C₃ perfluoroalkyl), —(C₁-C₃ alkylene)ₜ-O—(C₁-C₆ alkyl), —(C₁-C₃ alkylene)ₜ-OH, —(C₁-C₃ alkylene)ₜ-NH₂, —(C₁-C₃ alkylene)ₜ-NH(C₁-C₃ alkyl), —(C₁-C₃ alkylene)ₜ-N(C₁-C₃ alkyl)(C₁-C₃ alkyl), and t is 0 or 1;

R¹ is selected from the group consisting of C₆-C₁₂ aryl, 5 to 12 member heteroaryl, C₃-C₁₂ cycloalkyl, 3-12 member heterocyclyl and C₅-C₁₂ unsaturated nonaromatic carbocyclyl, and each R¹ is optionally further substituted with 1-5 R^{x};

R² is selected from the group consisting of —(C₁-C₆ alkylene)ₚ-R^{a}, —(C₁-C₆ alkylene)ₚ-C(O)—R^{a}, —(C₁-C₆ alkylene)ₚ-C(O)—O—R^{a}, —(C₁-C₆ alkylene)ₚ-C(O)—N(R^{a})₂, —(C₁-C₆ alkylene)ₚ-S(O)—R^{a}, —(C₁-C₆ alkylene)ₚ-S(O)₂R^{a}, —(C₁-C₆ alkylene)ₚ-S(O)₂—N(R^{a})₂ and —(C₁-C₆ alkylene)ₚ-S(O)₂—O—R^{a};

each R^{a} is independently selected from the group consisting of H, C₁-C₈ alkyl, C₂-C₈ alkenyl, C₂-C₈ alkynyl, C₁-C₈ perfluoroalkyl, —(C₁-C₆ alkylene)ₚ-(C₆-C₁₂ aryl), —(C₁-C₆ alkylene)ₚ-(5 to 12 member heteroaryl), —(C₁-C₆ alkylene)ₚ-(C₃-C₁₂ cycloalkyl), —(C₁-C₆ alkylene)-(3-12 member heterocyclyl), and —(C₁-C₆ alkylene)ₚ-(C₅-C₁₂ unsaturated nonaromatic carbocyclyl), R^{a} is optionally further substituted with 1-5 R^{x};

two R^{a} attached to the same nitrogen atom, together with the nitrogen atom, may optionally form a 3-12 member heterocyclyl or a 5-12 member heteroaryl; the said 3-12 member heterocyclyl and the said 5-12 member heteroaryl is optionally further substituted by 1-5 R^{x};

each p is independently 0 or 1;

each R^{x} is independently selected from the group consisting of -oxo-, —(C₁-C₄ alkylene)-, halogen, —CN, —OH, C₁-C₆ alkyl, C₂-C₆ alkenyl, C₂-C₆ alkynyl, C₁-C₆ perfluoroalkyl, —(C₁-C₆ alkylene)-halogen, —(C₁-C₆ alkylene)-OH, —(C₁-C₆ alkylene)-CN, —(C₁-C₆ alkylene)_{q}-(C₃-C₆ cycloalkyl), —(C₁-C₆ alkylene)_{q}-(3-6 member heterocyclyl), —(C₁-C₆ alkylene)_{q}-(5-6 member heteroaryl), —(C₁-C₆ alkylene)_{q}-C(O)—(C₁-C₆ alkyl), —(C₁-C₆ alkylene)_{q}-C(O)—(C₃-C₆ cycloalkyl), —(C₁-C₆ alkylene)_{q}-C(O)—(C₁-C₆ alkylene)-(C₃-C₆ cycloalkyl), —(C₁-C₆ alkylene)_{q}-C(O)—O—(C₁-C₆ alkyl), —(C₁-C₆ alkylene)_{q}-C(O)—NH—(C₁-C₆ alkyl), —(C₁-C₆ alkylene)_{q}-C(O)—N(C₁-C₆ alkyl)(C₁-C₆ alkyl), —(C₁-C₆ alkylene)_{q}-O—(C₁-C₆ alkyl), —(C₁-C₆ alkylene)_{q}-O—(C₁-C₆ alkylene)-halogen, —(C₁-C₆ alkylene)_{q}-O—(C₁-C₆ alkylene)-(C₁-C₃ perfluoroalkyl), —(C₁-C₆ alkylene)_{q}-O—(C₁-C₆ alkylene)_{q}-(C₃-C₆ cycloalkyl), —(C₁-C₆ alkylene)_{q}-O—(C₁-C₆ alkylene)_{q}-(3-6 member heterocyclyl), —(C₁-C₆ alkylene)_{q}-O—(C₁-C₆ alkylene)_{q}-(5-6 member heteroaryl), —(C₁-C₆ alkylene)_{q}-O—(C₁-C₆ alkylene)-NH₂, —(C₁-C₆ alkylene)_{q}-O—(C₁-C₆ alkylene)-NH—(C₁-C₆ alkyl), —(C₁-C₆ alkylene)_{q}-O—(C₁-C₆ alkylene)-NH—(C₃-C₆ cycloalkyl), —(C₁-C₆ alkylene)_{q}-O—(C₁-C₆ alkylene)-N(C₁-C₆ alkyl)₂, —(C₁-C₆ alkylene)_{q}-NH₂, —(C₁-C₆ alkylene)_{q}-NH—(C₁-C₆ alkyl), —(C₁-C₆ alkylene)_{q}-NH—(C₃-C₆ cycloalkyl), —(C₁-C₆ alkylene)_{q}-N(C₁-C₆ alkyl)(C₁-C₆ alkyl), (C₁-C₆ alkylene)_{q}-NHC(O)—(C₁-C₆ alkyl), —(C₁-C₆ alkylene)_{q}-NH—SO₂—(C₁-C₆ alkyl), —(C₁-C₆ alkylene)_{q}-SO₂—(C₁-C₆ alkyl), —(C₁-C₆ alkylene)_{q}-SO₂—(C₁-C₃ alkylene)_{q}-(C₃-C₆ cycloalkyl), —(C₁-C₆ alkylene)_{q}-SO₂—NH₂, —(C₁-C₆ alkylene)_{q}-SO₂—NH(C₁-C₃ alkyl), —(C₁-C₆ alkylene)_{q}-SO₂—NH—(C₁-C₃ alkylene)_{q}-(C₃-C₆ cycloalkyl) and —(C₁-C₆ alkylene)_{q}-SO₂—N(C₁-C₃ alkyl)₂; q is independently 0 or 1; where permissible, each carbon atom of R^{x} is optionally further substituted by 1-3 fluorine;

or a pharmaceutically acceptable salt thereof.

In the first particular aspect of this embodiment, and in combination with any other particular aspects not inconsistent, X is a bond or a diradical selected from the group consisting of —O— and unsubstituted —O—(C₁-C₃ alkylene)-; R^{x} is selected from the group consisting of F, Cl, Br, —OH, —CN, C₁-C₃ alkyl, C₁-C₃ perfluoroalkyl, —(C₁-C₆ alkylene)-OH, —O—(C₁-C₆ alkyl), —O—(C₁-C₆ alkylene)-halogen, —O—(C₁-C₆ alkylene)-(C₁-C₃ perfluoroalkyl), —O—(C₁-C₆ alkylene)-N(C₁-C₆ alkyl)₂, —O—(C₁-C₆ alkylene)-NH₂ and —O—(C₁-C₆ alkylene)-NH(C₁-C₆ alkyl. Preferably, X is a bond, R¹ is selected from the group consisting of phenyl, 5-12 member heteroaryl and 3-12 member heterocyclyl, R¹ is optionally further substituted by 1-5 R^{x}. More preferably, R¹ is phenyl optionally further substituted by 1-5 R^{x}.

In the second particular aspect of this embodiment, and in combination with any other particular aspects not inconsistent, X is a diradical —O—(C₁-C₃ alkylene)-, R¹ is phenyl or 5-12 member heteroaryl, R¹ is optionally further substituted by 1-5 R^{x}.

In the third particular aspect of this embodiment, and in combination with any other particular aspects not inconsistent, X is a diradical —O—, R¹ is phenyl optionally further substituted by 1-5 R^{x}.

In another embodiment, the present invention provides a pharmaceutical composition comprising a compound of formula I, II, III, IV, V or VI, or a pharmaceutically acceptable salt thereof, and a pharmaceutically acceptable carrier.

In another embodiment, the present invention provides a use of a compound of formula I, II, III, IV, V or VI, or a pharmaceutically acceptable salt thereof, in the preparation of a medicament for the treatment of cancer.

In another embodiment, the present invention provides a method of modulating the activity of HSP-90, comprising contacting a cell with a compound of formula I, II, III, IV, V or VI, or a pharmaceutically acceptable salt thereof.

This invention also relates to a method for the treatment of abnormal cell growth in a mammal, including a human, comprising administering to said mammal an amount of a compound of the Formula I, as defined above, or a pharmaceutically acceptable salt or solvate thereof, that is effective in treating abnormal cell growth.

In one embodiment of this method, the abnormal cell growth is cancer, including, but not limited to, mesothelioma, hepatobilliary (hepatic and billiary duct), a primary or secondary CNS tumor, a primary or secondary brain tumor, lung cancer (NSCLC and SCLC), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, non hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In another embodiment of said method, said abnormal cell growth is a benign proliferative disease, including, but not limited to, psoriasis, benign prostatic hypertrophy or restinosis.

In a preferred embodiment of the present invention the cancer is selected from lung cancer (NSCLC and SCLC), cancer of the head or neck, ovarian cancer, colon cancer, rectal cancer, cancer of the anal region, stomach cancer, breast cancer, cancer of the kidney or ureter, renal cell carcinoma, carcinoma of the renal pelvis, neoplasms of the central nervous system (CNS), primary CNS lymphoma, non hodgkins's lymphoma, spinal axis tumors, or a combination of one or more of the foregoing cancers.

In another preferred embodiment of the present invention the cancer is selected from lung cancer (NSCLC and SCLC), ovarian cancer, colon cancer, rectal cancer, cancer of the anal region, or a combination of one or more of the foregoing cancers.

In a more preferred embodiment of the present invention the cancer is selected from lung cancer (NSCLC and SCLC), ovarian cancer, colon cancer, rectal cancer, or a combination of one or more of the foregoing cancers.

In another embodiment of said method, said abnormal cell growth is a benign proliferative disease, including, but not limited to, psoriasis, benign prostatic hypertrophy or restinosis.

This invention also relates to a pharmaceutical composition for the treatment of abnormal cell growth in a mammal, including a human, comprising an amount of a compound of the Formula I, as defined above, or a pharmaceutically acceptable salt or solvate thereof, that is effective in treating abnormal cell growth, and a pharmaceutically acceptable carrier. In one embodiment of said composition, said abnormal cell growth is cancer, including, but not limited to, mesothelioma, hepatobilliary (hepatic and billiary duct), a primary or secondary CNS tumor, a primary or secondary brain tumor, lung cancer (NSCLC and SCLC), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, non hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers. In another embodiment of said pharmaceutical composition, said abnormal cell growth is a benign proliferative disease, including, but not limited to, psoriasis, benign prostatic hypertrophy or restinosis.

As used herein, the symbol [- - - - - -] when incorporated into the chemical structure of a substituent means that the atom to which [- - - - - -] is attached is the point of attachment of that substitutent to some position on another molecule. For example, X in the hypothetical molecule CH₃CH₂—X might be defined as X is

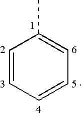

In which case, the placement of [- - - - - -] attached to the arbitrarily numbered position C-1, means that C-1 of the phenyl ring is attached to the methylene carbon.

The symbols “ [symbol] ” and “ [symbol] ”, when used together in a single molecule without further indication otherwise, for example, chemical name or accompanying description, merely indicate relative stereochemistry of trans or cis where applicable. The symbol “ [symbol] ” and the symbol “ [symbol] ”, used together or separately, in combination with an indication of them representing the absolute stereochemistry, for example, an indication of “S” or “R” in the corresponding chemical structure or the accompanying chemical name, indicate the absolute stereochemistry of the corresponding chiral center.

“Aliphatic” refers to straight-chain, branched or cyclic C₁-C₁₂ hydrocarbons which are completely saturated or which contains one or more units of unsaturation but which are not aromatic. Examples of aliphatic groups include linear, branched or cyclic alkyl, alkenyl, alkynyl groups and hybrids thereof such as (cycloalkyl)alkyl, (cycloalkenyl)alkyl, etc. An aliphatic group may be optionally substituted by 1-6 substituents. Suitable substituents on an aliphatic group include: 3-12 member heterocyclyl, C₆-C₁₀ aryl, 5-12 member heteroaryl, halogen, —NO₂, NH₂, NR₂, —CN, —COR, —COOR, —CONR₂, —OH, —OR, —OCOR, —SR, —SOR, —SO₂R, —SONR₂, —SO₂NR₂, wherein R is H, C₁-C₁₀ alkyl, 3-10 member heterocyclyl, C₆-C₁₀ aryl, 5-12 member heteroaryl.

“C₁-C₁₂ alkyl” refers to a straight chain or branched saturated hydrocarbon radical having from 1 to 12 carbon atoms. A C₁-C₁₂ alkyl group may be optionally substituted by at least one substituent. Suitable substituents on a C₁-C₁₂ alkyl group include, but are not limited to, 3-12 member heterocyclyl, C₆-C₁₀ aryl, 5-12 member heteroaryl, halogen, —NO₂, —NR₂, —CN, —COR, —COOR, —CONR₂, —OH, —OR, —OCOR, —SR, —SOR, —SO₂R, —SONR₂, —SO₂NR₂, wherein each R is independently —H, C₁-C₁₀ alkyl, 3-12 member heterocyclyl, C₆-C₁₀ aryl, 5-12 member heteroaryl. Examples of C₁-C₁₂ alkyl groups include, but are not limited to methyl, ethyl, propyl, isopropyl, butyl, sec-butyl, iso-butyl, tert-butyl, pentyl, neo-pentyl, sec-pentyl, hexyl, heptyl, octyl, and the like, including substituted forms thereof. Further, the term “alkyl” refers to a straight chain or branched saturated hydrocarbon radical of 1 to 20 carbon atoms, or 1 to 12 carbon atoms, or 1 to 8 carbon atoms, or 1 to 6 carbon atoms, or 1 to 4 carbon atoms. “Lower alkyl” refers specifically to an alkyl group having 1 to 4 carbon atoms. Alkyl may be substituted or unsubstituted. Suitable substituents on an alkyl group are the same as those described for a C₁-C₁₂ alkyl group.

“Cycloalkyl” refers to a cyclic saturated hydrocarbon radical having from 3 to 20 carbon atoms. A cycloalkyl group may be monocyclic and where permissible may be bicyclic or polycyclic. A cycloalkyl group may be optionally substituted by at least one substituent. Suitable substituents on a cycloalkyl group are the same as those described for an alkyl group. Examples of cycloalkyl groups include, but are not limited to cyclopropyl, cyclobutyl, cyclopentyl, cyclohexyl, cycloheptyl, cyclooctyl, nobornyl, adamantyl, and the like, including substituted forms thereof.

“Nonaromatic carbocyclyl” refers to a 3 to 12 member all-carbon monocyclic ring group, all-carbon bicyclic or multicyclic ring system group wherein one or more of the rings may contain one or more double bonds or an aromatic ring as part of the bicyclic or multicyclic ring system, but the monocyclic ring, the bicyclic or multicyclic ring system does not have a completely conjugated pi-electron system. Examples, without limitation, of nonaromatic carbocyclyl are cyclopropyl, cyclobutyl, cyclopentyl, cyclopentenyl, cyclohexyl, cyclohexadienyl, adamantanyl, cycloheptyl, cycloheptatrienyl and the like. A nonaromatic carbocyclyl may be substituted or unsubstituted. Typical substituent groups are the same with those of alkyl group, as defined herein. Illustrative examples of nonaromatic carbocyclyl are derived from, but not limited to, the following:

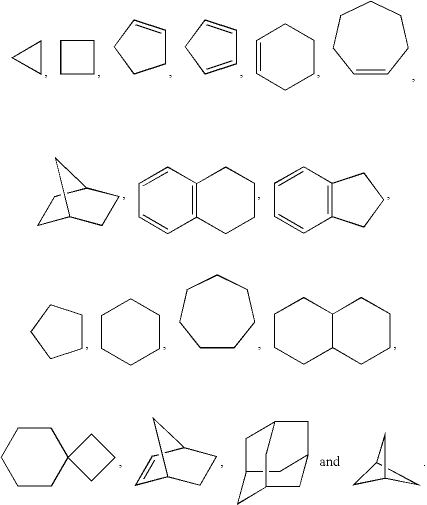

“Unsaturated nonaromatic carbocyclyl” or “nonaromatic unsaturated carbocyclyl” both refer to a nonaromatic carbocyclyl, as defined herein, that contains at least one carbon carbon double bond or one carbon carbon triple bond, or an aromatic ring as part of the bicyclic or multicyclic ring system.

“C₂-C₁₂ alkenyl” refers to a straight chain or branched unsaturated hydrocarbon radical having from 2 to 12 carbon atoms. A C₂-C₁₂ alkenyl group may have one or more points of unsaturation (i.e.—one or more carbon-carbon double bonds). In the case where C₂-C₁₂ alkenyl has more than one carbon-carbon double bond, the carbon-carbon double bonds can be conjugated or unconjugated. A C₂-C₁₂ alkenyl group may be optionally substituted by at least one substituent. Suitable substituents on a C₂-C₁₂ alkenyl group are the same as those described for a C₁-C₁₂ alkyl group. Examples of C₂-C₁₂ alkenyl include, but are not limited to, ethenyl, 1-propenyl, 2-propenyl, 1-butenyl, 2-butenyl, iso-butenyl, and the like, including substituted forms thereof. Further, the term “alkenyl” refers to a straight chain or branched unsaturated hydrocarbon radical having from 2 to 20 carbon atoms, or 2 to 12 carbon atoms, or 2 to 8 carbon atoms, or 2 to 6 carbon atoms, or 2 to 4 carbon atoms. An alkenyl group may have one or more points of unsaturation (i.e.—one or more carbon-carbon double bonds). In the case where an alkenyl group has more than one carbon-carbon double bond, the carbon-carbon double bonds can be conjugated or unconjugated. An alkenyl group may be substituted or unsubstituted. Suitable substituents on an alkenyl group are the same as those described for a C₁-C₁₂ alkyl group.

“Alkoxy” or “alkoxyl” refers to —OR^{c} wherein R^{c} is C₁-C₁₂ alkyl, C₂-C₁₂ alkenyl, C₂-C₁₂ alkynyl, C₃-C₁₂ cycloalkyl or (C₁-C₆ alkylene)-(C₃-C₁₂ cycloalkyl). A “C₁-C₁₂ alkoxy” or “C₁-C₁₂ alkoxyl” refers to an alkoxy group, as defined herein, wherein R^{c} has 1 to 12 total carbon atoms.

“Alkoxyalkyl” refers to an alkyl, as defined herein, that is substituted by at least one alkoxy group as defined herein. A “C₂-C₆ alkylalkoxy” refers an alkylalkoxy wherein the total carbon number of the alkyl and its alkoxy substituents are from 2 to 6.

“Alkylamino” refers to —NR^{p}R^{q} wherein each R^{p} and R^{q} is independently H, C₁-C₁₂ alkyl, C₂-C₁₂ alkenyl, C₂-C₁₂ alkynyl, C₃-C₁₂ cycloalkyl, (C₁-C₆ alkylene)-(C₃-C₁₂ cycloalkyl) provided R^{p} and R^{q} are not both H. A “monoalkylamino” refers to an alkylamino group, as defined herein, wherein one of R^{p} and R^{q} is H. A “dialkylamino” refers to an alkylamino group, as defined herein, wherein none of R^{p} and R^{q} is H. A “C₁₋₁₂ alkylamino” refers to an alkylamino group that contains 1 to 10 carbon atoms.

“C₂-C₁₂ alkynyl” refers to a straight chain or branched hydrocarbon radical having from 2-12 carbon atoms and at least one carbon-carbon triple bond. In the case where C₂-C₁₂ alkynyl has more than one carbon-carbon double bond, the carbon-carbon double bonds can be conjugated or unconjugated. A C₂-C₁₂ alkynyl group may be optionally substituted by at least one substituent. Suitable substituents on a C₂-C₁₂ alkynyl group are the same as those described for a C₁-C₁₂ alkyl group. Examples of C₂-C₁₂ alkynyl include, but are not limited to, ethynyl, 1-propynyl, 2-propynyl, 1-butynyl, 2-butynyl, and the like, including substituted forms thereof. Further, the term “alkynyl” refers to a straight chain or branched hydrocarbon radical of 2 to 20 carbon atoms, or 2 to 12 carbon atoms, or 2 to 8 carbon atoms, or 2 to 6 carbon atoms, or 2 to 4 carbon atoms, and having at least one carbon-carbon triple bond. Alkynyl may be substituted or unsubstituted. Suitable substituents on an alkynyl group are the same as those described for a C₁-C₁₂ alkyl group.

“Amino” refers to —NH₂.

“C₆-C₁₀ aryl” refers to an all-carbon monocyclic ring or polycyclic ring of 6 to 10 carbon atoms having a completely conjugated pi-electron system. A C₆-C₁₀ aryl group may be optionally substituted by at least one substituent. Suitable substituents on a C₆-C₁₀ aryl group are the same as those described for a C₁-C₁₂ alkyl group. Examples of C₆-C₁₀ aryl include, but are not limited to, phenyl and naphthyl. Further, the term “aryl” refers to an all-carbon monocyclic ring or polycyclic ring of 6 to 20 carbon atoms having a completely conjugated pi-electron system. The aryl group may be substituted or unsubstituted. Examples of aryl include, but are not limited to, anthracenyl, phenanthreneyl and perylenyl.

“Aralkyl” refers to alkyl, as defined herein, that is substituted with an C₆₋₁₀ aryl group as defined above; e.g., —CH₂phenyl, —(CH₂)₂phenyl, —(CH₂)₃phenyl, CH₃CH(CH₃)CH₂phenyl, and the like and derivatives thereof. A C₁-C₆ aralkyl refers to a C₁-C₆ alkyl that is substituted with a C₆-C₁₀ aryl group.

“Heteroaralkyl” group means alkyl, as defined herein, that is substituted with a 5-12 member heteroaryl group; e.g., —CH₂pyridinyl, —(CH₂)₂pyrimidinyl, —(CH₂)₃imidazolyl, and the like, and derivatives thereof. A C₁-C₆ heteroaralkyl refers to a C₁-C₆ alkyl that is substituted with an 5-12 member heteroaryl group.

“Heteroaryl” refers to a monocyclic or fused ring group of 5 to 12 ring atoms containing one, two, three or four ring heteroatoms selected from N, O, and S, the remaining ring atoms being C, and, in addition, having a completely conjugated pi-electron system. Examples, without limitation, of unsubstituted heteroaryl groups are pyrrole, furan, thiophene, imidazole, oxazole, thiazole, pyrazole, pyridine, pyrimidine, quinoline, isoquinoline, purine, tetrazole, triazine, and carbazole. The heteroaryl group may be substituted or unsubstituted. Typical substituents include C₁₋₁₂ aliphatic, 3-10 member heterocyclyl, 6-10 member aryl, halogen, —NO₂, NH₂, NR₂, —CN, —COR, —COOR, —CONR₂, —OH, —OR, —OCOR, —SR, —SOR, —SO₂R, —SONR₂, —SO₂NR₂, wherein R is a C₁₋₁₀ aliphatic, 3-10 member heterocyclyl, C₆₋₁₀ aryl, 5-10 member heteroaryl.

A “pharmaceutically acceptable heteroaryl” is one that is sufficiently stable to be attached to a compound of the invention, formulated into a pharmaceutical composition and subsequently administered to a patient in need thereof.

Examples of typical monocyclic heteroaryl groups include, but are not limited to:

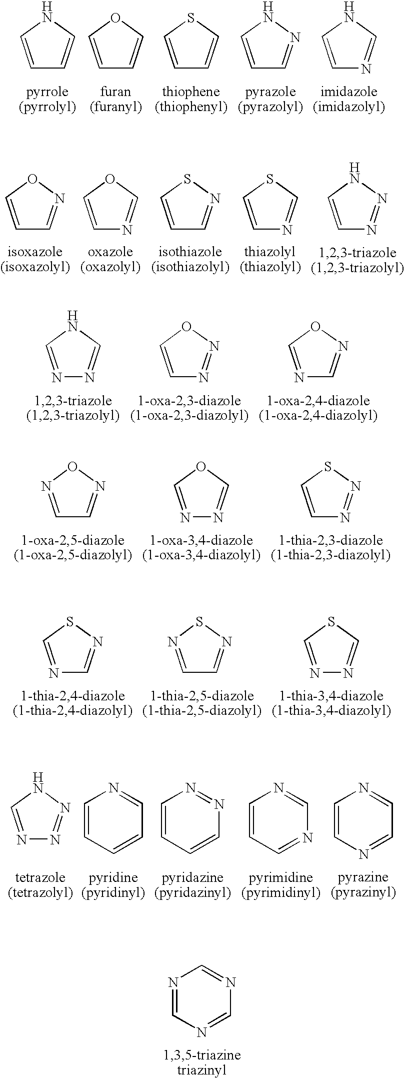

Examples of bicyclic heteroaryl groups include, but are not limited to:

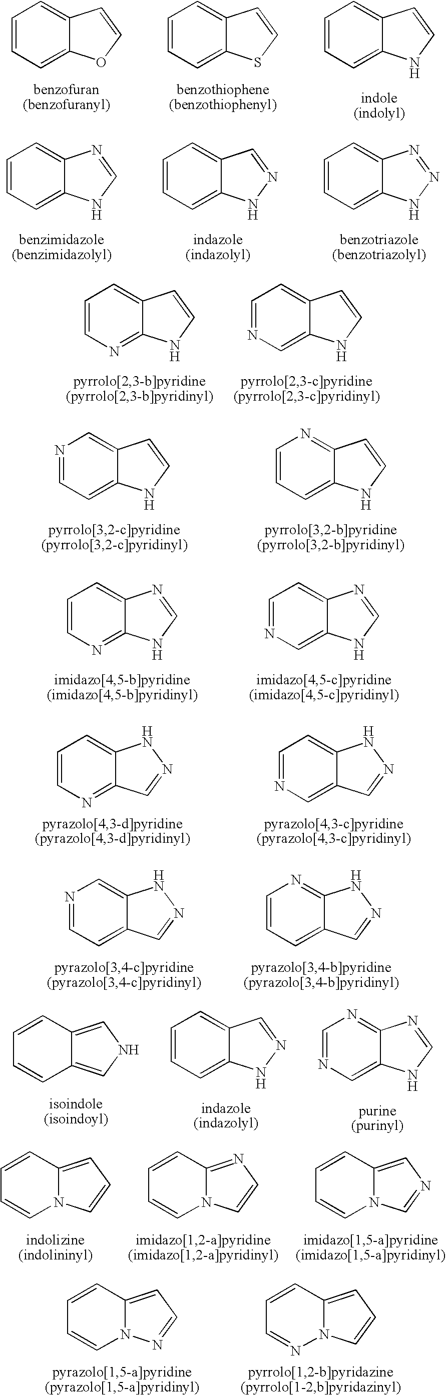

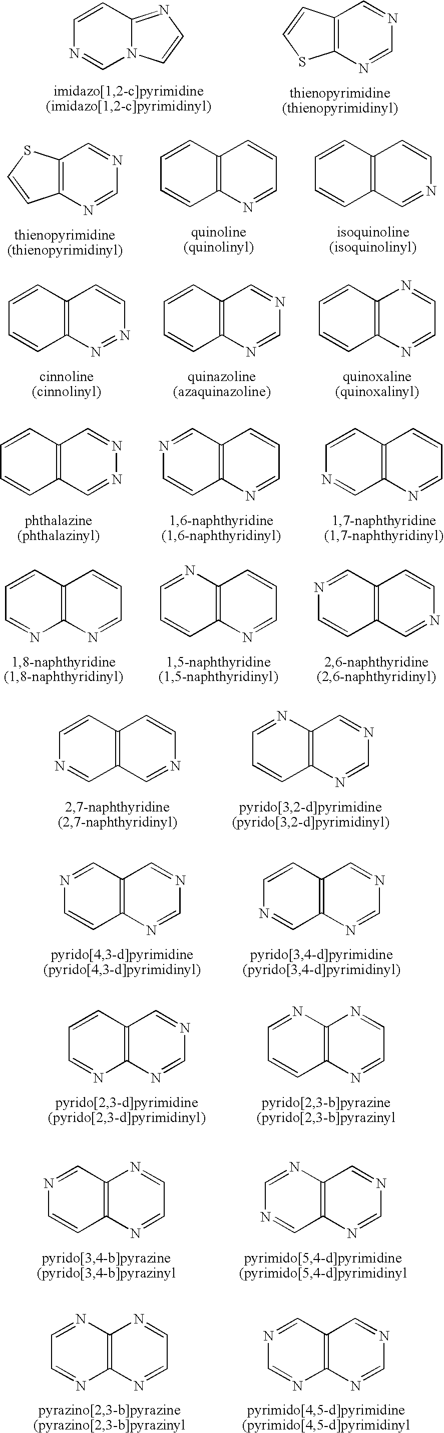

“Heteroalicyclic” or “heterocyclyl” refers to a monocyclic or polycyclic group having from 3 to 12 ring atoms, wherein from 1 to 4 ring atoms are heteroatoms selected from N, O, and S. “Heteroalicyclic” or “heterocyclyl” may also have one or more double bonds. However, “Heteroalicyclic” or “heterocyclyl” do not have a completely conjugated pi-electron system. “Heteroalicyclic” or “heterocyclyl” can be substituted or unsubstituted. Typical substituents include, but are not limited to, C₁-C₁₂ aliphatic, 6-10 member aryl, 6-10 member aryl, halogen, —NO₂, NH₂, NR₂, —CN, —COR, —COOR, —CONR₂, —OH, —OR, —OCOR, —SR, —SOR, —SO₂R, wherein R is a C₁-C₁₀ alkyl, 3-10 member heterocyclyl, C₆-C₁₀ aryl, 5-10 member heteroaryl.

Examples of saturated heterocyclyl groups include, but are not limited to:

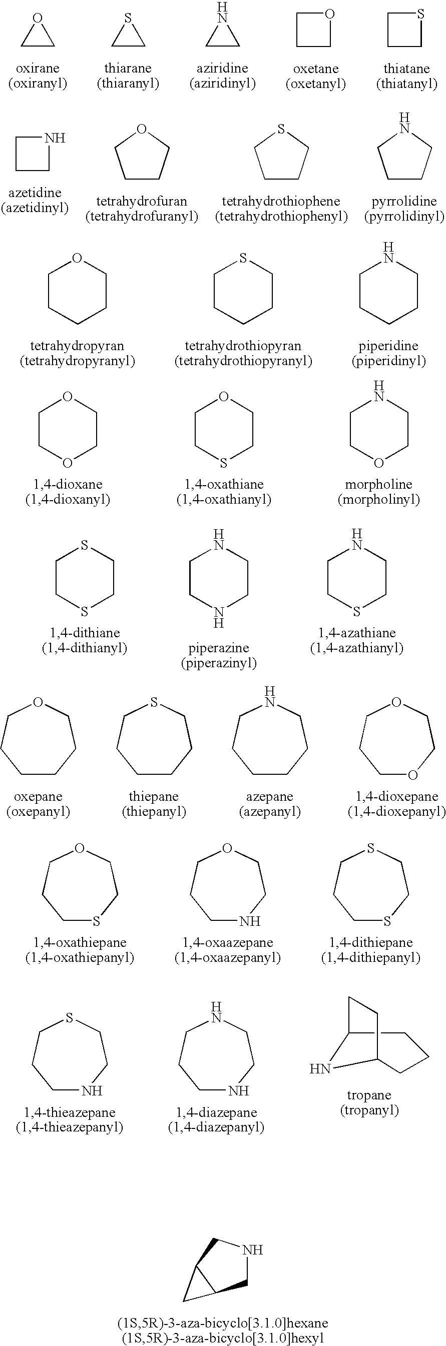

Examples of partially unsaturated heterocyclyl groups include, but are not limited to:

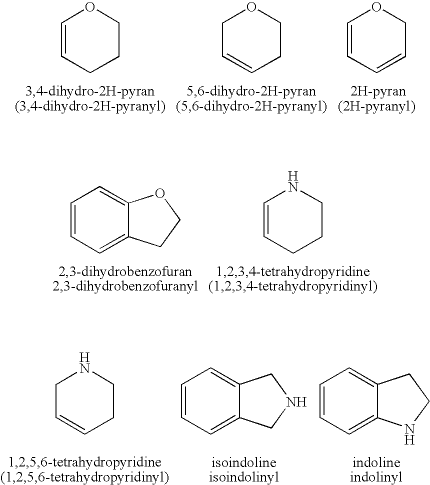

A “diradical” refers to a group that has two open valences and is further connected to two other groups, or forms a double bond with the same atom of one group, or forms two single bonds with the same atom of one group. Examples of diradicals are, but are not limited to —CH₂—, —O—, —O—CH₂—, —(C₁-C₃ alkylene)-NH— and —CH₂—CH₂—. When a diradical is referred to as, for example, —O—CH₂— or —(C₁-C₃ alkylene)-NH—, it is understood that each end of the diradical can equally connect to another moiety. For example, if K is defined as A-L-B, and L is a diradical selected from —O—CH₂— and —(C₁-C₃ alkylene)-, it is understood that K is therefore selected from A-O—CH₂—B, A-CH₂—O—B, and A-(C₁-C₃ alkylene)-B. A and B herein are referred to as different organic moieties.

When “ene” is added after the “yl” at the end of any of the previously defined terms to form a new term, the new term refers to a diradical formed by removing one hydrogen atom from the original term of which the new term derived from. For example, an alkylene refers to a diradical group formed by removing one hydrogen atom from an alkyl group and that a “methylene” refers to a divalent radical —CH₂— derived from removing one hydrogen atom from methyl. More examples of such diradicals include, but are not limited to: alkenylene, alkynylene, cycloalkylene, phenylene, heterocyclylene, heteroarylene and (nonaromatic unsaturated carbocyclylene), which are derived from alkenyl, alkynyl, cycloalkyl, phenyl, heterocyclyl, heteroaryl and (nonaromatic unsaturated carbocyclyl), respectively. For example, “cyclopropylene” refers to both

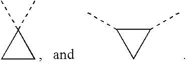

For example, “C₁-C₂ alkylene” refers to all of the following: —CH₂—, —CH(CH₃)— and —CH₂—CH₂—.

“oxo” or “-oxo-” refers to an oxygen double bond “═O” substitution.

“Hydroxy” or “hydroxyl” both refer to —OH.

“Perfluoroalkyl” refers to an alkyl group in which all of its hydrogen atoms are replaced by fluorine atoms.

“Optional” or “optionally” means that the subsequently described event or circumstance may but need not occur, and that the description includes instances where the event or circumstance occurs and instances in which it does not. For example, “heterocyclyl group optionally substituted with an alkyl group” means that the alkyl may but need not be present, and the description includes situations where the heterocyclyl group is substituted with an alkyl group and situations where the heterocyclyl group is not substituted with the alkyl group.

When a group is “optionally substituted” or “optionally further substituted” by some substituents, it means a carbon or a nitrogen atom of this group wherein one or more hydrogen atoms are attached to the carbon or nitrogen atom, such carbon or nitrogen atom is optionally substituted by some other substituents. For example, “R is H, C₁-C₃ alkyl or phenyl, and R is optionally further substituted by 1-3 groups selected from —F, oxo and C₁-C₃ perfluoroalkyl”, means that R is 1) H (when R is H, R cannot be further substituted); 2) C₁-C₃ alkyl optionally further substituted by 1-3 groups selected from —F, oxo and C₁-C₃ perfluoroalkyl; and 3) phenyl optionally further substituted by 1-3 groups selected from —F and C₁-C₃ perfluoroalkyl. Optional substitution of oxo does not apply when R is phenyl because no single atom of the phenyl group possess two hydrogen atoms to be substituted by oxo, i.e. ═O bond. When a group is further substituted by a “—(C₁-C₄ alkylene)-”, it means the “—(C₁-C₄ alkylene)-”, together with the nitrogen atom or the carbon atom of the group to which “C₁-C₄ alkylene” is attached to, form a carbo or hetero spiro cycle.

A “pharmaceutical composition” refers to a mixture of one or more of the compounds described herein, or physiologically/pharmaceutically acceptable salts, solvates, hydrates or prodrugs thereof, with other chemical components, such as physiologically/pharmaceutically acceptable carriers and excipients. The purpose of a pharmaceutical composition is to facilitate administration of a compound to an organism.

As used herein, a “physiologically/pharmaceutically acceptable carrier” refers to a carrier or diluent that does not cause significant irritation to an organism and does not abrogate the biological activity and properties of the administered compound.

A “pharmaceutically acceptable excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

As used herein, the term “pharmaceutically acceptable salt” refers to those salts that retain the biological effectiveness and properties of the parent compound. Such salts include:

(1) acid addition salts, which can be obtained by reaction of the free base of the parent compound with inorganic acids such as hydrochloric acid, hydrobromic acid, nitric acid, phosphoric acid, sulfuric acid, and perchloric acid and the like, or with organic acids such as acetic acid, oxalic acid, (D) or (L) malic acid, maleic acid, methanesulfonic acid, ethanesulfonic acid, p-toluenesulfonic acid, salicylic acid, tartaric acid, citric acid, succinic acid or malonic acid and the like; or

(2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

“PK” refers to receptor protein tyrosine kinase (RTKs), non-receptor or “cellular” tyrosine kinase (CTKs) and serine-threonine kinases (STKs).

“Modulation” or “modulating” refers to the alteration of the catalytic activity of RTKs, CTKs and STKs. In particular, modulating refers to the activation of the catalytic activity of RTKs, CTKs and STKs, preferably the activation or inhibition of the catalytic activity of RTKs, CTKs and STKs, depending on the concentration of the compound or salt to which the RTK, CTK or STK is exposed or, more preferably, the inhibition of the catalytic activity of RTKs, CTKs and STKs.

“Catalytic activity” refers to the rate of phosphorylation of tyrosine under the influence, direct or indirect, of RTKs and/or CTKs or the phosphorylation of serine and threonine under the influence, direct or indirect, of STKs.

“Contacting” refers to bringing a compound of the present teachings and a target PK together in such a manner that the compound can affect the catalytic activity of the PK, either directly, i.e., by interacting with the kinase itself, or indirectly, i.e., by interacting with another molecule on which the catalytic activity of the kinase is dependent. Such “contacting” can be accomplished “in vitro,” i.e., in a test tube, a petri dish or the like. In a test tube, contacting may involve only a compound and a PK of interest or it may involve whole cells. Cells may also be maintained or grown in cell culture dishes and contacted with a compound in that environment. In this context, the ability of a particular compound to affect a PK related disorder, i.e., the IC₅₀ of the compound, can be determined before use of compounds in vivo with more complex living organisms is attempted. For cells outside the organism, multiple methods exist, and are well-known to those skilled in the art, to get the PKs in contact with the compounds including, but not limited to, direct cell microinjection and numerous transmembrane carrier techniques.

“In vitro” refers to procedures performed in an artificial environment such as, e.g., without limitation, in a test tube or culture medium.

“In vivo” refers to procedures performed within a living organism such as, without limitation, a mouse, rat or rabbit.

“PK related disorder,” “PK driven disorder,” and “abnormal PK activity” all refer to a condition characterized by inappropriate, i.e., under or, more commonly, over, PK catalytic activity, where the particular PK can be an RTK, a CTK or an STK.

Inappropriate catalytic activity can arise as the result of either: (1) PK expression in cells which normally do not express PKs, (2) increased PK expression leading to unwanted cell proliferation, differentiation and/or growth, or, (3) decreased PK expression leading to unwanted reductions in cell proliferation, differentiation and/or growth. Over-activity of a PK refers to either amplification of the gene encoding a particular PK or production of a level of PK activity which can correlate with a cell proliferation, differentiation and/or growth disorder (that is, as the level of the PK increases, the severity of one or more of the symptoms of the cellular disorder increases). Under-activity is, of course, the converse, wherein the severity of one or more symptoms of a cellular disorder increase as the level of the PK activity decreases.

“Treat”, “treating” and “treatment” refer to a method of alleviating or abrogating a PK mediated cellular disorder and/or its attendant symptoms. With regard particularly to cancer, these terms simply mean that the life expectancy of an individual affected with a cancer will be increased or that one or more of the symptoms of the disease will be reduced.

“Organism” refers to any living entity comprised of at least one cell. A living organism can be as simple as, for example, a single eukariotic cell or as complex as a mammal, including a human being.

“Therapeutically effective amount” refers to that amount of the compound being administered which will relieve to some extent one or more of the symptoms of the disorder being treated. In reference to the treatment of cancer, a therapeutically effective amount refers to that amount which has at least one of the following effects:

(1) reducing the size of the tumor;

(2) inhibiting (that is, slowing to some extent, preferably stopping) tumor metastasis;

(3) inhibiting to some extent (that is, slowing to some extent, preferably stopping) tumor growth, and

(4) relieving to some extent (or, preferably, eliminating) one or more symptoms associated with the cancer.

“Monitoring” means observing or detecting the effect of contacting a compound with a cell expressing a particular PK. The observed or detected effect can be a change in cell phenotype, in the catalytic activity of a PK or a change in the interaction of a PK with a natural binding partner. Techniques for observing or detecting such effects are well-known in the art. The effect is selected from a change or an absence of change in a cell phenotype, a change or absence of change in the catalytic activity of said protein kinase or a change or absence of change in the interaction of said protein kinase with a natural binding partner in a final aspect of this invention.

“Cell phenotype” refers to the outward appearance of a cell or tissue or the biological function of the cell or tissue. Examples, without limitation, of a cell phenotype are cell size, cell growth, cell proliferation, cell differentiation, cell survival, apoptosis, and nutrient uptake and use. Such phenotypic characteristics are measurable by techniques well-known in the art.

“Natural binding partner” refers to a polypeptide that binds to a particular PK in a cell. Natural binding partners can play a role in propagating a signal in a PK-mediated signal transduction process. A change in the interaction of the natural binding partner with the PK can manifest itself as an increased or decreased concentration of the PK/natural binding partner complex and, as a result, in an observable change in the ability of the PK to mediate signal transduction.

The term “stereoisomers” refers to compounds that have identical chemical constitution, but differ with regard to the arrangement of their atoms or groups in space. In particular, the term “enantiomers” refers to two stereoisomers of a compound that are non-superimposable mirror images of one another. The terms “racemic” or “racemic mixture,” as used herein, refer to a 1:1 mixture of enantiomers of a particular compound. The term “diastereomers”, on the other hand, refers to the relationship between a pair of stereoisomers that comprise two or more asymmetric centers and are not mirror images of one another.

### DETAILED DESCRIPTION

The compounds of the current invention, i.e., the compounds of formula I, as well as compounds of formula II, III and IV, can be made following reaction Scheme 1, 2 and 3.

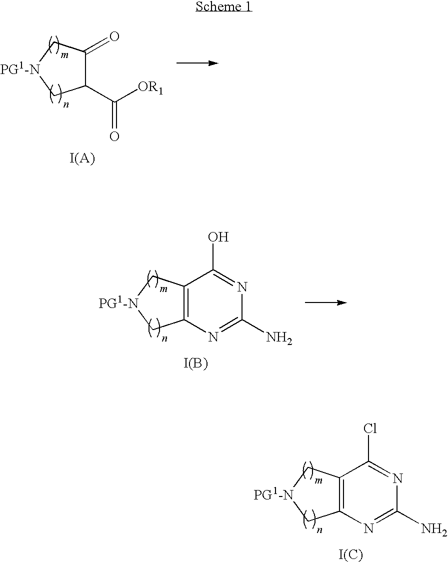

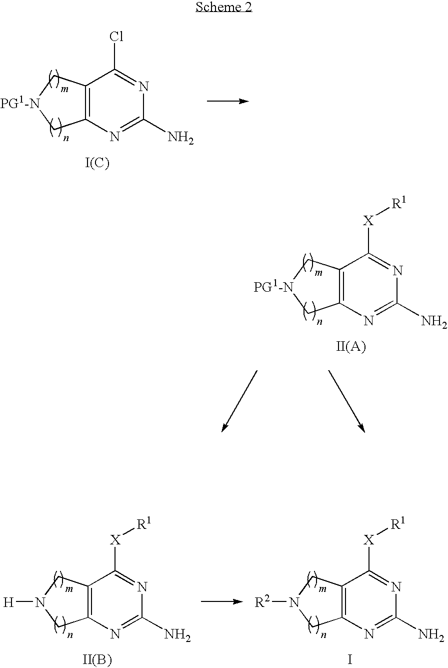

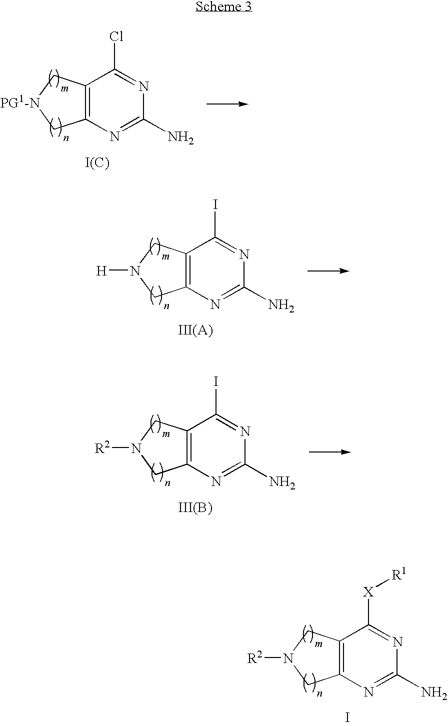

Scheme 1 illustrates the synthesis of intermediate I(C) used to make compounds of formula I. The beta keto ester I(A) can be prepared based on a known procedures (see, e.g. Viscontini and Buhler Helvetica Chimica Acta, 50(5): 1289-93; (1967), Rosowsky et. al. J. Heterocyclic Chem., 26: 509-16 (1989)). PG¹, the nitrogen protecting group, can be selected for compatibility with subsequent chemistry. Protecting groups and general considerations for their use are described in T. Greene and P. Wuts, “Protective Groups in Organic Synthesis”, 3^{rd} Edition 1999, John Wiley & Sons and are well known to those skilled in the art. Compound I(A) is condensed with guanidine to give compound I(B). This can typically be done by heating compound I(A) with guanidine or guanidine equivalent a protic solvent. A typical reaction condition would be to reflux compound I(A) with guanidine carbonate in tent-butanol as a solvent. Conversion of the hydroxyl group of compound I(B) to chloro gives I(C). This can typically be done by heating compound I(B) with POCl₃ in an aprotic solvent. A typical reaction condition would be to reflux compound I(B) together with excess POCl₃ either neat or in dry acetonitrile as solvent.

Scheme 2 illustrates the route through which compounds of formula I can be made from intermediate I(C). In Scheme 2, the chloro leaving group of compound I(C) is replaced by a R¹—X— group to give compound II(A). This reaction can typically be carried out by displacement of the chloro leaving group using a variety of nucleophiles such as carbon, oxygen, nitrogen, sulfur, or other nucleophiles. A typical nucleophilic displacement reaction for the transformation of compound I(C) to compound II(A) would be to treat compound I(C) with a nucleophile in the presence of base such as cesium carbonate using DMF or DMSO as solvent to give compound II(A). Alternatively, the displacement of the chloro leaving group of compound I(C) by R¹—X— to give compound II(A) can also be carried out using cross coupling methodology utilizing Suzuki, Stille, Negishi or similar conditions. A typical cross coupling reaction for the transformation of compound I(C) to compound II(A) would be to treat compound I(C) with a boronic acid or ester in the presence of a base such as sodium carbonate and Pd(0) catalyst in a solvent mixture such as water and 1,4-dioxane to give compound II(A). The nitrogen protecting group, PG¹, of compound II(A) is then removed to give compound II(B). This can typically be done, when PG¹ forms an ethyl carbamate protecting group, by refluxing compound II(A) with trimethylsilyliodide in a solvent such as CH₃CN. Alternatively HBr in acetic acid or KOH in isopropanol can also be used. A typical condition for the transformation of compound II(A) to compound II(B) would be to treat compound II(A) with TMSI (5 equivalents) in refluxing CH₃CN to give compound II(B). The dihydropyrrolo amino moiety of compound II(B) then acts as a nucleophile in reactions with an electrophilic R² moiety to give compound I. This nucleophilic reaction can be alkylation, acylation, sulfonylation, and other reactions applicable to secondary alkyl amines. An alkylation reaction of compound II(B) can be carried out by reacting compound II(B) with an alkylating R² moiety. A typical alkylation reaction condition is to react compound II(B) with an R² alkyl bromide moiety in the presence of TEA at room temperature to give compound I as an N-alkyl. An acylation reaction of compound II(B) can be carried out by reacting compound II(B) with an acylating R² moiety. A typical acylation reaction condition is to react compound II(B) with an R² activated ester moiety or R² acyl halide in the presence of TEA to give compound I as an amide. Another typical acylation reaction condition is to react compound II(B) with an R² isocyanate or isocyanate equivalent moiety in the presence of TEA to give compound I as a urea. A sulfonylation reaction of compound II(B) can be carried out by reacting compound II(B) with an sulfonylating R² moiety. A typical sulfonylation reaction condition is to react compound II(B) with an R² sulfonyl chloride moiety in the presence of TEA to give compound I as a sulfonamide.

Another method for the preparation of compound I as a urea takes compound II(A), when PG¹ forms an ethyl carbamate protecting group, and directly converts compound II(A) into compound I. This reaction can typically be carried out using a nucleophilic amine in the presence of trimethylaluminum in a solvent such as toluene. A typical condition is to treat a nucleophilic amine in toluene at 0° C. with a solution of trimethylaluminum in hexanes. After warming to room temperature, compound II(A) is added and the mixture is heated under microwave radiation to prepare compound I.

Scheme 3 illustrates another route through which compounds of formula I can be made from intermediate I(C). In Scheme 3 when PG¹ forms an ethyl carbamate protecting group, the protecting group, PG¹, of compound I(C) is removed with concurrent conversion of chloro to iodo in one step. This can typically be carried out by heating compound I(C) with TMSI in an aprotic solvent. A typical reaction condition is to reflux compound I(C) in CH₃CN with five equivalents of TMSI. Following a methanol quench, compound III(A) is obtained as an HI salt. The dihydropyrrolo amino moiety of compound III(A) then acts as a nucleophile in reactions with an electrophilic R² moiety to give compound III(B). This nucleophilic reaction can be alkylation, acylation, sulfonylation, and other reactions applicable to secondary alkyl amines. An alkylation reaction of compound III(A) can be carried out by reacting compound III(A) with an alkylating R² moiety. A typical alkylation reaction condition is to react compound III(A) with an R² alkyl bromide moiety in the presence of TEA at room temperature to give compound III(B) as an N-alkyl. An acylation reaction of compound III(A) can be carried out by reacting compound III(A) with an acylating R² moiety. A typical acylation reaction condition is to react compound III(A) with an R² activated ester moiety or R² acyl halide in the presence of TEA to give compound III(B) as an amide. Another typical acylation reaction condition is to react compound III(A) with an R² isocyanate or isocyanate equivalent moiety in the presence of TEA to give compound III(B) as a urea. A sulfonylation reaction of compound III(A) can be carried out by reacting compound III(A) with an sulfonylating R² moiety. A typical sulfonylation reaction condition is to react III(A) with an R² sulfonyl chloride moiety in the presence of TEA at 0° C. to give compound III(B) as a sulfonamide. The iodo group of compound III(B) is then displaced by R¹—X— using cross coupling methodology to give compound I. This reaction can typically be carried out using Suzuki, Stille, Negishi or similar conditions. A typical cross coupling reaction for the transformation of compound III(B) to compound I would be to treat compound III(B) with a boronic acid or ester in the presence of a base such as sodium carbonate and Pd(0) catalyst in a solvent mixture such as water and 1,4-dioxane to give compound I.

The compounds of the present invention may have asymmetric carbon atoms. The carbon-carbon bonds of the compounds of the present invention may be depicted herein using a solid line (——) a solid wedge ( [symbol] ) or a dotted wedge ( [symbol] ). The use of a solid line to depict bonds to asymmetric carbon atoms is meant to indicate that all possible stereoisomers (e.g. specific enantiomers, racemic mixtures, etc.) at that carbon atom are included. The use of either a solid or dotted wedge to depict bonds to asymmetric carbon atoms is meant to indicate that only the stereoisomer shown is meant to be included. It is possible that compounds of the invention may contain more than one asymmetric carbon atom. In those compounds, the use of a solid line to depict bonds to asymmetric carbon atoms is meant to indicate that all possible stereoisomers are meant to be included. For example, unless stated otherwise, it is intended that the compounds of the present invention can exist as enantiomers and diastereomers or as racemates and mixtures thereof. The use of a solid line to depict bonds to one or more asymmetric carbon atoms in a compound of the invention and the use of a solid or dotted wedge to depict bonds to other asymmetric carbon atoms in the same compound is meant to indicate that a mixture of diastereomers is present.

Conventional techniques for the preparation/isolation of individual enantiomers include chiral synthesis from a suitable optically pure precursor or resolution of the racemate using, for example, chiral high pressure liquid chromatography (HPLC). Alternatively, the racemate (or a racemic precursor) may be reacted with a suitable optically active compound, for example, an alcohol, or, in the case where the compound contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to one skilled in the art. Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture. Stereoisomeric conglomerates may be separated by conventional techniques known to those skilled in the art. See, e.g. “Stereochemistry of Organic Compounds” by E. L. Eliel (Wiley, New York, 1994), the disclosure of which is incorporated herein by reference in its entirety.

Where a compound of the invention contains an alkenyl or alkenylene group, geometric cis/trans (or Z/E) isomers are possible. Cis/trans isomers may be separated by conventional techniques well known to those skilled in the art, for example, chromatography and fractional crystallization. Where structural isomers are interconvertible via a low energy barrier, tautomeric isomerism (‘tautomerism’) can occur. This can take the form of proton tautomerism in compounds of the present invention containing, for example, an imino, keto, or oxime group, or so-called valence tautomerism in compounds which contain an aromatic moiety. It follows that a single compound may exhibit more than one type of isomerism. Included within the scope of the invention are all stereoisomers, geometric isomers and tautomeric forms of the inventive compounds, including compounds exhibiting more than one type of isomerism, and mixtures of one or more thereof.

Salts of the present invention can be prepared according to methods known to those of skill in the art. Examples of salts include, but are not limited to, acetate, acrylate, benzenesulfonate, benzoate (such as chlorobenzoate, methylbenzoate, dinitrobenzoate, hydroxybenzoate, and methoxybenzoate), bicarbonate, bisulfate, bisulfite, bitartrate, borate, bromide, butyne-1,4-dioate, calcium edetate, camsylate, carbonate, chloride, caproate, caprylate, clavulanate, citrate, decanoate, dihydrochloride, dihydrogenphosphate, edetate, edislyate, estolate, esylate, ethylsuccinate, formate, fumarate, gluceptate, gluconate, glutamate, glycollate, glycollylarsanilate, heptanoate, hexyne-1,6-dioate, hexylresorcinate, hydrabamine, hydrobromide, hydrochloride, γ-hydroxybutyrate, iodide, isobutyrate, isothionate, lactate, lactobionate, laurate, malate, maleate, malonate, mandelate, mesylate, metaphosphate, methane-sulfonate, methylsulfate, monohydrogenphosphate, mucate, napsylate, naphthalene-1-sulfonate, naphthalene-2-sulfonate, nitrate, oleate, oxalate, pamoate (embonate), palmitate, pantothenate, phenylacetates, phenylbutyrate, phenylpropionate, phthalate, phosphate/diphosphate, polygalacturonate, propanesulfonate, propionate, propiolate, pyrophosphate, pyrosulfate, salicylate, stearate, subacetate, suberate, succinate, sulfate, sulfonate, sulfite, tannate, tartrate, teoclate, tosylate, triethiodode, and valerate salts.

The compounds of the present invention that are basic in nature are capable of forming a wide variety of salts with various inorganic and organic acids. Although such salts must be pharmaceutically acceptable for administration to animals, it is often desirable in practice to initially isolate the compound of the present invention from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention can be prepared by treating the base compound with a substantially equivalent amount of the selected mineral or organic acid in an aqueous solvent medium or in a suitable organic solvent, such as methanol or ethanol. Upon evaporation of the solvent, the desired solid salt is obtained. The desired acid salt can also be precipitated from a solution of the free base in an organic solvent by adding an appropriate mineral or organic acid to the solution.

Those compounds of the present invention that are acidic in nature are capable of forming base salts with various pharmacologically acceptable cations. Examples of such salts include the alkali metal or alkaline-earth metal salts and particularly, the sodium and potassium salts. These salts are all prepared by conventional techniques. The chemical bases which are used as reagents to prepare the pharmaceutically acceptable base salts of this invention are those which form non-toxic base salts with the acidic compounds of the present invention. Such non-toxic base salts include those derived from such pharmacologically acceptable cations as sodium, potassium calcium and magnesium, etc. These salts may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium. These salts can also be prepared by treating the corresponding acidic compounds with an aqueous solution containing the desired pharmacologically acceptable cations, and then evaporating the resulting solution to dryness; preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum yields of the desired final product.

If the inventive compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

In the case of compounds that are solids, it is understood by those skilled in the art that the inventive compounds and salts may exist in different crystalline or polymorphic forms, or in an amorphous form, all of which are intended to be within the scope of the present invention.

The invention also includes isotopically-labeled compounds of the invention, wherein one or more atoms is replaced by an atom having the same atomic number, but an atomic mass or mass number different from the atomic mass or mass number usually found in nature. Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S. Certain isotopically-labeled compounds of the invention, for example, those incorporating a radioactive isotope, are useful in drug and/or substrate tissue distribution studies. The radioactive isotopes tritium, ³H, and carbon-14, ¹⁴C, are particularly useful for this purpose in view of their ease of incorporation and ready means of detection. Substitution with heavier isotopes such as deuterium, ²H, may afford certain therapeutic advantages resulting from greater metabolic stability, for example, increased in vivo half-life or reduced dosage requirements, and hence may be preferred in some circumstances. Substitution with positron emitting isotopes, such as ¹¹C, ¹⁸F, ¹⁵O and ¹³N, can be useful in Positron Emission. Topography (PET) studies for examining substrate receptor occupancy.

Isotopically-labeled compounds of the invention can generally be prepared by conventional techniques known to those skilled in the art or by processes analogous to those described herein, using an appropriate isotopically-labeled reagent in place of the non-labeled reagent otherwise employed.

The compounds of the invention may exist in both unsolvated and solvated forms. The term ‘solvate’ is used herein to describe a molecular complex comprising a compound of the invention and an amount of one or more pharmaceutically acceptable solvent molecules. The term ‘hydrate’ is employed when said solvent is water. Examples of solvate forms include, but are not limited to, compounds of the invention in association with water, isopropanol, ethanol, methanol, dimethylsulfoxide (DMSO), ethyl acetate, acetic acid, ethanolamine, or mixtures thereof. It is specifically contemplated that in the present invention one solvent molecule can be associated with one molecule of the compounds of the present invention, such as a hydrate.

Furthermore, it is specifically contemplated that in the present invention, more than one solvent molecule may be associated with one molecule of the compounds of the present invention, such as a dihydrate. Additionally, it is specifically contemplated that in the present invention less than one solvent molecule may be associated with one molecule of the compounds of the present invention, such as a hemihydrate. Furthermore, solvates of the present invention are contemplated as solvates of compounds of the present invention that retain the biological effectiveness of the non-hydrate form of the compounds.

Prodrugs of the compounds described herein are also within the scope of the invention. Thus certain derivatives of the compounds of the present invention, which derivatives may have little or no pharmacological activity themselves, when administered into or onto the body may be converted into compounds of the present invention having the desired activity, for example, by hydrolytic cleavage. Such derivatives are referred to as ‘prodrugs’. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (ed. E. B. Roche, American Pharmaceutical Association).

Prodrugs in accordance with the invention can, for example, be produced by replacing appropriate functionalities present in the compounds of the present invention with certain moieties known to those skilled in the art as ‘pro-moieties’ as described, for example, in Design of Prodrugs by H. Bundgaard (Elsevier, 1985).

Some examples of prodrugs in accordance with the invention include:

(i) where the compounds of the present invention contain a carboxylic acid functionality (—COOH), a prodrug compound wherein the hydrogen of the carboxylic acid functionality of the compound is replaced by (C₁-C₈)alkyl to form the corresponding ester;

(ii) where the compounds of the present invention contain an alcohol functionality (—OH), a prodrug compound wherein the hydrogen of the alcohol functionality of the compound is replaced by (C₁-C₆) alkanoyloxymethyl to form the corresponding ether; and

(iii) where the compounds of the present invention contain a primary or secondary amino functionality (—NH₂ or —NHR where R≠H), a prodrug compound wherein, as the case may be, one or both hydrogens of the amino functionality of the compound I is/are replaced by (C₁-C₁₀) alkanoyl to form the corresponding amide.

Further examples of replacement groups in accordance with the foregoing examples and examples of other prodrug types may be found in the aforementioned references. Moreover, certain compounds of the present invention may themselves act as prodrugs of other compounds of the present invention.

Also included within the scope of the invention are metabolites of compounds of the present invention, that is, compounds formed in vivo upon administration of the drug.

Some examples of metabolites in accordance with the invention include:

(i) where the compounds of the present invention contain a methyl group, a hydroxymethyl derivative thereof (e.g. —CH₃->—CH₂OH);

(ii) where the compounds of the present invention contain an alkoxy group, a hydroxy derivative thereof (e.g. —OR->—OH);

(iii) where the compounds of the present invention contain a tertiary amino group, a secondary amino derivative thereof (e.g. —NR¹R²->—NHR¹ or —NHR²);

(iv) where the compounds of the present invention contain a secondary amino group, a primary derivative thereof (e.g. —NHR¹->—NH₂);

(v) where the compounds of the present invention contain a phenyl moiety, a phenol derivative thereof (e.g. -Ph->-PhOH); and

(vi) where the compounds of the present invention contain an amide group, a carboxylic acid derivative thereof (e.g. —CONH₂->COOH).

Compounds of the invention intended for pharmaceutical use may be administered as crystalline or amorphous products, or mixtures thereof. They may be obtained, for example, as solid plugs, powders, or films by methods such as precipitation, crystallization, freeze drying, spray drying, or evaporative drying. Microwave or radio frequency drying may be used for this purpose.

The compounds can be administered alone or in combination with one or more other compounds of the invention, or in combination with one or more other drugs (or as any combination thereof). Generally, they will be administered as a formulation in association with one or more pharmaceutically acceptable excipients. The term “excipient” is used herein to describe any ingredient other than the compound(s) of the invention. The choice of excipient will to a large extent depend on factors such as the particular mode of administration, the effect of the excipient on solubility and stability, and the nature of the dosage form.

Pharmaceutical compositions suitable for the delivery of compounds of the invention and methods for their preparation will be readily apparent to those skilled in the art. Such compositions and methods for their preparation can be found, for example, in. ‘Remington's Pharmaceutical Sciences’, 19th Edition (Mack Publishing Company, 1995), the disclosure of which is incorporated herein by reference in its entirety.

### Oral Administration

The compounds of the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films (including muco-adhesive), ovules, sprays and liquid formulations.

Liquid formulations include suspensions, solutions, syrups and elixirs. Such formulations may be used as fillers in soft or hard capsules and typically include a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986 by Liang and Chen (2001), the disclosure of which is incorporated herein by reference in its entirety.

For tablet dosage forms, depending on dose, the drug may make up from 1 wt % to 80 wt % of the dosage form, more typically from 5 wt % to 60 wt % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinized starch and sodium alginate. Generally, the disintegrant will comprise from 1 wt % to 25 wt %, preferably from 5 wt % to 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally include surface active agents, such as sodium lauryl sulfate and polysorbate 80, and glidants such as silicon dioxide and talc. When present, surface active agents are typically in amounts of from 0.2 wt % to 5 wt % of the tablet, and glidants typically from 0.2 wt % to 1 wt % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally are present in amounts from 0.25 wt % to 10 wt %, preferably from 0.5 wt % to 3 wt % of the tablet.

Other conventional ingredients include anti-oxidants, colorants, flavoring agents, preservatives and taste-masking agents.

Exemplary tablets contain up to about 80 wt % drug, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet blends may be compressed directly or by roller to form tablets. Tablet blends or portions of blends may alternatively be wet-, dry-, or melt-granulated, melt congealed, or extruded before tabletting. The final formulation may include one or more layers and may be coated or uncoated; or encapsulated.

The formulation of tablets is discussed in detail in “Pharmaceutical Dosage Forms: Tablets, Vol. 1”, by H. Lieberman and L. Lachman, Marcel Dekker, N.Y., N.Y., 1980 (ISBN 0-8247-6918-X), the disclosure of which is incorporated herein by reference in its entirety.

Solid formulations for oral administration may be formulated to be immediate and/or modified release. Modified release formulations include delayed-, sustained-, pulsed-, controlled-, targeted and programmed release.

Suitable modified release formulations are described in U.S. Pat. No. 6,106,864. Details of other suitable release technologies such as high energy dispersions and osmotic and coated particles can be found in Verma et al., Pharmaceutical Technology On-line, 25(2), 1-14 (2001). The use of chewing gum to achieve controlled release is described in WO 00/35298. The disclosures of these references are incorporated herein by reference in their entireties.

### Parenteral Administration

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilization, may readily be accomplished using standard pharmaceutical techniques well known to those skilled in the art.

The solubility of compounds of the invention used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus compounds of the invention may be formulated as a solid, semi-solid, or thixotropic liquid for administration as an implanted depot providing modified release of the active compound. Examples of such formulations include drug-coated stents and PGLA microspheres.

### Topical Administration

The compounds of the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated; see, for example, J Pharm Sci, 88 (10), 955-958 by Finnin and Morgan (October 1999). Other means of topical administration include delivery by electroporation, iontophoresis, phonophoresis, sonophoresis and microneedle or needle-free (e.g. Powderject™, Bioject™, etc.) injection. The disclosures of these references are incorporated herein by reference in their entireties.

Formulations for topical administration may be formulated to be immediate and/or modified release. Modified release formulations include delayed-, sustained-, pulsed-, controlled-, targeted and programmed release.

### Inhaled/Intranasal Administration

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may include a bioadhesive agent, for example, chitosan or cyclodextrin.

The pressurized container, pump, spray, atomizer, or nebulizer contains a solution or suspension of the compound(s) of the invention comprising, for example, ethanol, aqueous ethanol, or a suitable alternative agent for dispersing, solubilizing, or extending release of the active, a propellant(s) as solvent and an optional surfactant, such as sorbitan trioleate, oleic acid, or an oligolactic acid.

Prior to use in a dry powder or suspension formulation, the drug product is micronized to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or HPMC), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomizer using electrohydrodynamics to produce a fine mist may contain from tug to 20 mg of the compound of the invention per actuation and the actuation volume may vary from 1 μL to 100 μL. A typical formulation includes a compound of the invention, propylene glycol, sterile water, ethanol and sodium chloride. Alternative solvents which may be used instead of propylene glycol include glycerol and polyethylene glycol.

Suitable flavors, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, poly(DL-lactic-coglycolic acid (PGLA). Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” containing a desired mount of the compound of the invention. The overall daily dose may be administered in a single dose or, more usually, as divided doses throughout the day.

### Rectal/Intravaginal Administration

Compounds of the invention may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

### Ocular Administration

Compounds of the invention may also be administered directly to the eye or ear, typically in the form of drops of a micronized suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted, or programmed release.

### Other Technologies

Compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubilizer. Most commonly used for these purposes are alpha-, beta- and gamma-cyclodextrins, examples of which may be found in PCT Publication Nos. WO 91/11172, WO 94/02518 and WO 98/55148, the disclosures of which are incorporated herein by reference in their entireties.

The amount of the active compound administered will be dependent on the subject being treated, the severity of the disorder or condition, the rate of administration, the disposition of the compound and the discretion of the prescribing physician. However, an effective dosage is typically in the range of about 0.001 to about 100 mg per kg body weight per day, preferably about 0.01 to about 35 mg/kg/day, in single or divided doses. For a 70 kg human, this would amount to about 0.07 to about 7000 mg/day, preferably about 0.7 to about 2500 mg/day. In some instances, dosage levels below the lower limit of the aforesaid range may be more than adequate, while in other cases still larger doses may be used without causing any harmful side effect, with such larger doses typically divided into several smaller doses for administration throughout the day.

This invention also relates to a method for the treatment of abnormal cell growth in a mammal which comprises administering to said mammal an amount of a compound of the present invention, or a salt or solvate thereof, that is effective in treating abnormal cell growth in combination with an anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, antibodies, cytotoxics, anti-hormones, and anti-androgens.

In one embodiment of the present invention the anti-tumor agent used in conjunction with a compound of the present invention and pharmaceutical compositions described herein is an anti-angiogenesis agent, kinase inhibitor, pan kinase inhibitor or growth factor inhibitor. Preferred pan kinase inhibitors include Sutent™ (sunitinib), described in U.S. Pat. No. 6,573,293 (Pfizer, Inc, NY, USA). Anti-angiogenesis agents, include but are not limited to the following agents, such as EGF inhibitors, EGFR inhibitors, VEGF inhibitors, VEGFR inhibitors, TIE2 inhibitors, IGF1R inhibitors, COX-II (cyclooxygenase II) inhibitors, MMP-2 (matrix-metalloprotienase 2) inhibitors, and MMP-9 (matrix-metalloprotienase 9) inhibitors.

Preferred VEGF inhibitors, include for example, Avastin (bevacizumab), an anti-VEGF monoclonal antibody of Genentech, Inc. of South San Francisco, Calif. Additional VEGF inhibitors include CP-547,632 (Pfizer Inc., NY, USA), AG13736 (Pfizer Inc.), ZD-6474 (AstraZeneca), AEE788 (Novartis), AZD-2171, VEGF Trap (Regeneron/Aventis), Vatalanib (also known as PTK-787, ZK-222584: Novartis & Schering AG), Macugen (pegaptanib octasodium, NX-1838, EYE-001, Pfizer Inc./Gilead/Eyetech), IM862 (Cytran Inc. of Kirkland, Wash., USA); and angiozyme, a synthetic ribozyme from Ribozyme (Boulder, Colo.) and Chiron (Emeryville, Calif.) and combinations thereof.

VEGF inhibitors useful in the practice of the present invention are described in U.S. Pat. Nos. 6,534,524 and 6,235,764, both of which are incorporated in their entirety for all purposes. Additional VEGF inhibitors are described in, for example in WO 99/24440, in WO 95/21613, WO 99/61422, U.S. Pat. No. 5,834,504, WO 98/50356, U.S. Pat. No. 5,883,113 U.S. Pat. No. 5,886,020, U.S. Pat. No. 5,792,783, U.S. Pat. No. 6,653,308, WO 99/10349, WO 97/32856, WO 97/22596, WO 98/54093, WO 98/02438, WO 99/16755, and WO 98/02437, all of which are herein incorporated by reference in their entirety.

Other anti-angiogenic compounds include acitretin, fenretinide, thalidomide, zoledronic acid, angiostatin, aplidine, cilengtide, combretastatin A-4, endostatin, halofuginone, rebimastat, removab, Revlimid, squalamine, ukrain, Vitaxin and combinations thereof.

Other antiproliferative agents that may be used in combination with the compounds of the present invention include inhibitors of the enzyme farnesyl protein transferase and inhibitors of the receptor tyrosine kinase PDGFr, including the compounds disclosed and claimed in the following: U.S. Pat. No. 6,080,769; U.S. Pat. No. 6,194,438; U.S. Pat. No. 6,258,824; U.S. Pat. No. 6,586,447; U.S. Pat. No. 6,071,935; U.S. Pat. No. 6,495,564; and U.S. Pat. No. 6,150,377; U.S. Pat. No. 6,596,735; U.S. Pat. No. 6,479,513; WO 01/40217; U.S. 2003-0166675. Each of the foregoing patents and patent applications is herein incorporated by reference in their entirety.

PDGRr inhibitors include but are not limited to those disclosed in international patent application publication numbers WO01/40217 and WO2004/020431, the contents of which are incorporated in their entirety for all purposes. Preferred PDGFr inhibitors include Pfizer's CP-673,451 and CP-868,596 and its salts.

Preferred GARF inhibitors include Pfizer's AG-2037 (pelitrexol and its salts). GARF inhibitors useful in the practice of the present invention are disclosed in U.S. Pat. No. 5,608,082 which is incorporated in its entirety for all purposes.

Examples of useful COX-II inhibitors which can be used in conjunction with a compound of Formula (I) and pharmaceutical compositions disclosed herein include CELEBREX™ (celecoxib), parecoxib, deracoxib, ABT-963, MK-663 (etoricoxib), COX-189 (Lumiracoxib), BMS 347070, RS 57067, NS-398, Bextra (valdecoxib), paracoxib, Vioxx (rofecoxib), SD-8381, 4-Methyl-2-(3,4-dimethylphenyl)-1-(4-sulfamoyl-phenyl)-1H-pyrrole, 2-(4-Ethoxyphenyl)-4-methyl-1-(4-sulfamoylphenyl)-1H-pyrrole, T-614, JTE-522, S-2474, SVT-2016, CT-3, SC-58125 and Arcoxia (etoricoxib). Additionally, COX-II inhibitors are disclosed in U.S. Patent Applications US 2005-0148627 and US 2005-0148777, the contents of which are incorporated in their entirety for all purposes.

In a particular embodiment the anti-tumor agent is celecoxib (U.S. Pat. No. 5,466,823), valdecoxib (U.S. Pat. No. 5,633,272), parecoxib (U.S. Pat. No. 5,932,598), deracoxib (U.S. Pat. No. 5,521,207), SD-8381 (U.S. Pat. No. 6,034,256, Example 175), ABT-963 (WO 2002/24719), rofecoxib (CAS No. 162011-90-7), MK-663 (or etoricoxib) as disclosed in WO 1998/03484, COX-189 (Lumiracoxib) as disclosed in WO 1999/11605, BMS-347070 (U.S. Pat. No. 6,180,651), NS-398 (CAS 123653-11-2), RS 57067 (CAS 17932-91-3), 4-Methyl-2-(3,4-dimethylphenyl)-1-(4-sulfamoyl-phenyl)-1H-pyrrole, 2-(4-Ethoxyphenyl)-4-methyl-1-(4-sulfamoylphenyl)-1H-pyrrole, or meloxicam.

Other useful inhibitors as anti-tumor agents used in combination with a compound of the present invention and pharmaceutical compositions disclosed herein include aspirin, and non-steroidal anti-inflammatory drugs (NSAIDs) which inhibit the enzyme that makes prostaglandins (cyclooxygenase I and II), resulting in lower levels of prostaglandins, include but are not limited to the following, Salsalate (Amigesic), Diflunisal (Dolobid), Ibuprofen (Motrin), Ketoprofen (Orudis), Nabumetone (Relafen), Piroxicam (Feldene), Naproxen (Aleve, Naprosyn), Diclofenac (Voltaren), Indomethacin (Indocin), Sulindac (Clinoril), Tolmetin (Tolectin), Etodolac (Lodine), Ketorolac (Toradol), Oxaprozin (Daypro) and combinations thereof.

Preferred COX-I inhibitors include ibuprofen (Motrin), nuprin, naproxen (Aleve), indomethacin (Indocin), nabumetone (Relafen) and combinations thereof.

Targeted agents used in combination with a compound of the present invention and pharmaceutical compositions disclosed herein include EGFr inhibitors such as Iressa (gefitinib, AstraZeneca), Tarceva (erlotinib or OSI-774, OSI Pharmaceuticals Inc.), Erbitux (cetuximab, Imclone Pharmaceuticals, Inc.), EMD-7200 (Merck AG), ABX-EGF (Amgen Inc. and Abgenix Inc.), HR3 (Cuban Government), IgA antibodies (University of Erlangen-Nuremberg), TP-38 (IVAX), EGFR fusion protein, EGF-vaccine, anti-EGFr immunoliposomes (Hermes Biosciences Inc.) and combinations thereof. Preferred EGFr inhibitors include Iressa, Erbitux, Tarceva and combinations thereof.

Other anti-tumor agents include those selected from pan erb receptor inhibitors or ErbB2 receptor inhibitors, such as CP-724,714 (Pfizer, Inc.), CI-1033 (canertinib, Pfizer, Inc.), Herceptin (trastuzumab, Genentech Inc.), Omitarg (2C4, pertuzumab, Genentech Inc.), TAK-165 (Takeda), GW-572016 (Ionafarnib, GlaxoSmithKline), GW-282974 (GlaxoSmithKline), EKB-569 (Wyeth), PKI-166 (Novartis), dHER2 (HER2 Vaccine, Corixa and GlaxoSmithKline), APC8024 (HER2 Vaccine, Dendreon), anti-HER2/neu bispecific antibody (Decof Cancer Center), B7.her2.IgG3 (Agensys), AS HER2 (Research Institute for Rad Biology & Medicine), trifunctional bispecific antibodies (University of Munich) and mAB AR-209 (Aronex Pharmaceuticals Inc) and mAB 2B-1 (Chiron) and combinations thereof.

Preferred erb selective anti-tumor agents include Herceptin, TAK-165, CP-724,714, ABX-EGF, HER3 and combinations thereof. Preferred pan erbb receptor inhibitors include GW572016, CI-1033, EKB-569, and Omitarg and combinations thereof.

Additional erbB2 inhibitors include those disclosed in WO 98/02434, WO 99/35146, WO 99/35132, WO 98/02437, WO 97/13760, WO 95/19970, U.S. Pat. No. 5,587,458, and U.S. Pat. No. 5,877,305, each of which is herein incorporated by reference in its entirety. ErbB2 receptor inhibitors useful in the present invention are also disclosed in U.S. Pat. Nos. 6,465,449, and 6,284,764, and in WO 2001/98277 each of which are herein incorporated by reference in their entirety.

Additionally, other anti-tumor agents may be selected from the following agents, BAY-43-9006 (Onyx Pharmaceuticals Inc.), Genasense (augmerosen, Genta), Panitumumab (Abgenix/Amgen), Zevalin (Schering), Bexxar (Corixa/GlaxoSmithKline), Abarelix, Alimta, EPO 906 (Novartis), discodermolide (XAA-296), ABT-510 (Abbott), Neovastat (Aeterna), enzastaurin (Eli Lilly), Combrestatin A4P (Oxigene), ZD-6126 (AstraZeneca), flavopiridol (Aventis), CYC-202 (Cyclacel), AVE-8062 (Aventis), DMXAA (Roche/Antisoma), Thymitaq (Eximias), Temodar (temozolomide, Schering Plough) and Revilimd (Celegene) and combinations thereof.

Other anti-tumor agents may be selected from the following agents, CyPat (cyproterone acetate), Histerelin (histrelin acetate), Plenaixis (abarelix depot), Atrasentan (ABT-627), Satraplatin (JM-216), thalomid (Thalidomide), Theratope, Temilifene (DPPE), ABI-007 (paclitaxel), Evista (raloxifene), Atamestane (Biomed-777), Xyotax (polyglutamate paclitaxel), Targetin (bexarotine) and combinations thereof.

Additionally, other anti-tumor agents may be selected from the following agents, Trizaone (tirapazamine), Aposyn (exisulind), Nevastat (AE-941), Ceplene (histamine dihydrochloride), Orathecin (rubitecan), Virulizin, Gastrimmune (G17DT), DX-8951f (exatecan mesylate), Onconase (ranpirnase), BEC2 (mitumoab), Xcytrin (motexafin gadolinium) and combinations thereof.

Further anti-tumor agents rimy be selected from the following agents, CeaVac (CEA), NeuTrexin (trimetresate glucuronate) and combinations thereof. Additional anti-tumor agents may be selected from the following agents, OvaRex (oregovomab), Osidem (IDM-1), and combinations thereof. Additional anti-tumor agents may be selected from the following agents, Advexin (ING 201), Tirazone (tirapazamine), and combinations thereof. Additional anti-tumor agents may be selected from the following agents, RSR13 (efaproxiral), Cotara (131I chTNT 1/b), NBI-3001 (IL-4) and combinations thereof. Additional anti-tumor agents may be selected from the following agents, Canvaxin, GMK vaccine, PEG Interon A, Taxoprexin (DHA/paciltaxel), and combinations thereof.

Other anti-tumor agents include Pfizer's MEK1/2 inhibitor PD325901, Array Biopharm's MEK inhibitor ARRY-142886, Bristol Myers' CDK2 inhibitor BMS-387,032, Pfizer's CDK inhibitor PD0332991 and AstraZeneca's AXD-5438, and combinations thereof.

Additionally, mTOR inhibitors may also be utilized such as CCI-779 (Wyeth) and rapamycin derivatives RAD001 (Novartis) and AP-23573 (Ariad), HDAC inhibitors, SAHA (Merck Inc./Aton Pharmaceuticals) and combinations thereof. Additional anti-tumor agents include aurora 2 inhibitor VX-680 (Vertex), and Chk1/2 inhibitor XL844 (Exilixis).

The following cytotoxic agents, e.g., one or more selected from the group consisting of epirubicin (Ellence), docetaxel (Taxotere), paclitaxel, Zinecard (dexrazoxane), rituximab (Rituxan) imatinib mesylate (Gleevec), and combinations thereof, may be used in combination with a compound of the present invention and pharmaceutical compositions disclosed herein.

The invention also contemplates the use of the compounds of the present invention together with hormonal therapy, including but not limited to, exemestane (Aromasin, Pfizer Inc.), leuprorelin (Lupron or Leuplin, TAP/Abbott/Takeda), anastrozole (Arimidex, Astrazeneca), gosrelin (Zoladex, AstraZeneca), doxercalciferol, fadrozole, formestane, tamoxifen citrate (tamoxifen, Nolvadex, AstraZeneca), Casodex (AstraZeneca), Abarelix (Praecis), Trelstar, and combinations thereof.

The invention also relates to the use of the compounds of the present invention together with hormonal therapy agents such as anti-estrogens including, but not limited to fulvestrant, toremifene, raloxifene, lasofoxifene, letrozole (Femara, Novartis), anti-androgens such as bicalutamide, flutamide, mifepristone, nilutamide, Casodex™ (4′-cyano-3-(4-fluorophenylsulphonyl)-2-hydroxy-2-methyl-3′-(trifluoromethyl) propionanilide, bicalutamide) and combinations thereof.

Further, the invention provides a compound of the present invention alone or in combination with one or more supportive care products, e.g., a product selected from the group consisting of Filgrastim (Neupogen), ondansetron (Zofran), Fragmin, Procrit, Aloxi, Emend, or combinations thereof.

Particularly preferred cytotoxic agents include Camptosar, Erbitux, Iressa, Gleevec, Taxotere and combinations thereof.

The following topoisomerase I inhibitors may be utilized as anti-tumor agents: camptothecin; irinotecan HCl (Camptosar); edotecarin; orathecin (Supergen); exatecan (Daiichi); BN-80915 (Roche); and combinations thereof. Particularly preferred toposimerase II inhibitors include epirubicin (Ellence).

Alkylating agents include, but are not limited to, nitrogen mustard N-oxide, cyclophosphamide, ifosfamide, melphalan, busulfan, mitobronitol, carboquone, thiotepa, ranimustine, nimustine, temozolomide, AMD-473, altretamine, AP-5280, apaziquone, brostallicin, bendamustine, carmustine, estramustine, fotemustine, glufosfamide, ifosfamide, KW-2170, mafosfamide, and mitolactol; platinum-coordinated alkylating compounds include but are not limited to, cisplatin, Paraplatin (carboplatin), eptaplatin, lobaplatin, nedaplatin, Eloxatin (oxaliplatin, Sanofi) or satrplatin and combinations thereof. Particularly preferred alkylating agents include Eloxatin (oxaliplatin).

Antimetabolites include but are not limited to, methotrexate, 6-mercaptopurine riboside, mercaptopurine, 5-fluorouracil (5-FU) alone or in combination with leucovorin, tegafur, UFT, doxifluridine, carmofur, cytarabine, cytarabine ocfosfate, enocitabine, S-1, Alimta (premetrexed disodium, LY231514, MTA), Gemzar (gemcitabine, Eli Lilly), fludarabin, 5-azacitidine, capecitabine, cladribine, clofarabine, decitabine, eflornithine, ethynylcytidine, cytosine arabinoside, hydroxyurea, TS-1, melphalan, nelarabine, nolatrexed, ocfosfate, disodium premetrexed, pentostatin, pelitrexol, raltitrexed, triapine, trimetrexate, vidarabine, vincristine, vinorelbine; or for example, one of the preferred anti-metabolites disclosed in European Patent Application No. 239362 such as N-(5-[N-(3,4-dihydro-2-methyl-4-oxoquinazolin-6-ylmethyl)-N-methylamino]-2-thenoyl)-L-glutamic acid and combinations thereof.

Antibiotics include intercalating antibiotics and include, but are not limited to: aclarubicin, actinomycin D, amrubicin, annamycin, adriamycin, bleomycin, daunorubicin, doxorubicin, elsamitrucin, epirubicin, galarubicin, idarubicin, mitomycin C, nemorubicin, neocarzinostatin, peplomycin, pirarubicin, rebeccamycin, stimalamer, streptozocin, valrubicin, zinostatin and combinations thereof.

Plant derived anti-tumor substances include for example those selected from mitotic inhibitors, for example vinblastine, docetaxel (Taxotere), paclitaxel and combinations thereof.

Cytotoxic topoisomerase inhibiting agents include one or more agents selected from the group consisting of aclarubicn, amonafide, belotecan, camptothecin, 10-hydroxycamptothecin, 9-aminocamptothecin, diflomotecan, irinotecan HCl (Camptosar), edotecarin, epirubicin (Ellence), etoposide, exatecan, gimatecan, lurtotecan, mitoxantrone, pirarubicin, pixantrone, rubitecan, sobuzoxane, SN-38, tafluposide, topotecan, and combinations thereof.

Preferred cytotoxic topoisomerase inhibiting agents include one or more agents selected from the group consisting of camptothecin, 10-hydroxycamptothecin, 9-aminocamptothecin, irinotecan HCl (Camptosar), edotecarin, epirubicin (Ellence), etoposide, SN-38, topotecan, and combinations thereof.

Immunologicals include interferons and numerous other immune enhancing agents. Interferons include interferon alpha, interferon alpha-2a, interferon, alpha-2b, interferon beta, interferon gamma-1a, interferon gamma-1b (Actimmune), or interferon gamma-n1 and combinations thereof. Other agents include filgrastim, lentinan, sizofilan, TheraCys, ubenimex, WF-10, aldesleukin, alemtuzumab, BAM-002, dacarbazine, daclizumab, denileukin, gemtuzumab ozogamicin, ibritumomab, imiquimod, lenograstim, lentinan, melanoma vaccine (Corixa), molgramostim, OncoVAX-CL, sargramostim, tasonermin, tecleukin, thymalasin, tositumomab, Virulizin, Z-100, epratuzumab, mitumomab, oregovomab, pemtumomab (Y-muHMFG1), Provenge (Dendreon) and combinations thereof.

Biological response modifiers are agents that modify defense mechanisms of living organisms or biological responses, such as survival, growth, or differentiation of tissue cells to direct them to have anti-tumor activity. Such agents include krestin, lentinan, sizofuran, picibanil, ubenimex and combinations thereof.

Other anticancer agents that can be used in combination with a compound of the present invention include alitretinoin, ampligen, atrasentan bexarotene, bortezomib. Bosentan, calcitriol, exisulind, finasteride, fotemustine, ibandronic acid, miltefosine, mitoxantrone, 1-asparaginase, procarbazine, dacarbazine, hydroxycarbamide, pegaspargase, pentostatin, tazarotne, Telcyta (TLK-286, Telik Inc.), Velcade (bortemazib, Millenium), tretinoin, and combinations thereof.

Platinum-coordinated compounds include but are not limited to, cisplatin, carboplatin, nedaplatin, oxaliplatin, and combinations thereof.

Camptothecin derivatives include but are not limited to camptothecin, 10-hydroxycamptothecin, 9-aminocamptothecin, irinotecan, SN-38, edotecarin, topotecan and combinations thereof.

Other antitumor agents include mitoxantrone, 1-asparaginase, procarbazine, dacarbazine, hydroxycarbamide, pentostatin, tretinoin and combinations thereof.

Anti-tumor agents capable of enhancing antitumor immune responses, such as CTLA4 (cytotoxic lymphocyte antigen 4) antibodies, and other agents capable of blocking CTLA4 may also be utilized, such as MDX-010 (Medarex) and CTLA4 compounds disclosed in U.S. Pat. No. 6,682,736; and anti-proliferative agents such as other farnesyl protein transferase inhibitors, for example the farnesyl protein transferase inhibitors. Additionally, specific CTLA4 antibodies that can be used in combination with compounds of the present invention include those disclosed in U.S. Pat. Nos. 6,682,736 and 6,682,736 both of which are herein incorporated by reference in their entirety.

Specific IGF1R antibodies that can be used in the combination methods of the present invention include those disclosed in WO 2002/053596, which is herein incorporated by reference in its entirety.

Specific CD40 antibodies that can be used in the present invention include those disclosed in WO 2003/040170 which is herein incorporated by reference in its entirety. Gene therapy agents may also be employed as anti-tumor agents such as TNFerade (GeneVec), which express TNFalpha in response to radiotherapy.

In one embodiment of the present invention statins may be used in combination with a compound of the present invention and pharmaceutical compositions thereof. Statins (HMG-CoA reducatase inhibitors) may be selected from the group consisting of Atorvastatin (Lipitor™, Pfizer Inc.), Provastatin (Pravachol™, Bristol-Myers Squibb), Lovastatin (Mevacor™, Merck Inc.), Simvastatin (Zocor™, Merck Inc.), Fluvastatin (Lescol™, Novartis), Cerivastatin (Baycol™, Bayer), Rosuvastatin (Crestor™, AstraZeneca), Lovostatin and Niacin (Advicor™, Kos Pharmaceuticals), derivatives and combinations thereof.

In a preferred embodiment the statin is selected from the group consisting of Atovorstatin and Lovastatin, derivatives and combinations thereof. Other agents useful as anti-tumor agents include Caduet.

Inasmuch as it may desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for coadministration of the compositions. Thus the kit of the invention includes two or more separate pharmaceutical compositions, at least one of which contains a compound of the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically includes directions for administration and may be provided with a memory aid.

### EXAMPLES

In the following examples molecules with a single chiral center, unless otherwise noted or indicated by the structural formula or chemical name, exist as a racemic mixture. Those molecules with two or more chiral centers, unless otherwise noted or indicated by the structural formula or chemical name, exist as a racemic mixture of diastereomers. Single enantiomers/diastereomers may be obtained by methods known to those skilled in the art.

¹H-NMR spectra were recorded on a Bruker instrument operating either at 300 MHz, or 400 MHz and ¹³C-NMR spectra were recorded operating at 75 MHz.

The following abbreviations may be used herein: Et₂O (diethyl ether); DMF (N,N-dimethylformamide); THF (tetrahydrofuran); DCM (dichloro-methane); DMA (dimethyl acetal); DBU (1,8-diazabicyclo[5.4.0]undec-7-ene); LiHMDS or LHMDS (lithium hexamethyldisilazide); TBME (tert-butyl methyl ether); LDA (lithium diisopropylamide); DMSO (dimethylsulfoxide); MeOH (methanol); EtOH (ethanol); BuOH (butanol); EtOAc (ethyl acetate); THF (tetrahydrofuran); Ac (acetyl); Me (methyl); Et (ethyl); Ph (phenyl); TMSI (trimethylsilyliodide); DSC (N,N′-disuccinimidyl carbonate); CDI (1,1′-carbonyldiimidazole); Boc (tert-butoxycarbonyl); nBuLi (n-butyl lithium); EDC (1-(3-dimethylaminopropyl)-3-ethylcarbodiimide hydrochloride); HOBt (N-hydroxybenzitriazole hydrate); DME (1,2-dimethoxyethane); Pd(dba)₂ (bis(dibenzylideneacetone)palladium(0)); and RT or rt (room temperature).

### Preparation of Compound (I): ethyl 2-amino-4-chloro-5,7-dihydro-6H-pyrrolo[3,4-d]pyrimidine-6-carboxylate

Intermediate (f) was prepared in three steps using known procedures (see, e.g. Viscontini and Buhler Helvetica Chimica Acta, 50(5): 1289-93; (1967)) and then converted to compound (I) in two additional steps.

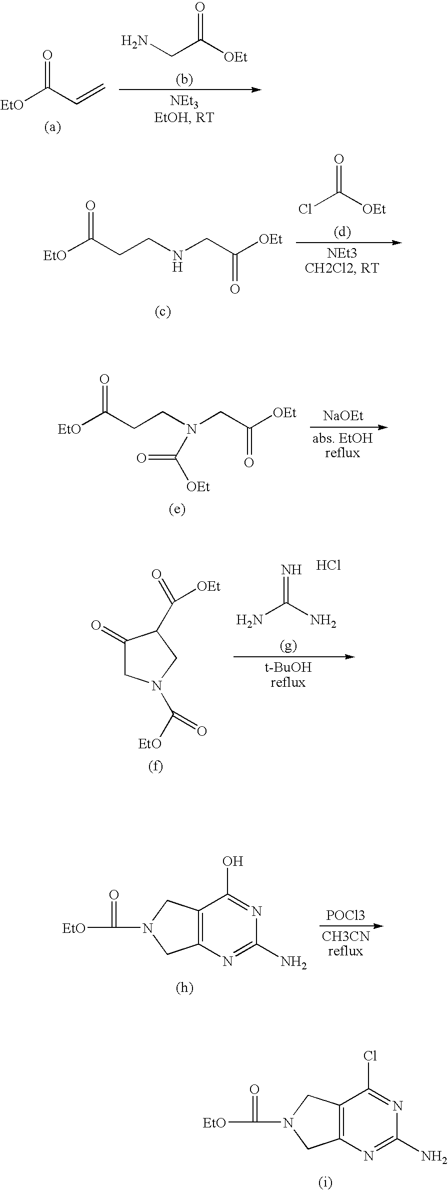

### Step 1. ethyl N-(ethoxycarbonyl)-β-alaninate (c)

Ethyl acrylate (a) (50 mL, 460 mmol, 1.1 eq), glycine ethyl ester hydrochloride (b) (58.4 g, 418 mmol, 1 eq), and triethylamine (58.3 mL, 418 mmol, 1 eq) in absolute EtOH (960 mL) was stirred at ambient temperature for approximately 72 h. After reaction was complete, volatile components were removed under vacuum and the crude intermediate (c) was carried on directly.

### Step 2. ethyl N-(ethoxycarbonyl)-N-(2-ethoxy-2-oxoethyl)-β-alaninate (e)

Crude intermediate (c) (418 mmol) was dissolved in CH₂Cl₂ (275 mL) and triethylamine (58.3 mL, 418 mmol) was added followed by ethyl chloroformate (d) (39.8 mL, 418 mmol). The reaction was stirred at ambient temperature for about 24 h. After the reaction was complete, the volatile components were removed under vacuum. The crude product was then distilled under vacuum (about 5 mmHg) and dissolved in EtOAc which was washed with aqueous saturated KHSO₄ ×3, with brine ×1 and dried over Na₂SO₄. Following filtration, the volatile components were removed under vacuum to afford intermediate (e) as a clear oil (74.8 g, 272 mmol) in 65% yield over two steps.

### Step 3. diethyl 4-oxopyrrolidine-1,3-dicarboxylate (f)

Intermediate (e) (18.0 g, 65.2 mmol) was added to an ice bath cooled solution of NaOEt (32.6 mL) (21% by weight in EtOH) in absolute EtOH (41.7 mL) under a nitrogen atmosphere. The ice bath was removed and the mixture was heated at 80° C. for about 12 h until the condensation was complete as observed by TLC. The mixture was poured onto ice/water and extracted into EtOAc. The solvent was dried with Na₂SO₄, filtered, and evaporated to afford crude intermediate (f) as an off white solid (14.05 g) which was carried on without purification.

### Step 4. ethyl 2-amino-4-hydroxy-5,7-dihydro-6H-pyrrolo[3,4-d]pyrimidine-6-carboxylate (h)

A suspension of intermediate (f) (14.05 g) and guanidine carbonate (g) (16.6 g, 91.9 mmol) was refluxed in t-butanol (147 mL) for about 6 h. The mixture was allowed to cool to ambient temperature for about 2 h. The volatile components were removed under vacuum and water was added. The pH was adjusted to about 6-7 using KHSO₄. The resulting slurry was filtered to collect the solids which were washed with water followed by EtOAc. The solids were dried under vacuum to afford intermediate (h) as cream solids (11.9 g, 53.1 mmol) in 87% yield. ¹H NMR (400 MHz, DMSO-D6) δppm 11.01 (s, 1H), 6.97 (s, 0.5H, possible tautomer), 6.70 (s, 2H), 4.25 (s, 4H), 4.13-4.03 (m, 2H), 1.22 (t, 3H). LCMS (M+H)⁺: 225.2.

### Step 5. ethyl 2-amino-4-chloro-5,7-dihydro-6H-pyrrolo[3,4-c]pyrimidine-6-carboxylate (i)

Intermediate (h) (11 g, 49 mmol) was azeotroped ×2 with toluene on the rotovap to assure dryness. Anhydrous acetonitrile (250 mL) and POCl₃ (25 mL, 270 mmol) were added and the mixture was refluxed for about 2.5 h. Additional POCl₃ (50 mL) was added and the mixture was refluxed for an additional 2 h. The volatile components were concentrated under vacuum at 40° C. to give a red solution. A minimum amount of dry acetonitrile was added until the solution was readily transferable whereupon it was poured onto ice in a large beaker. The flask was further rinsed with a small amount of acetonitrile which was added to the ice. Water (about 50 mL) was added to the ice mixture to help it with stirring. Concentrated NH₄OH (25 mL) was added slowly with stirring until the ice slurry mixture was strongly basic, then 50% aqueous NaOH (25 mL) was also added to the still stirring slurry of ice. Additional ice was added. After about 5 minutes stirring as ice slurry, EtOAc was added. After stirring in the beaker for several more minutes, water was added to help melt the ice. The mixture was poured into a separatory funnel and the layers were allowed to partition. The aqueous layer was extracted with EtOAc ×3. The combined EtOAc extracts were washed ×2 with saturated aqueous KHSO₄, ×2 with saturated aqueous NaHCO₃, ×1 with brine, dried over Na₂SO₄, filtered and evaporated to afford a pale pink powder which was triturated with ethyl acetate to give compound (i) as pale pink solids (6.8 g, 28 mmol) in 57% yield. HPLC/LCMS purity was greater than 90%. ¹H NMR (400 MHz, DMSO-D6) δ ppm 7.20 (s, 2H), 4.48 (s, 2H), 4.45 (s, 2H), 4.17-4.08 (m, 2H), 1.24 (t, 3H). LCMS (M+H)⁺: 243.2, 245.2.

### Example 1

### ethyl 2-amino-4-(4-bromo-2-chloro-5-methoxyphenyl)-5,7-dihydro-6H-pyrrolo[3,4-d]pyrimidine-6-carboxylate

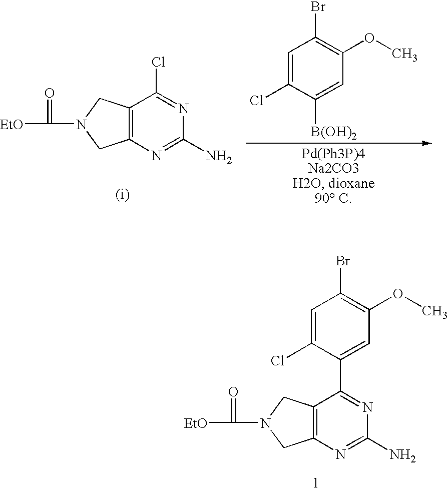

To compound (i) (437 mg, 1.80 mmol), (4-bromo-2-chloro-5-methoxyphenyl)boronic acid (340 mg, 1.28 mmol), and sodium carbonate (382 mg, 3.60 mmol) in an Ar purged round bottom flask was added 1,4-dioxane (8.6 mL) and water (5.2 mL). Preceding the addition, Ar had been bubbled through both solvents to deoxygenate. Palladium tetrakis(triphenylphosphine) (220 mg, 0.19 mmol) was then added and Ar was bubbled through the mixture using a needle and septum for about 3 min. at which point the Ar needle was removed and the mixture was placed in a 90° C. oil bath and heated 2.5 h. After cooling to ambient temperature, water was added and the resulting solid was collected by filtration. The solid was washed with water ×2, EtOAc ×2 and dried at 35° C. for 72 h to afford the title compound 1 (300 mg, 0.70 mmol) as a tan powder. ¹H NMR (400 MHz, DMSO-D6) δ ppm 7.88 (s, 1H), 7.16 (s, 1H), 6.98 (br s, 2H), 4.55-4.47 (m, 2H), 4.39-4.32 (m, 2H), 4.17-4.03 (m, 2H), 3.87 (s, 3H), 1.27-1.15 (m, 3H). LCMS (M+H)⁺: 429.0, 427.2.

### Example 2

### 4-(4-bromo-2-chloro-5-methoxyphenyl)-6,7-dihydro-5H-pyrrolo[3,4-d]pyrimidin-2-amine

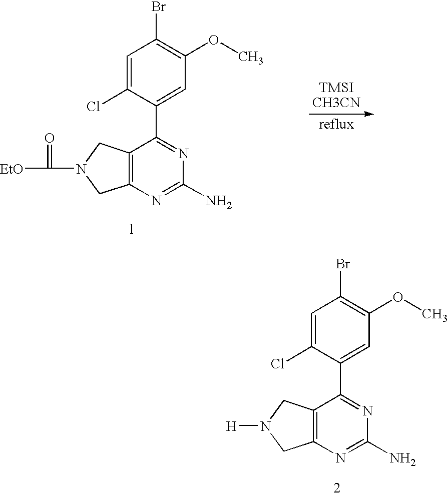

To compound 1 (300 mg, 0.70 mmol) was added anhydrous CH₃CN (4 mL) followed by trimethylsilyliodide (0.5 mL, 4.0 mmol). The mixture was refluxed for about 1 h and then cooled to ambient temperature whereupon a small amount of methanol was added to quench. The volatile components were then reduced under vacuum and a small amount of CH₃CN was added followed by Et₂O to precipitate yellow solids which gradually turned orange. The solids were washed with Et₂O and then dried under vacuum at 30° C. overnight. Assuming a salt containing 2 HI, the title compound 2 (404 mg, 0.66 mmol) was obtained as orange solids in about 95% yield and was carried on without further purification. ¹H NMR (400 MHz, DMSO-D6) δ ppm 3.81-3.90 (m, 3H) 4.30 (s, 2H) 4.39 (s, 2H) 7.12 (s, 1H) 7.16 (s, 2H) 7.89 (s, 1H) 9.40 (s, 1H). LCMS (M+H)⁺: 357.0, 355.0.

### Example 3

### 2-amino-4-(4-bromo-2-chloro-5-methoxyphenyl)-N-cyclobutyl-5,7-dihydro-6H-pyrrolo[3,4-d]pyrimidine-6-carboxamide

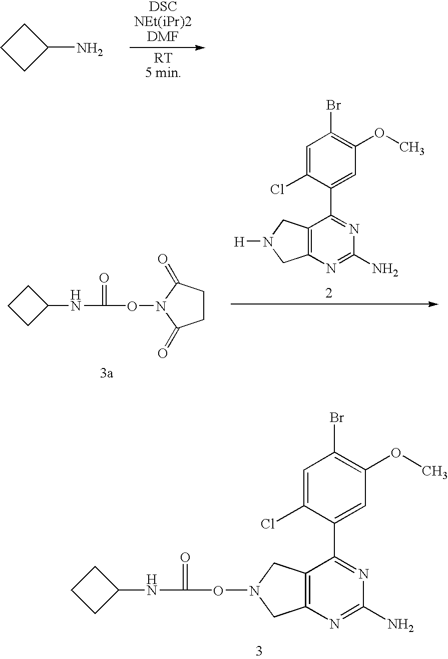

At ambient temperature to stirring N,N′-disuccinimidyl carbonate (67 mg, 0.26 mmol) in DMF (1 mL) was added cyclobutylamine (0.022 mL, 0.26 mmol). Diisopropyl ethylamine (0.152 mL, 0.873 mmol) was then added and the mixture was allowed to stir for about 5 min. With continued stirring, compound 2 (177 mg, 0.289 mmol) in DMF (2 mL) was added. The mixture was allowed to stir overnight and then purified by preparative HPLC. Following lyophilization of the combined purified fractions, the title compound 3 was obtained (57 mg, 0.13 mmol) as a fluffy white solid in 48% yield. ¹H NMR (400 MHz, DMSO-D6) 5 ppm 7.80 (s, 1H), 7.09 (s, 1H), 6.83 (s, 2H), 6.46 (d, 1H), 4.35 (s, 2H), 4.20 (s, 2H), 4.06 (m, 1H), 3.79m (s, 3H), 2.07-1.97 (m, 2H), 1.92-1.79 (m, 2H), 1.53-1.41 (m, 2H). LCMS (M+H)⁺: 454.2, 452.2.

### Example 4

### 4-(4-chloro-2-methylphenyl)-6-(propylsulfonyl)-6,7-dihydro-5H-pyrrolo[3,4-d]pyrimidin-2-amine

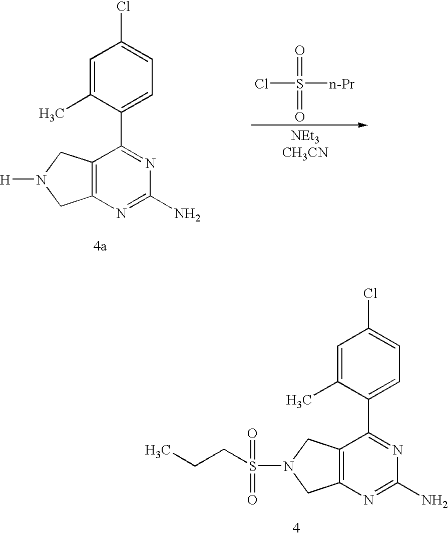

The preparation of 4-(4-chloro-2-methylphenyl)-6,7-dihydro-5H-pyrrolo[3,4-d]pyrimidin-2-amine 4a from compound (i) and 4-chloro-2methylphenylboronic acid was carried out in two steps in a manner analogous to that described for Examples 1 and 2. To a suspension of crude 4a (100.7 mg, ˜0.200 mmol) in acetonitrile (4.0 mL) at 0° C. was added triethylamine (0.15 mL, 1.1 mmol) and 1-propanesulfonyl chloride (0.025 mL, 0.22 mmol). After 30 minutes at 0° C., the reaction mixture was concentrated under vacuum, redissolved in dichloromethane (10 mL), washed with water (10 mL), and concentrated. The residue was purified by preparative HPLC and following lyophilization of the combined purified fractions, the title compound was obtained (16 mg, 0.04 mmol) as a white solid in 22% yield. ¹H NMR (300 MHz, DMSO-D6) δ ppm 7.43 (d, J=1.9 Hz, 1H), 7.38 (d, J=8.1 Hz, 1H), 7.33 (dd, J=8.1 and 1.7 Hz, 1H), 6.86 (br s, 2H), 4.49 (s, 2H), 4.36 (s, 2H), 3.22-3.17 (m, 2H), 2.26 (s, 3H), 1.75-1.63 (m, 2H), 0.97 (t, J=7.4 Hz, 3H). LCMS (M+H)⁺: 367.2.

### Example 5

### 6-benzyl-4-(4-bromo-2-chloro-5-methoxyphenyl)-6,7-dihydro-5H-pyrrolo[3,4-d]pyrimidin-2-amine trifluoroacetate

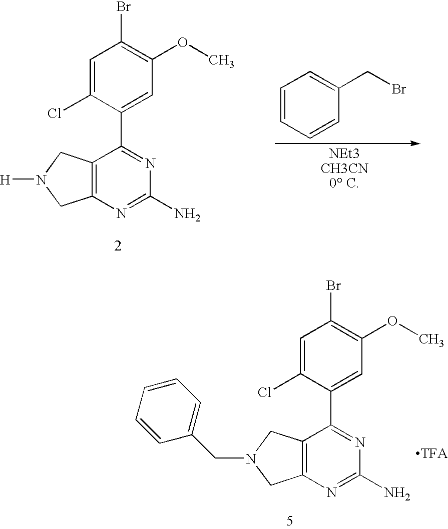

To compound 2 (91 mg, 0.15 mmol) in DMSO (1 mL), cooled in ice bath, was added N,N-diisopropylethylamine (0.078 mL, 0.45 mmol) followed by benzyl bromide (0.016 mL, 0.13 mmol). The frozen mixture was allowed to thaw and warmed to ambient temperature with stirring. After about 20 min., the clear red solution was analyzed by LCMS. An approximately 3:2 mixture of the desired mono alkylated product and the quaternary salt was observed. The mixture was purified by preparative HPLC and following lyophilization of the combined purified fractions, the title compound 5 was obtained (29 mg, 0.05 mmol) as a white solid TFA salt in 32% yield. The bis-alkylated quaternary salt was also isolated as a by-product (15 mg). ¹H NMR (400 MHz, DMSO-D6) δ ppm 7.90 (s, 1H), 7.58-7.39 (m, 5H), 7.29-7.14 (m, 2H), 7.08 (s, 1H), 4.68-4.25 (m, 6H), 3.89 (s, 3H). LCMS (M+H)⁺: 447.2, 445.2.

### Example 6

### 4-(2,4-dichlorophenyl)-6-[(35-difluoropyridin-2-yl)carbonyl]-6,7-dihydro-5H-pyrrolo[3,4-d]pyrimidin-2-amine

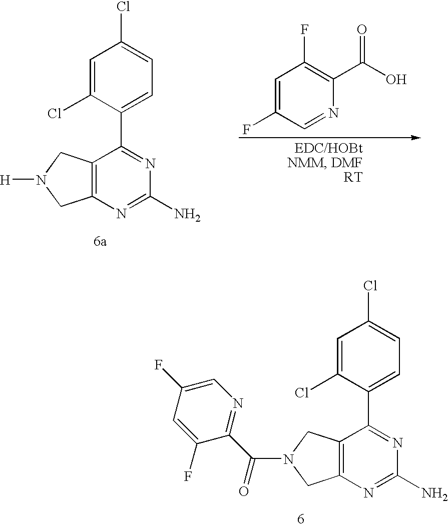

The preparation of 4-(2,4-dichlorophenyl)-6,7-dihydro-5H-pyrrolo[3,4-d]pyrimidin-2-amine 6a from compound (i) and 2,4-dichlorophenylboronic acid was carried out in two steps in a manner analogous to that described for Examples 1 and 2. To a mixture of compound 6a (54 mg, 0.1 mmol) and 3,5-difluoropyridine-2-carboxylic acid (24 mg, 0.15 mmol) in DMF (2.0 mL) was added 4-methylmorpholine (0.1 mL, 1.0 mmol), EDC (58 mg, 0.3 mmol) and HOBt (46 mg, 0.3 mmol). The resulting mixture was stirred at room temperature for 16 hours. The mixture was partitioned between EtOAc (200 mL) and washed with saturated aqueous NaHCO₃ (50 mL) and brine (50 mL). The organic layer was dried over Na₂SO₄, filtered and concentrated under vacuum. The residue was purified by preparative HPLC and following lyophilization of the combined purified fractions, the title compound 6 was obtained (25 mg, 0.06 mmol) as a white solid (solvated with 0.4 equivalents of HOAc) in 56% yield. ¹H NMR (400 MHz, DMSO-d6) δ ppm 4.54 (d, J=32.08 Hz, 2H) 4.70 (d, J=29.56 Hz, 2H) 6.98 (d, J=16.93 Hz, 2H) 7.38-7.62 (m, 2H) 7.77 (dd, J=36.38, 2.02 Hz, 1H) 8.02-8.21 (m, 1H) 8.57 (dd, J=30.69, 2.15 Hz, 1H). LCMS (M+H)⁺: 424, 422.

### Example 7

### 2-amino-4-(4-chloro-2-methylphenyl)-N-cyclopropyl-5,7-dihydro-6H-pyrrolo[3,4-d]pyrimidine-6-carboxamide

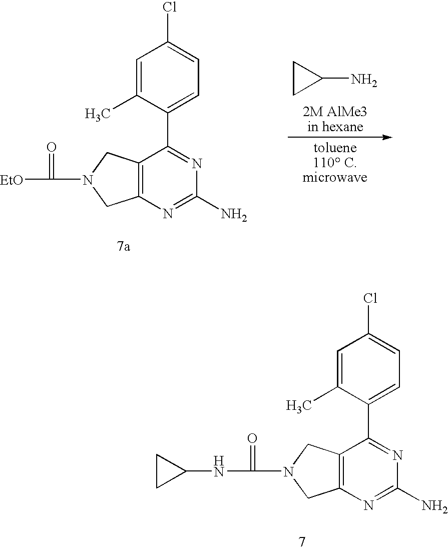

The preparation of ethyl 2-amino-4-(4-chloro-2-methylphenyl)-5,7-dihydro-6H-pyrrolo[3,4-d]pyrimidine-6-carboxylate 7a from compound (i) and 4-chloro-2-methylphenylboronic acid was carried out in a manner analogous to that described for Example 1. To a solution of cyclopropanamine (0.15 mL, 2.2 mmol) in toluene (2.5 mL) at 0° C. was added trimethyl aluminum (2.0 M in hexanes, 0.60 mL, 1.2 mmol) dropwise and the reaction mixture was allowed to warm to room temperature. After 30 minutes, compound 7a (75.0 mg, 0.225 mmol) was added in one portion, and the reaction was warmed to 110° C. in the microwave for 30 minutes. The mixture was then cooled to room temperature, quenched with THF/water (2.5 mL/0.5 mL), and the precipitate was removed by filtration. The filtrate was concentrated under vacuum and purified by preparative HPLC. Following lyophilization of the combined purified fractions, the title compound 7 was obtained (55 mg, 0.16 mmol) as a white solid in 71% yield. ¹H NMR (300 MHz, DMSO-D6) a ppm 7.43 (d, J=1.9 Hz, 1H), 7.34 (dd, J=8.3 and 1.9 Hz, 1H), 7.31 (d, J=8.1 Hz, 1H), 6.77 (br s, 2H), 6.46 (d, J=2.8 Hz, 1H), 4.38 (s, 2H), 4.21 (s, 2H), 2.55-2.48 (m, 1H), 2.22 (s, 3H), 0.56-0.50 (m, 2H), 0.41-0.36 (m, 2H). LCMS (M+H)⁺: 344.2.

### Example 8

### 2-amino-4-(4-bromo-2-chlorophenyl)-N-isopropyl-5,7-dihydro-6H-pyrrolo[3,4-d]pyrimidine-6-carboxamide hydrochloride

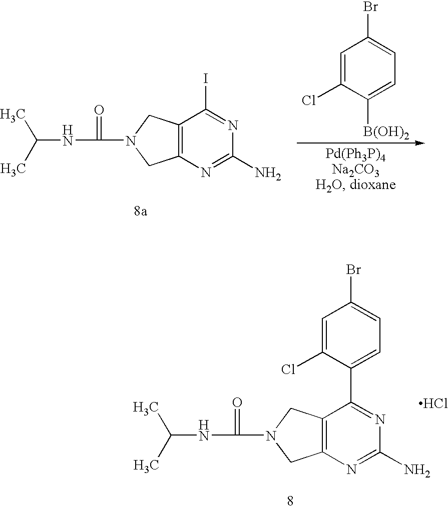

4-Bromo-2-chlorophenylboronic acid was prepared by treating 1-bromo-3-chloro-4-iodobenzene (1.339 g, 4.219 mmol) in THF (20.0 mL) at −78° C. with nBuLi (2.30 mL, 4.60 mmol) dropwise. After 1 h, trimethylborate (1.00 mL, 8.97 mmol) was added dropwise and the reaction was allowed to warm to rt. The reaction mixture was diluted with water (20 mL) and 3N NaOH (10 mL) and stirred for 30 minutes. The mixture was poured into Et₂O (50 mL) and the layers were separated. The aqueous layer was then acidified with 1N aq. KH₂PO₄ (15 mL) and boronic acid extracted into EtOAc. Following isolation, the 4-bromo-2-chlorophenylboronic acid (26.3 mg, 0.112 mmol) was combined with compound 8a (43.1 mg, 0.124 mmol), and aqueous sodium carbonate (2.0M, 0.125 mL, 3.4 mmol) in DME (2.0 mL). Nitrogen gas was bubbled into the mixture for ten minutes to deoxygenate. Tetrakis(triphenylphosphino)palladium(0) (12.1 mg, 0.0105 mmol) was added and the reaction was warmed to 85° C. After 4 h the reaction mixture was cooled to ambient temperature, poured into EtOAc (50 mL), washed with water (50 mL), concentrated under vacuum and purified by preparative HPLC. The purified fractions were combined and 1N HCl (about 3 eq.) was added before lyophilization to prepare the hydrochloride. The title compound 8 was obtained (15 mg, 0.11 mmol) in 30% yield. ¹H NMR (300 MHz, DMSO-D6) δ ppm 7.92 (d, J=1.6 Hz, 1H), 7.69 (dd, J=8.0 and 1.9 Hz, 1H), 7.41 (d, J=8.0 Hz, 1H), 4.41 (s, 2H), 4.24 (s, 2H), 3.82-3.74 (m, 1H), 1.05 (d, J=6.6 Hz, 6H). LCMS (M+H)⁺: 412.2, 410.2.

### Preparation of Compound 8a, 2-amino-4-iodo-N-isopropyl-5,7-dihydro-6H-pyrrolo[3,4-d]pyrimidine-6-carboxamide

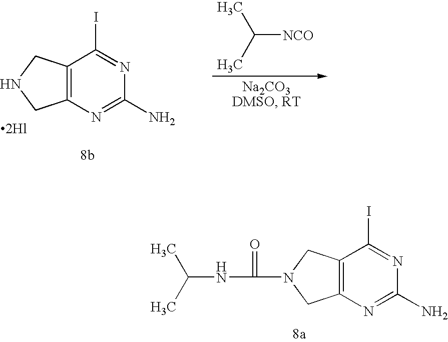

To a solution of 4-iodo-6H-pyrrolo[3,4-d]pyrimidin-2-amine 8b (520 mg, 1.0 mmol) in DMSO at ambient temperature was added sodium carbonate (256 mg, 2.4 mmol) and isopropyl isocyanate (0.13 mL, 1.1 mmol). After 3 h, the reaction was complete. The DMSO solution was filtered and the filtrate purified by preparative HPLC. Following lyophilization of the combined purified fractions, the title compound 8a was obtained (204 mg, 0.59 mmol) in 59% yield. ¹H NMR (30.0 MHz, DMSO-D6) δ ppm 6.96 (br s, 2H), 6.08 (d, J=7.7 Hz, 1H), 4.34 (s, 2H), 4.20 (s, 2H), 3.78-3.67 (m, 1H), 1.02 (d, J=6.6 Hz, 6H). LCMS (M+H)⁺: 348.2.

### Preparation of Compound 8b, 4-iodo-6H-pyrrolo[3,4-d]pyrimidin-2-amine

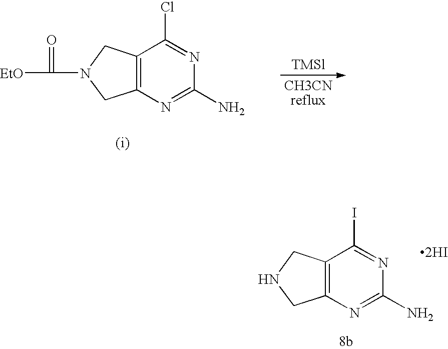

To a suspension compound (i) (1.00 g, 4.12 mmol) in CH₃CN (25.0 mL) was added iodotrimethylsilane TMSI (3.0 mL, 21.1 mmol). The mixture was refluxed 5 h and then cooled to ambient temperature. Methanol (5 mL) was added to quench the excess TMSI and the mixture was concentrated under vacuum to about 10 mL. Diethyl ether (40 mL) was added and the resulting precipitate was collected by filtration. The solids were then stirred in refluxing EtOAc (50 mL) for a few minutes at which point the mixture was cooled. The light tan solids (1.8 g, 3.4 mmol) were collected by filtration to afford the title compound 8b in 83% yield (assuming.2 HI salt). ¹H NMR (300 MHz, DMSO-D6) δ ppm 9.47 (br s, 2H), 4.37 (s, 2H), 425 (s, 2H). LCMS (M+H)⁺: 263.2.

### Example 9

### tert-butyl 2-amino-4-(4-bromo-2-chloro-5-methoxyphenyl)-5,7-dihydro-6H-pyrrolo[3,4-d]pyrimidine-6-carboxylate

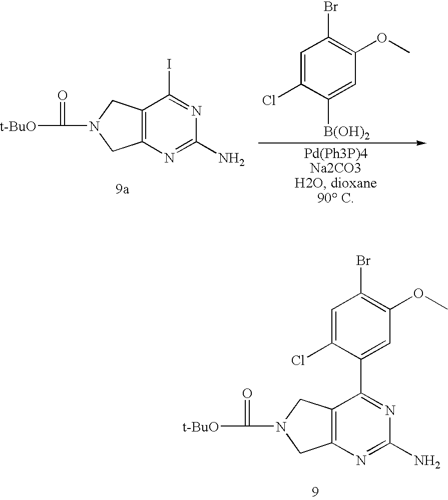

Impure compound 9a (113 mg) in 1,4-dioxane (2 mL) and water (1.2 mL) was treated with (4-bromo-2-chloro-5-methoxyphenyl)boronic acid (385 mg, 0.32 mmol), sodium carbonate (89 mg, 0.84 mmol) and tetrakis(triphenylphosphino)palladium(0) (50 mg, 0.04 mmol) and heated under conditions similar to those described in Example 1. After cooling to ambient temperature, water and EtOAc was added. The water layer was extracted twice with EtOAc and the combined extracts were washed with brine, dried with Na₂SO₄, filtered, and volatile components removed under vacuum. The residue was purified by preparative HPLC. Following lyophilization of the combined purified fractions, the tert-butoxycarbonyl (Boc) protected title compound 9 was obtained (33 mg, 0.07 mmol) as a white solid in greater than 23% yield. ¹H NMR (400 MHz, DMSO-D6) δ ppm 7.88 (s, 1H), 7.17 (s, 1H), 6.95 (s, 2H), 4.48-4.42 (m, 2H), 4.33-4.27 (m, 2H), 3.87 (s, 3H), 1.43 (d, 9H). LCMS (M+H)⁺: 457.2, 455.2.

### Preparation of Compound 9a, tert-butyl 2-amino-4-iodo-5,7-dihydro-6H-pyrrolo[3,4-d]pyrimidine-6-carboxylate

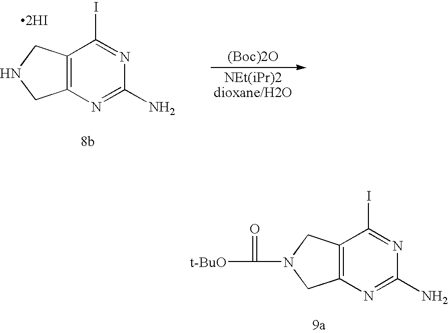

To a stirring mixture of impure compound 8b (113 mg) in 1,4 dioxane (1 mL) and water (1 mL) was added di-tert-butyldicarbonate (0.16 mL, 0.70 mmol) and diisopropylethylamine (0.25 mL, 1.4 mmol). After stirring over night the volatile components were reduced under vacuum and EtOAC and aqueous saturated KHSO₄ were added. The resulting two phase mixture was extracted twice with EtOAC and the combined extracts were washed with aqueous saturated KHSO₄ ×1, with aqueous saturated NaHCO₃ ×2, with brine ×1, and dried over Na₂SO₄. Following filtration, the volatile components were removed under vacuum to afford the title compound 9a as a tan solid (77 mg) which was combined with additional material 9a (36 mg) from a previous reaction and carried on directly to the next step. ¹H NMR (400 MHz, DMSO-D6) δ ppm 1.44 (d, J=4.80 Hz, 9H) 4.25 (d, J=13.14 Hz, 2H) 4.42 (d, J=8.34 Hz, 2H) 7.06 (s, 2H). LCMS (M+H)⁺: 348.2.

### Example 10

### ethyl 2-amino-4-(4-chlorophenoxy)-5,7-dihydro-6H-pyrrolo[3,4-d]pyrimidine-6-carboxylate

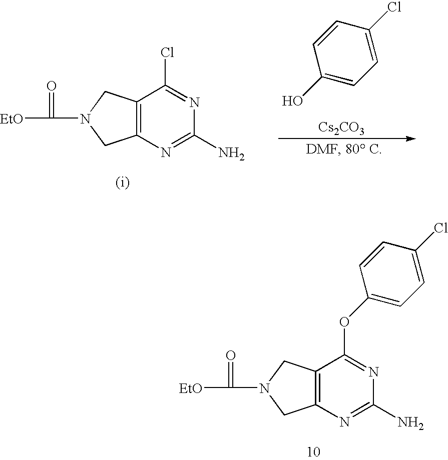

To compound (i) (99 mg, 0.41 mmol) and p-chlorophenol (52 mg, 0.41 mmol) in DMF (2 mL) was added cesium carbonate (266 mg, 0.82 mmol). The mixture was heated at 80° C. for 1.5 h and allowed to cool to ambient temperature. The mixture was filtered through a 0.45 μM teflon syringe filter and the filtrate submitted for preparative RPHPLC. Following lyophilization of the combined purified fractions, the title compound was obtained (48 mg, 0.14 mmol) as a white solid in 34% yield. ¹H NMR (400 MHz, DMSO-D6) δ ppm 7.49 (d, 2H), 7.26 (d, 2H), 6.75 (s, 2H), 4.50 (s, 1H), 4.46-4.39 (m, 3H), 4.17-4.08 (m, 2H), 1.24 (t, 3H). LCMS (M+H)⁺: 335.2.

### Example 11

### ethyl 2-amino-4-[(4-methoxy-3,5-dimethylpyridin-2-yl)methoxy]-5,7-dihydro-6H-pyrrolo[3,4-d]pyrimidine-6-carboxylate

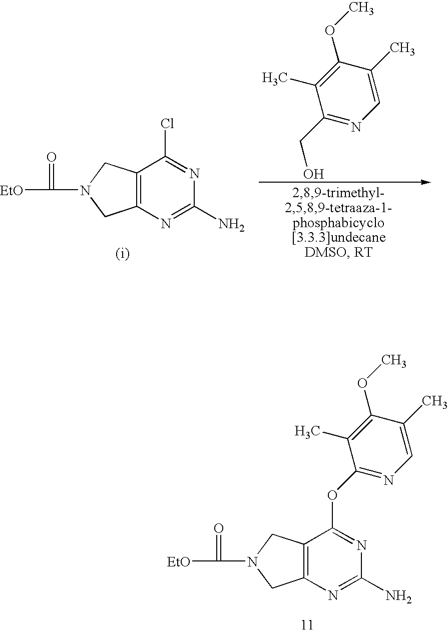

Compound (i) (54 mg, 0.22 mmol), 3,5-dimethyl-4-methoxy-2-pyridinemethanol (45 mg, 0.268 mmol) and 2,8,9-trimethyl-2,5,8,9-tetraaza-1-phosphabicyclo[3.3.3]undecane (58 mg, 0.27 mmol) were stirred in DMSO (1.5 mL) at ambient temperature for 2 h. The mixture was then purified by preparative HPLC, and following lyophilization of the combined purified fractions, the title compound 11 was obtained (37 mg, 0.10 mmol) as a white solid in 44% yield. ¹H NMR (300 MHz, DMSO-D6) (conformers) δ ppm 8.20 (s, 1H), 6.72 (br s, 2H), 5.39 (s, 1H), 5.38 (s, 1H), 4.36-4.29 (m, 4H), 4.11-4.03 (m, 2H), 3.75 (s, 3H), 2.22 (s, 6H), 1.23-1.17 (m, 3H). LCMS (M+H)⁺: 374.2.

### Example 12

### ethyl 2-amino-4-(1,3-dihydro-2H-isoindol-2-yl)-5,7-dihydro-6H-pyrrolo[3,4-d]pyrimidine-6-carboxylate

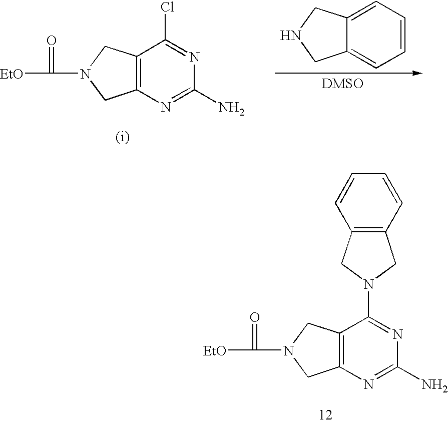

Compound (i) (74.8 mg, 0.308 mmol) and isoindoline (105 mg, 0.881 mmol) in DMSO (1 mL) were stirred and heated to 80° C. for 20 minutes. After cooling, the mixture was poured into water and extracted into CH₂Cl₂. Following evaporation of the volatile components, the residue was dissolved in DMSO and submitted for preparative RPHPLC. Following lyophilization of the combined purified fractions, the title compound 12 was obtained (68 mg, 0.19 mmol) in 62% yield.

¹H NMR (300 MHz, DMSO-D6) (conformers) δ ppm 7.42-7.39 (m, 2H), 7.32-7.29 (m, 2H), 6.13 (br s, 2H), 4.96-4.87 (br m, 4H), 4.28-4.23 (m, 2H), 4.16-4.10 (m, 2H), 3.31 (br s, partially obscured by H2O peak, 2H), 1.28-1.21 (m, 3H). LCMS (M+H)⁺: 326.2.

### Example 13

### ethyl 2-amino-4-(4-bromo-2-chloro-5-hydroxyphenyl)-5,7-dihydro-6H-pyrrolo[3,4-d]pyrimidine-6-carboxylate

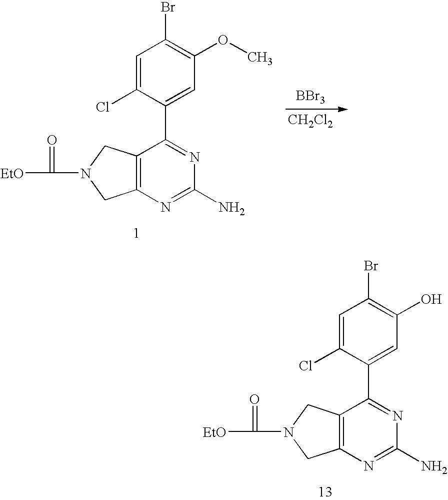

To a stirring suspension of compound 1 (428 mg, 1.0 mmol) in CH₂Cl₂ (10 mL) at 0° C. was added BBr₃ (0.5 mL, 5.0 mmol). The mixture was stirred overnight and allowed to warm to ambient temperature whereupon ice and water (10 mL) were added and the resulting precipitate was collected by filtration. After washing with water and CH₂Cl₂, the solid was dried under vacuum to give the title compound 13 (375 mg, 0.91 mmol) as a yellow solid in 91% yield. ¹H NMR (300 MHz, DMSO-D6) (conformers) δ ppm 1.09-1.28 (m, 3H) 3.99-4.16 (m, 2H) 4.33 (d, J=8.34 Hz, 2H) 4.47 (d, J=11.12 Hz, 2H) 6.93 (d, J=1.77 Hz, 1H) 7.74 (s, 1H) 10.89 (s, 1H). LCMS (M+H)⁴: 417.0, 415.0.

### Example 14

### ethyl 2-amino-4-[4-bromo-2-chloro-5-(2-chloroethoxy)phenyl]-5,7-dihydro-6H-pyrrolo[3,4-d]pyrimidine-6-carboxylate

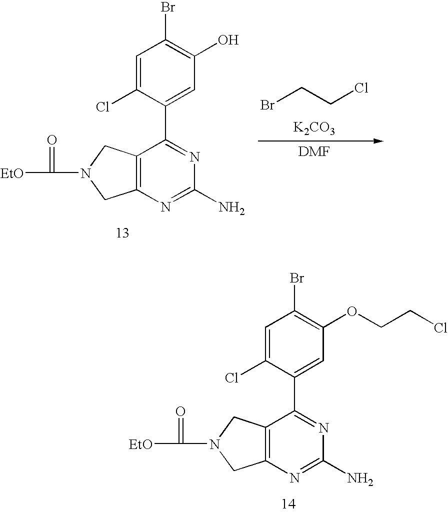

To a stirring mixture of compound 13 (360 mg, 0.87 mmol) and potassium carbonate (361 mg, 2.61 mmol) in anhydrous DMF (4 mL) was added 1-bromo-3-chloropropane (0.15 mL, 1.74 mmol). The mixture was heated at 50° C. for 12 hours whereupon the mixture was cooled and filtered to remove suspended solids, and the solids were washed well with EtOAc. The washes and filtrate were combined and additional EtOAc (200 mL) was added. The combined organic layer was washed with saturated aqueous NaHCO₃ (50 mL), and brine (50 mL), and then dried over Na₂SO₄, filtered, and concentrated under vacuum. The yellow residue was treated with CH₂Cl₂ (30 mL), and the resulting solids were collected by filtration. After washing with CH₂Cl₂ and hexane, the solids were dried under vacuum to give the title compound 14 (240 mg, 0.5 mmol) as a white solid in 58% yield. ¹H NMR (300 MHz, DMSO-D6) δ ppm 1.11-1.27 (m, 3H) 3.90-4.00 (m, 2H) 4.08 (dd, J=14.53, 7.20 Hz, 2H) 4.29-4.40 (m, 4H) 4.48 (d, J=10.61 Hz, 2H) 6.95 (s, 2H) 7.20 (s, 1H) 7.88 (s, 1H). LCMS (M+H)⁺: 477.0, 475.0.

### Example 15

### 2-amino-4-{4-bromo-2-chloro-5-[2-(dimethylamino)ethoxy]phenyl}-N-isopropyl-5,7-dihydro-6H-pyrrolo[3,4-d]pyrimidine-6 carboxamide

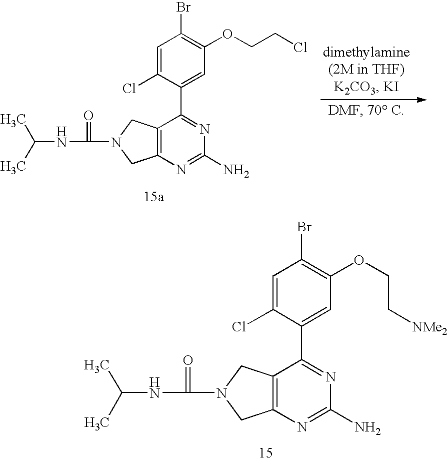

To a mixture of compound 15a (90 mg, 0.18 mmol), K₂CO₃ (76 mg, 0.55 mmol), and KI (61 mg, 0.37 mmol) in DMF (2.0 mL) was added a 2M solution of dimethylamine in THF (0.3 mL, 0.55 mmol). After stirring at 70° C. for 12 h, the mixture was cooled and filtered to remove suspended solids. The solids were washed with EtOAc. The washes and filtrate were combined and additional EtOAc (200 mL) was added. The combined organic layer was washed with saturated aqueous NaHCO₃ (50 mL), and brine (50 mL), and then dried over Na₂SO₄, filtered, and concentrated under vacuum. The residue was purified by preparative HPLC and following lyophilization of the combined purified fractions, the title compound 15 was obtained (16 mg, 0.03 mmol) as a yellow solid in 17% yield. ¹H NMR (400 MHz, DMSO-d6) δ ppm 1.06 (t, J=7.07 Hz, 6H) 2.22 (s, 6H) 2.65 (t, J=5.68 Hz, 2H) 3.78 (dd, J=13.89, 6.82 Hz, 1H) 4.14 (t, J=5.56 Hz, 2H) 4.25 (s, 2H) 4.42 (s, 2H) 6.08 (d, J=7.83 Hz, 1H) 6.88 (s, 2H) 7.19 (s, 1H) 7.85 (s, 1H). LCMS (M+H)⁺: 501.2, 499.2.

### Preparation of Compound 15a, 2-amino-4-[4-bromo-2-chloro-5-(2-chloroethoxy)phenyl]-N-isopropyl-5,7-dihydro-6H-pyrrolo[3,4-d]pyrimidine-6-carboxamide

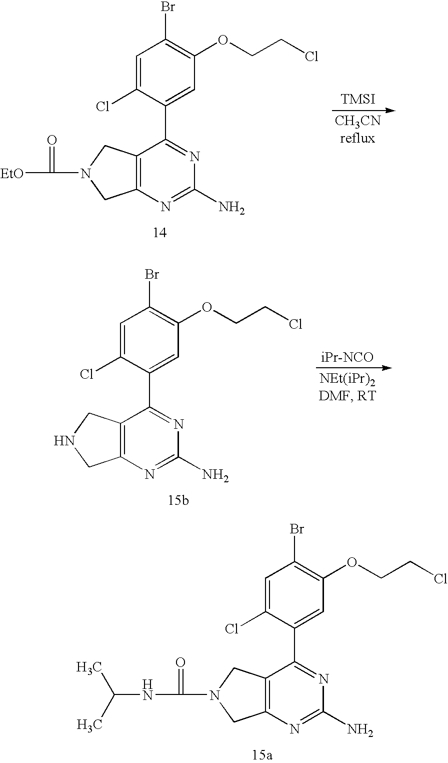

Compound 14 was deprotected in a manner similar to that described for Example 2. Extractive work up from EtOAc and saturated aqueous NaHCO₃ gave compound 15b, 4-[4-bromo-2-chloro-5-(2-chloroethoxy)phenyl]-6,7-dihydro-5H-pyrrolo[3,4-d]pyrimidin-2-amine, (191 mg, 0.473 mmol) which was dissolved in DMF (2.0 mL) and treated sequentially with diisopropylethylamine (0.4 mL, 2.36 mmol) and isopropylisocyanate (40 mg, 0.473 mmol) at ambient temperature. After 3 h, methanol was added and the mixture was concentrated under vacuum. The residue was dissolved in EtOAc and washed with saturated aqueous NaHCO₃ and brine. The organic layers were dried over Na₂SO₄, filtered, and concentrated under vacuum. The residue was chromatographed on silica gel eluting with 10% methanol in CH₂Cl₂. The purest fractions were combined and the volatile components removed under vacuum to give the title compound 15a (180 mg, 0.37 mmol) as a brown foam in 78% yield. ¹H NMR (400 MHz, DMF-d7) δ ppm 1.04-1.11 (m, 6H) 3.81 (s, 1H) 3.92-4.04 (m, 2H) 4.28 (s, 2H) 4.39 (d, J=10.11 Hz, 2H) 4.44 (s, 2H) 6.09 (d, J=8.08 Hz, 1H) 6.91 (s, 2H) 7.24 (s, 1H) 7.91 (s, 1H). LCMS (M+H)⁺: 488.0, 490.0.

### Example 17

### 6-[(4-chlorophenyl)acetyl]-4-(4-fluoro-2-methoxyphenyl)-5,6,7,8-tetrahydropyrido[4,3-d]pyrimidin-2-amine

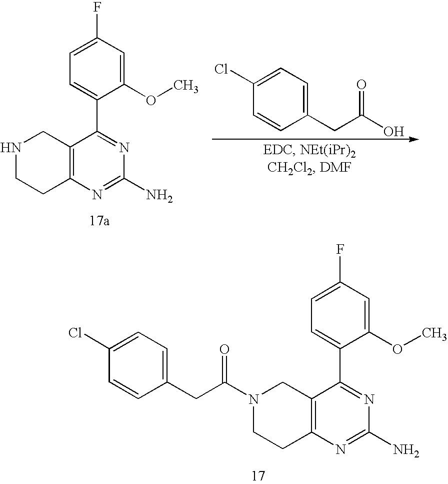

Compound 17a (32 mg, 0.19 mmol) was coupled to (4-chlorophenyl)acetic acid (32 mg, 0.19 mmol), using EDC (40 mg, 0.21 mmol) and NEt(iPr)₂ (0.051 mL, 0.29 mmol) in DMF (0.5 mL) and CH₂Cl₂ (0.5 mL) in a manner similar to that described for Example 6. Purification on silica gel (eluting with 10% MeOH in CH₂Cl₂) gave, after isolation, the title compound 17 (48 mg, 0.11 mmol) in 59% yield. ¹H NMR (400 MHz, DMSO-D6) (conformers) δ ppm 7.41-6.82 (m, 7H), 6.58-6.47 (m, 2H), 4.17 (s, 2H), 3.85-3.55 (m, 7H), 2.71 (br s, 2H). LCMS (M+H)⁺: 429.2, 427.2.

### Preparation of Compound 17a, 4-(4-fluoro-2-methoxyphenyl)-5,6,7,8-tetrahydropyrido[4,3-d]pyrimidin-2-amine

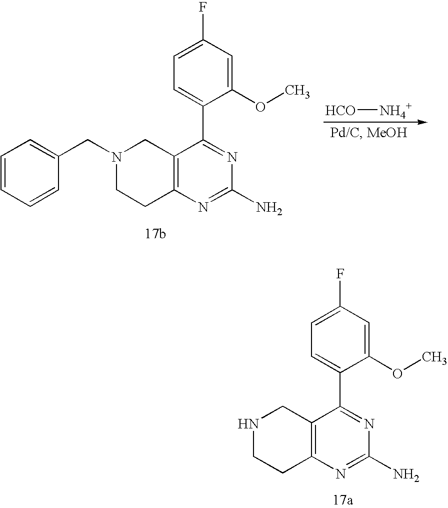

Compound 17b (971 mg, 2.66 mmol) in methanol was treated with ammonium formate (1.9 g, 29.4 mmol) and 10% palladium on carbon (490 mg). After stirring for several days, the palladium on carbon was removed through two successive filtrations—first through filter paper and then through a 0.45 micron teflon syringe filter. The filtrate was reduced under vacuum and a small amount of water was added. The resulting precipitate was collected by filtration to afford the title compound 17a (438 mg, 1.6 mmol) in 60% yield after drying under vacuum. ¹H NMR (400 MHz, DMSO-D6) (conformers) δ ppm 8.26 (s, 1H), 7.19 (m, 1H), 7.03 (m, 1H), 6.87 (m, 1H), 6.41 (s, 2H), 3.78 (s, 3H), 3.53-3.39 (m, 2H, partially obscured), 3.12-3.02 (m, 2H), 2.71-2.62 (m, 2H). LCMS (M+H)⁺: 275.0.

### Preparation of Compound 17b, 6-benzyl-4-(4-fluoro-2-methoxyphenyl)-5,6,7,8-tetrahydropyrido[4,3-d]pyrimidin-2-amine

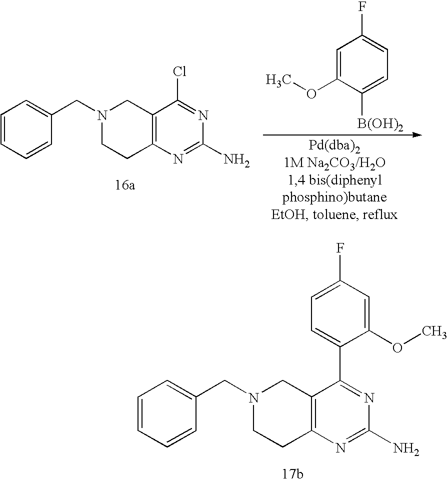

Argon was bubbled through a mixture of compound 16a (286 mg, 1.04 mmol), 4-fluoro-2-methoxyphenyl)boronic acid (194 mg, 1.14 mmol), 1M aqueous Na₂CO₃ (1 mL) in toluene (2 mL) and ethanol (0.5 mL). Pd(dba)₂ (30 mg, 0.052 mmol) and 1,4 bis (diphenylphosphino)butane (44 mg, 0.104 mmol) were added and additional argon was bubbled through the mixture which was then refluxed for approximately 19 h. After cooling to ambient temperature, water was added and the crude product was extracted into EtOAc, washed with brine, dried over Na₂SO₄ and filtered. The volatile components were removed under vacuum and the residue was purified on silica eluting with EtOAc followed by acetone. The purest fractions were combined and reduced to dryness whereupon the solids were dissolved in THF and filtered through a 0.45 micron teflon filter. Again the volatile components were removed under vacuum to afford the title compound 17b (228 mg, 0.62 mmol) in 60% yield. ¹H NMR (400 MHz, DMSO-D6) (conformers) δ ppm 7.32-7.20 (m, 5H), 7.15 (m, 1H), 6.94 (d, 1H), 6.80 9m, 1H), 6.37 (s, 2H), 3.63-3.42 (m, 5H, (contains 3.63 (s, 3H)), 3.31-2.92 (m, 2H), 2.74-2.59 (m, 4H (contains 2.66 (s, 2H)). LCMS (M+H)⁺: 365.0.

### Example 18

### 2-amino-N-cyclobutyl-4-[2,4-dichloro-6-(4,4,4-trifluorobutoxy)phenyl]-5,7-dihydro-6H-pyrrolo[3,4-d]pyrimidine-6-carboxamide

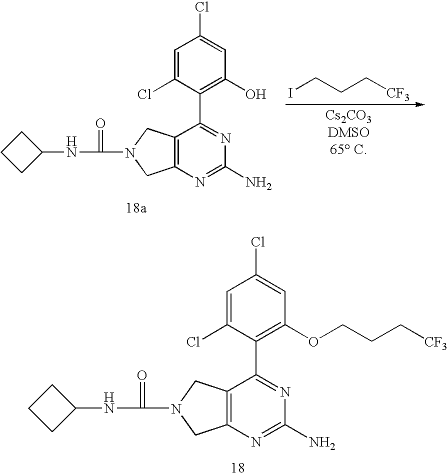

A solution of compound 18a 1 (91 mg, 0.231 mmol) in DMSO (1.5 mL) was treated with Cs₂CO₃ (174 mg, 0.533 mmol) and 1-iodo-4,4,4-trifluorobutane (89.2 mg, 0.375 mmol) and warmed to 65° C. After 30 minutes, the reaction mixture was filtered and the DMSO solution was subjected to preparative HPLC. The purified fractions were combined and lyophilized. The resulting white powder was then dissolved in a solution of CH₃CN, (2 mL), 1N HCl (0.45 mL) and water (5 mL). Following lyophilization, compound 18 (46 mg, 0.085 mmol) was obtained as a white powder in 37% yield. ¹H NMR (300 MHz, DMSO-D6) δ ppm 7.32 (d, J=1.8 Hz, 1H), 7.26 (d, J=1.8 Hz, 1H), 6.46 (d, J=7.3 Hz, 1H), 4.40-4.30 (m, 2H), 4.11-4.02 (m, 5H), 2.12-1.99 (m, 4H), 1.91-1.81 (m, 2H), 1.74-1.67 (m, 2H), 1.52-1.43 (m, 2H). LCMS (M+H)⁺: 504.0, 506.0. Anal. Calcd for C₂₁H₂₂N₅Cl₂O₂F₃.0.7HCl.0.6H2O: C, 46.65; H, 4.46; N, 12.95; Cl, 17.70. Found: C, 46.75; H, 4.26; N, 12.69; Cl, 17.43.

### Preparation of Compound 18a, 2-amino-N-cyclobutyl-4-(2,4-dichloro-6-hydroxyphenyl)-5,7-dihydro-6H-pyrrolo[3,4-d]pyrimidine-6-carboxamide

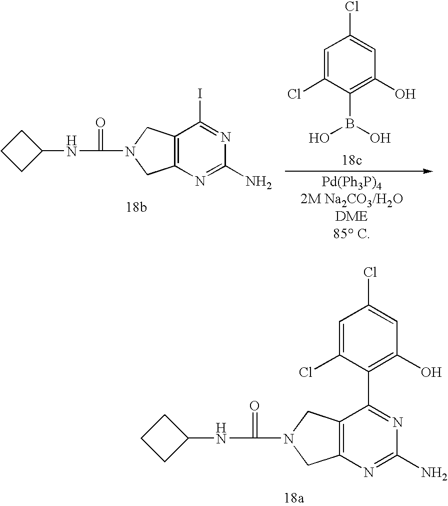

Nitrogen was bubbled through a solution of compound 18b (1350 mg, 3.75 mmol), compound 18c (775 mg, 3.75 mmol) and 2.0M aqueous sodium carbonate (0.60 mL, 1.20 mmol) in 1,4-dioxane (7.0 mL) for 15 minutes. To this was added tetrakis(triphenylphosphino)palladium(0) (431 mg, 0.373 mmol) and the mixture was warmed to 85° C. After 5 h, the mixture was cooled to ambient temperature and EtOAc (20 mL) and water (20 mL) were added. The aqueous layer was extracted with EtOAc and the EtOAc layer was discarded. The pH of the aqueous solution was adjusted to about 5 with 1N KH₂PO₃ and then extracted with a mixture of EtOAc and iPrOH. The volatile components were remove under vacuum to give compound 18a (869 mg, 2.2 mmol) in 59% yield which was carried on without further purification. ¹H NMR (300 MHz, DMSO-D6) δ ppm 10.64 (s, 1H), 7.16 (d, J=1.7 Hz, 1H), 6.97 (d, J=1.7 Hz, 1H), 6.79 (br, 2H), 6.52 (d, J=7.5 Hz, 1H), 4.40 (br s, 2H), 4.15 (br s, 2H), 4.08-3.99 (m, 1H), 2.15-2.03 (m, 2H), 1.97-1.86 (m, 2H), 1.60-1.47 (m, 2H). LCMS (M+H)⁺: 394.2 and 396.2

### Preparation of Compound 18b: 2-amino-N-cyclobutyl-4-iodo-5,7-dihydro-6H-pyrrolo[3,4-d]pyrimidine-6-carboxamide

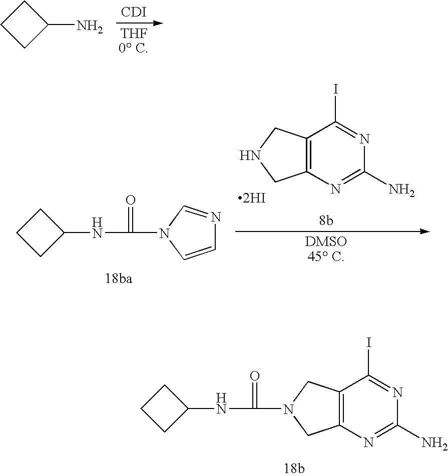

To a solution of 1,1-carbonyldiimidazole (3.54 g, 21.8 mmol) in THF (100 mL) at 0° C. was added a solution of cyclobutylamine (1.00 g, 14.1 mmol) in THF (9.0 mL) dropwise over 10 minutes. The mixture was allowed to warm to ambient temperature and stirred for 2 h. The volatile components were removed under vacuum and the resulting oil was purified on silica gel, eluting with a gradient of 0-10% MeOH in CH₂Cl₂. The purest fractions were combined and the volatile components removed under vacuum to give compound 18ba, N-cyclobutyl-1H-imidazole-1-carboxamide, (2.2 g, 13.4 mmol) as clear oil. Compound 18ba (1.39 g, 8.41 mmol) and compound 8b (3.79 g, 7.32 mmol) were then dissolved in DMSO (20 mL) and Cs₂CO₃ (4.92 g, 15.1 mmol) was added. The mixture was stirred for 1 h at ambient temperature and then heated at 45° C. for 4 h whereupon the mixture was cooled to ambient temperature and water (150 mL) was added. The aqueous suspension was extracted with a mixture of EtOAc and EtOH (200 mL) and the volatile components were removed under vacuum to give the title compound 18b (2 g, 5.6 mmol) in 66% yield which was carried on without further purification. ¹H NMR (300 MHz, DMSO-D6) δ ppm 7.02 (br s, 2H), 6.60 (d, J=7.7 Hz, 1H), 4.41 (br s, 2H), 4.26 (br s, 2H), 4.19-4.07 (m, 1H), 2.18-2.07 (m, 2H), 2.04-1.91 (m, 2H), 1.64-1.50 (m, 2H). LCMS (M+H)⁺360.2.

### Preparation of Compound 18c, 2,4-dichloro-6-hydroxyphenylboronic acid

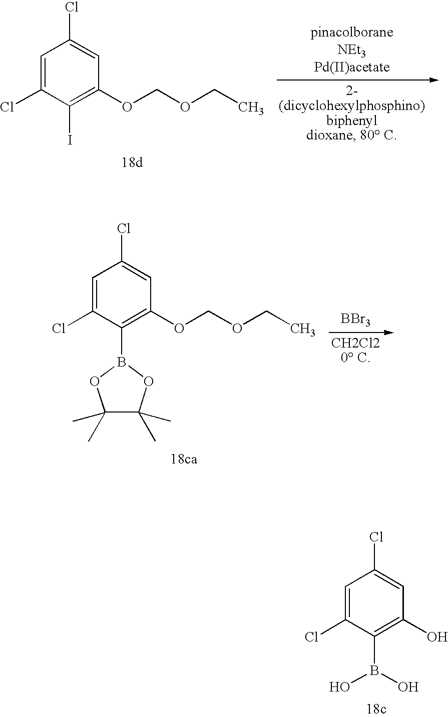

A solution of compound 18d (4.33 g, 12.5 mmol), pinacolborane (3.80 mL, 26.2 mmol), and triethylamine (5.2 mL, 37 mmol) in 1,4-dioxane (80 mL) was purged with N₂ for 15 minutes. Palladium (II) acetate (142 mg, 0.632 mmol) and 2-(dicyclohexylphosphino)biphenyl (439 mg, 1.25 mmol) were added and the mixture was warmed to 80° C. for 1.5 h. After cooling to room temperature, the mixture was poured into EtOAc (200 mL), and washed with saturated aqueous NH4Cl (100 mL) and water (200 mL). Following concentration under vacuum, the residue was purified on silica gel, eluting with a gradient of 10-70% CH₂Cl₂ in hexane. The purest fractions were combined and the volatile components removed under vacuum to give the compound 18ca, 2,4-dichloro-6-(ethoxymethoxy)phenylboronic acid pinacol ester, (1.55 g, 4.47 mmol) which was then dissolved in dichloromethane (50.0 mL) and cooled to 0° C. A 1.0 M solution of boron tribromide in CH₂Cl₂ (10.0 mL, 10.0 mmol) was added slowly over 5 minutes. After 15 minutes, the mixture was poured into ice water. The biphasic mixture was stirred vigorously and the pH of the aqueous phase was adjusted to about 10 with 3N NaOH. The layers were separated and the dichloromethane phase discarded. The pH of the aqueous phase was then adjusted to about 3 with 1N HCl and extracted with a mixture of EtOAc and iPrOH. The organic layer was then concentrated to give the title compound 18c (778 mg, 3.8 mmol) as a tan powder that was carried on without further purification.

### Preparation of Compound 18d, 1,5-dichloro-3-(ethoxymethoxy)-2-iodobenzene

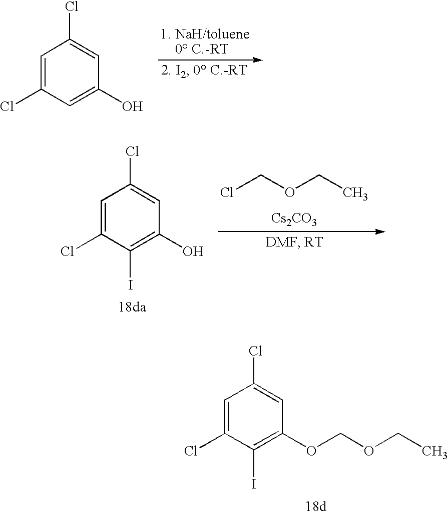

To a solution of 3,5-dichlorophenol (5.98 g, 36.7 mmol) in toluene (200 mL) at 0° C. was added NaH (60% in oil, 4.50 g, 117 mmol) in portions. The reaction mixture was then allowed to warm to ambient temperature and stir for 20 minutes. The suspension was then cooled back to 0° C., and iodine (7.91 g, 31.2 mmol) was added slowly. The mixture was allowed to warm to ambient temperature and stirred overnight. Aqueous 1N HCl (200 mL) was added, and the mixture was extracted twice with Et₂O (300 mL total). The combined organic extracts were concentrated under vacuum, and the residue was purified on silica gel, eluting with a gradient of 0-30% CH₂Cl₂ in hexane. The purest fractions were combined, and the volatile components removed under vacuum to give 3,5-dichloro-2-iodophenol, compound 18da, (6.53 g, 22.7 mmol) in 62% yield. Compound 18da (3.726 g, 12.90 mmol) in DMF (50 mL) was then treated with Cs₂CO₃ (3.45 g, 10.6 mmol) and chloromethylethyl ether (1.50 mL, 16.2 mmol). After 3 h at ambient temperature, Et₂O (150 mL) was added, and the organic layer was washed with water (3×100 mL). The organic layer was concentrated under vacuum and the residue was purified on silica gel eluting with a gradient of 0-40% CH₂Cl₂ in hexane. The purest fractions were combined and the volatile components removed under vacuum to give the title compound 18d (4.3 g, 12.5 mmol) as a clear oil. Estimated purity (by NMR) was 86%. ¹H NMR (300 MHz, CDCl₃) δ ppm 7.00 (d, J=1.6 Hz, 1H), 6.98 (d, J=1.5 Hz, 1H), 5.18 (s, 2H), 3.71 (q, J=7.2 Hz, 2H), 1.39 (s, 12H), 1.22 (t, J=7.2 Hz, 3H).

Compounds of Examples 20-98 were prepared following the methods of Examples 1-18, as shown in the following Table 1.

TABLE 1

| Ex. | | | Synthetic | | |
|---|---|---|---|---|---|
| No. | Structure | Name | Method | ¹H NMR | MS |
| 20 | 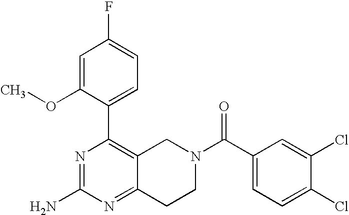 | 6-(3,4-dichlorobenzoyl)-4- (4-fluoro-2- methoxyphenyl)-5,6,7,8- tetrahydropyrido[4,3- d]pyrimidin-2-amine | Example 17 | (400 MHz, DMSO-D6) (conformers) δ ppm 7.80-7.57 (m, 2H), 7.46 (m, 1H), 7.27 (m, 1H), 7.11 (m, 1H), 6.89 (m, 1H), 6.59 (s, 2H), 4.29 br s, 2H), 3.82, br s, 3H), 3.64-3.37 (m, 2H), 2.85-2.73 (m, 2H). | 449.2, 447.2 |
| 22 | 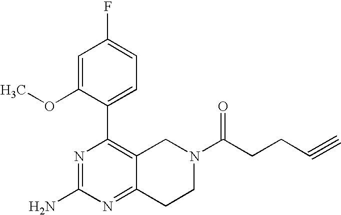 | 4-(4-fluoro-2- methoxyphenyl)-6-pent-4- ynoyl-5,6,7,8- tetrahydropyrido[4,3- d]pyrimidin-2-amine | Example 17 | (400 MHz, DMSO-D6) (conformers) δ ppm 7.22 (m, 1H), 7.08 (m, 1H), 6.90 (m, 1H), 6.52 (m, 2H), 4.15 (br s, 2H), 3.86-3.67 (m, 5H, contains 3.78 (s)), 2.84-2.49 (m, 5H, partially obscured), 2.45-2.27 (m, 2H). | 355.2 |
| 23 | 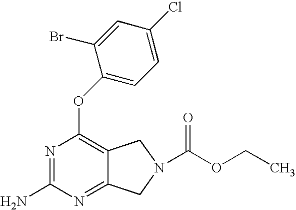 | ethyl 2-amino-4-(2-bromo- 4-chlorophenoxy)-5,7- dihydro-6H-pyrrolo[34- d]pyrimidine-6-carboxylate | Example 10 | (400 MHz, DMSO-D6) δ ppm 7.91 (s, 1H), 7.56 (m, 1H), 7.43 (m, 1H), 6.82 (br s, 2H), 4.55 (s, 1H), 4.52-4.42 (m, 3H), 4.19-4.09 (m, 2H), 1.24 (t, 3H). | 412.8, 414.8 |
| 24 | 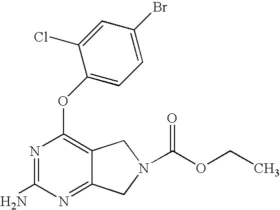 | ethyl 2-amino-4-(4-bromo- 2-chlorophenoxy)-5,7- dihydro-6H-pyrrolo[34- d]pyrimidine-6-carboxylate | Example 10 | (400 MHz, DMSO-D6) δ ppm 7.92 (s, 1H), 7.66 (d, 1H), 7.39 (d, 1H), 6.82 (br s, 2H), 4.54 (s, 1H), 4.50 (s, 1H), 4.44 (d, 2H), 4.18-4.09 (m, 2H), 1.25 (t, 3H). | 412.8, 414.8 |
| 25 | 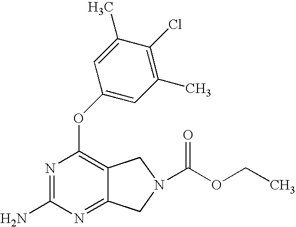 | ethyl 2-amino-4-(4-chloro- 35-dimethylphenoxy)-5,7- dihydro-6H-pyrrolo[34- d]pyrimidine-6-carboxylate hydrochloride | Example 10 | (400 MHz, DMSO-D6) δ ppm 7.09 (s, 2H), 4.50 (s, 1H), 4.46-4.42 (m, 3H), 4.17-4.10 (m, CH2, partially obscured), 2.35 (s, 6H), 1.25 (t, 3H). | 363.2 |
| 26 | 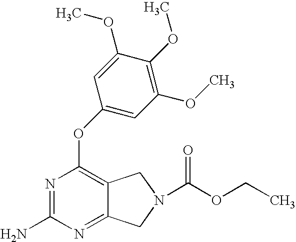 | ethyl 2-amino-4-(345- trimethoxyphenoxy)-5,7- dihydro-6H-pyrrolo[34- d]pyrimidine-6-carboxylate | Example 10 | (400 MHz, DMSO-D6) δ ppm 6.59 (s, 2H), 4.49-4.40 (m, 4H), 4.17-4.09 (m, 2H, partially obscured), 3.78 (s, 6H), 3.67 (s, 3H), 1.28-1.21 (m, 3H). | 391.2 |
| 27 | 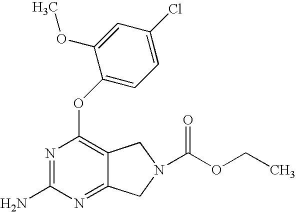 | ethyl 2-amino-4-(4-chloro- 2-methoxyphenoxy)-5,7- dihydro-6H-pyrrolo[3,4- d]pyrimidine-6-carboxylate | Example 10 | (400 MHz, DMSO-D6) δ ppm 7.30-7.18 (m, 2H), 7.05 (m, 1H), 6.71 (s, 2H), 4.49 9s, 1H), 4.43-4.38 (m, 3H), 4.17-4.08 (m, 2H), 3.77 (s, 3H), 1.23 (t, 3H). | 365.0 |
| 29 | 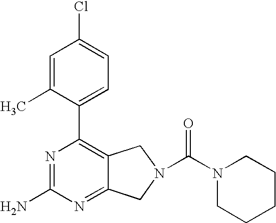 | 4-(4-chloro-2- methylphenyl)-6-(piperidin- 1-ylcarbonyl)-6,7-dihydro- 5H-pyrrolo[3,4- d]pyrimidin-2-amine | Example 7 | (300 MHz, DMSO-D6) δ ppm 7.42 (br s, 1H), 7.35-7.33 (m, 2H), 6.75 (br s, 2H), 4.52 (s, 2H), 4.39 (s, 2H), 3.18-3.15 (m, 4H), 2.24 (s, 3H), 1.56-1.39 (m, 6H). | 372.2 |
| 30 | 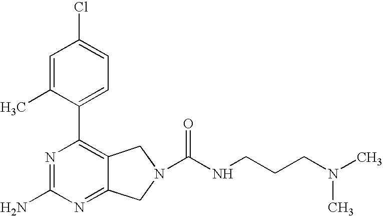 | 2-amino-4-(4-chloro-2- methylphenyl)-N-[3- (dimethylamino)propyl]- 5,7-dihydro-6H- pyrrolo[3,4-d]pyrimidine-6- carboxamide | Example 7 | (300 MHz, DMSO-D6) δ ppm 7.43 (br s, 1H), 7.35-7.32 (m, 2H), 6.77 (br s, 2H), 6.44 (t, J = 5.5 Hz, 1H), 4.40 (s, 2H), 4.24 (s, 2H), 3.07-3.00 (m, 2H), 2.23 (s, 3H), 2.18 (t, J = 7.1 Hz, 2H), 2.09 (s, 6H), 1.57-1.48 (m, 2H). | 389.2 |
| 31 | 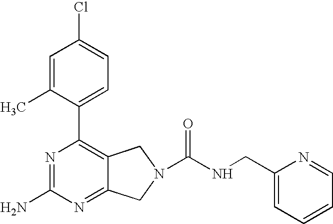 | 2-amino-4-(4-chloro-2- methylphenyl)-N-(pyridin- 2-ylmethyl)-5,7-dihydro- 6H-pyrrolo[3,4- d]pyrimidine-6- carboxamide | Example 7 | (300 MHz, DMSO-D6) δ ppm 8.48-8.44 (m, 1H), 7.72 (dt, J = 7.7 and 1.9 Hz, 1H), 7.43 (br s, 1H), 7.36-7.33 (m, 2H), 7.31 (d, J = 7.9 Hz, 1H), 7.25-7.18 (m, 1H), 7.07 (t, J = 5.9 Hz, 1H), 6.79 (br s, 2H), 4.48 (s, 2H), 4.36-4.31 (m, 4H), 2.24 (s, 3H). | 395.2 |
| 32 | 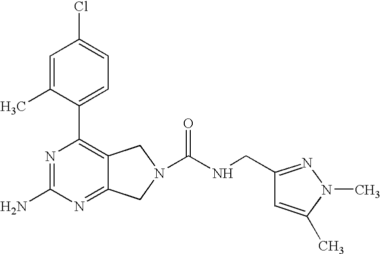 | 2-amino-4-(4-chloro-2- methylphenyl)-N-[(15- dimethyl-1H-pyrazol-3- yl)methyl]-5,7-dihydro-6H- pyrrolo[3,4-d]pyrimidine-6- carboxamide | Example 7 | (300 MHz, DMSO-D6) δ ppm 7.42 (br s, 1H), 7.34-7.32 (m, 2H), 6.77 (br s, 2H), 6.73 (t, J = 5.8 Hz, 1H), 5.88 (s, 1H), 4.42 (s, 2H), 4.26 (s, 2H), 4.08 (d, J = 5.7 Hz, 2H), 3.62 (s, 3H), 2.23 (s, 3H), 2.17 (s, 3H). | 412.2 |
| 33 | 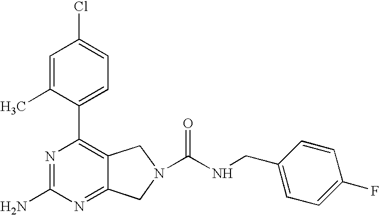 | 2-amino-4-(4-chloro-2- methylphenyl)-N-(4- fluorobenzyl)-5,7-dihydro- 6H-pyrrolo[3,4- d]pyrimidine-6- carboxamide | Example 7 | (300 MHz, DMSO-D6) δ ppm 7.42 (br s, 1H), 7.34-7.27 (m, 4H), 7.14-7.06 (m, 2H), 6.99 (t, J = 5.8 Hz, 1H), 6.78 (br s, 2H), 4.45 (s, 2H), 4.30 (s, 2H), 4.22 (d, J = 5.7 Hz, 2H), 2.23 (s, 3H). | 412.2 |
| 34 | 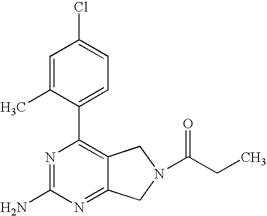 | 4-(4-chloro-2- methylphenyl)-6-propionyl- 6,7-dihydro-5H- pyrrolo[3,4-d]pyrimidin-2- amine | Example 6 | (300 MHz, DMSO-D6) δ ppm 7.43 (br s, 1H), 7.35-7.32 (m, 2H), 6.79 (br s, 2H), 6.79 (br s, 2H), 4.67 (s, 1H), 4.52 (s, 1H), 4.45 (s, 1H), 4.29 (s, 1H), 2.36-2.23 (m, 5H), 1.03-0.95 (m, 3H). | 317.2 |
| 35 | 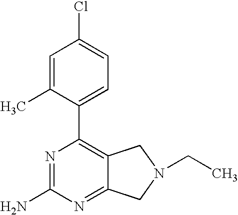 | 4-(4-chloro-2- methylphenyl)-6-ethyl-6,7- dihydro-5H-pyrrolo[3,4- d]pyrimidin-2-amine | Example 5 | (300 MHz, DMSO-D6) δ ppm 7.40 (d, J = 2.1 Hz, 1H), 7.31 (dd, J = 8.3 and 2.1 Hz, 1H), 7.25 (d, J = 8.3 Hz, 1H), 6.58 (br s, 2H), 3.70 (s, 2H), 3.58 (s, 2H), 2.65 (q, J = 7.2 Hz, 2H), 2.24 (s, 3H), 1.04 (t, J = 7.2 Hz, 3H). | 289.2 |
| 36 | 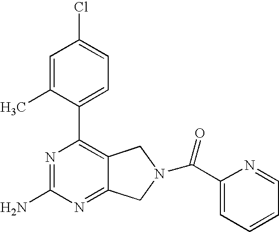 | 4-(4-chloro-2- methylphenyl)-6-(pyridin-2- ylcarbonyl)-6,7dihydro-5H- pyrrolo[3,4-d]pyrimidin-2- amine | Example 6 | (300 MHz, DMSO-D6) (conformers) δ ppm 8.68 (d, J = 4.3 Hz, 0.5H), 8.57 (d, J = 4.3 Hz, 0.5H), 8.01-7.91 (m, 1H), 7.88-7.78 (m, 1H), 7.58-7.37 (m, 3H), 7.29 (s, 1H), 6.82 (br s, 1H), 6.79 (br s, 1H), 4.99 (s, 1H), 4.80 (s, 1H), 4.74 (s, 1H), 4.60 (s, 1H), 2.27 (s, 1.5H), 2.22 (s, 1.5H). | 366.2 |
| 37 | 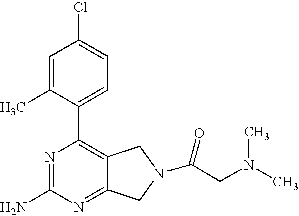 | 4-(4-chloro-2- methylphenyl)-6- [(dimethylamino)acetyl]- 6,7-dihydro-5H- pyrrolo[3,4-d]pyrimidin-2- amine | Example 6 | (300 MHz, DMSO-D6) (conformers) δ ppm 7.43 (m, 1H), 7.35-7.32 (m, 2H), 6.80 and 6.78 (2 overlapping br s, 2H), 4.75 (s, 1H), 4.58 (s, 1H), 4.46 (s, 1H), 4.32 (s, 1H), 3.12 (s, 1H), 3.08 (s, 1H), 2.25 (s, 1.5H), 2.24 (s, 1.5H), 2.23 (s, 3H), 2.18 (s, 3H). | 346.2 |
| 38 | 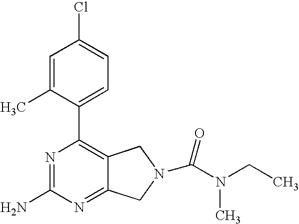 | 2-amino-4-(4-chloro-2- methylphenyl)-N-ethyl-N- methyl-5,7-dihydro-6H- pyrrolo[3,4-d]pyrimidine-6- carboxamide | Example 7 | (300 MHz, DMSO-D6) δ ppm 7.42 (s, 1H), 7.35-7.32 (m, 2H), 6.73 (br s, 2H), 4.51 (s, 2H), 4.38 (s, 2H), 3.15 (q, J = 7.0 Hz, 2H), 2.76 (s, 3H), 2.25 (s, 3H), 2.25 (s, 3H), 1.05 (t, J = 7.0 Hz, 3H). | 346.2 |
| 39 | 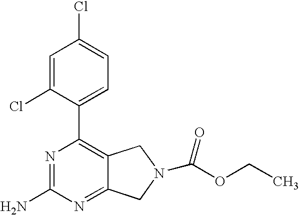 | ethyl 2-amino-4-(2,4- dichlorophenyl)-5,7- dihydro-6H-pyrrolo[3,4- d]pyrimidine-6-carboxylate | Example 1 | (300 MHz, DMSO-D6) (conformers) δ ppm 7.78 (d, J = 1.9 Hz, 1H), 7.56 (dd, J = 8.3 and 1.9 Hz, 1H), 7.48 (d, J = 8.3 Hz, 1H), 6.92 (br s, 2H), 4.49 (s, 1H), 4.47 (s, 1H), 4.34 (s, 1H), 4.31 (s, 1H), 4.13-4.03 (m, 2H), 1.24-1.15 (m, 3H). | 355.2, 353.2 |
| 40 | 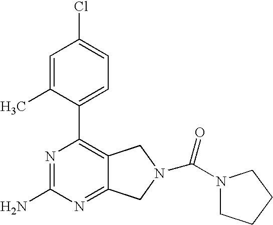 | 4-(4-chloro-2- methylphenyl)-6- (pyrrolidin-1-ylcarbonyl)- 6,7-dihydro-5H- pyrrolo[3,4-d]pyrimidin-2- amine | Example 7 | (300 MHz, DMSO-D6) δ ppm 7.42 (s, 1H), 7.35-7.32 (m, 2H), 6.73 (br s, 2H), 4.53 (s, 2H), 4.39 (s, 2H), 3.32-3.27 (m, 4H), 2.24 (s, 3H), 1.79-1.73 (m, 4H). | 358.2 |
| 41 | 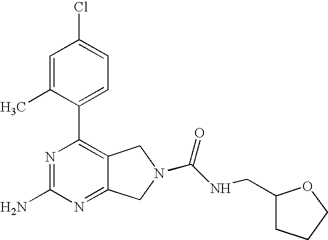 | 2-amino-4-(4-chloro-2- methylphenyl)-N- (tetrahydrofuran-2- ylmethyl)-5,7-dihydro-6H- pyrrolo[3,4-d]pyrimidine-6- carboxamide | Example 7 | (300 MHz, DMSO-D6) δ ppm 7.43 (br s, 1H), 7.35-7.32 (m, 2H), 6.75 (br s, 2H), 6.41 (t, J = 5.8 Hz, 1H), 4.41 (s, 2H), 4.26 (s, 2H), 3.87-3.80 (m, 1H), 3.76-3.69 (m, 1H), 3.62-3.54 (m, 1H), 3.07 (dt, J = 5.8 and 2.3 Hz, 2H), 2.23 (s, 3H), 1.88-1.72 (m, 3H), 1.58-1.50 (m, 1H). | 388.2 |
| 42 | 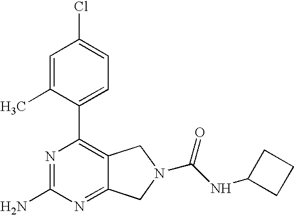 | 2-amino-4-(4-chloro-2- methylphenyl)-N- cyclobutyl-5,7-dihydro-6H- pyrrolo[3,4-d]pyrimidine-6- carboxamide | Example 7 | (300 MHz, DMSO-D6) δ ppm 7.43 (br s, 1H), 7.43 (br s, 1H), 7.35-7.32 (m, 2H), 6.75 (br s, 2H), 6.50 (d, J = 8.1 Hz, 1H), 4.40 (s, 2H), 4.24 (s, 2H), 4.18-4.08 (m, 1H), 2.22 (s, 3H), 2.14-2.03 (m, 2H), 1.96-1.89 (m, 2H), 1.59-1.50 (m, 2H). | 358.2 |
| 43 | 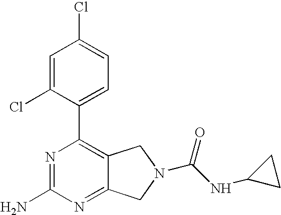 | 2-amino-N-cyclopropyl-4- (24-dichlorophenyl)-5,7- dihydro-6H-pyrrolo[3,4- d]pyrimidine-6- carboxamide | Example 7 | (300 MHz, DMSO-D6) δ ppm 7.79 (d, J = 1.9 Hz, 1H), 7.55 (dd, J = 8.3 and 2.0 Hz, 1H), 7.47 (d, J = 8.3 Hz, 1H), 6.87 (br s, 2H), 6.46 (d, J = 2.6 Hz, 1H), 4.40 (s, 2H), 4.22 (s, 2H), 0.57-0.50 (m, 2H), 0.41-0.36 (m, 2H). | 364.2, 366.2 |
| 44 | 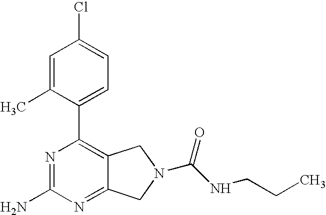 | 2-amino-4-(4-chloro-2- methylphenyl)-N-propyl- 5,7-dihydro-6H- pyrrolo[3,4-d]pyrimidine-6- carboxamide | Example 7 | (300 MHz, DMSO-D6) δ ppm 7.43 (br s, 1H), 7.34-7.32 (m, 2H), 6.77 (br s, 2H), 6.36 (t, J = 6.4 Hz, 1H), 4.40 (s, 2H), 4.24 (s, 2H), 2.98 (m, 2H), 2.23 (s, 3H), 1.47-1.34 (m, 2H), 0.82 (t, J = 7.4 Hz, 3H). | 346.2 |
| 45 | 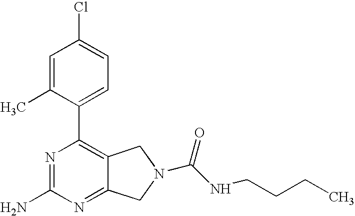 | 2-amino-N-butyl-4-(4- chloro-2-methylphenyl)- 5,7-dihydro-6H- pyrrolo[3,4-d]pyrimidine-6- carboxamide | Example 7 | (300 MHz, DMSO-D6) δ ppm 7.34-7.32 (m, 2H), 6.77 (br s, 2H), 6.32 (t, J = 5.5 Hz, 1H), 4.39 (s, 2H), 4.23 (s, 2H), 3.01 (m, 2H), 2.22 (s, 3H), 1.45-1.36 (m, 2H), 1.33-1.24 (m, 2H), 0.88 (t, J = 7.4 Hz, 3H | 360.2 |
| 46 | 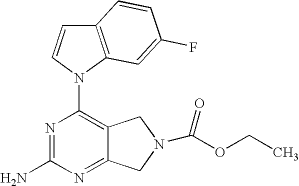 | ethyl 2-amino-4-(6-fluoro- 1H-indol-1-yl)-5,7-dihydro- 6H-pyrrolo[3,4- d]pyrimidine-6-carboxylate | Example 12 | (300 MHz, DMSO-D6) δ ppm 8.50-8.44 (m, 1H), 7.70-7.63 (m, 2H), 7.14-7.07 (m, 3H), 6.84 (d, J = 3.6 Hz, 1H), 4.82 (s, 1H), 4.79 (s, 1H), 4.47 (s, 1H), 4.44 (s, 1H), 4.13 (q, J = 7.2 Hz, 2H), 1.24 (t, J = 7.2 Hz, 3H) | 342.2 |
| 47 | 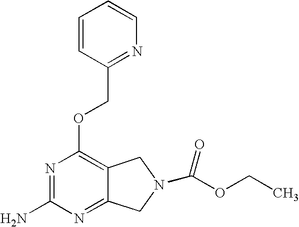 | ethyl 2-amino-4-(pyridin-2- ylmethoxy)-5,7-dihydro- 6H-pyrrolo[3,4- d]pyrimidine-6-carboxylate | Example 11 | (300 MHz, DMSO-D6) (conformers) δ ppm 8.56 (m, 1H), 7.82 (dt, J = 7.7 and 1.7 Hz, 1H), 7.47 (d, J = 7.9 Hz, 1H), 7.36-7.32 (m, 1H), 6.72 (br s, 2H), 5.46 (s, 1H), 5.45 (s, 1H), 4.46-4.34 (m, 4H), 4.14-4.05 (m, 2H), 1.25-1.18 (m, 3H). | 316.2 |
| 48 | 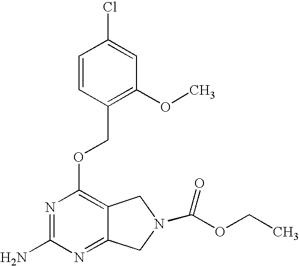 | ethyl 2-amino-4-[(4-chloro- 2-methoxybenzyl)oxy]-5,7- dihydro-6H-pyrrolo[3,4- d]pyrimidine-6-carboxylate | Example 11 | (300 MHz, DMSO-D6) (conformers) δ ppm 7.41 (d, J = 8.1 Hz, 1H), 7.13 (d, J = 2.0 Hz, 1H), 7.03 (dd, J = 8.1 and 1.8 Hz, 1H), 6.73 (br s, 2H), 5.32 (s, 1H), 5.31 (s, 1H), 4.40-4.32 (m, 4H), 4.12-4.05 (m, 2H), 3.84 (s, 3H), 1.23-1.19 (m, 3H). | 379.2 |
| 49 | 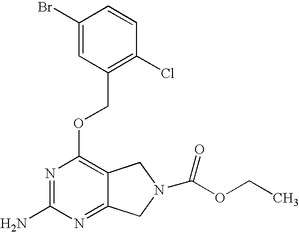 | ethyl 2-amino-4-[(5-bromo- 2-chlorobenzyl)oxy]-5,7- dihydro-6H-pyrrolo[3,4- d]pyrimidine-6-carboxylate | Example 11 | (300 MHz, DMSO-D6) (conformers) δ ppm 7.78-7.76 (m, 1H), 7.62-7.59 (m, 1H), 7.49 (d, J = 8.6, 1H), 6.79 (br s, 2H), 5.42 (s, 1H), 5.41 (s, 1H), 4.42-4.33 (m, 4H), 4.12-4.06 (m, 2H), 1.23-1.19 (m, 3H). | 427.0, 429.0 |
| 50 | 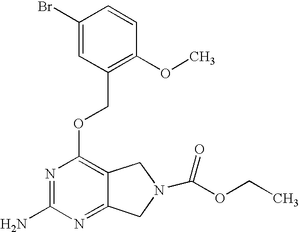 | ethyl 2-amino-4-[(5-bromo- 2-methoxybenzyl)oxy]-5,7- dihydro-6H-pyrrolo[3,4- d]pyrimidine-6-carboxylate | Example 11 | (300 MHz, DMSO-D6) (conformers) δ ppm 7.53-7.48 (m, 2H), 7.05-7.00 (m, 1H), 6.75 (br s, 2H), 5.33 (s, 1H), 5.32 (s, 1H), 4.41-4.32 (m, 4H), 4.13-4.05 (m, 2H), 3.81 (s, 3H), 1.24-1.19 (m, 3H). | 423.2, 425.2 |
| 51 | 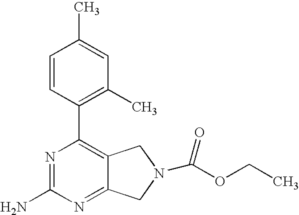 | ethyl 2-amino-4-(2,4- dimethylphenyl)-5,7- dihydro-6H-pyrrolo[3,4- d]pyrimidine-6-carboxylate | Example 1 | (300 MHz, DMSO-D6) (conformers) δ ppm 7.21-7.07 (m, 3H), 4.48 and 4.45 (2 overlapping br s, 2H), 4.32 and 4.29 (2 overlapping br s, 2H), 4.12-4.01 (m, 2H), 2.31 (s, 3H), 2.21 and 2.20 (2 overlapping s, 3H), 1.23-1.14 (m, 3H) | 313.2 |
| 52 | 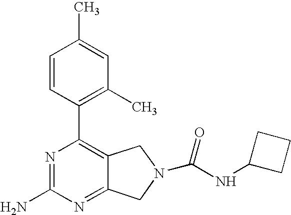 | 2-amino-N-cyclobutyl-4- (2,4-dimethylphenyl)-5,7- dihydro-6H-pyrrolo[3,4- d]pyrimidine-6- carboxamide | Example 7 | (300 MHz, DMSO-D6) δ ppm 7.17-7.06 (m, 3H), 6.67 (br s, 2H), 6.50 (d, J = 7.5 Hz, 1H), 4.38 (br s, 2H), 4.24 (br s, 2H), 4.16-4.08 (m, 1H), 2.31 (s, 3H), 2.19 (s, 3H), 2.13-2.04 (m, 2H), 1.99-1.85 (m, 2H), 1.58-1.48 (m, 2H). | 338.2 |
| 53 | 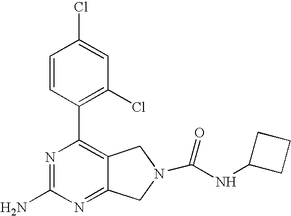 | 2-amino-N-cyclobutyl-4- (2,4-dichlorophenyl)-5,7- dihydro-6H-pyrrolo[3,4- d]pyrimidine-6- carboxamide | Example 3 | (300 MHz, DMSO-D6) δ ppm 7.80 (d, J = 2.0 Hz, 1H), 7.56 (dd, J = 8.1 and 2.0 Hz, 1H), 7.48 (d, J = 8.3 Hz, 1H), 6.89 (br s, 2H), 6.53 (d, J = 7.8 Hz, 1H), 4.40 (br s, 2H), 4.24 (br s, 2H), 4.17-4.07 (m, 1H), 2.12-2.05 (m, 2H), 1.97-1.86 (m, 2H), 1.58-1.48 (m, 2H) | 378 |
| 54 | 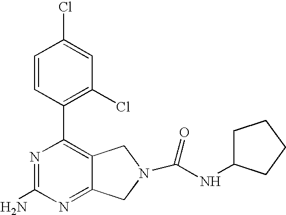 | 2-amino-N-cyclopentyl-4- (2,4-dichlorophenyl)-5,7- dihydro-6H-pyrrolo[3,4- d]pyrimidine-6- carboxamide | Example 15a | (300 MHz, DMSO-D6) δ ppm 7.80 (d, J = 2.0 Hz, 1H), 7.56 (dd, J = 8.3 and 2.0 Hz, 1H), 7.48 (d, J = 8.3 Hz, 1H), 6.88 (br s, 2H), 6.13 (d, J = 7.3 Hz, 1H), 4.42 (br s, 2H), 4.25 (br s, 2H), 3.97-3.88 (m, 1H), 1.81-1.73 (m, 2H), 1.65-1.57 (m, 2H), 1.50-1.34 (m, 4H) | 378.2, 380.2 |
| 55 | 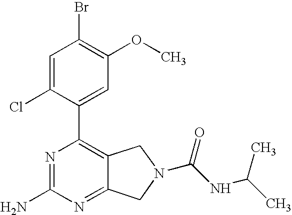 | 2-amino-4-(4-bromo-2- chloro-5-methoxyphenyl)- N-isopropyl-5,7-dihydro- 6H-pyrrolo[3,4- d]pyrimidine-6- carboxamide | Example 15a | (300 MHz, DMSO-D6) δ ppm 7.86 (s, 1H), 7.16 (s, 1H), 6.89 (br s, 2H), 6.07 (d, J = 7.7 Hz, 1H), 4.42 (br s, 2H), 4.25 (br s, 2H), 4.25 (br s, 2H), 3.86 (s, 3H), 3.82-3.75 (m, 1H), 1.05 (d, J = 6.4 Hz, 6H) | 440.2, 442.2 |
| 56 | 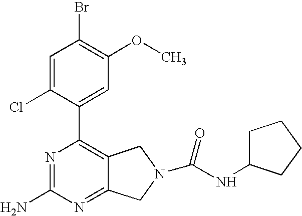 | 2-amino-4-(4-bromo-2- chloro-5-methoxyphenyl)- N-cyclopentyl-5,7-dihydro- 6H-pyrrolo[3,4- d]pyrimidine-6- carboxamide | Example 15a | (300 MHz, DMSO-D6) δ ppm 7.86 (s, 1H), 7.16 (s, 1H), 6.90 (br s, 2H), 6.13 (d, J = 7.5 Hz, 1H), 4.42 (s, 2H), 4.26 (s, 2H), 3.97-3.87 (m, 1H), 3.86 (s, 3H), 1.84-1.71 (m, 2H), 1.67-1.56 (m, 2H), 1.52-1.32 (m, 4H) | 466.2, 468.2 |
| 57 | 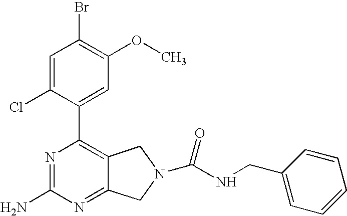 | 2-amino-N-benzyl-4-(4- bromo-2-chloro-5- methoxyphenyl)-5,7- dihydro-6H-pyrrolo[3,4- d]pyrimidine-6- carboxamide | Example 15a | (300 MHz, DMSO-D6) δ ppm 7.86 (s, 1H), 7.31-7.24 (m, 4H), 7.22-7.17 (m, 1H), 7.17 (s, 1H), 7.00 (t, J = 5.7 Hz, 1H), 6.91 (br s, 2H), 4.47 (s, 2H), 4.33 (s, 2H), 4.25 (d, J = 5.7 Hz, 2H), 3.86 (s, 3H) | 488.2, 490.2 |
| 58 | 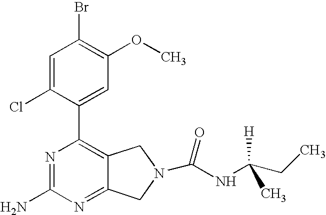 | 2-amino-4-(4-bromo-2- chloro-5-methoxyphenyl)- N-[(1R)-1-methylpropyl]- 5,7-dihydro-6H- pyrrolo[3,4-d]pyrimidine-6- carboxamide | Example 3 | (300 MHz, DMSO-D6) δ ppm 7.86 (s, 1H), 7.17 (s, 1H), 6.90 (br s, 2H), 6.00 (d, J = 7.8 Hz, 1H), 4.43 (s, 2H), 4.27 (s, 2H), 3.86 (s, 3H), 3.62-3.49 (m, overlaps with H2O peak, 1H), 1.46-1.31 (m, 2H), 1.02 (d, J = 6.6 Hz, 3H), 0.81 (t, J = 7.3 Hz, 3H) | 454.2, 456.2 |
| 59 | 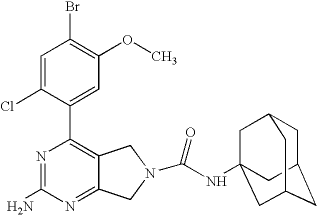 | N-1-adamantyl-2-amino-4- (4-bromo-2-chloro-5- methoxyphenyl)-5,7- dihydro-6H-pyrrolo[3,4- d]pyrimidine-6- carboxamide | Example 15a | (300 MHz, DMSO-D6) δ ppm 7.86 (s, 1H), 7.16 (s, 1H), 6.97 (br s, 2H), 5.45 (s, 1H), 4.41 (s, 2H), 4.26 (s, 2H), 3.86 (s, 3H), 1.99 (br s, 3H), 1.94 (br s, 6H), 1.60 (br s, 6H) | 532.2, 534.2 |
| 60 | 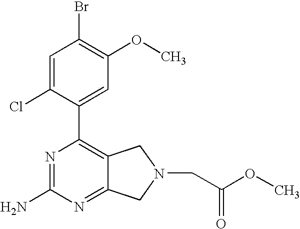 | methyl [2-amino-4-(4- bromo-2-chloro-5- methoxyphenyl)-57- dihydro-6H-pyrrolo[34- d]pyrimidin-6-yl]acetate | Example 5 | (300 MHz, CDCl3) δ ppm 7.66 (s, 1H), 6.98 (s, 1H), 5.76 (br s, 2H), 4.45 (br s, 2H), 4.38 (br s, 2H), 3.97 (s, 3H), 3.88 (s, 2H), 3.82 (s, 3H) | 427.2, 429.2 |
| 61 | 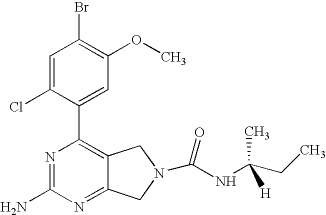 | 2-amino-4-(4-bromo-2- chloro-5-methoxyphenyl)- N-[(1S)-1- methylpropyl]5,7-dihydro- 6H-pyrrolo[3,4- d]pyrrolo[3,4-d]pyrimidine- 6-carboxamide | Example 3 | (300 MHz, DMSO-D6) δ ppm 7.86 (s, 1H), 7.17 (s, 1H), 6.89 (br s, 2H), 6.00 (d, J = 8.3 Hz, 1H), 4.42 (s, 2H), 4.26 (s, 2H), 3.86 (s, 3H), 3.64-3.51 (m, overlaps with H2O peak, 1H), 1.47-1.30 (m, 2H), 1.02 (d, J = 6.4 Hz, 3H), 0.80 (t, J = 7.3 Hz, 3H). | 454.2, 456.2 |
| 62 | 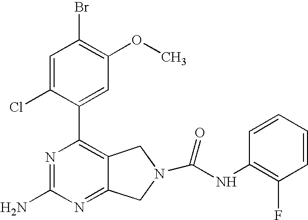 | 2-amino-4-(4-bromo-2- chloro-5-methoxyphenyl)- N-(2-fluorophenyl)-5,7- dihydro-6H-pyrrolo[3,4- d]pyrimidine-6- carboxamide | Example 15a | (300 MHz, DMSO-D6) δ ppm 8.18 (s, 1H), 7.88 (s, 1H), 7.50-7.44 (m, 1H), 7.23-7.10 (m, 4H), 6.95 (br s, 2H), 4.61 (br s, 2H), 4.48 (br s, 2H), 3.88 (s, 3H). | 492.2, 494.2 |
| 63 | 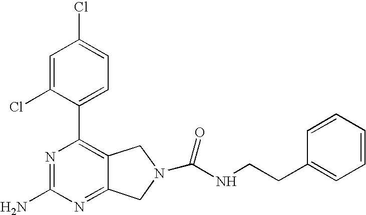 | 2-amino-4-(2,4- dichlorophenyl)-N-(2- phenylethyl)-5,7-dihydro- 6H-pyrrolo[3,4- d]pyrimidine-6- carboxamide | Example 15a | (300 MHz, DMSO-D6) δ ppm 7.80 (s, 1H), 7.57 (d, J = 8.3 Hz, 1H), 7.49 (d, J = 8.1 Hz, 1H), 7.32-7.26 (m, 2H), 7.23-7.15 (m, 3H), 6.90 (br s, 2H), 6.52 (m, 1H), 4.42 (br s, 2H), 4.25 (br s, 2H), 3.32-3.19 (m, overlaps with H2O peak, 2H), 2.72 (t, J = 7.3 Hz, 2H). | 428.2, 430.2 |
| 64 | 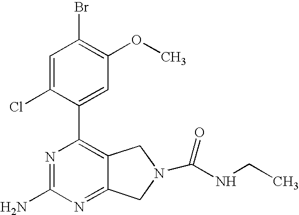 | 2-amino-4-(4-bromo-2- chloro-5-methoxyphenyl)- N-ethyl-5,7-dihydro-6H- pyrrolo[3,4-d]pyrimidine-6- carboxamide | Example 15a | (300 MHz, DMSO-D6) δ ppm 7.86 (s, 1H), 7.17 (s, 1H), 6.90 (br s, 2H), 6.36 (t, J = 5.2 Hz, 1H), 4.42 (br s, 2H), 4.26 (br s, 2H), 3.86 (s, 3H), 3.10-3.01 (m, 2H), 1.01 (t, J = 7.2 Hz, 3H). | 426.2, 428.2 |
| 65 | 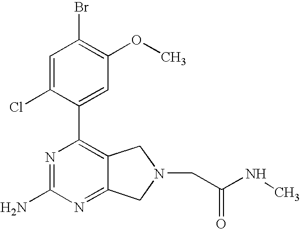 | 2-[2-amino-4-(4-bromo-2- chloro-5-methoxyphenyl)- 5,7-dihydro-6H-pyrrolo[34- d]pyrimidin-6-yl]-N- methylacetamide | Example 5 | (300 MHz, DMSO-D6) δ ppm 7.82 (s, 1H), 7.81-7.75 (m, 1H), 7.07 (s, 1H), 6.76 (br s, 2H), 6.76 (br s, 2H), 3.85 (s, 5H), 3.73 (s, 2H), 3.28 (s, 2H), 2.57 (d, J = 4.7 Hz, 3H). | 426.2, 428.2 |
| 66 | 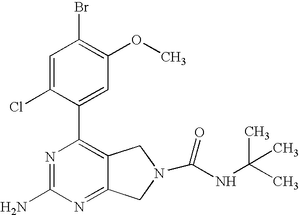 | 2-amino-4-(4-bromo-2- chloro-5-methoxyphenyl)- N-(tert-butyl)-5,7-dihydro- 6H-pyrrolo[3,4- d]pyrimidine-6- carboxamide | Example 15a | (300 MHz, DMSO-D6) δ ppm 7.86 (s, 1H), 7.16 (s, 1H), 6.88 (br s, 2H), 5.58 (s, 1H), 4.42 (s, 2H), 4.26 (s, 2H), 3.86 (s, 3H), 1.26 (s, 9H). | 454.2, 456.2 |
| 67 | 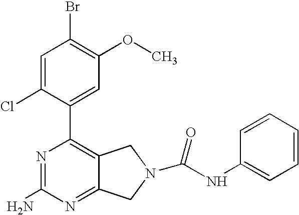 | 2-amino-4-(4-bromo-2- chloro-5-methoxyphenyl)- N-phenyl-5,7-dihydro-6H- pyrrolo[3,4-d]pyrimidine-6- carboxamide | Example 15a | (300 MHz, DMSO-D6) δ ppm 8.34 (s, 1H), 7.89 (s, 1H), 7.51 (d, J = 7.9 Hz, 2H), 7.26-7.19 (m, 3H), 6.98-6.91 (m, 3H), 4.60 (s, 2H), 4.48 (s, 2H), 3.88 (s, 3H). | 474.2, 476.2 |
| 68 | 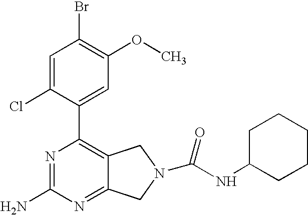 | 2-amino-4-(4-bromo-2- chloro-5-methoxyphenyl)- N-cyclohexyl-5,7-dihydro- 6H-pyrrolo[3,4- d]pyrimidine-6- carboxamide | Example 15a | (300 MHz, DMSO-D6) δ ppm 7.86 (s, 1H), 7.16 (s, 1H), 6.89 (br s, 2H), 6.07 (d, J = 7.9 Hz, 1H), 4.42 (s, 2H), 4.25 (s, 2H), 3.49-3.44 (m, under H2O peak, 1H), 3.86 (s, 3H), 1.78-1.00 (M, 10H), | 480.2, 482.2 |
| 69 | 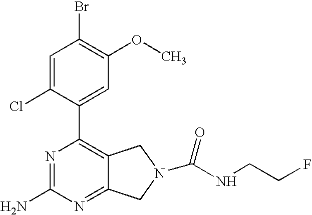 | 2-amino-4-(4-bromo-2- chloro-5-methoxyphenyl)- N-(2-fluoroethyl)-5,7- dihydro-6H-pyrrolo[3,4- d]pyrimidine-6- carboxamide | Example 3 | (300 MHz, DMSO-D6) δ ppm 7.87 (s, 1H), 7.17 (s, 1H), 6.91 (br s, 2H), 6.65 (t, J = 5.4 Hz, 1H), 4.50-4.43 (m, 3H), 4.35-4.28 (m, 3H), 3.86 (s, 3H), 3.36-3.25 (m, under H2O peak, 2H). | 444.2, 446.2 |
| 70 | 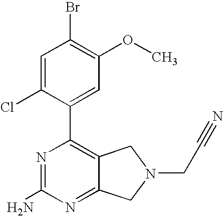 | [2-amino-4-(4-bromo-2- chloro-5-methoxyphenyl)- 5,7-dihydro-6H- pyrrolo[3,4-d]pyrimidin-6- yl]acetonitrile | Example 5 | (300 MHz, DMSO-D6) δ ppm 7.84 (s, 1H), 7.14 (s, 1H), 6.87 (br s, 2H), 3.96 (s, 2H), 3.86 (s, 3H), 3.85 (s, 2H), 3.74 (s, 2H). | 394.2, 396.2 |
| 71 | 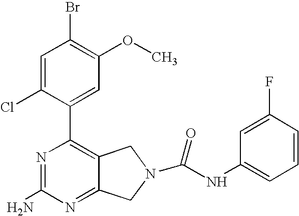 | 2-amino-4-(4-bromo-2- chloro-5-methoxyphenyl)- N-(3-fluorophenyl)-57- dihydro-6H-pyrrolo[34- d]pyrimidine-6- carboxamide | Example 15a | (300 MHz, DMSO-D6) δ ppm 8.54 (s, 1H), 7.88 (s, 1H), 7.50 (dt, J = 12.2 and 1.9 Hz, 1H), 7.33-7.21 (m, 2H), 7.19 (s, 1H), 6.95 (br s, 2H), 6.78-6.72 (m, 1H), 4.60 (s, 2H), 4.48 (s, 2H), 3.87 (s, 3H). | 492.2, 494.2 |
| 72 | 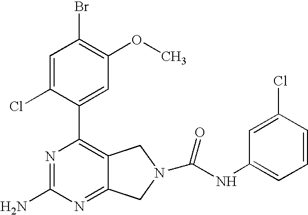 | 2-amino-4-(4-bromo-2- chloro-5-methoxyphenyl)- N-(3-chlorophenyl)-5,7- dihydro-6H-pyrrolo[3,4- d]pyrimidine-6- carboxamide | Example 15a | (300 MHz, DMSO-D6) δ ppm 8.52 (s, 1H), 7.88 (s, 1H), 7.70 (t, J = 1.8 Hz, 1H), 7.48 (d, J = 7.5 Hz, 1H), 7.25 (t, J = 8.10 Hz, 1H), 7.19 (s, 1H), 7.01-6.94 (m, 3H), 4.60 (s, 2H), 4.47 (s, 2H), 3.87 (s, 3H). | 508.0, 510.2, 512.0 |
| 73 | 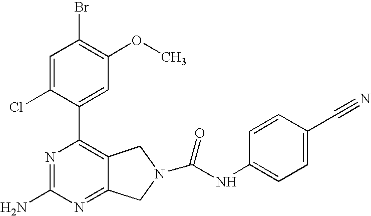 | 2-amino-4-(4-bromo-2- chloro-5-methoxyphenyl)- N-(4-cyanophenyl)-5,7- dihydro-6H-pyrrolo[3,4- d]pyrimidine-6- carboxamide | Example 15a | (300 MHz, DMSO-D6) δ ppm 8.80 (s, 1H), 7.89 (s, 1H), 7.76 (d, J = 8.9 Hz, 2H), 7.69 (d, J = 8.9 Hz, 2H), 7.19 (s, 1H), 6.97 (br s, 2H), 4.63 (s, 2H), 4.51 (s, 2H), 3.87 (s, 3H). | 499.2, 501.2 |
| 74 | 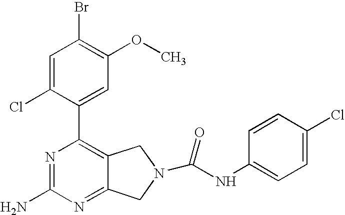 | 2-amino-4-(4-bromo-2- chloro-5-methoxyphenyl)- N-(4-chlorophenyl)-5,7- dihydro-6H-pyrrolo[3,4- d]pyrimidine-6- carboxamide | Example 15a | (300 MHz, DMSO-D6) δ ppm 8.48 (s, 1H), 7.89 (s, 1H), 7.56 (d, J = 8.9 Hz, 2H), 7.29 (d, J = 8.9 Hz, 2H), 7.19 (s, 1H), 6.95 (br s, 2H), 4.60 (s, 2H), 4.47 (s, 2H), 3.87 (s, 3H). | 508.0, 510.0, 512.0 |
| 75 | 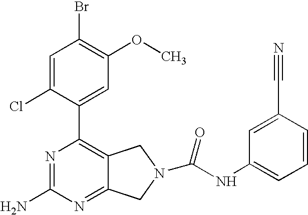 | 2-amino-4-(4-bromo-2- chloro-5-methoxyphenyl)- N-(3-cyanophenyl)-5,7- dihydro-6H-pyrrolo[3,4- d]pyrimidine-6- carboxamide | Example 15a | (300 MHz, DMSO-D6) δ ppm 8.69 (s, 1H), 8.01-7.99 (m, 1H), 7.89 (s, 1H), 7.85-7.80 (m, 1H), 7.49-7.38 (m, 2H), 7.20 (s, 1H), 6.97 (br s, 2H), 4.61 (s, 2H), 4.49 (s, 2H), 3.88 (s, 3H). | 499.2, 501.2 |
| 76 | 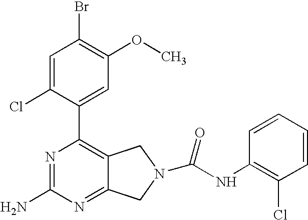 | 2-amino-4-(4-bromo-2- chloro-5-methoxyphenyl)- N-(2-chlorophenyl)-5,7- dihydro-6H-pyrrolo[3,4- d]pyrimidine-6- carboxamide | Example 15a | (300 MHz, DMSO-D6) δ ppm 8.07 (s, 1H), 7.87 (s, 1H), 7.55 (d, J = 7.5 Hz, 1H), 7.46 (d, J = 7.4 Hz, 1H), 7.29 (t, J = 7.3 Hz, 1H), 7.20 (s, 1H), 7.15 (t, J = 7.5 Hz, 1H), 6.95 (br s, 2H), 4.61 (s, 2H), 4.49 (s, 2H), 3.87 (s, 3H). | 508.0, 510.0, 512.0 |
| 77 | 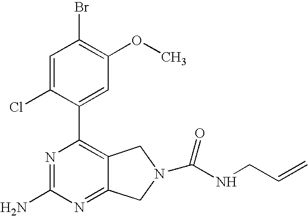 | N-allyl-2-amino-4-(4- bromo-2-chloro-5- methoxyphenyl)- 5,7 dihydro-6H- pyrrolo[3,4-d]pyrimidine-6- carboxamide | Example 15a | (300 MHz, DMSO-D6) δ ppm 7.86 (s, 1H), 7.17 (s, 1H), 6.92 (br s, 2H), 6.59 (t, J = 5.4 Hz, 1H), 5.87-5.74 (m, 1H), 5.12-4.97 (m, 2H), 4.45 (s, 2H), 4.30 (s, 2H), 3.86 (s, 3H), 3.67 (m, 2H). | 438.2, 440.2 |
| 78 | 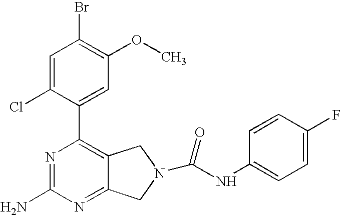 | 2-amino-4-(4-bromo-2- chloro-5-methoxyphenyl)- N-(4-fluorophenyl)-5,7- dihydro-6H-pyrrolo[3,4- d]pyrimidine-6- carboxamide | Example 15a | (300 MHz, DMSO-D6) δ ppm 8.40 (s, 1H), 7.88 (s, 1H), 7.54-7.47 (M, 2H), 7.19 (s, 1H), 7.07 (t, J = 8.7 Hz, 2H), 6.95 (br s, 2H), 4.59 (s, 2H), 4.46 (s, 2H), 3.87 (s, 3H). | 492.2, 494.2 |
| 79 | 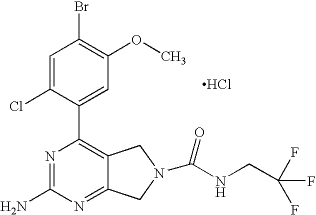 | 2-amino-4-(4-bromo-2- chloro-5-methoxyphenyl)- N-(2,2,2-trifluoroethyl)-5,7- dihydro-6H-pyrrolo[3,4- d]pyrimidine-6- carboxamide hydrochloride | Example 3 | (300 MHz, DMSO-D6) δ ppm 7.86 (s, 1H), 7.17 (s, 1H), 7.09 (t, J = 6.0 Hz, 1H), 6.93 (br s, 2H), 4.47 (s, 2H), 4.32 (s, 2H), 3.86 (s, 3H), 3.86-3.76 (m, 2H). | 480.2, 482.2 |
| 80 | 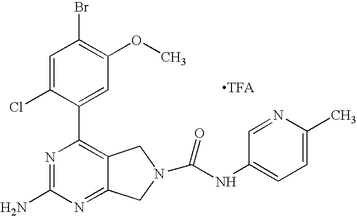 | 2-amino-4-(4-bromo-2- chloro-5-methoxyphenyl)- N-(6-methylpyridin-3-yl)- 5,7-dihydro-6H- pyrrolo[3,4-d]pyrimidine-6- carboxamide trifluoroacetate | Example 3 | (300 MHz, DMSO-D6) δ ppm 8.98 (s, 1H), 8.84 (s, 1H), 8.23 (d, J = 8.5 Hz, 1H), 7.90 (s, 1H), 7.61 (d, J = 8.3 Hz, 1H), 7.20 (s, 1H), 6.99 (br s, 2H), 4.63 (s, 2H), 4.51 (s, 2H), 3.88 (s, 3H), 2.55 (s, 3H). | 489.2, 491.2 |
| 81 | 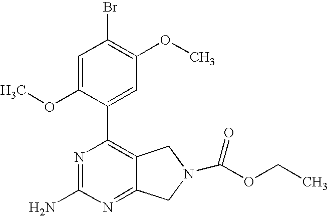 | ethyl 2-amino-4-(4-bromo- 2,5-dimethoxyphenyl)-5,7- dihydro-6H-pyrrolo[3,4- d]pyrimidine-6-carboxylate | Example 1 | (300 MHz, DMSO-D6) (conformers) δ ppm 7.39 (s, 1H), 7.05 (s, 1H), 6.82 (br s, 2H), 4.45 (br s, 1H), 4.43 (br s, 1H), 4.31 (br s, 1H), 4.29 (br s, 1H), 4.12-4.02 (m, 2H), 3.79 (s, 3H), 3.77 (s, 3H), 1.23-1.15 (m, 3H). | 423.2, 425.2 |
| 82 | 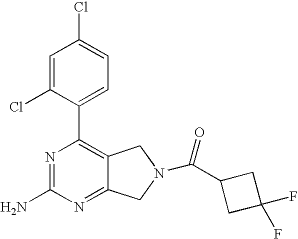 | 4-(2,4-dichlorophenyl)-6- [3,3- difluorocyclobutyl) carbonyl]-6,7-dihydro-5H- pyrrolo[3,4-d]pyrimidin-2- amine | Example 6 | (400 MHz, DMSO-d6) δ ppm 2.68-2.88 (m, 4 H) 3.14-3.28 (m, 1 H) 4.35 (s, 1 H) 4.52 (d, J = 14.40 Hz, 2 H) 4.69 (s, 1 H) 6.95 (s, 2 H) 7.44-7.52 (m, 1 H) 7.53-7.61 (m, 1 H) 7.80 (d, J = 1.77 Hz, 1 H). | 399, 401 |
| 83 | 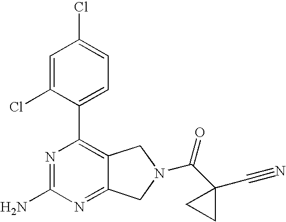 | 1-{[2-amino-4-(2,4- dichlorophenyl)- 5,7-dihydro-6H- pyrrolo[3,4-d]pyrimidin-6- yl]carbonyl} cyclopropanecarbonitrile | Example 6 | (400 MHz, DMSO-d6) δ ppm 1.47-1.67 (m, 4 H) 4.49 (d, J = 58.86 Hz, 2 H) 4.94 (d, J = 43.96 Hz, 2 H) 7.00 (s, 2 H) 7.42-7.63 (m, 2 H) 7.81 (dd, J = 17.94, 2.02 Hz, 1 H). | 374, 376 |
| 84 | 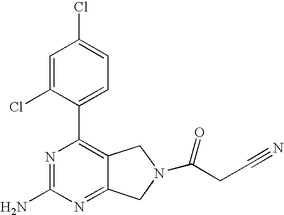 | 3-[2-amino-4-(2,4- dichlorophenyl)-5,7- dihydro-6H-pyrrolo[3,4- d]pyrimidin-6-yl]-3- oxopropanenitrile | Example 6 | (400 MHz, DMSO-d6) δ ppm 4.03 (d, J = 27.79 Hz, 2 H) 4.35 (s, 1 H) 4.51 (d, J = 3.54 Hz, 2 H) 4.68 (s, 1 H) 6.99 (s, 2 H) 7.48 (dd, J = 8.34, 6.32 Hz, 1 H) 7.52-7.61 (m, 1 H) 7.80 (dd, J = 7.33, 2.02 Hz, 1 H). | 348, 350 |
| 85 | 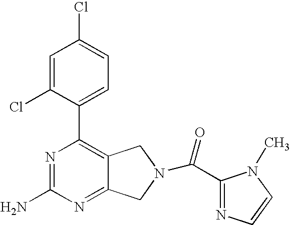 | 4-(2,4-dichlorophenyl)-6- [(1-methyl-1H-imidazol-2- yl)carbonyl]-6,7-dihydro- 5H-pyrrolo[3,4- d]pyrimidin-2-amine | Example 6 | (400 MHz, DMSO-d6) δ ppm 3.86 (d, J = 1.77 Hz, 3 H) 4.62 (d, J = 61.39 Hz, 2 H) 5.04 (d, J = 61.64 Hz, 2 H) 6.94 (s, 2 H) 7.03 (dd, J = 34.61, 1.01 Hz, 1 H) 7.35 (d, J = 14.91 Hz, 1 H) 7.44-7.52 (m, 1 H) 7.54-7.62 (m, 1 H) 7.80 (d, J = 1.77 Hz, 1 H). | 389, 391 |
| 86 | 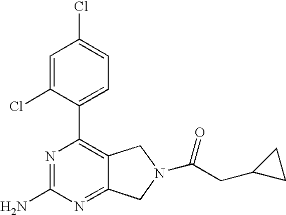 | 6-(cyclopropylacetyl)-4- (2,4-dichlorophenyl)-6,7- dihydro-5H-pyrrolo[3,4- d]pyrimidin-2-amine | Example 6 | (400 MHz, DMSO-d6) δ ppm 0.01-0.18 (m, 2 H) 0.36-0.52 (m, 2 H) 0.99 (dd, J = 7.33, 4.80 Hz, 1 H) 2.25 (dd, J = 30.06, 6.57 Hz, 2 H) 4.31 (s, 1 H) 4.48 (d, J = 9.85 Hz, 2 H) 4.67 (s, 1 H) 6.93 (s, 2 H), 7.43-7.51 (m, 1 H) 7.52-7.61 (m, 1 H) 7.79 (d, J = 1.26 Hz, 1 H). | 363, 365 |
| 87 | 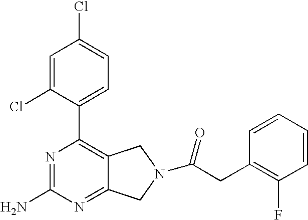 | 4-(2,4-dichlorophenyl)-6- [(2-fluorophenyl)acetyl]- 6,7-dihydro-5H- pyrrolo[3,4-d]pyrimidin-2- amine | Example 6 | (400 MHz, DMSO-d6) δ ppm 3.77 (d, J = 32.34 Hz, 2 H) 4.35 (s, 1 H) 4.51 (s, 1 H) 4.70 (s, 1 H) 4.86 (s, 1 H) 6.97 (s, 2 H) 7.08-7.21 (m, 2 H) 7.24-7.37 (m, 2 H) 7.46-7.63 (m, 2 H) 7.80 (dd, J = 9.85, 1.77 Hz, 1 H). | 417, 419 |
| 88 | 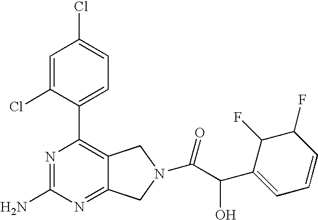 | 2-[2-amino-4-(2,4- dichlorophenyl)-5,7- dihydro-6H-pyrrolo[3,4- d]pyrimidin-6-yl]-1-(2,3- difluorophenyl)-2- oxoethanol | Example 6 | (400 MHz, DMSO-d6) δ ppm 4.44-4.60 (m, 2 H) 4.75-5.02 (m, 2 H) 5.59 (dd, J = 26.02, 7.07 Hz, 1 H) 6.18 (dd, J = 6.82, 2.27 Hz, 1 H), 6.96 (d, J = 8.59 Hz, 2 H) 7.16-7.33 (m, 2 H) 7.33-7.44 (m, 1 H) 7.44-7.54 (m, 1 H) 7.53-7.61 (m, 1 H) 7.79 (dd, J = 3.03, 2.02 Hz, 1 H). | 451, 453 |
| 89 | 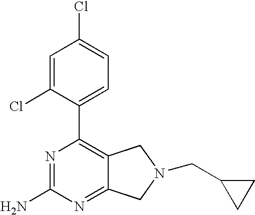 | 6-(cyclopropylmethyl)-4- (2,4-dichlorophenyl)-6,7- dihydro-5H-pyrrolo[3,4- d]pyrimidin-2-amine | Example 5 | (400 MHz, MeOH-d4) δ ppm 0.29 (d, J = 6.06 Hz, 2 H) 0.55-0.69 (m, 2 H) 0.93-1.10 (m, 1 H) 2.84 (d, J = 7.07 Hz, 2 H) 4.04 (s, 2 H) 4.14 (s, 2 H) 7.35-7.54 (m, 2 H) 7.64 (d, J = 1.77 Hz, 1 H). | 335, 337 |
| 90 | 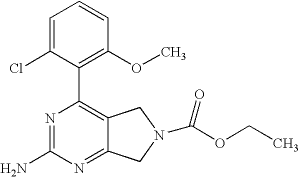 | ethyl 2-amino-4-(2-chloro- 6-methoxyphenyl)-5,7- dihydro-6H-pyrrolo[3,4- d]pyrimidine-6-carboxylate | Example 1 | (400 MHz, DMSO-D6) δ ppm 7.45 (dd, 1H, J = 8.4, 8.3 Hz), 7.16-7.12 (m, 2H), 6.84 (br s, 2H), 4.55-4.45 (m, 2H), 4.20-3.98 (m, 4H), 3.74 (s, 3H), 1.27-1.12 (m, 3H) | 349.2 351.2 |
| 91 | 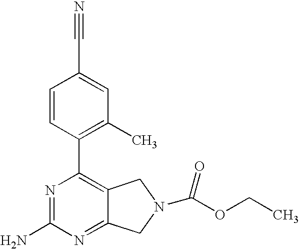 | ethyl 2-amino-4-(4-cyano- 2-methylphenyl)-5,7- dihydro-6H-pyrrolo[3,4- d]pyrimidine-6-carboxylate | Example 1 | (400 MHz, DMSO-D6) δ ppm 7.84 (s, 1H), 7.76 (d, 1H, J = 7.9 Hz), 7.68-7.48 (m, 1H), 6.89 (br s, 2H), 4.55-4.40 (m, 2H), 4.35-4.20 (m, 2H), 4.15-3.95 (m, 2H), 2.26 (s, 3H), 1.27-1.12 (m, 3H) | 324 |
| 92 | 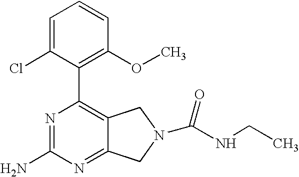 | 2-amino-4-(2-chloro-2- methylphenyl)-57-dihydro- 6H-pyrrolo[34- d]pyrimidine-6-carboxylate | Example 1 | (400 MHz, DMSO-D6) δ ppm 7.44 (dd, 1H, J = 8.4, 8.3 Hz), 7.16-7.12 (m, 2H), 6.79 (br s, 2H), 6.36 (t, 1H), 4.42 (br s, 2H), 4.10 (br s, 2H), 3.74 (s, 3H), 3.08-2.95 (m, 2H), 1.00 (t, 3H) | 348.2, 350.2 |
| 93 | 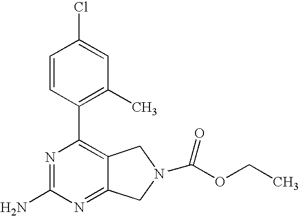 | ethyl 2-amino-4-(4-chloro- 2-methylphenyl)-57- dihydro-6H-pyrrolo[34- d]pyrimidine-6-carboxylate | Example 1 | (400 MHz, DMSO-D6) δ ppm 7.43 (s, 1H), 7.34 (s, 2H), 6.82 (bs, 2H), 4.47 (d, 2H, J = 8), 4.32 (d, 2H, J = 12 Hz), 4.07 (m, 2H), 2.24 (s, 3H), 1.22 (t, 3H, J = 7.1 Hz). | 333.2, 335.2 |
| 94 | 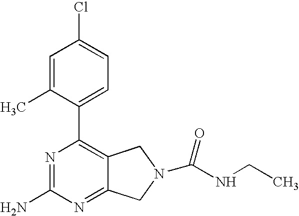 | 2-amino-4-(4-chloro-2- methylphenyl)-N-ethyl-57- dihydro-6H-pyrrolo[34- d]pyrimidine-6- carboxamide | Example 15a | (400 MHz, CD3OD) δ ppm 7.27 (s, 1H), 7.20 (s, 2H), 4.44 (s, 2H), 4.27 (s, 2H), 3.11 (m, 2H), 2.17 (s, 3H), 1.01 (t, 3H, 8 Hz) | 332.4, 334.2 |
| 95 | 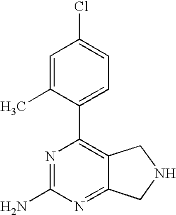 | 4-(4-chloro-2- methylphenyl)-67-dihydro- 5H-pyrrolo[34-d]pyrimidin- 2-amine | Example 2 | (400 MHz, DMSO-D6) δ ppm 7.41 (s, 1H), 7.32 (d, 1H, J = 8 Hz), 7.26 (d, 1H, J = 8 hz), 6.56 (bs, 2H), 3.88 (s, 2H), 3.80 (s, 2H), 2.24 (s, 3H) | 261 |
| 96 | 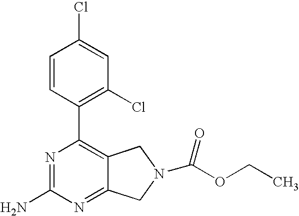 | ethyl 2-amino-4-(2,4- dichlorophenyl)-5,7- dihydro-6H-pyrrolo[3,4- d]pyrimidine-6-carboxylate | Example 1 | (300 MHz, DMSO-D6) (conformers) δ ppm 7.78 (d, J = 1.9 Hz, 1H), 7.56 (dd, J = 8.3 and 1.9 Hz, 1H), 7.48 (d, J = 8.3 Hz, 1H), 6.92 (br s, 2H), 4.49 (s, 1H), 4.47 (s, 1H), 4.34 (s, 1H), 4.31 (s, 1H), 4.13-4.03 (m, 2H), 1.24-1.15 (m, 3H). | 355.2, 353.2 |
| 97 | 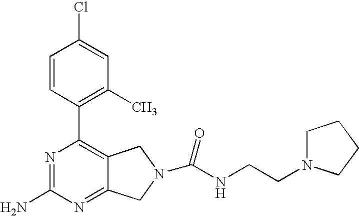 | 2-amino-4-(4-chloro-2- methylphenyl)-N-(2- pyrrolidin-1-ylethyl)-57- dihydro-6H-pyrrolo[34- d]pyrimidine-6- carboxamide | Example 7 | (300 MHz, DMSO-D6) δ ppm 7.43-7.42 (m, 1H), 7.36-7.30 (m, 2H), 6.77 (br s, 2H), 6.35 (t, J = 5.8 Hz, 1H), 4.40 (s, 2H), 4.24 (s, 2H), 3.17-3.10 (m, 2H), 2.46-2.39 (m, 6H), 2.22 (s, 3H), 1.66-1.62 (m, 4H). | 401.2 |
| 98 | 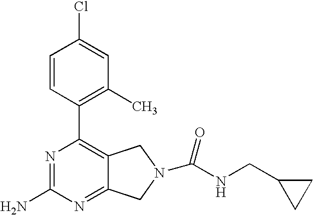 | 2-amino-4-(4-chloro-2- methylphenyl)-N- (cyclopropylmethyl)-57- dihydro-6H-pyrrolo[34- d]pyrimidine-6- carboxamide | Example 7 | (300 MHz, DMSO-D6) δ ppm 7.43-7.42 (m, 1H), 7.35-7.33 (m, 2H), 6.77 (br s, 2H), 6.46 (t, J = 5.6 Hz, 1H), 4.41 (s, 2H), 4.25 (s, 2H), 2.90 (dd, J = 6.0 and 6.0 Hz, 2H), 2.23 (s, 3H), 0.97-0.87 (m, 1H), 0.38-0.32 (m, 2H), 0.16-0.11 (m, 2H) | 358.2 |

### Example 100

### N-allyl-2-amino-4-{2,4-dichloro-6-[2-(1H-pyrazol-1-yl)ethoxy]phenyl}-5,7-dihydro-6H-pyrrolo[3,4-d]pyrimidine-6-carboxamide

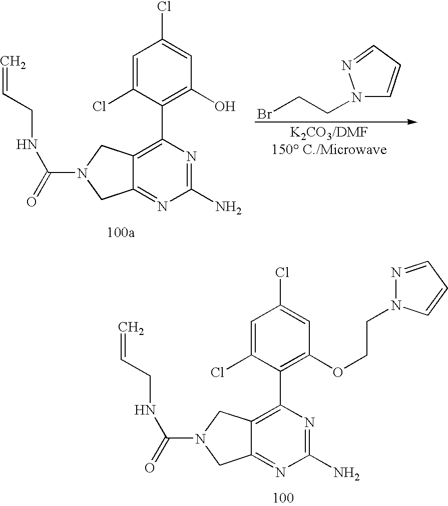

Compound 100a, N-allyl-2-amino-4-(2,4-dichloro-6-hydroxyphenyl)-5,7-dihydro-6H-pyrrolo[3,4-d]pyrimidine-6-carboxamide, was prepared in a manner similar to Example 18a except that allyl isocyanate was substituted for cyclobutylamine and CDI in Example 18b. Compound 100a (60 mg, 0.16 mmol), potassium carbonate (131 mg, 0.95 mmol.), 1-(2-bromoethyl)-1H-pyrazole (56 mg, 0.316 mmol) and DMF (2.0 mL) were microwaved at 120° C. for 30 min. Isolation by preparative HPLC gave compound 100 (39 mg, 0.08 mmol) as a white solid in 52% yield. ¹H NMR (400 MHz, DMSO-d6) δ ppm 3.60 (d, J=12.88 Hz, 2H), 3.67 (t, J=5.05 Hz, 2H), 3.94 (d, J=13.14 Hz, 2H), 4.27-4.37 (m, 4H), 4.41 (d, J=5.81 Hz, 2H), 5.74-5.90 (m, 1H), 6.00 (t, J=2.02 Hz, 1H), 6.45 (br. s., 1H), 6.78 (s, 2H), 7.18 (d, J=2.02 Hz, 1H), 7.25 (d, J=1.77 Hz, 1H), 7.29 (d, J=1.52 Hz, 1H), 7.32 (d, J=1.77 Hz, 1H). LCMS (M+H)⁺474.0, 476.0.

### Example 101

### 2-amino-N-cyclopropyl-4-[2,4-dichloro-6-(2-morpholin-4-ylethoxy)phenyl]-5,7-dihydro-6H-pyrrolo[3,4-d]pyrimidine-6-carboxamide

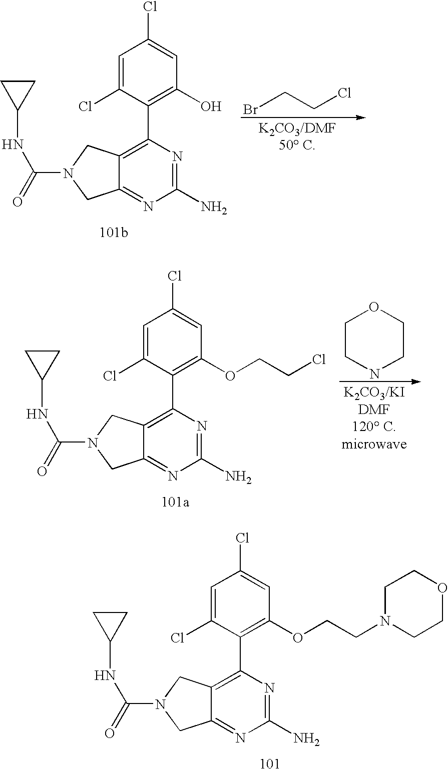

Compound 101b, 2-amino-N-cyclopropyl-4-(2,4-dichloro-6-hydroxyphenyl)-5,7-dihydro-6H-pyrrolo[3,4-d]pyrimidine-6-carboxamide, was prepared in a manner similar to Example 18a except that cyclopropylamine was substituted for cyclobutylamine in Example 18b. Compound 101b (800 mg, 2.1 mmol), potassium carbonate (872 mg, 6.31 mmol), 1-bromo-2-chloroethane (0.4 mL, 4.2 mmol) in DMF (8.0 mL) were heated at 50° C. for 12 h and the mixture was filtered. The collected insoluble material was washed with EtOAc. The combined filtrate/washings plus additional EtOAc (200 mL) were washed with saturated aqueous NaHCO₃ (50 mL), brine (50 ml), dried (Na₂SO₄), filtered and concentrated. Isolation by silica gel chromatography (gradient of 0-10% MeOH in CH₂Cl₂) gave compound 101a, 2-amino-N-cyclopropyl-4-[2,4-dichloro-6-(2-chloroethoxy)phenyl]-5,7-dihydro-6H-pyrrolo[3,4-d]pyrimidine-6-carboxamide, (786 mg, 1.79 mmole) as brown grease in 85% yield. ¹H NMR (400 MHz, DMSO-d6) δ ppm 0.32-0.43 (m, 2H), 0.47-0.58 (m, 2H), 3.69-3.86 (m, 1H), 4.00-4.12 (m, 2H), 4.19 (d, J=13.14 Hz, 2H), 4.23-4.31 (m, 2H), 4.32-4.45 (m, 2H), 6.42 (d, J=2.78 Hz, 1H), 6.80 (s, 2H), 7.38 (d, J=1.77 Hz, 1H), 7.96 (s, 1H). LCMS (M+H)⁺444.0, 446.0. Compound 101a (50 mg, 0.11 mmol), K₂CO₃ (94 mg, 0.68 mmol), KI (38 mg, 0.23 mmol), morpholine (30 mg, 0.34 mmol) and DMF (2.0 mL) were then microwaved at 130° C. for 45 min. Isolation by preparative HPLC gave compound 101 (32 mg, 0.063 mmole) as a white solid in 57% yield. ¹H NMR (400 MHz, DMSO-d6) δ ppm 0.32-0.45 (m, 2H), 0.48-0.58 (m, 2H), 2.07-2.37 (m, 4H), 2.52-2.58 (m, 1H), 3.39 (br. s., 4H), 4.02-4.22 (m, 5H), 4.34-4.46 (m, 3H), 6.46 (d, J=2.27 Hz, 1H), 6.81 (br. s., 2H), 7.32 (d, J=15.66 Hz, 2H). LCMS (M+H)⁺493.1, 495.1.

### Example 102

### 2-amino-N-(1-cyanocyclopropyl)-4-{2,4-dichloro-6-[2-(1H-pyrazol-1-yl)ethoxy]phenyl}-5,7-dihydro-6H-pyrrolo[3,4-d]pyrimidine-6-carboxamide

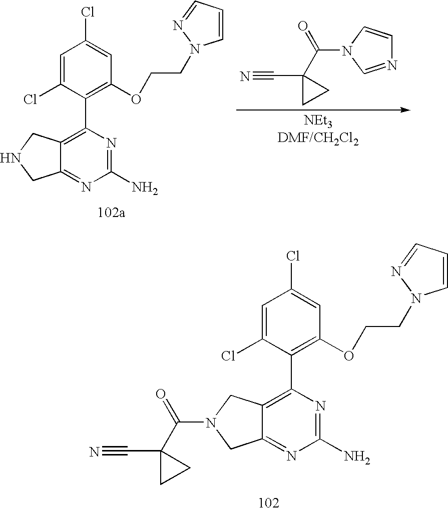

Compound 102.HCl (45 mg, 0.12 mmol) was treated with the preformed adduct of 1-amino-cyclopropanecarbonitrile hydrochloride (59 mg, 0.5 mmol) and CDI (126 mg, 0.775 mmol) in a manner similar to that described for Example 18b. Following extractive work up from EtOAc, washing with saturated aqueous NaHCO₃, the product was isolated by preparative HPLC to give compound 102 (38 mg, 0.08 mmole) as a white solid in 66% yield. ¹H NMR (400 MHz, DMSO-d6) δ ppm 1.12 (d, J=7.33 Hz, 2H), 1.41 (br. s., 2H), 3.57 (s, 2H), 3.87 (d, J=12.63 Hz, 1H), 4.33 (s, 4H), 4.41 (d, J=7.33 Hz, 2H), 6.02 (t, J=1.89 Hz, 1H), 6.82 (s, 2H), 7.21 (d, J=2.02 Hz, 1H), 7.26 (d, J=1.52 Hz, 1H), 7.32 (dd, J=3.66, 1.64 Hz, 2H). LCMS (M+H)⁺499.2.

### Preparation of Compound 102a, 4-{2,4-dichloro-6-[2-(1H-pyrazol-1-yl)ethoxy]phenyl}-6,7-dihydro-5H-pyrrolo[3,4-d]pyrimidin-2-amine

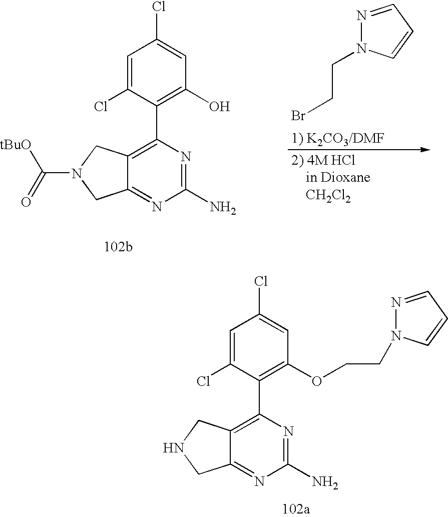

Compound 102b, tert-butyl 2-amino-4-(2,4-dichloro-6-hydroxyphenyl)-5,7-dihydro-6H-pyrrolo[3,4-d]pyrimidine-6-carboxylate, was prepared from compound 9a (1090 mg, 3.0 mmol), compound 18c (682 mg, 3.3 mmol), Pd(PPh₃)₄ (347 mg, 0.3 mmol.), and 2M Na₂CO₃ (4.5 ml, 9.0 mmol) in 1,4-dioxane (25 mL) in a manner similar to that described for Example 1. ¹H NMR (400 MHz, DMSO-d6) δ ppm 1.37-1.47 (m, J=12.88 Hz, 9H) 4.07-4.24 (m, 2H) 4.42 (d, J=7.58 Hz, 2H) 6.82 (s, 2H) 6.95 (d, J=1.77 Hz, 1H) 7.08-7.21 (m, 1H) 10.67 (s, 1H). LCMS (M+H)⁺397.1 399.1. Compound 102b (1.627 g, 4.1 mmol) was O-alkylated using potassium carbonate (3.4 g, 24.6 mmol.), 1-(2-bromoethyl)-1H-pyrazole (1.43 g, 8.19 mmol) in DMF (10 mL) in a manner similar to that described for Example 100. Boc deprotection was carried out using 4M HCl in 1,4-dioxane (10 mL) and MeOH (20 mL)—stirring at ambient temperature for about 16 h. Following extractive work up from EtOAc, washing with saturated aqueous NaHCO₃, the crude product was isolated by preparative HPLC to give compound 102a, 4-{2,4-dichloro-6-[2-(1H-pyrazol-1-yl)ethoxy]phenyl}-6,7-dihydro-5H-pyrrolo[3,4-d]pyrimidin-2-amine, (405 mg, 0.95 mmol) as a white HCl salt in 26% yield. ¹H NMR (400 MHz, DMSO-d6) δ ppm 3.51 (d, J=13.39 Hz, 2H), 3.93-4.07 (m, 2H), 4.24-4.44 (m, 4H), 6.14 (br. s., 1H), 6.65 (br. s., 2H), 7.19 (br. s., 1H), 7.24 (br. s., 1H), 7.29 (br. s., 1H), 7.40 (s, 1H), 8.24 (s, 1H). LCMS (M+H)⁺391.2, 393.2.

### Alternate Preparation of Compound 102a, 4-{2,4-dichloro-6-[2-(1H-pyrazol-1-yl)ethoxy]phenyl}-6,7-dihydro-5H-pyrrolo[3,4-d]pyrimidin-2-amine

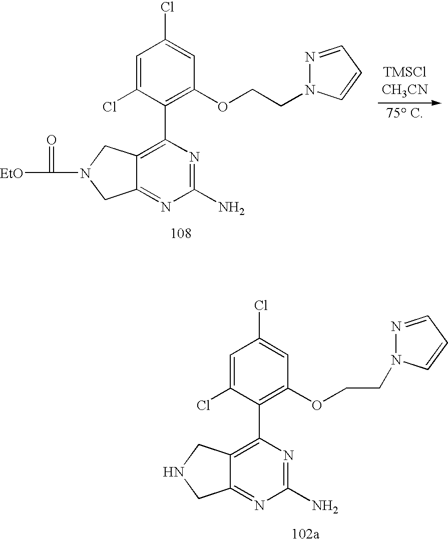

Deprotection of the ethyl carbamate of compound 108 (5.06 g, 10.3 mmol) using iodotrimethylsilane (8.0 mL, 56 mmol) in CH₃CN (80.0 mL) was carried out in a similar manner to Example 2. Compound 102a (8.46 g) was obtained as a crude HI salt. LCMS (M+H)⁺: 393.2, 391.3.

### Example 103

### 2-amino-N-cyclobutyl-4-[4-(hydroxymethyl)-2-methyl-6-(4,4,4-trifluorobutoxy)phenyl]-5,7-dihydro-6H-pyrrolo[3,4-d]pyrimidine-6-carboxamide

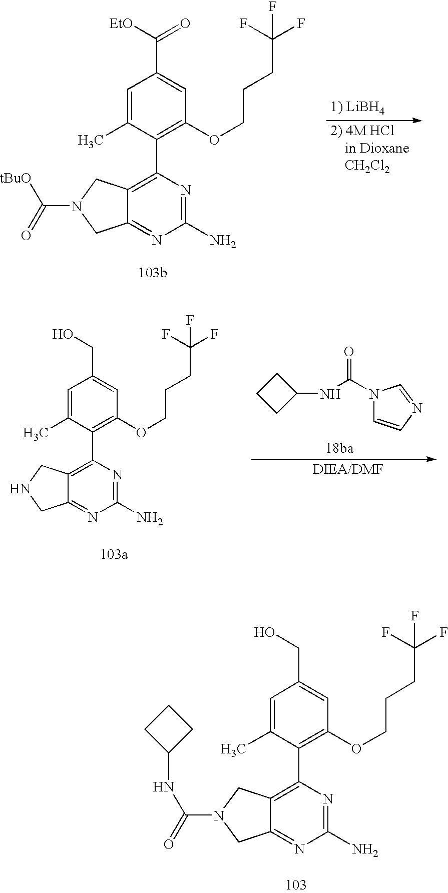

To compound 103b (205 mg, 0.39 mmol) in THF (4 mL) at 0° C. was added 2M LiBH₄ in THF (0.6 mL, 1.17 mmol) and MeOH (0.5 mL). The mixture was stirred at 0° C. and allowed to warm to ambient temperature overnight. Following extractive work up from EtOAc (300 ml), washing with saturated aqueous NaHCO₃ (50 ml) then brine (50 ml), the EtOAc solution was dried (Na₂SO₄), filtered, and the volatile components removed to give the alcohol (185 mg) as a clear grease which was carried on directly. LCMS (M+H)⁺ 483.2, 484.2. For Boc deprotection, CH₂Cl₂ (5 mL) was added followed by 4M HCl in dioxane (1.0 ml, 3.83 mmol). After stirring 12 h, the mixture was concentrated and ether and hexane were added. The volatile components were removed to give compound 103a, 4-(2-amino-6,7-dihydro-5H-pyrrolo[3,4-d]pyrimidin-4-yl)-3-methyl-5-(4,4,4-trifluorobutoxy)benzoic acid, (189 mg) as a yellow hydrochloride which was carried on without purification. LCMS (M+H)⁺383.2, 384.2. Compound 103a in DMF (2.0 mL) was reacted with compound 18ba (76 mg, 0.46 mmol) and DIEA (0.4 ml, 6.0 mmol) in a manner similar to Example 18b. Isolation by preparative HPLC gave compound 103 (33 mg, 0.083 mmole) as a white solid in 21% yield. ¹H NMR (400 MHz, DMSO-d6) δ ppm 1.44-1.61 (m, 2H), 1.69-1.81 (m, 2H), 1.84-1.98 (m, 2H), 2.00-2.16 (m, 7H), 3.92-4.05 (m, 3H), 4.06-4.20 (m, 2H), 4.37 (d, J=5.31 Hz, 2H), 4.49 (d, J=5.56 Hz, 2H), 5.25 (t, J=5.68 Hz, 1H), 6.51 (d, J=7.83 Hz, 1H), 6.65 (s, 2H), 6.85 (s, 1H), 6.89 (s, 1H). LCMS (M+H)⁺480.4, 481.4.

### Preparation of Compound 103b, 2-amino-N-cyclobutyl-4-[4-(hydroxymethyl)-2-methyl-6-(4,4,4-trifluorobutoxy)phenyl]-5,7-dihydro-6H-pyrrolo[3,4-d]pyrimidine-6-carboxamide

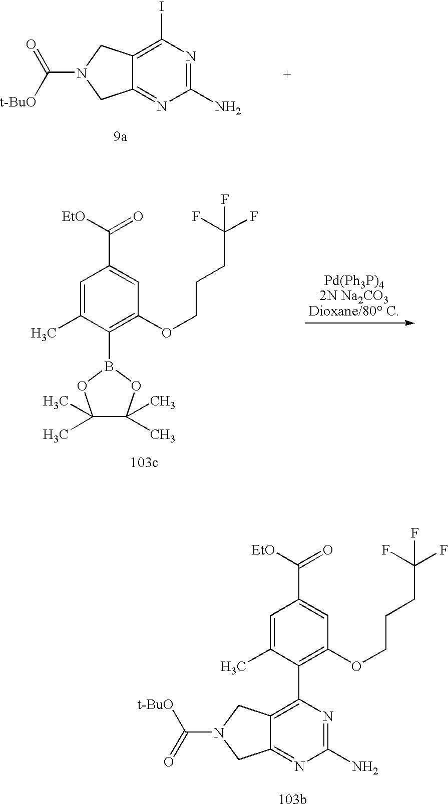

Compound 9a (200 mg, 0.552 mmol) and compound 103c (253 mg, 0.61 mmol), Pd(PPh₃)₄ (32 mg, 0.028 mmol) and 2M Na₂CO₃ solution (0.8 ml, 1.66 mmol) in 1,4-dioxane (6 mL) were reacted for 48 h in a manner similar to Example 1. Monitoring (LCMS) indicated that the reaction was incomplete so the mixture was microwaved at 160° C. for 1 h whereupon the suspension was filtered and the solids washed with MeOH. The combined filtrate/washings were concentrated and subjected to EtOAc (300 ml) extractive work up washing with saturated aqueous NaHCO₃ (50 ml) and brine (50 ml). Isolation by silica gel chromatography (gradient of 0-60% EtOAc in CH₂Cl₂) gave compound 103b (205 mg, 0.433 mmole) as a clear grease in 71% yield. ¹H NMR (400 MHz, CHLOROFORM-d) δ ppm 1.21-1.28 (m, 9H), 1.38-1.54 (m, 4H), 1.85-1.98 (m, 2H), 1.98-2.12 (m, 2H), 2.20 (d, J=4.55 Hz, 3H), 3.98-4.13 (m, 2H), 4.41 (q, J=7.07 Hz, 2H), 4.60 (d, J=22.48 Hz, 2H), 7.39-7.52 (m, 2H), 7.59-7.73 (m, 1H). LCMS (M+H)⁺524.2, 526.2.

### Preparation of Compound 103c, ethyl 3-methyl-4-(4,4,5,5-tetramethyl-1,3,2-dioxaborolan-2-yl)-5-(4,4,4-trifluorobutoxy)benzoate

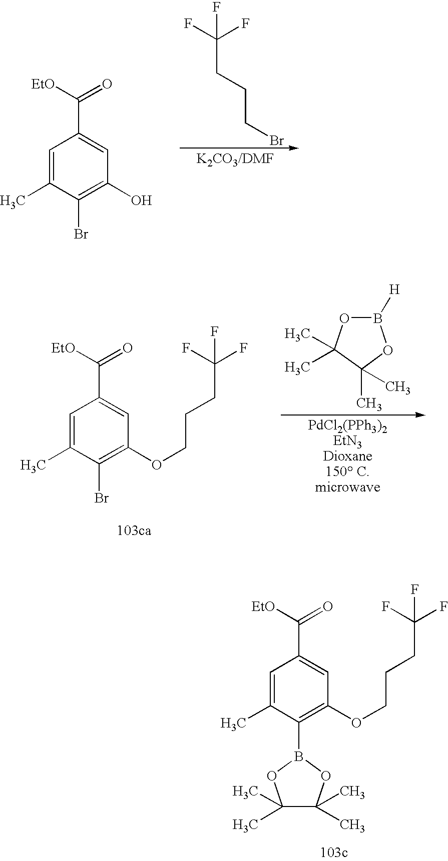

Ethyl 4-bromo-3-hydroxy-5-methylbenzoate (650 mg, 2.5 mmol) was O-alkylated with 4-bromo-1,1,1-trifluorobutane (950 mg, 5.0 mmol) using potassium carbonate (1040 mg, 7.5 mmol) in DMF (5.0 mL) in a manner similar to that described for Example 100. Isolation using silica gel chromatography (0-20% gradient of EtOAc in hexane) gave compound 103ca, ethyl 4-bromo-3-methyl-5-(4,4,4-trifluorobutoxy)benzoate, (858 mg, 2.33 mmole) as a white solid in 93% yield. ¹H NMR (400 MHz, CHLOROFORM-d) δ ppm 1.41 (t, J=7.20 Hz, 3H), 2.04-2.21 (m, 2H), 2.32-2.44 (m, 2H), 2.44-2.50 (m, 3H), 4.15 (t, J=5.94 Hz, 2H), 4.38 (q, J=7.07 Hz, 2H), 7.35 (d, J=1.77 Hz, 1H), 7.57 (d, J=1.01 Hz, 1H). Compound 103ca (561 mg, 1.52 mmol), pinacolborane (0.7 ml, 4.6 mmol), Et₃N (0.9 ml, 6.1 mmol) in 1,4-dioxane (6.0 mL) were purged with N₂ for 15 min, then Pd(II)Cl₂(PPh₃)₂ (107 mg, 0.1 mmol) was added. The mixture was microwaved at 150° C. for 1 h, cooled and filtered through celite washing with EtOAc. The combined filtrate/washings were washed with water (50 ml), brine (50 ml), dried (Na₂SO₄), filtered and concentrated. Isolation using silica gel chromatography (0-20% gradient of EtOAc in hexane) gave compound 103c (252 mg, 0.61 mmole) in 40% yield. ¹H NMR (400 MHz, CHLOROFORM-d) δ ppm 1.37-1.41 (m, 15H), 2.05 (dd, J=10.74, 5.68 Hz, 2H), 2.31 2.39 (m, 2H), 2.39-2.41 (m, 3H), 4.06 (t, J=5.81 Hz, 2H), 4.36 (q, J=7.24 Hz, 2H), 7.25 (s, 1H), 7.46 (s, 1H).

### Example 104

### 2-amino-4-(2-chloro-4-cyclopropyl-5-methoxyphenyl)-N-isopropyl-5,7-dihydro-6H-pyrrolo[3,4-d]pyrimidine-6-carboxamide

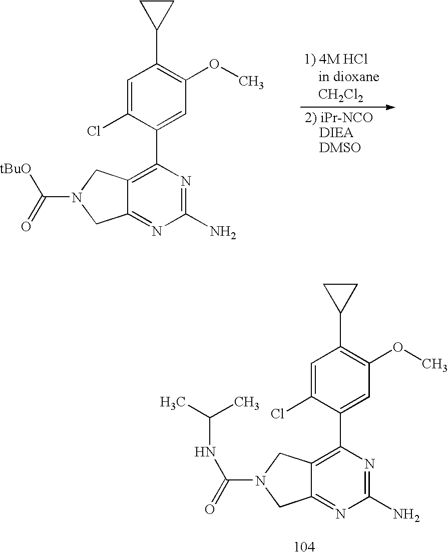

To compound 104a (67 mg, 0.16 mmol) in CH₂Cl₂ (4.0 mL) was added 4M HCl in 1,4-dioxane (0.2 ml, 0.804 mmol). After stirring at ambient temperature for 12 h, the mixture was concentrated and treated with ether and hexane. Removal of the volatile components gave the Boc-deprotected intermediate (65 mg) as a yellow hydrochloride which was carried on directly. LCMS (M+H)⁺317.2, 319.2. Dimethylformamide (2.0 mL) and diisopropylethylamine (0.14, 0.8 mmol) were added followed by isopropyl isocyanate (14.0 mg, 0.16 mmol) to form the urea in a manner similar to Example 15a. Isolation by preparative HPLC gave compound 104 (13 mg, 0.032 mmol) as brown foam in 20% yield. ¹H NMR (400 MHz, DMSO-d6) δ ppm 0.69-0.78 (m, 2H), 0.90-0.98 (m, 2H), 1.05 (d, J=6.57 Hz, 6H), 2.04-2.18 (m, 1H), 3.77 (t, J=6.95 Hz, 1H), 3.81 (s, 3H), 4.25 (s, 2H), 4.40 (s, 2H), 6.09 (d, J=7.83 Hz, 1H), 6.82 (s, 2H), 6.95 (s, 2H). LCMS (M+H)⁺ 402.2, 404.2.

### Preparation of Compound 104a, tert-butyl 2-amino-4-(2-chloro-4-cyclopropyl-5-methoxyphenyl)-5,7-dihydro-6H-pyrrolo[3,4-d]pyrimidine-6-carboxylate

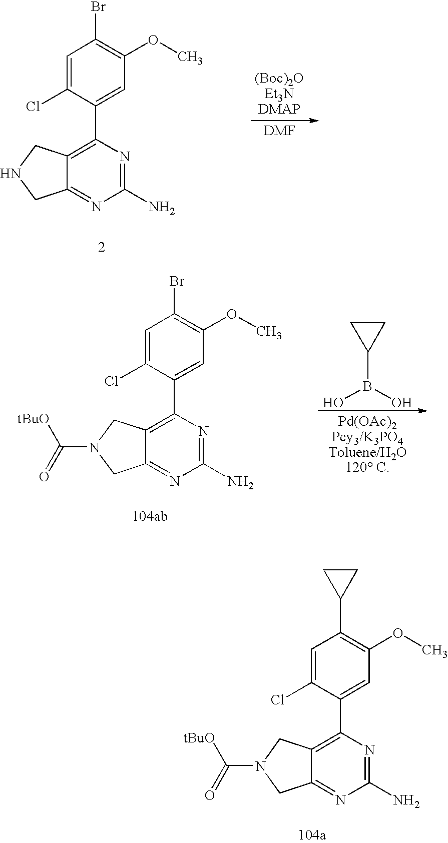

To compound 2 (612 mg, 1.0 mmol) and di-tert-butyldicarbonate (655 mg, 3.0 mmol.) in DMF (5.0 mL) was added triethylamine (0.84 ml, 6.0 mmol) and DMAP (25 mg, 0.2 mmol). After stirring 4 h at ambient temperature, the mixture was subjected EtOAc (200 ml) extractive work up washing with saturated aqueous NaHCO₃ (50 ml) and brine (50 ml). After drying (Na₂SO₄), filtering and concentrating, isolation using silica gel chromatography (0-50% gradient of EtOAc in CH₂Cl₂) gave compound 104ab, tert-butyl 4-(4-bromo-2-chloro-5-methoxyphenyl)-2-[(tert-butoxycarbonyl)amino]-5,7-dihydro-6H-pyrrolo[3,4-d]pyrimidine-6-carboxylate, (320 mg, 0.58 mmole) as yellowish foam in 58% yield. ¹H NMR (400 MHz, DMF-d7) δ ppm 1.34-1.50 (m, 18H), 3.84-3.90 (m, 3H), 4.30 (d, J=7.33 Hz, 2H), 4.45 (d, J=6.06 Hz, 2H), 7.16 (d, J=3.79 Hz, 1H), 7.88 (d, J=3.54 Hz, 1H), 7.97 (s, 1H). LCMS (M+H)⁺ 556.4, 558.4. Compound 104ab (167 mg, 0.3 mmol), cyclopropylboronic acid (52 mg, 0.6 mmol), K₃PO₄ (255 mg, 1.20 mmol) and tricyclohexylphosphine (9.0 mg, 0.03 mmol) in toluene (4.0 mL) with H₂O (0.1 mL) were purged with N₂ for 15 min then Pd(OAc)₂ (4.0 mg, 0.02 mmol) was added. After heating at 120° C. for 6 h, the mixture was filtered through celite washing with EtOAc. The filtrate/washings were subjected to EtOAc (500 ml) extractive work up washing with saturated aqueous NaHCO₃ (100 ml) and brine (100 ml). Following drying (Na₂SO₄), filtration and concentration, isolation using silica gel chromatography (0-50% gradient of EtOAc in CH₂Cl₂) gave compound 104a (67 mg, 0.324 mmole) as a yellowish foam in 54% yield. ¹H NMR (400 MHz, CHLOROFORM-d) δ ppm 0.79-0.94 (m, 2H), 0.94-1.06 (m, 2H), 1.43-1.54 (m, 9H), 2.08-2.25 (m, 1H), 3.84-3.93 (m, 3H), 4.48 (d, J=19.70 Hz, 2H), 4.58 (d, J=23.75 Hz, 2H), 5.28 (s, 2H), 6.77 (d, J=10.11 Hz, 1H), 6.87 (d, J=11.12 Hz, 1H). LCMS (M+H)⁺417.2, 419.2.

### Example 105

### 4-(4-bromo-2-chloro-5-methoxyphenyl)-6-(2-methoxyethyl)-6,7-dihydro-5H-pyrrolo[3,4-d]pyrimidin-2-amine

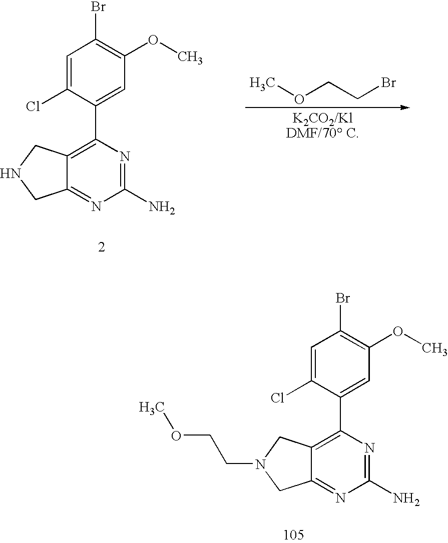

The hydrochloride of compound 2 (70 mg, 0.16 mmol), K₂CO₃ (225 mg, 1.63 mmol), and 2-bromoethylmethyl ether (113 mg, 0.82 mmol) in DMF (3 mL) were microwaved at 100° C. for 2 h. Isolation by preparative HPLC gave compound 105 (13 mg, 0.03 mmole) as a white solid in 19% yield. ¹H NMR (400 MHz, DMSO-d6) δ ppm 3.27-3.32 (m, 3H), 3.47-3.60 (m, 2H), 3.66 (br. s., 2H), 3.84-3.93 (m, 3H), 4.35-4.62 (m, 4H), 7.05-7.12 (m, 1H), 7.14-7.25 (m, 2H), 7.91 (s, 1H). LCMS (M+H)⁺415.0, 417.0.

### Example 106

### 2-amino-4-(2-chloro-4-ethenyl-5-methoxyphenyl)-N-(1-methylethyl)-5,7-dihydro-6H-pyrrolo[3,4-d]pyrimidine-6-carboxamide

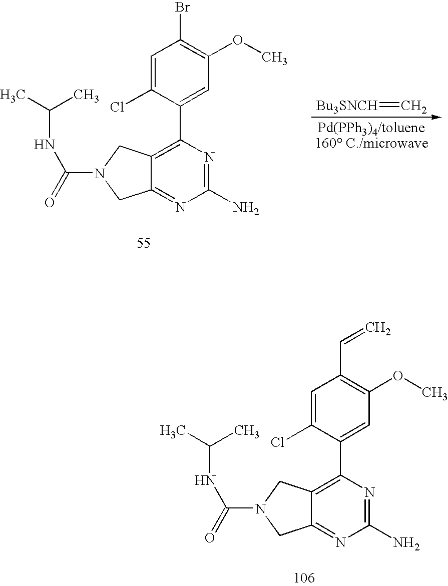

Compound 55 (66 mg, 0.15 mmol) and ethenyltributylstannane (62 mg, 0.2 mmol) in toluene (1 mL) and dioxane (1 mL) were purged with N₂ and Pd(PPh₃)₄ (9 mg, 0.008 mmol) was added and the mixture was microwaved at 160° C. for 30 min. The mixture was filtered through celite and washed with MeOH. The filtrate was concentrated and subjected to EtOAc (200 ml) extractive work up washing with saturated aqueous NaHCO₃ (50 ml) and brine (50 ml). Following drying (Na₂SO₄) and concentration, isolation by preparative HPLC gave compound 106 (11 mg, 0.029 mmole) as a white solid in 19% yield. ¹H NMR (400 MHz, DMSO-d6) δ ppm 1.02-1.10 (m, 6H), 3.74-3.80 (m, 1H), 3.79-3.86 (m, 3H), 4.28 (s, 2H), 4.43 (s, 2H), 5.40 (d, J=12.38 Hz, 1H), 5.98 (dd, J=17.94, 1.01 Hz, 1H), 6.09 (d, J=6.82 Hz, 2H), 6.93 (dd, J=17.68, 11.37 Hz, 1H), 7.06 (s, 1H), 7.70 (s, 1H). LCMS (M+H)⁺386.2, 386.2.

### Example 107

### 2-amino-4-(2-chloro-4,6-dimethoxyphenyl)-N-prop-2-en-1-yl-5,7-dihydro-6H-pyrrolo[3,4-d]pyrimidine-6-carboxamide

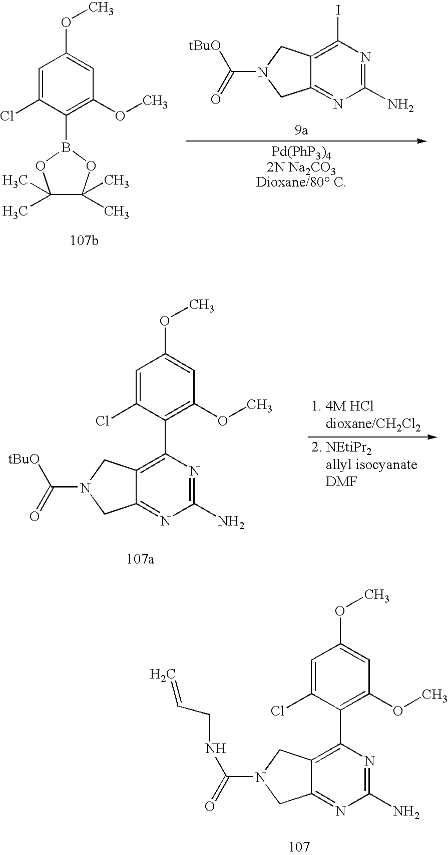

Compound 9a (199 mg, 0.549 mmol), compound 107b (164 mg, 0.549 mmol), Pd(PPh₃)₄ (32 mg, 0.027 mmol) and 2M aqueous Na₂CO₃ (0.8 ml, 1.65 mmol) in 1,4-dioxane (4 mL) were reacted in a manner similar to Example 9. Isolation using silica gel chromatography (gradient of 0-10% MeOH in CH₂Cl₂) gave compound 107a, tert-butyl 2-amino-4-(2-chloro-4,6-dimethoxyphenyl)-5,7-dihydro-6H-pyrrolo[3,4-c]pyrimidine-6-carboxylate, (204 mg, 0.52 mmole) as a yellowish solid. LCMS (M+H)⁺407.4, 409.4. To Boc-deprotect, treatment of compound 107a (204 mg, 0.5 mmol) in CH₂Cl₂ (5 mL) with 4M HCl in 1,4-dioxane (1.3 mL) in a manner similar to Example 102a gave the hydrochloride (200 mg) as a yellow grease which was carried on without purification. LCMS (M+H)⁺ 307.2, 309.2. A portion of the Boc-deprotected hydrochloride (95 mg, 0.25 mmol) in DMF (2.0 mL) was treated with diisopropylethylamine (0.3 ml, 1.5 mmol) and allyl isocyanate (21 mg, 0.25 mmol) in a manner similar to Example 15a. Isolation by preparative HPLC gave compound 107 (15 mg, 0.038 mmol) as a white solid in 15% yield. ¹H NMR (400 MHz, DMSO-d6) δ ppm 3.62-3.70 (m, 2H), 3.73 (s; 3H), 3.83 (s, 3H), 4.09-4.18 (m, 2H), 4.43 (s, 2H), 5.00 (dd, J=10.36, 1.52 Hz, 1H), 5.10 (dd, J=17.18, 1.52 Hz, 1H), 5.72-5.88 (m, 1H), 6.57 (t, J=5.56 Hz, 1H), 6.67 (d, J=2.27 Hz, 1H), 6.72-6.78 (m, 2H). LCMS (M+H)⁺390.1, 392.

### Preparation of Compound 107b, 2-(2-chloro-4,6-dimethoxyphenyl)-4,4,5,5-tetramethyl-1,3,2-dioxaborolane

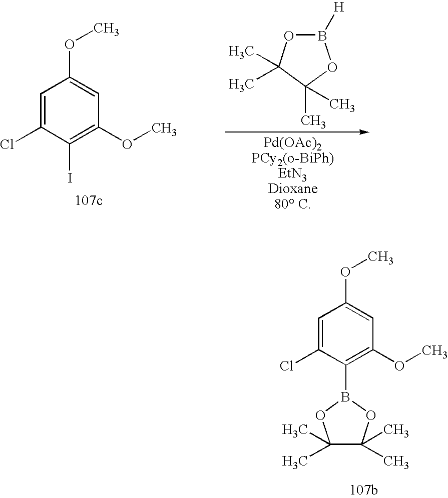

Compound 107c (1.45 g, 4.86 mmol), pinacolborane (1.4 ml, 9.72 mmol) and Et₃N (2.0 ml, 14.6.0 mmol), palladium (II) acetate (55 mg, 0.243 mmol) and 2-(dicyclohexylphosphino)biphenyl (170 mg, 0.7 mmol.) in 1,4-dioxane (25 mL). were reacted in a manner similar to Example 18ca. Isolation using silica gel chromatography (gradient of 0-20% EtOAc in hexane) gave compound 107b (1.104 g, 3.74 mmole) as a yellowish solid in 77% yield. NMR data: ¹H NMR (400 MHz, CHLOROFORM-D) δ ppm 1.37 (s, 12H), 3.73 (s, 3H), 3.76 (s, 3H), 6.25 (s, 1H), 6.44 (s, 1H).

### Preparation of Compound 107c, 1-chloro-2-iodo-3,5-dimethoxybenzene

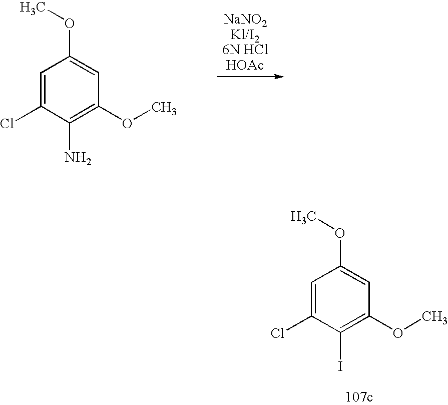

To (2-chloro-4,6-dimethoxyphenyl)amine (1.9 g, 10.0 mmol) in acetic acid (40 mL) was added 6M HCl (10 mL). With stirring & cooling in an ice salt bath, sodium nitrite (828 mg, 12.0 mmol) in water (6 mL) was added slowly keeping the reaction temperature <5° C. After addition, the mixture was stirred for about 30 min and a solution of potassium iodide (3320 mg, 20.0 mmol) and iodine (761 mg, 3.0 mmol) in water (35 mL) was added dropwise. The mixture was allowed to warm to ambient temperature over about 90 min and water (120 mL) was added. The mixture was subjected to EtOAc (2×300 mL) extractive work up and the combined extracts were washed with 10% aqueous Na₂S₂O₃ solution (2×100 ml) and brine (100 ml). After drying (Na₂SO₄), filtering and concentrating, isolation using silica gel chromatography (0-30% gradient of EtOAc in hexane) gave compound 107c (1.45 g, 4.9 mmole) as a yellowish solid in 49% yield. ¹H NMR (400 MHz, CHLOROFORM-D) d ppm 3.79 (s, 3H), 3.84 (s, 3H), 6.30 (d, J=2.53 Hz, 1H), 6.68 (d, J=2.53 Hz, 1H).

### Example 108

### ethyl 2-amino-4-{2,4-dichloro-6-[2-(1H-pyrazol-1-yl)ethoxy]phenyl}-5,7-dihydro-6H-pyrrolo[3,4-d]pyrimidine-6-carboxylate

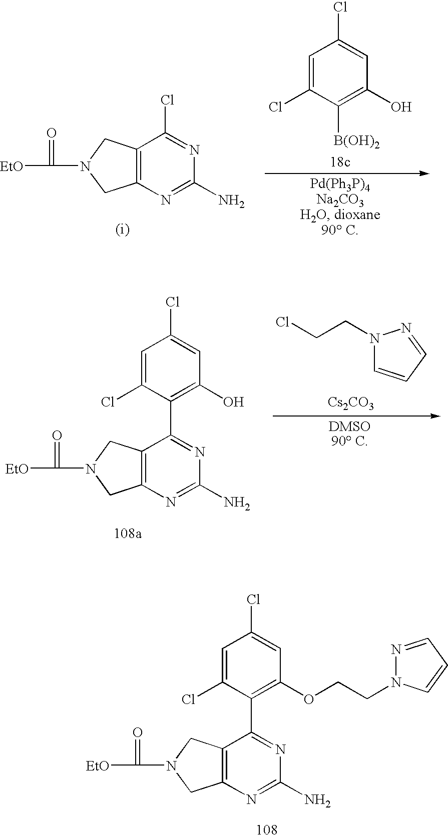

Compound (i) (4.04 g, 16.6 mmol), compound 18c (4.82 g, 23.3 mmol), 2M aqueous sodium carbonate (21 ml, 42 mmol), and tetrakis(triphenylphosphino)palladium(0) (1.82 g, 1.58 mmol) in 1,4 dioxane (200 mL) were reacted in a manner similar to Example 1. Extractive work up with EtOAc was carried out in a manner similar to Example 18a first using 0.5N aq NaOH (300 mL) to extract the phenol product into the basic aqueous layer followed by acidification to pH 5 with 1N KH₂PO₄ (75 mL) and extraction of phenol product into EtOAc (400 mL). The EtOAc layer was concentrated to give compound 108a, ethyl 2-amino-4-(2,4-dichloro-6-hydroxyphenyl)-5,7-dihydro-6H-pyrrolo[3,4-d]pyrimidine-6-carboxylate (6.15 g) which was carried on without purification. ¹H NMR (400 MHz, DMSO-d6) d ppm 1.07-1.34 (m, 3H) 3.99-4.26 (m, 4H) 4.48 (d, J=11.87 Hz, 2H) 6.84 (s, 2H) 7.14 (d, J=1.77 Hz, 1H) 7.48-7.79 (m, 1H) 10.71 (s, 1H). LCMS (M+H)⁺: 369.0 371.0. Compound 108a (5.11 g, 13.8 mmol), 1-(2-chloro-ethyl)-1H-pyrazole (5.02 g, 38.4 mmol) and cesium carbonate (15.8 g, 48.4 mmol) in DMSO (50.0 mL) were reacted at 90° C. in a manner similar to Example 18. Following extractive work up from EtOAc (500 mL), washing with water (200 mL), the product was isolated using silica gel chromatography (100% EtOAc followed by a gradient of 0-13% MeOH in dichloromethane) to give compound 108 (5.06 g, 13.8 mmol) as a tan powder in 77% yield. ¹H NMR (300 MHz, DMSO-d₆) δ ppm 1.13-1.29 (m, 3H) 3.49-3.64 (m, 1H) 3.91 (d, J=13.19 Hz, 1H) 4.02-4.19 (m, 2H) 4.34 (s, 4H) 4.40-4.48 (m, 2H) 5.99-6.04 (m, 1H) 6.83 (s, 2H) 7.23-7.28 (m, 2H) 7.28-7.33 (m, 2H). LCMS (M+H)⁺: 465.2, 463.2.

### Example 109

### 2-amino-N-bicyclo[1.1.1]pent-1-yl-4-{2,4-dichloro-6-[2-(1H-pyrazol-1-yl)ethoxy]phenyl}-5,7-dihydro-6H-pyrrolo[3,4-d]pyrimidine-6-carboxamide

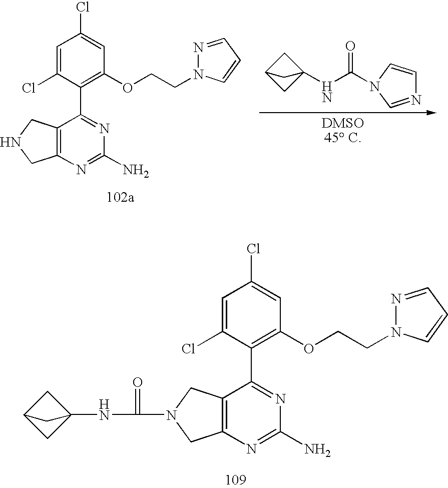

Bicyclo[1.1.1]pentan-1-amine-HCl (637 mg, 5.33 mmol) followed by triethylamine (4.00 mL, 28.7 mmol) were added to 1,1-carbonyldiimidazole (870 mg, 5.37 mmol) in DMF (50 mL) at 0° C. The mixture was stirred at ambient temperature for 1 h and crude compound 102a.HI salt (4.650 g, 5.150 mmol) was added. The mixture heated to 45° C. for 0.5 h. Following EtOAc extractive work up, washing with water, the crude product was subjected to silica gel chromatography (gradient of 10-15% MeOH in dichloromethane). After isolation, 10% CH₃CN in water was added. Following lyophilization; compound 109 (1.54 g, 3.08 mmol) was obtained as a white powder in 58% yield. ¹H NMR (300 MHz, DMSO-d6) δ ppm 1.95 (s, 6H) 2.36 (s, 1H) 3.53 (d, J=13.19 Hz, 1H) 3.89 (d, J=13.00 Hz, 1H) 4.33 (s, 6H) 5.99-6.02 (m, 1H) 6.77 (br. s., 2H) 6.85 (br. s., 1H) 7.19 (d, J=2.07 Hz, 1H) 7.25 (d, J=1.51 Hz, 1H) 7.30 (d, J=1.70 Hz, 1H) 7.32 (d, J=1.70 Hz, 1H). LCMS (M+H)⁺: 502.2, 500.2.

### Example 110

### 2-amino-4-{2,4-dichloro-6-[2-(1H-pyrazol-1-yl)ethoxy]phenyl}-N-(2,2,2-trifluoroethyl)-5,7-dihydro-6H-pyrrolo[3,4-d]pyrimidine-6-carboxamide

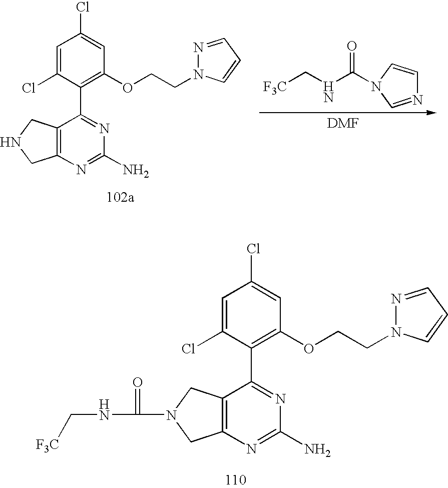

2,2,2-Trifluoroethylamine (19.8 mg, 0.200 mmol), 1,1-carbonyldiimidazole (32.4 mg, 0.200 mmol), compound 102a.HCl (89.9 mg, 0.180 mmol) and triethylamine (0.080 mL, 0.57 mmol) in DMF (2 mL) were reacted in a manner similar to Example 18b. Isolation using preparative HPLC gave compound 110 (37 mg, 0.72 mmol) as a white powder in 40% yield. ¹H NMR (400 MHz, DMSO-d6) δ ppm 3.54 (d, J=12.38 Hz, 1H) 3.77-3.92 (m, 2H) 3.94 (d, J=12.88 Hz, 1H) 4.42-4.50 (m, 2H) 5.98 (t, J=2.15 Hz, 1H) 6.82 (s, 2H) 7.21 (d, J=2.02 Hz, 1H) 7.27 (s, 2H) 7.33 (d, J=1.77 Hz, 1H). LCMS (M+H)⁺: 518.0, 516.0.

### Example 111

### 2-amino-N-bicyclo[1.1.1]pent-1-yl-4-{4-bromo-2-chloro-5-[2-(1H-pyrazol-1-yl)ethoxy]phenyl}-5,7-dihydro-6H-pyrrolo[3,4-d]pyrimidine-6-carboxamide

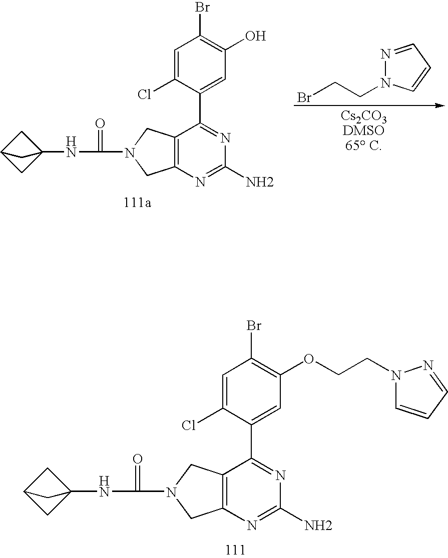

Compound 111a (44.0 mg, 0.0976 mmol) was alkylated with 1-(2-bromo-ethyl)-1H-pyrazole (32.4 mg, 0.185 mmol) and cesium carbonate (122 mg, 0.374 mmol) in DMSO (1.5 mL) in a manner similar to that described in Example 18. Isolation using preparative HPLC gave compound III (29 mg, 0.053 mmol) as a white powder in 55% yield. ¹H NMR (300 MHz, DMSO-d6) d ppm 1.92 (s, 6H) 2.34 (s, 1H) 4.21 (s, 2H) 4.36-4.42 (m, 4H) 4.52 (t, J=4.71 Hz, 2H) 6.24 (t, J=1.98 Hz, 1H) 6.88 (s, 2H) 6.99 (s, 1H) 7.15 (s, 1H) 7.44 (d, J=1.70 Hz, 1H) 7.78 (d, J=2.26 Hz, 1H) 7.84 (s, 1H). LCMS (M+H)⁺: 546.2, 544.2.

### Preparation of Compound 111a, 2-amino-N-bicyclo[1.1.1]pent-1-yl-4-(4-bromo-2-chloro-5-hydroxyphenyl)-5,7-dihydro-6H-pyrrolo[3,4-d]pyrimidine-6-carboxamide

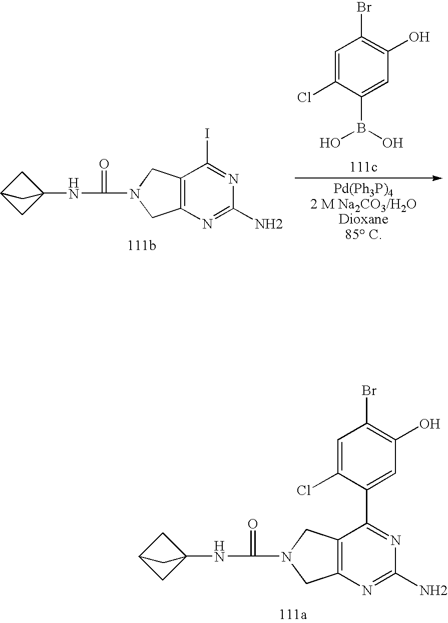

Compound 111b, 2-amino-N-bicyclo[1.1.1]pent-1-yl-4-iodo-5,7-dihydro-6H-pyrrolo[3,4-d]pyrimidine-6-carboxamide, was prepared from compound 8b in a manner similar to Example 18b except that bicyclo[1.1.1]pentan-1-amine-HCl was substituted for cyclobutylamine. The coupling of compound 111b (0.2995 g, 0.8070 mmol) with compound 111c (0.201 g, 0.800 mmol) was carried out using tetrakis(triphenylphosphino)palladium(0) (0.065 g, 0.070 mmol) and 2M aqueous sodium carbonate (0.80 mL, 1.6 mmol) in 1,4-dioxane (5.0 mL) in a manner similar to that described for Example 1. Extractive work up involved aqueous base extraction followed by acidification and EtOAc extraction in a manner similar to Example 108a. Isolation gave compound 111a (0.316 g) as a tan solid which was carried on without purification. ¹H NMR (300 MHz, DMSO-d₆) δ ppm 1.93 (s, 6H) 2.34 (s, 1H) 4.22 (s, 2H) 4.38 (s, 2H) 6.87 (br. s., 2H) 6.91 (s, 1H) 7.01 (s, 1H) 7.74 (s, 1H) 10.83 (br. s., 1H). LCMS (M+H)⁺: 452.0, 450.0.

### Preparation of Compound 111c, (4-bromo-2-chloro-5-hydroxyphenyl)boronic acid

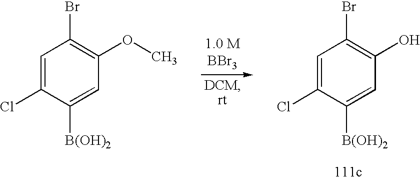

To a suspension of (4-bromo-2-chloro-5-methoxyphenyl)boronic acid (4.96 g, 18.7 mmol) in dichloromethane (125 mL) at 0° C. was added 1 M boron tribromide (52 mL, 52 mmol) in dichloromethane. The reaction mixture was allowed to warm to ambient temperature and stir for 48 h. The reaction mixture was then poured over ice, 3N NaOH (52 mL) was added, and the mixture was stirred vigorously for 30 minutes. The layers were separated, and the dichloromethane layer was discarded. The aqueous layer, a slurry, was acidified to pH 5 with concentrated HCl and EtOAc extractive work up gave compound 111c (4.614 g, 18.36 mmol) as a white powder which was carried on without purification.

### Example 112

### 2-amino-N-bicyclo[1.1.1]pent-1-yl-4-[4-bromo-2-chloro-5-(difluoromethoxy)phenyl]-5,7-dihydro-6H-pyrrolo[3,4-d]pyrimidine-6-carboxamide

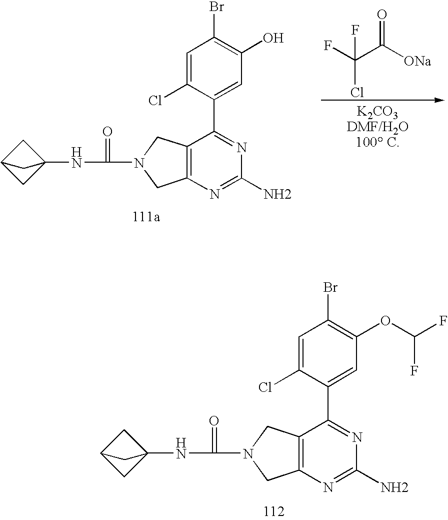

Nitrogen was bubbled through a solution of compound 111a (53 mg, 0.12 mmol) in DMF (1.0 mL) and H₂O (0.13 mL) for 10 minutes. Potassium carbonate (0.092 g, 0.67 mmol) and sodium chlorodifluoroacetate (0.067 g, 0.44 mmol) were added. The reaction mixture was heated to 100° C. for 4 h. Isolation using preparative HPLC gave compound 112 (0.019 g, 0.038 mmol) as a white powder in 32% yield. ¹H NMR (300 MHz, DMSO-d6) δ ppm 1.93 (s, 6H) 2.35 (s, 1H) 4.23 (s, 2H) 4.39 (s, 2H) 6.94 (s, 1H) 6.99-7.03 (m, 1H) 7.36 (t, J=72.90 Hz, 1H) 7.43 (s, 1H) 8.10 (s, 1H). LCMS (M+H)⁺: 502.2, 500.0.

### Example 113

### 2-amino-N-bicyclo[1.1.1]pent-1-yl-4-{2,4-dichloro-6-[2-(1,3-thiazol-2-yl)ethoxy]phenyl}-5,7-dihydro-6H-pyrrolo[3,4-d]pyrimidine-6-carboxamide

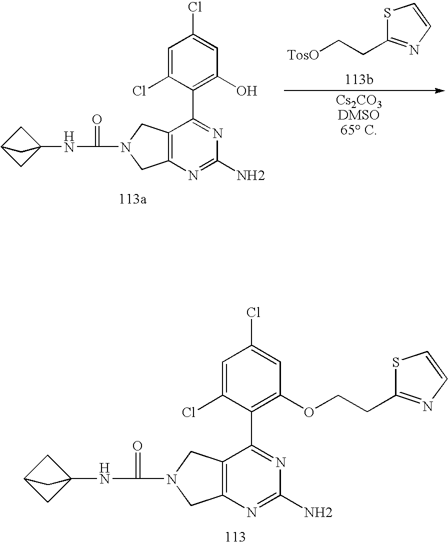

Compound 113a, 2-amino-N-bicyclo[1.1.1]pent-1-yl-4-(2,4-dichloro-6-hydroxyphenyl)-5,7-dihydro-6H-pyrrolo[3,4-d]pyrimidine-6-carboxamide, was prepared in a similar manner as Example 18a except that bicyclo[1.1.1]pentan-1-amine-HCl was substituted for cyclobutylamine. Compound 113a (65 mg, 0.16 mmol), cesium carbonate (201 mg, 0.617 mmol) and compound 113b (73 mg, 0.26 mmol) in DMSO (1.5 mL) were heated to 65° C. After 0.5 h, additional compound 113b (196 mg, 0.629 mmol) was added in portions over 5 h with continuing heat. Extractive work up from EtOAc, washing with water, followed by isolation using preparative HPLC gave compound 113 (0.0101 g, 0.0195 mmol) as a white solid in 12% yield. ¹H NMR (400 MHz, DMSO-d6) δ ppm 1.95 (s, 6H) 2.36 (s, 1H) 3.26 (t, J=5.81 Hz, 2H) 3.57 (d, J=12.63 Hz, 1H) 3.90 (d, J=13.14 Hz, 1H) 4.31-4.39 (m, 4H) 6.75 (s, 2H) 6.87 (s, 1H) 7.31 (d, J=1.77 Hz, 1H) 7.33 (d, J=1.77 Hz, 1H) 7.35 (d, J=3.28 Hz, 1H) 7.58 (d, J=3.28 Hz, 1H). LCMS (M+H)⁺: 519.2, 517.2.

### Preparation of Compound 113b, 2-(1,3-thiazol-2-yl)ethyl 4-methylbenzenesulfonate

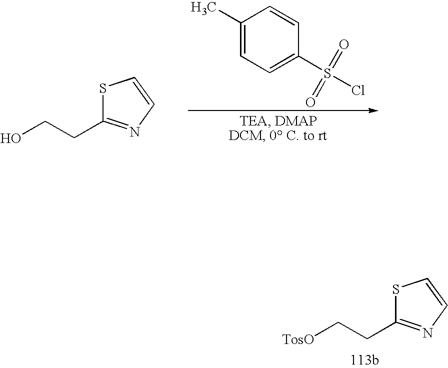

To 2-(1,3-thiazol-2-yl)ethanol (123 mg, 0.952 mmol) (Young et. al. Eur. J. Med. Chem., 28: 201-211; (1993)) and triethylamine (0.15 mL, 1.1 mmol) in dichloromethane (5.0 mL) at 0° C. was added 4-toluensulfonyl chloride (189 mg, 0.991 mmol) and an unmeasured amount of catalytic DMAP. The mixture was stirred overnight and allowed warm to warm to ambient temperature. Extractive work up from dichloromethane (30 mL), washing with saturated aqueous NH₄Cl, gave compound 113b (0.255 g, 0.901 mmol) in 95% crude yield. ¹H NMR (300 MHz, DMSO-d₆) δ ppm 2.42 (s, 3H) 3.34 (t, J=6.03 Hz, 2H) 4.36 (t, J=6.03 Hz, 2H) 7.46 (d, J=8.48 Hz, 2H) 7.60 (d, J=3.39 Hz, 1H) 7.69 (d, J=3.39 Hz, 1H) 7.72 (d, J=8.10 Hz, 2H). LCMS (M+H)⁺: 284.1.

### Example 114

### 2-amino-N-bicyclo[1.1.1]pent-1-yl-4-{2,4-dichloro-6-[2-(4-methyl-1H-pyrazol-1-yl)ethoxy]phenyl}-5,7-dihydro-6H-pyrrolo[3,4-d]pyrimidine-6-carboxamide

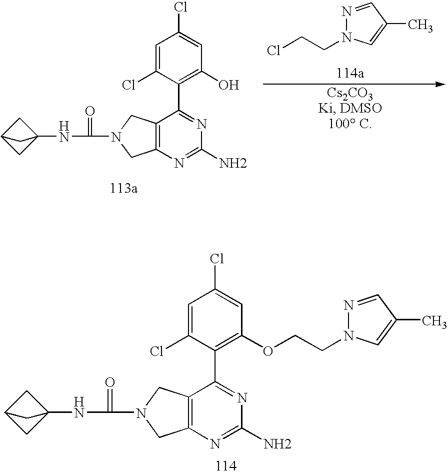

Compound 113a (59 mg, 0.15 mmol), compound 114a (49 mg, 0.34 mmol), cesium carbonate (177 mg, 0.543 mmol), and potassium iodide (7 mg) in DMSO (1.5 mL) were heated at 100° C. for 6 h. Isolation by preparative HPLC gave compound 114 (0.0258 g, 0.0501 mmol) as a white solid in 35% yield. ¹H NMR (300 MHz, DMSO-d6) δ ppm 1.90 (s, 3H) 1.94 (s, 6H) 2.35 (s, 1H) 3.69 (d, J=13.38 Hz, 1H) 3.95 (d, J=12.24 Hz, 1H) 4.20-4.39 (m, 6H) 6.80 (s, 2H) 6.89-6.93 (m, 1H) 6.97 (s, 1H) 7.10 (s, 1H) 7.25 (d, J=1.51 Hz, 1H) 7.33 (d, J=1.13 Hz, 1H). LCMS (M+H)⁺: 516.2, 514.2.

### Preparation of Compound 114a, 1-(2-chloroethyl)-4-methyl-1H-pyrazole

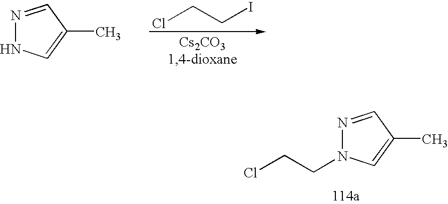

4-Methyl pyrazole (0.177 g, 2.16 mmol), 1-chloro-2-iodoethane (1.20 g, 6.30 mmol) and cesium carbonate (1.10 g, 3.38 mmol) in 1,4-dioxane (5.0 mL) were heated at 95° C. for 24 h. After cooling to ambient temperature, dichloromethane (10 mL) was added. Following filtration to remove solids, the filtrate was reduced and subjected to silica gel chromatography (gradient of 0-70% EtOAc in hexane) which gave compound 114a (0.049 g, 0.34 mmol) as clear oil in 16% yield. ¹H NMR (300 MHz, CHLOROFORM-d) d ppm 2.12 (s, 3H) 3.92 (t, J=5.84 Hz, 2H) 4.49 (t, J=5.93 Hz, 2H) 7.33 (s, 1H) 7.46 (s, 1H).

### Example 115

### 2-amino-N-bicyclo[1.1.1]pent-1-yl-4-{2,4-dichloro-6-[3-(1H-pyrazol-1-yl)propoxy]phenyl}-5,7-dihydro-6H-pyrrolo[3,4-d]pyrimidine-6-carboxamide

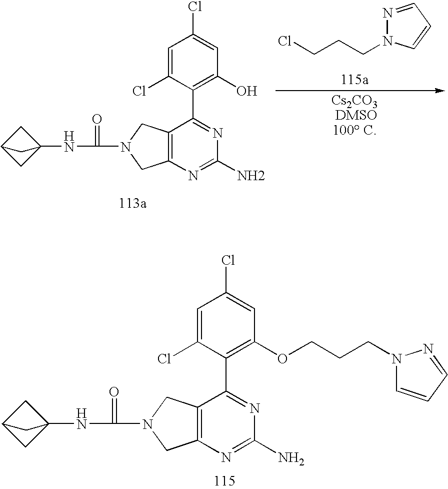

Compound 115a, 1-(3-chloropropyl)-1H-pyrazole, was prepared in a manner similar to compound 114a except that pyrazole was substituted for 4-methylpyrazole and 1-bromo-3-chloropropane was substituted for 1-chloro-2-iodoethane. ¹H NMR (300 MHz, CHLOROFORM-d) δ ppm 2.33-2.42 (m, 2H) 3.47 (t, J=6.03 Hz, 2H) 4.41 (t, J=6.40 Hz, 2H) 6.31 (t, J=2.17 Hz, 1H) 7.49 (d, J=2.07 Hz, 1H) 7.61 (d, J=1.51 Hz, 1H). Compound 113a (0.0442 g, 0.109 mmol) was then O-alkylated with compound 115a (0.054 g, 0.37 mmol) and cesium carbonate (0.122 g, 0.374 mmol) in DMSO (1.5 mL) at 100° C. in a manner similar to Example 18. Isolation using preparative HPLC gave compound 115 (0.0234, 0.0372 mmol) as a TFA salt in 34% yield. ¹H NMR (300 MHz, METHANOL-d4) d ppm 2.03 (s, 6H) 2.12-2.22 (m, J=2.45 Hz, 2H) 2.37 (s, 1H) 3.86-3.94 (m, 1H) 3.98-4.07 (m, 1H) 4.12 (t, J=6.59 Hz, 2H) 4.31 (s, 2H) 4.57 (s, 2H) 6.23-6.25 (m, 1H) 7.14 (d, J=1.70 Hz, 1H) 7.25 (d, J=1.70 Hz, 1H) 7.45 (d, J=1.51 Hz, 1H) 7.53 (d, J=2.07 Hz, 1H). LCMS (M+H)⁺: 516.2, 514.2.

### Example 116

### 2-amino-N-cyclobutyl-4-[2,4-dichloro-6-(2-pyridin-2-ylethoxy)phenyl]-5,7-dihydro-6H-pyrrolo[3,4-d]pyrimidine-6-carboxamide

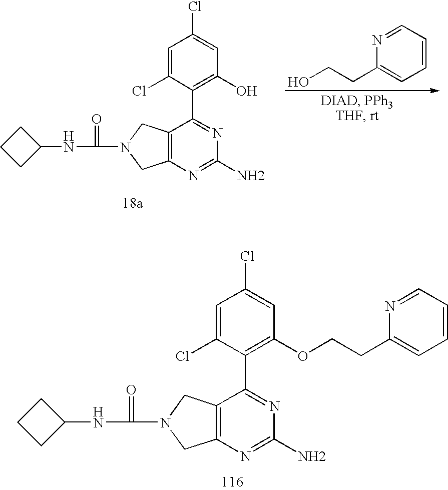

To compound 18a (0.032 g, 0.081 mmol), 2-(2-hydroxyethyl)pyridine, and triphenylphosphine (0.053 g, 0.20 mmol) in THF (1.5 mL) was added diisopropyl azodicarboxylate (40 μL, 0.207 mmol). After 3 h, DMSO (1.5 mL) was added and the volatile components removed under vacuum. Isolation using preparative HPLC gave compound 116 (0.0208 g, 0.0416 mmol) as a tan powder in 51% yield. ¹H NMR (300 MHz, DMSO-d6) δ ppm 1.49-1.63 (m, 2H) 1.88-2.00 (m, 2H) 2.05-2.21 (m, 2H) 2.93-3.02 (m, 2H) 3.38 (d, 1H, overlaps with water peak) 3.86 (d, J=13.00 Hz, 1H) 4.08-4.23 (m, 1H) 4.28-4.45 (m, 4H) 6.37 (d, J=8.10 Hz, 1H) 6.77 (s, 2H) 6.91 (d, J=7.72 Hz, 1H) 6.99-7.06 (m, 1H) 7.28-7.32 (m, 2H) 7.50 (dt, J=7.58, 1.79 Hz, 1H) 8.36 (d, J=4.14 Hz, 1H). LCMS (M+H)⁺: 501.2, 499.2.

### Example 117

### 2-amino-N-cyclobutyl-4-(4,6-dichloro-2,3-dimethoxyphenyl)-5,7-dihydro-6H-pyrrolo[3,4-d]pyrimidine-6-carboxamide

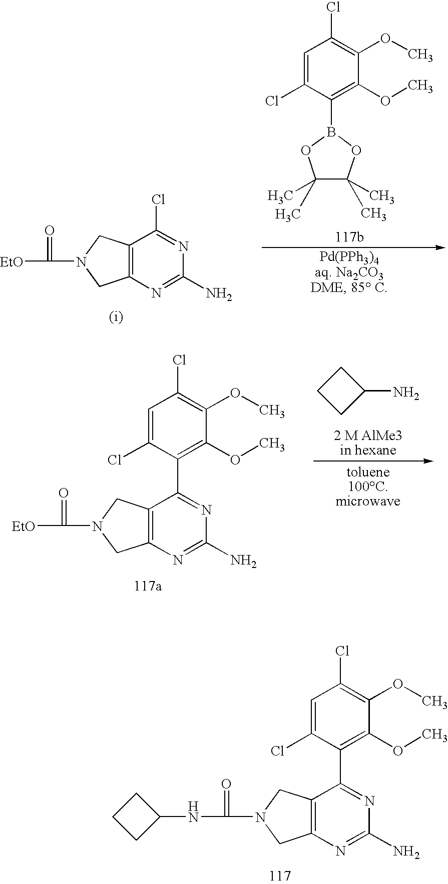

Compound (i) (0.263 g, 1.08 mmol), compound 117b (0.525 g, 1.58 mmol), 2M aqueous sodium carbonate (0.80 mL, 1.6 mmol) and tetrakis(triphenylphosphino)palladium(0) (0.075 g, 0.065 mmol) in DME (10 mL) were reacted in a manner similar to Example 1. Extractive work up with EtOAc (30 mL), washing with water (30 mL) followed by isolation using silica gel chromatography (gradient of 0-25% MeOH in dichloromethane) gave compound 117a, ethyl 2-amino-4-(4,6-dichloro-2,3-dimethoxyphenyl)-5,7-dihydro-6H-pyrrolo[3,4-d]pyrimidine-6-carboxylate, (0.034 mg, 0.082 mmol) in 8% yield. Compound 117a (0.034 g, 0.082 mmol), cyclobutylamine (0.040 g, 0.60 mmol) and 2M trimethyl aluminum in hexane (0.25 mL, 0.50 mmol) were reacted in a manner similar to Example 7. Isolation using preparative HPLC gave compound 117 (0.010 mmol) in 28% yield. ¹H NMR (300 MHz, DMSO-d6) δ ppm 1.49-1.61 (m, 2H) 1.85-2.00 (m, 2H) 2.03-2.15 (m, 2H) 3.69 (s, 3H) 3.85 (s, 3H) 4.07-4.24 (m, 3H) 4.43 (s, 2H) 6.51 (d, J=7.91 Hz, 1H) 6.89 (br. s., 2H) 7.60 (s, 1H). LCMS (M+H)⁺: 440.2, 438.2.

### Preparation of Compound 117b, 2-(4,6-dichloro-2,3-dimethoxyphenyl)-4,4,5,5-tetramethyl-1,3,2-dioxaborolane

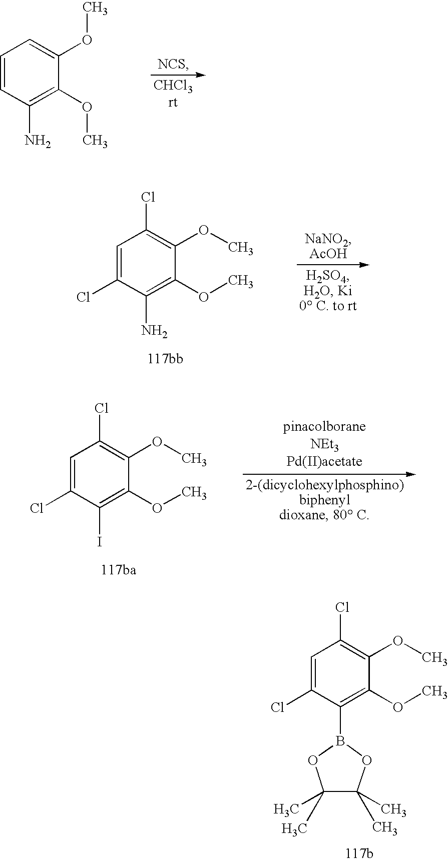

N-chlorosuccinimide (6.87 g, 50.4 mmol) was added to 2,3-dimethoxyaniline (3.86 g, 25.2 mmol) in chloroform (100 mL) at 0° C. After allowing to warm to ambient temperature and stir for an additional 4 h, isolation by silica gel chromatography (dichloromethane) gave compound 117bb, 4,6-dichloro-2,3-dimethoxyaniline, (1.97 g, 8.87 mmol) as an orange oil in 35% yield. ¹H NMR (300 MHz, DMSO-d₆) δ ppm 3.74 (s, 3H) 3.77 (s, 3H) 5.27 (br. s., 2H) 7.12 (s, 1H). With stirring, sodium nitrite (799 mg, 11.6 mmol) in H₂O (4.0 mL) was added dropwise to compound 117bb (1.97 g, 8.87 mmol) in acetic acid (20.0 mL), H₂O (6.0 mL) and concentrated H₂SO₄ (6.0 mL) at 0° C. After 3 h at 0° C., the mixture was poured into a potassium iodide (10.4 g, 62.7 mmol) solution in H₂O (20.0 mL) at 0° C. rinsing with additional H₂O (20 mL). The mixture was allowed to slowly warm to ambient temperature and stirred for an additional 16 h. The mixture was then extracted with diethyl ether (100 mL) and the Et₂O layer washed with saturated aqueous Na₂S₂O₃ (100 mL), 3N NaOH (100 mL) and water (100 mL). Isolation using silica gel chromatography (gradient of 0-20% dichloromethane in hexane) gave compound 117ba, 1,5-dichloro-2-iodo-3,4-dimethoxybenzene, (2.46 g, 7.39 mmol) in 83% yield. ¹H NMR (300 MHz, DMSO-d₆) δ ppm 3.81 (s, 3H) 3.82 (s, 3H) 7.62 (s, 1H). Compound 117ba (2.45 g, 7.36 mmol), pinacolborane (2.1 mL, 14.5 mmol), and triethyl amine (3.00 mL, 21.5 mmol) in 1,4-dioxane (40 mL) were purged with N₂ for 15 min and palladium (II) acetate (86 mg, 0.38 mmol) and 2-(dicyclohexylphosphino)biphenyl (263 mg, 0.750 mmol) were added. The mixture was heated to 80° C. for 2.5 h, cooled, and subjected to EtOAc extractive work up, washing with saturated aqueous NH₄Cl. Isolation using silica gel chromatography (gradient of 0-50% dichloromethane in hexane) gave compound 117b (1.155 g, 3.468 mmol) in 47% yield. ¹H NMR (300 MHz, DMSO-d₆) d ppm 1.32 (s, 12H) 3.79 (s, 6H) 7.36 (s, 1H).

### Example 118

### 2-amino-4-(4-bromo-2-chloro-5-methoxyphenyl)-N-(6-cyanopyridin-3-yl)-5,7-dihydro-6H-pyrrolo[3,4-d]pyrimidine-6-carboxamide

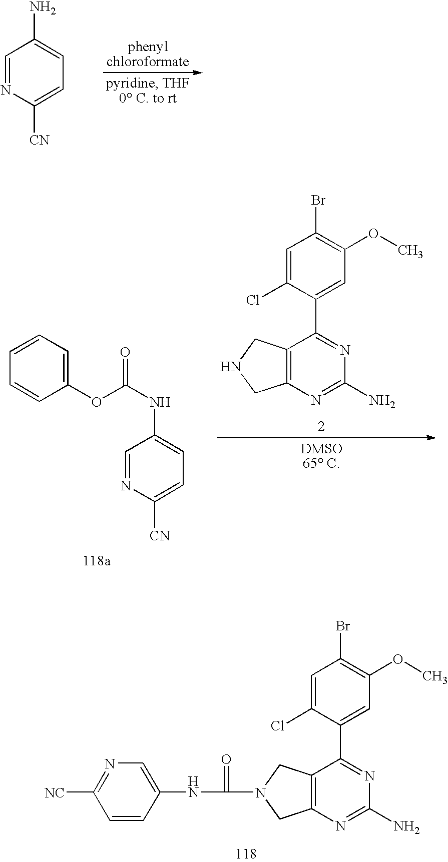

Phenylchloroformate (0.70 mL, 5.6 mmol) in THF (10 mL) was added dropwise to 5-amino-2-cyanopyridine (655 mg. 5.50 mmol) and pyridine (0.50 mL, 6.2 mmol) in THF (10.0 mL) at 0° C. After allowing to warm to ambient temperature and stirring for 1 h, the mixture was again cooled to 0° C., and the precipitate removed by filtration. Removal of the volatile components from the filtrate gave compound 118a, phenyl (6-cyanopyridin-3-yl)carbamate, (0.921, 3.85 mmol) as a pale orange solid in 70% crude yield. ¹H NMR (300 MHz, DMSO-d₆) δ ppm 7.24-7.33 (m, 3H) 7.45 (t, J=7.82 Hz, 2H) 8.01 (d, J=8.48 Hz, 1H) 8.13 (dd, J=8.48, 2.55 Hz, 1H) 8.81 (d, J=2.45 Hz, 1H) 11.01 (s, 1H). Compound 2 (0.093 g, 0.152 mmol), compound 118a (0.038 g, 0.160 mmol), and cesium carbonate (0.154 g, 0.473 mmol) in DMSO (1.5 mL) were heated to 65° C. for 20 min. Isolation using preparative HPLC gave compound 118 (0.013 g, 0.026 mmol) as an orange solid in 17% yield. ¹H NMR (300 MHz, DMSO-d6) δ ppm 3.87 (s, 3H) 4.52 (br. s., 2H) 4.64 (br. s., 2H) 6.97 (br. s., 2H) 7.19 (s, 1H) 7.87-7.94 (m, 2H) 8.20 (dd, J=8.57, 2.17 Hz, 1H) 8.87 (d, J=2.45 Hz, 1H) 9.04 (br. s., 1H). LCMS (M+H)⁺: 502.2, 500.2.

### Example 119

### 2-amino-N-cyclobutyl-4-[(E)-2-phenylvinyl]-5,7-dihydro-6H-pyrrolo[3,4-d]pyrimidine-6-carboxamide

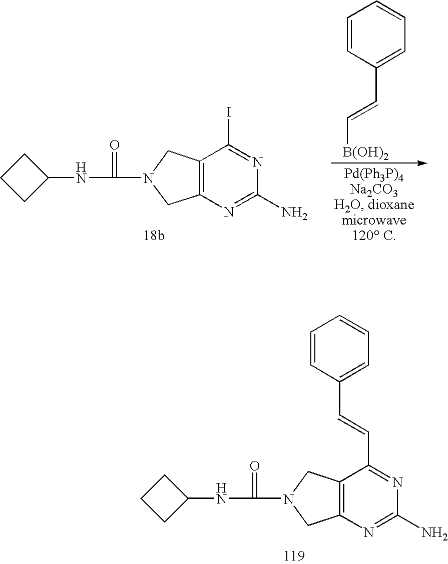

Compound 18b (0.103 g, 0.287 mmol), trans-2-phenylvinylboronic acid (0.0602 mg, 0.407 mmol) and 2M aqueous sodium carbonate (0.30 mL, 0.60 mmol) in 1,4-dioxane (3.0 mL) were purged with N₂. Tetrakis(triphenylphosphino)palladium(0) (0.024 g, 0.021 mmol) was added and the mixture microwaved at 120° C. for 1.5 h. After EtOAc (20 mL) extractive work up, washing with water (20 mL), isolation using preparative HPLC gave compound 119 (0.022 g, 0.066 mmol) in 23% yield. ¹H NMR (300 MHz, DMSO-d6) δ ppm 1.52-1.68 (m, 2H) 1.92-2.07 (m, 2H) 2.09-2.23 (m, 2H) 4.11-4.28 (m, 1H) 4.37 (s, 2H) 4.60 (s, 2H) 6.52 (d, J=7.72 Hz, 1H) 6.59 (br. s., 2H) 7.02 (d, J=16.01 Hz, 1H) 7.34-7.48 (m, 3H) 7.63-7.77 (m, 3H). LCMS (M+H)⁺: 336.4.

### Example 120

### 2-{[2-amino-4-(4-bromo-2-chloro-5-methoxyphenyl)-5,7-dihydro-6H-pyrrolo[3,4-d]pyrimidin-6-yl]methyl}benzonitrile

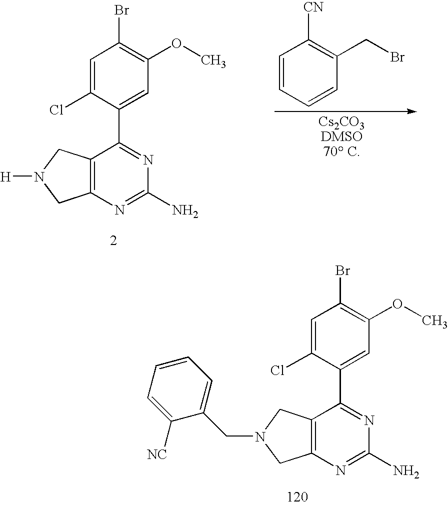

Compound 2 (0.067 g, 0.110 mmol), 2-(bromomethyl)-benzonitrile (0.0215 mg, 0.110 mmol) and cesium carbonate (0.1189 g, 0.365 mmol) in DMSO (1.5 mL) were heated at 70° C. for 10 min. Isolation using preparative HPLC gave compound 120 (0.007 g, 0.01 mmol) in 10% yield. ¹H NMR (300 MHz, DMSO-d6) δ ppm 3.67 (s, 2H) 3.83 (s, 2H) 3.84 (s, 3H) 4.03 (s, 2H) 6.79 (br. s., 2H) 7.10 (s, 1H) 7.47 (t, J=7.44 Hz, 1H) 7.60-7.71 (m, 2H) 7.79-7.84 (m, 2H). LCMS (M+H)⁺: 472.2, 470.2.

### Example 121

### 2-amino-4-(2-chloro-4-cyano-5-methoxyphenyl)-N-isopropyl-5,7-dihydro-6H-pyrrolo[3,4-d]pyrimidine-6-carboxamide

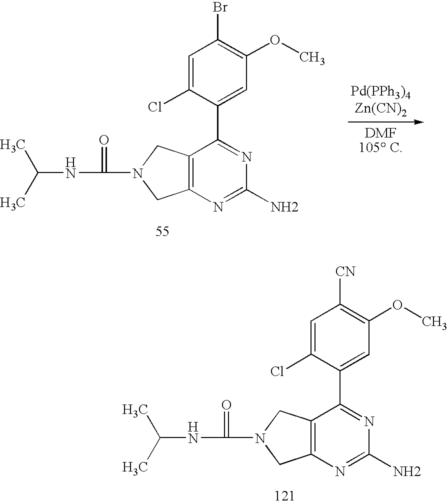

Compound 55 (0.0247 g, 0.056 mmol) and zinc cyanide (0.0212 g, 0.181 mmol) in DMF (1.5 mL) were purged with N₂. Tetrakis(triphenylphosphino)palladium(0) (0.0102 mg, 0.00883 mmol) was added and the mixture was heated at 105° C. for 5 h. Ethyl acetate (50 mL) extractive work up, washing with water (50 mL) followed by isolation using preparative HPLC gave compound 121 (0.008 g) as the hydrochloride in 30% yield. ¹H NMR (300 MHz, DMSO-d6) d ppm 1.06 (d, J=6.59 Hz, 6H) 3.71-3.85 (m, 1H) 3.93 (s, 3H) 4.25 (s, 2H) 4.43 (s, 2H) 6.07 (d, J=7.54 Hz, 1H) 6.95 (br. s., 2H) 7.34 (s, 1H) 8.12 (s, 1H). LCMS (M+H)⁺: 387.2.

### Example 122

### 2-amino-4-(4-bromo-2-chloro-5-methoxyphenyl)-5,7-dihydro-6H-pyrrolo[3,4-d]pyrimidine-6-carboximidamide

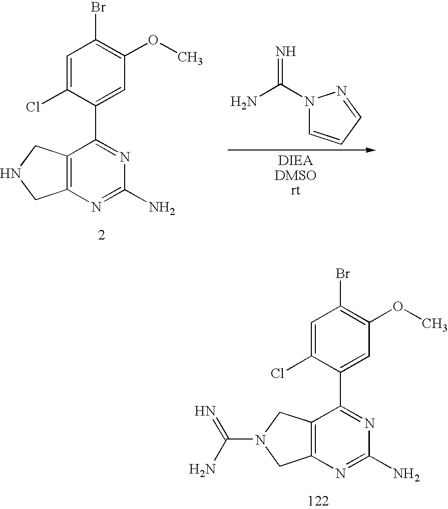

To compound 2 (0.0779 g, 0.105 mmol) in DMSO (1.0 mL) at ambient temperature was added diisopropyl ethylamine (0.10 mL, 0.57 mmol) and (1H)-pyrazole-1-carboxamidine hydrochloride (0.0201 g, 0.137 mmol). After stirring for 16 h, isolation using preparative

HPLC gave compound 122 (0.016 g, 0.040 mmol) as a white solid in 38% yield. ¹H NMR (300 MHz, DMSO-d6) δ ppm 3.88 (s, 3H) 4.45 (s, 2H) 4.60 (s, 2H) 7.07 (br. s., 2H) 7.17 (s, 1H) 7.31-7.47 (br. m, 3H) 7.90 (s, 1H). LCMS (M+H)⁺: 399.0, 397.2.

### Example 123

### ethyl 2-amino-4-(2,4-dichloro-5-ethylphenyl)-5,7-dihydro-6H-pyrrolo[3,4-d]pyrimidine-6-carboxylate

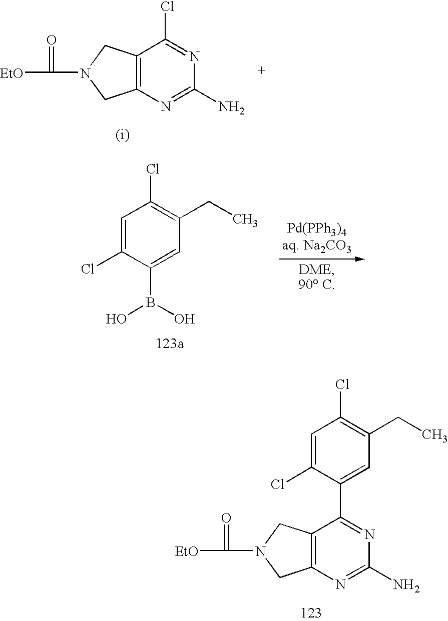

Compound (i) (0.0953 g, 0.393 mmol), compound 123a (0.086 mg, 0.39 mmol), 2M aqueous sodium carbonate (0.4 mL, 0.8 mmol), and tetrakis(triphenylphosphino)palladium(0) (0.0227 mg, 0.0196 mmol) in DME (4 mL) were reacted in a manner similar to Example 1. Isolation using preparative HPLC gave compound 123 (0.047 g, 0.12 mmol) as a white powder in 31% yield. ¹H NMR (300 MHz, DMSO-d6) δ ppm 1.13-1.24 (m, 6H) 2.73 (q, J=7.41 Hz, 2H) 4.01-4.14 (m, 2H) 4.30-4.36 (m, 2H) 4.44-4.51 (m, 2H) 6.94 (br. s., 2H) 7.42 (s, 1H) 7.73 (s, 1H). LCMS (M+H)⁺: 383.2, 381.2.

### Preparation of Compound 123a, (2,4-dichloro-5-ethylphenyl)boronic acid

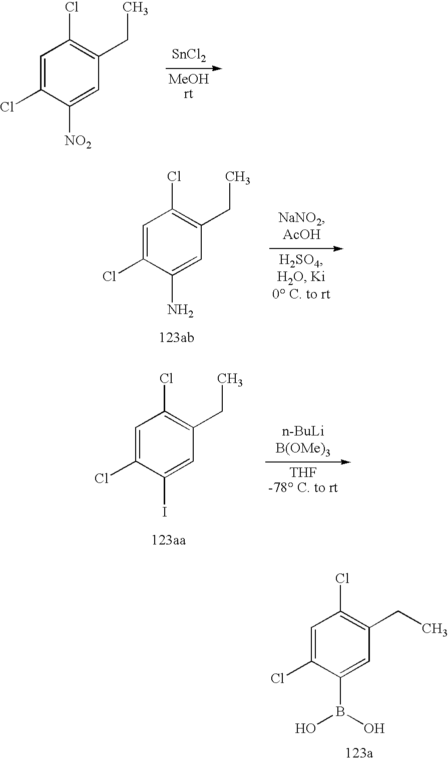

1,5-Dichloro-2-ethyl-4-nitrobenzene (2.1 g, 9.5 mmol) and SnCl₂ (8.1 g, 42.8 mmol) were refluxed in anhydrous methanol until the reduction was complete. Aqueous 10% NaOH and EtOAc were added, and the resulting suspended solids were allowed to settle and most of the EtOAc was decanted. This procedure was repeated several times and the EtOAc batches were combined and filtered through celite, dried (Na₂SO₄), filtered, and the volatile components removed. The residue was dissolved in Et₂O and excess 4M HCl in 1,4-dioxane was added. Evaporation of the volatile components gave compound 123ab, 2,4-dichloro-5-ethylaniline, (2.1 g, 9.27 mmol) as a yellow hydrochloride in 97% yield. Compound 123ab was converted to compound 123aa, 1,5-dichloro-2-ethyl-4-iodobenzene, (0.577 g, 1.92 mmol) in 36% yield in a manner similar to Example 117ba except that compound 123ab was substituted for compound 117bb. ¹H NMR (300 MHz, CHLOROFORM-d) δ ppm 1.22 (t, J=7.54 Hz, 3H) 2.69 (q, J=7.54 Hz, 2H) 7.44 (s, 1H) 7.70 (s, 1H). To compound 123aa (0.576 g, 1.91 mmol) in THF (15.0 mL) at −78° C. was added 2M n-BuLi in cyclohexane (1.00 mL, 2.00 mmol) dropwise. After 1 h, trimethyl borate (0.30 mL, 2.7 mmol) was added dropwise, and the reaction was allowed to warm to ambient temperature. Water (50 mL) followed by ether/hexanes (25 mL/25 mL) were added, and the layers separated. The organic layer was then extracted with 0.5N NaOH (30 mL), and the aqueous extraction was brought to pH 6 with 1N KH₂PO₄ (15 mL) and extracted with EtOAc (50 mL). Removal of the volatile components of the EtOAc layer gave compound 123a (0.088 g) as white waxy solid which was carried on without purification.

### Example 124

### ethyl 2-amino-4-[2-chloro-6-(4,4,4-trifluorobutoxy)phenyl]-5,7-dihydro-6H-pyrrolo[3,4-d]pyrimidine-6-carboxylate

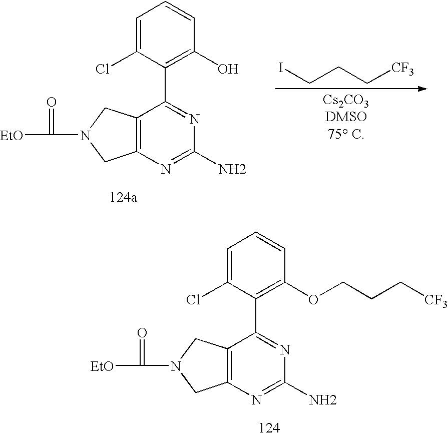

Compound 124a (0.0763 g, 0.228 mmol) was O-alkylated with 1-iodo-4,4,4-trifluorobutane (0.0801 g, 0.337 mmol) and cesium carbonate (0.0992 mg, 0.304 mmol) in DMSO (1.0 mL) in a manner similar to that described for Example 18. Isolation using preparative HPLC gave compound 124 (0.0353 g, 0.0794 mmol) as a white powder in 35% yield. ¹H NMR (300 MHz, DMSO-d6) δ ppm 1.11-1.25 (m, 3H) 1.70-1.82 (m, 2H) 2.03-2.23 (m, 2H) 3.99-4.21 (m, 6H) 4.37-4.57 (m, 2H) 6.83 (br. s., 2H) 7.11-7.20 (m, 2H) 7.44 (t, J=8.19 Hz, 1H). LCMS (M+H)⁺: 445.2.

### Preparation of Compound 124a, ethyl 2-amino-4-(2-chloro-6-hydroxyphenyl)-5,7-dihydro-6H-pyrrolo[3,4-d]pyrimidine-6-carboxylate

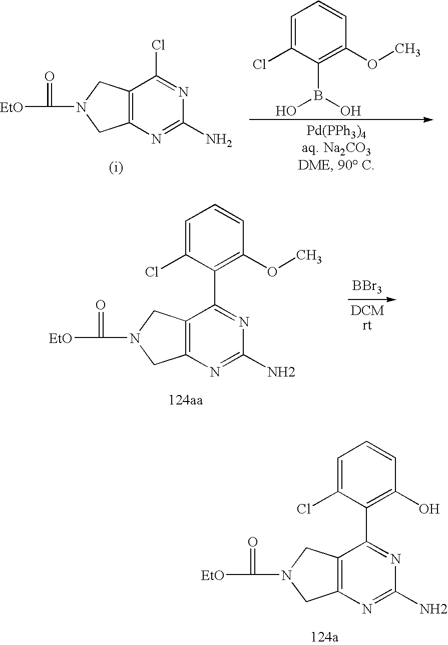

Compound (i) (0.300 g, 1.24 mmol), 2-chloro-6-methoxyphenylboronic acid (0.351 g, 1.88 mmol), 2M aqueous sodium carbonate (1.85 mL, 3.70 mmol) and tetrakis(triphenylphosphino) palladium(0) (0.0789 g, 0.0683 mmol) in DME (10.0 mL) were reacted in a manner similar to that described for Example 1 to give crude compound 124aa, ethyl 2-amino-4-(2-chloro-6-methoxyphenyl)-5,7-dihydro-6H-pyrrolo[3,4-d]pyrimidine-6-carboxylate, (0.481 g) which was carried on without purification. LCMS (M+H)⁺: 349.2. To compound 124aa (0.407 g) in dichloromethane (10.0 mL) at 0° C. was added dropwise 1M boron tribromide (0.20 mL, 2.1 mmol) in dichloromethane. The mixture was allowed to warm to ambient temperature and stir for 2 h. Additional boron tribromide (0.20 mL, 2.1 mmol) in dichloromethane was added and the mixture stirred for an additional 4 h. The mixture was then poured into cold saturated aqueous NaHCO₃ and extracted with dichloromethane/MeOH. Removal of the volatile components gave compound 124a (0.249 g) as an orange residue which was carried on without purification. LCMS (M+H)⁺: 335.2.

### Example 125

### ethyl 2-amino-4-(4-bromo-2-ethylphenyl)-5,7-dihydro-6H-pyrrolo[3,4-d]pyrimidine-6-carboxylate

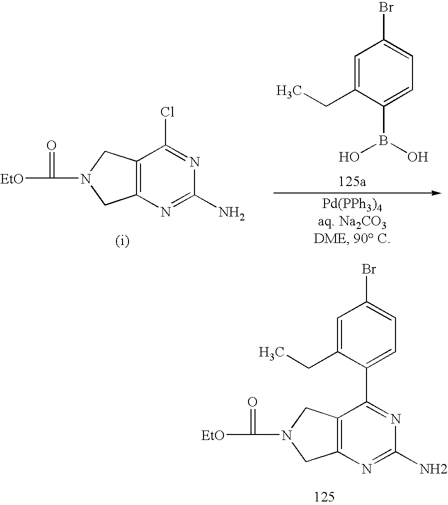

Compound 125a, 4-bromo-2-ethylphenylboronic acid, was prepared in a similar manner to Example 123a except that 4-bromo-2-ethyl-1-iodobenzene was substituted for compound 123aa. ¹H NMR (300 MHz, DMSO-d₆) δ ppm 1.19 (t, J=7.35 Hz, 3H) 3.02 (q, J=7.41 Hz, 2H) 7.33-7.41 (m, 2H) 7.74 (d, J=7.91 Hz, 1H). Compound (i) (0.114 g, 0.470 mmol), compound 125a (0.0852 g, 0.372 mmol), 2M aqueous sodium carbonate (0.50 mL, 1.0 mmol) and tetrakis(triphenylphosphino)palladium(0) (0.031 g, 0.027 mmol) in DME (3.5 mL) were reacted in a manner similar to that described for Example 1. Isolation using preparative HPLC gave compound 125 (0.029 g, 0.074 mmol) in 15% yield. ¹H NMR (300 MHz, DMSO-d6) δ ppm 1.02 (t, J=7.54 Hz, 3H) 1.13-1.25 (m, 3H) 2.55-2.63 (m, 2H) 4.00-4.13 (m, 2H) 4.26-4.32 (m, 2H) 4.44-4.50 (m, 2H) 6.82 (br. s., 2H) 7.24 (d, J=7.91 Hz, 1H) 7.48 (dd, J=8.01, 1.60 Hz, 1H) 7.57 (d, J=1.70 Hz, 1H). LCMS (M+H)⁺: 393.2, 391.2.

### Example 126

### 2-amino-N-cyclobutyl-4-(2,5-dimethoxy-4-methylphenyl)-5,7-dihydro-6H-pyrrolo[3,4-d]pyrimidine-6-carboxamide

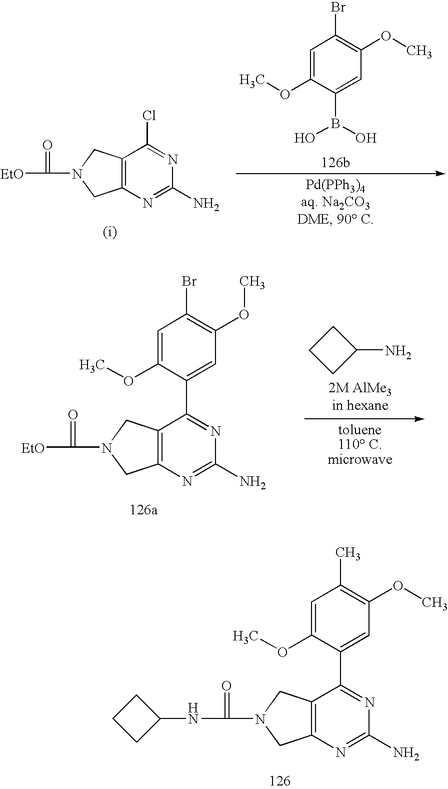

Compound 126b, 4-dibromo-2,5-dimethoxyphenylboronic acid, was prepared in a similar manner as Example 123a except that 1,4-dibromo-2,5-dimethoxybenzene was substituted for compound 123aa. ¹H NMR (300 MHz, DMSO-d₆) δ ppm 3.77 (s, 3H) 3.78 (s, 3H) 7.20 (s, 1H) 7.22 (s, 1H) 7.83 (s, 2H). Compound (i) (0.114 g, 0.470 mmol), compound 126b (0.112 g, 0.429 mmol), 2M aqueous sodium carbonate (0.50 mL, 1.0 mmol) and tetrakis(triphenylphosphino)palladium(0) (0.030 g, 0.026 mmol) in DME (3.5 mL) were reacted in a manner similar to Example 1 to give compound 126a, ethyl 2-amino-4-(4-bromo-2,5-dimethoxyphenyl)-5,7-dihydro-6H-pyrrolo[3,4-d]pyrimidine-6-carboxylate, (0.065 g, 0.154 mmol) in 36% yield which was carried on without purification. ¹H NMR (300 MHz, DMSO-d₆) δ ppm 1.14-1.25 (m, 3H) 3.78 (s, 3H) 3.80 (s, 3H) 4.02-4.14 (m, 2H) 4.27-4.33 (m, 2H) 4.42-4.48 (m, 2H) 6.82 (br. s., 2H) 7.05 (s, 1H) 7.39 (s, 1H). LCMS (M+H)⁺: 425.2, 423.2. Cyclobutylamine (0.10 mL, 1.2 mmol), 2.0 M trimethylaluminum (0.30 mL, 0.6 mmol) in hexane, and compound 126a (49.2 mg, 0.116 mmol) in toluene (2.0 mL) were microwaved for 1 h in a manner similar to Example 7. Work up, also in a similar manner, and isolation using preparative HPLC gave compound 126 (0.0174 g, 0.0454 mmol) in 30% yield as a TFA salt (conversion of the bromine in compound 126a to the methyl group of compound 126 was unanticipated). ¹H NMR (300 MHz, DMSO-d6) δ ppm 1.50-1.63 (m, 2H) 1.88-2.03 (m, 2H) 2.06-2.17 (m, 2H) 2.24 (s, 3H) 3.77 (s, 6H) 4.10-4.20 (m, 1H) 4.28 (s, 2H) 4.42 (s, 2H) 6.53 (d, J=7.72 Hz, 1H) 6.91 (s, 1H) 7.04 (s, 1H). LCMS (M+H)⁺: 384.4.

### Example 127

### 2-amino-N-cyclopropyl-4-{2,4-dichloro-5-[2-(1H-imidazol-1-yl)ethoxy]phenyl}-5,7-dihydro-6H-pyrrolo[3,4-d]pyrimidine-6-carboxamide

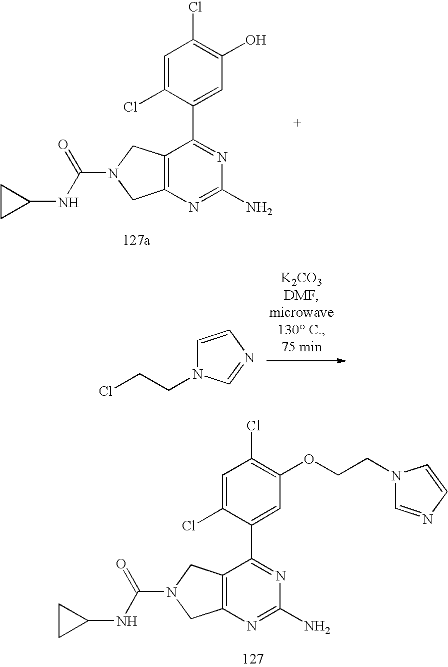

Compound 127a (70 mg, 0.184 mmol), 1-(2-chloroethyl)-1H-imidazole HCl (84 mg, 0.55 mmol), and potassium carbonate (127 mg, 0.92 mmol) in DMF (2 mL) were reacted in a manner similar to Example 100. Isolation using preparative HPLC gave compound 127 (9.0 mg, 0.02 mmol) as a powder in 11% yield. ¹H-NMR (DMSO-d6, 300 MHz): 7.71 (s, 1H), 7.63 (s, 1H), 7.19 (s, 1H), 7.16 (s, 1H), 6.85 (m, 3H), 6.43 (s, 1H) 4.37 (s, 4H), 4.31 (m, 2H), 4.18 (s, 2H), 2.69 (bs, 1H), 0.50 (m, 2H), 0.36 (m, 2H).

### Preparation of Compound 127a, 2-amino-N-cyclopropyl-4-(2,4-dichloro-5-hydroxyphenyl)-5,7-dihydro-6H-pyrrolo[3,4-d]pyrimidine-6-carboxamide

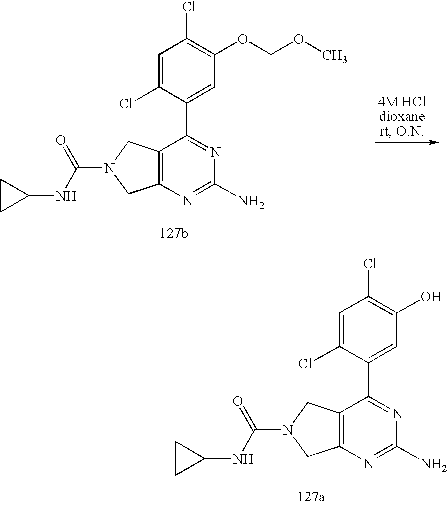

To a suspension of compound 127b (640 mg, 1.51 mol) in MeOH (4 mL) was added 4 M HCl in 1,4-dioxane (4 mL, 16.0 mmol). After stirring overnight, the volatile components were removed and the residue subjected to EtOAc extractive work up, washing with saturated aqueous NaHCO₃. Drying (Na₂SO₄), filtration, and evaporation of the volatile components gave compound 127a (426 mg, 1.12 mmol) as a powder in 74% yield which was carried on without purification. ¹H NMR (400 MHz, DMSO-d6) d ppm: 10.77 (s, 1H), 7.62 (s, 1H), 6.94 (s, 1H), 6.87 (s, 2H), 6.47 (d, J=2.78 Hz, 1H), 4.39 (s, 2H), 4.23 (s, 2H), 2.51-2.56 (m, 1H), 0.50-0.57 (m, 2H), 0.36-0.43 (m, 2H). LCMS (M+H)⁺: 380.2, 382.2

### Preparation of Compound 127b, 2-amino-N-cyclopropyl-4-[2,4-dichloro-5-(methoxymethoxy)phenyl]-5,7-dihydro-6H-pyrrolo[3,4-d]pyrimidine-6-carboxamide

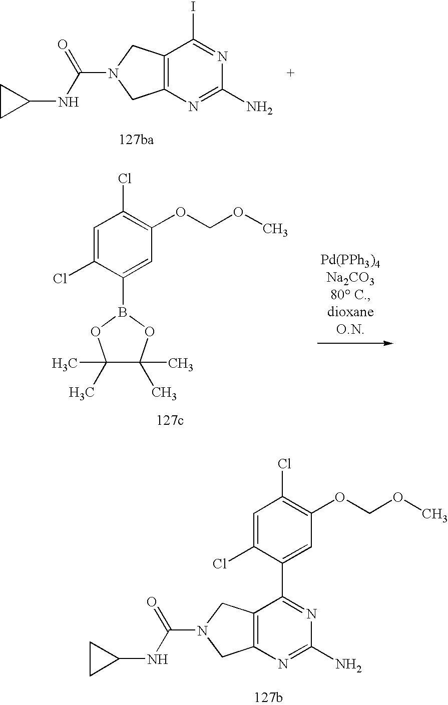

Compound 127ba, 2-amino-N-cyclopropyl-4-iodo-5,7-dihydro-6H-pyrrolo[3,4-d]pyrimidine-6-carboxamide, was prepared from compound 8b in a manner similar to example 18b except that cyclopropylamine was substituted for cyclobutylamine. Compound 127ba (825 mg, 2.39 mmol), compound 127c (876 mg, 2.63 mmol), 2M aqueous sodium carbonate (3.6 mL, 7.18 mmol) and tetrakis(triphenylphosphine) palladium(0) (276 mg, 0.239 mmol) in 30 mL 1,4-dioxane were reacted in a manner similar to Example 18a. After filtering and washing the collected insoluble material with EtOAc, the combined filtrate/washings were concentrated and subjected to EtOAc (100 mL) extractive work up, washing with water (200 mL), brine (2×100 mL), and drying (Na₂SO₄). Isolation using silica gel chromatography (gradient 0-5% MeOH in EtOAc) gave compound 127b (656 mg, 1.55 mmol) as a solid in 65% yield. ¹H NMR (400 MHz, DMSO-d6) d ppm: 7.78 (s, 1H), 7.28 (s, 1H), 6.90 (s, 2H), 6.46 (d, J=2.78 Hz, 1H), 5.32 (s, 2H), 4.40 (s, 2H), 4.23 (s, 2H), 3.41 (s, 3H), 0.49-0.60 (m, 2H), 0.36-0.44 (m, 2H). LCMS (M+H)⁺: 424.2, 426.2.

### Preparation of Compound 127c, 2-(2,4-dchloro-5-methoxymethoxy-phenyl)-4,4,5,5-tetramethyl-[1,3,2]dioxaborolane

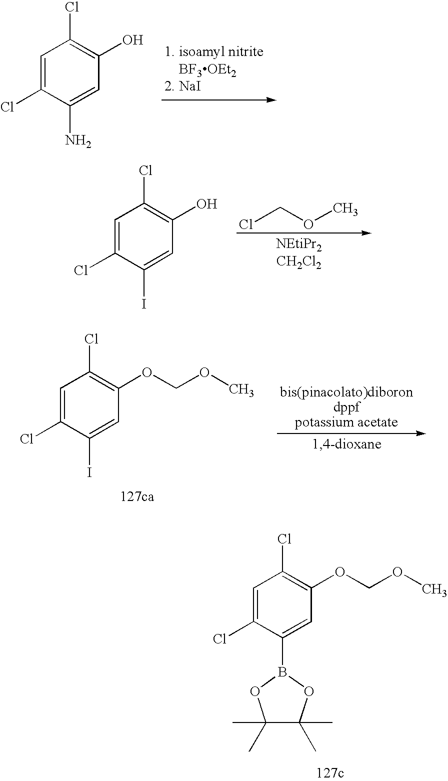

BF₃.OEt₂ (70.6 mL, 560 mmol) was cooled to 0° C. and 5-amino-2,4-dichlorophenol (50.0 g, 280 mmol) in THF (700 mL) was added over 45 min. Then isoamyl nitrite (48.8 mL, 365 mmol) in THF (150 mL) was added over 15 min. After stirring for 30 min at 0° C. a yellow precipitate formed, more Et₂O was added and the yellow solid was collected by filtration, washed with Et₂O, and air dried. The collected solid was added in portions to a solution of NaI (54.7 g, 365 mmol) in acetone (1.2 L) and stirred at ambient temperature overnight. The volatile components were reduced, water was added and the mixture was subjected to EtOAc extractive work up, washing with saturated aqueous NaHSO₃ and dried (Na₂SO₄). Isolation after silica gel chromatography plug (CH₂Cl₂) gave a mixture contaminated by the des-iodo impurity. Recrystallization from hexanes (3 crops) gave compound 127cb, 2,4-dichloro-5-iodophenol, (43.4 g, 150 mmol) as a yellow solid in 54% yield. ¹H NMR (400 MHz, CHLOROFORM-d) d ppm 3.77 (q, J=7.07 Hz, 3H) 5.27 (s, 2H) 7.45 (s, 1H) 7.66 (s, 1H). Compound 127cb in CH₂Cl₂ (400 mL) was cooled to 0° C. and methoxymethyl chloride (13.7 mL, 180 mmol) followed by diisopropylethyl amine (31.5 mL, 23.3 g, 180 mmol) were added. After warming to ambient temperature overnight, the CH₂Cl₂ solution was washed with saturated aqueous NaHCO₃ and dried over Na₂SO₄. Filtration and evaporation of the volatile components gave compound 127ca, 1,5-dichloro-2-iodo-4-(methoxymethoxy)benzene, (46.8 g, 141 mmol) as a yellow solid in 94% yield. Compound 127ca (41.8 g, 125 mmol), bis(pinacolato)diboron (35.1 g, 138 mmol), potassium acetate (24.6 g, 250 mmol), bis(diphenylphosphino)ferrocene-dichloropalladium dichloromethane adduct (6.15 g, 7.5 mmol), and dry 1,4-dioxane (375 mL) were purged with N₂ for 1 h and heated to 90° C. After 5 days, the mixture was cooled to ambient temperature, filtered, and the collected solid washed with Et₂O. The combined filtrate/washings were subjected to silica gel chromatography (stepwise gradient of 0 to 17% EtOAc in hexane) and isolation gave compound 127c (30.2 g, 91 mmol) as a yellow solid in 73% yield (>94% purity by GCMS).

### Example 128

### tert-butyl 3-[(2-{2-amino-6-[(cyclopropylamino)carbonyl]-6,7-dihydro-5H-pyrrolo[3,4-d]pyrimidin-4-yl}-3,5-dichlorophenoxy)methyl]azetidine-1-carboxylate

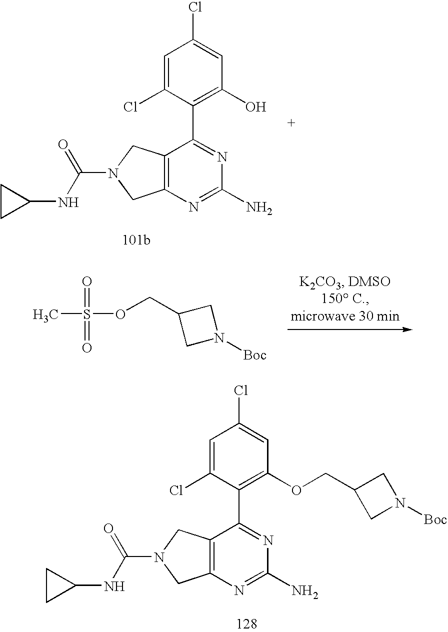

Compound 101b (300 mg, 0.789 mmol), tert-butyl 3-{[(methylsulfonyl)oxy]methyl}azetidine-1-carboxylate (419 mg, 1.58 mmol), and potassium carbonate (327 mg, 2.37 mmol) in DMF (10 mL) were microwaved at 150° C. for 30 min. Following, EtOAc extractive work up, washing with water and brine, and drying (Na₂SO₄), isolation using silica gel chromatography (10% MeOH in EtOAc) gave compound 128 (272 mg, 0.495 mmol) in 63% yield. ¹H NMR (400 MHz, CHLOROFORM-d) d ppm: 7.15 (d, J=1.77 Hz, 1H), 6.90 (d, J=1.77 Hz, 1H), 5.15 (s, 2 H), 4.55 (s, 3H), 4.35-4.42 (m, 1H), 4.21-4.29 (m, 1H), 4.01-4.13 (m, 2H), 3.86-3.96 (m, 2H), 3.53-3.62 (m, 2H), 2.74-2.86 (m, 1H), 2.64-2.74 (m, 1H), 1.43 (s, 9H), 0.71-0.79 (m, 2H), 0.48-0.56 (m, 2H). LCMS (M+H)⁺: 549.2, 550.2.

### Example 129

### 2-amino-4-[2-(azetidin-3-ylmethoxy)-4,6-dichlorophenyl]-N-cyclopropyl-5,7-dihydro-6H-pyrrolo[3,4-d]pyrimidine-6-carboxamide

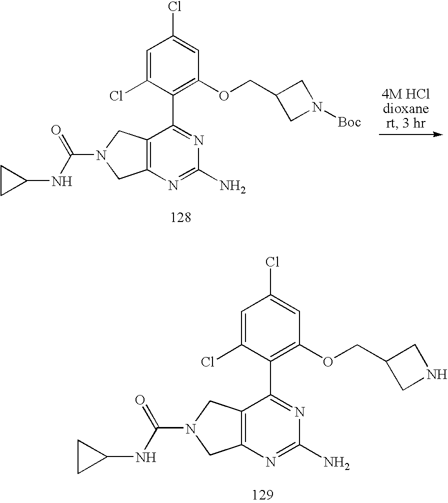

To compound 128 (490 mg, 0.892 mmol, 1.0 eq) in 1,4-dioxane (5 mL) was added 4 M HCl in 1,4-dioxane (5 mL). After stirring 3 h, isolation by preparative HPLC gave compound 129 (25 mg, 0.056 mmol) as a solid in 6% yield. ¹H NMR (400 MHz, MeOD) ppm 8.55 (s, 1H), 7.15-7.35 (m, 2H), 4.44-4.62 (m, 2H), 4.15-4.40 (m, 4H), 3.85-4.03 (m, 2H), 3.66-3.83 (m, 2H), 3.13-3.27 (m, 1H), 2.47-2.63 (m, 1H), 0.58-0.75 (m, 2H), 0.49 (d, J=2.02 Hz, 2H). LCMS (M+H)⁺: 449.2, 450.2.

### Example 130

### 2-amino-4-(2,4-dichloro-6-{[1-(cyanomethyl)azetidin-3-yl]methoxy}phenyl)-N-ethyl-5,7-dihydro-6H-pyrrolo[3,4-d]pyrimidine-6-carboxamide

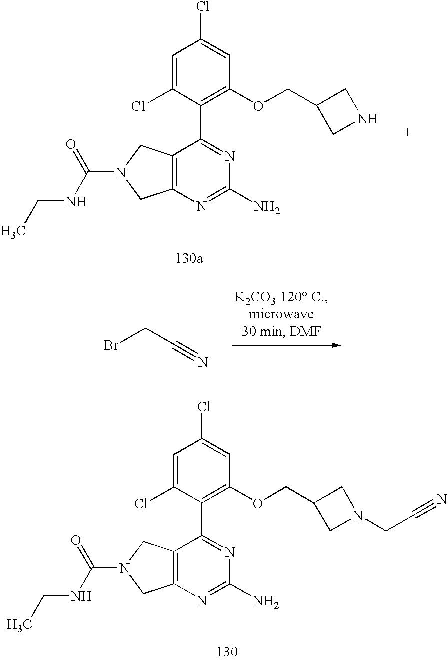

Compound 130a, 2-amino-4-[2-(azetidin-3-ylmethoxy)-4,6-dichlorophenyl]-N-ethyl-5,7-dihydro-6H-pyrrolo[3,4-c]pyrimidine-6-carboxamide was prepared from compound 131 in a similar manner to Example 129. A mixture of compound 130a, (78 mg, 0.180 mmol), bromoacetonitrile (26 mg, 0.216 mmol), and potassium carbonate (124 mg, 0.899 mmol) in DMF (2.5 mL) were microwaved at 120° C. for 30 min. Isolation by preparative HPLC gave compound 130 (20.0 mg, 0.042 mmol) as a powder in 23% yield. ¹H NMR (400 MHz, DMSO-d6) d ppm: 7.35 (d, J=1.26 Hz, 1H), 7.32 (d, J=1.77 Hz, 1H), 6.81 (br. s., 2H), 6.34 (t, J=5.31 Hz, 1H), 4.42 (s, 2H), 4.04-4.20 (m, 4H), 3.43-3.48 (m, 2H), 3.25-3.34 (m, 2H), 3.00-3.10 (m, 2H), 2.87-2.99 (m, 2H), 2.62-2.75 (m, 1H), 1.01 (t, J=7.07 Hz, 3H). LCMS (M+H)⁺: 476.0, 478.0.

Compounds of Examples 131-2xx were prepared following the methods of Examples 1-18 and 100-130, as shown in the following Table 2.

TABLE 2

| Ex. | | | Synthetic | | |
|---|---|---|---|---|---|
| No. | Structure | Name | Method | ¹H NMR | MS |
| 131 | 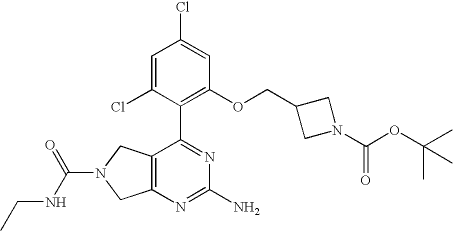 | tert-butyl 3-[(2-{2-amino-6- [(ethylamino)carbonyl]-6,7-dihydro-5 H- pyrrolo[3,4-d]pyrimidin-4-yl}-3,5- dichlorophenoxy)methyl]azetidine-1- carboxylate | Ex. 128 | ¹H NMR (400 MHz, CHLOROFORM-d) d ppm: 7.15 (d, J = 1.77 Hz, 1 H), 6.90 (d, J = 1.77 Hz, 1 H), 5.17 (s, 2 H), 4.58 (s, 2 H), 4.37-4.45 (m, 1 H), 4.20-4.32 (m, 2 H), 4.01-4.13 (m, 2 H), 3.87-3.96 (m, 2 H), 3.53-3.62 (m, 2 H), 3.27-3.38 (m, 2 H), 2.73-2.87 (m, 1 H), 1.42 (s, 9 H), 1.17 (t, J = 7.20 Hz, 3 H). | 537.1, 539.1 |
| 132 | 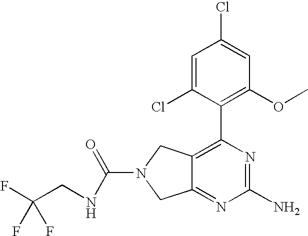 | 2-amino-4-(2,4-dichloro-6- methoxyphenyl)-N-(2,2,2- trifluoroethyl)-5,7-dihydro-6H- pyrrolo[3,4-d]pyrimidine-6- carboxamide | Ex. 102 | 1H NMR (400 MHz, DMSO-d6) d ppm 3.78 (s, 3 H), 3.82 (dd, J = 9.60, 6.57 Hz, 2 H), 4.16 (s, 2 H), 4.48 (s, 2 H), 6.87 (s, 2 H), 7.07 (br. s., 1 H), 7.28 (d, J = 1.52 Hz, 1 H), 7.34 (d, J = 1.77 Hz, 1 H) | 436.0, 438.0 |
| 133 | 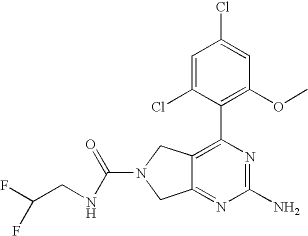 | 2-amino-4-(2,4-dichloro-6- methoxyphenyl)-N-(2,2-difluoroethyl)- 5,7-dihydro-6H-pyrrolo[3,4- d]pyrimidine-6-carboxamide | Ex. 102 | 1H NMR (400 MHz, DMSO-d6) d ppm 3.38-3.48 (m, 2 H), 3.72-3.82 (m, 3 H), 4.11-4.18 (m, 2 H), 4.46 (s, 2 H), 5.74-6.15 (m, 1 H), 6.82 (t, J = 5.68 Hz, 1 H), 6.86 (s, 2 H), 7.28 (d, J = 1.77 Hz, 1 H), 7.34 (d, J = 1.77 Hz, 1 H) | 418.0, 420.0 |
| 134 | 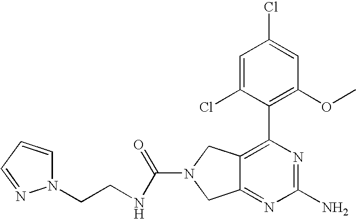 | 2-amino-4-(2,4-dichloro-6- methoxyphenyl)-N-[2-(1H-pyrazol-1- yl)ethyl]-5,7-dihydro-6H-pyrrolo[3,4- d]pyrimidine-6-carboxamide | Ex. 102 | 1H NMR (400 MHz, DMSO-d6) d ppm 3.35-3.40 (m, 2 H), 3.78 (s, 3 H), 4.09 (s, 2 H), 4.15 (t, J = 6.57 Hz, 2 H), 4.41 (s, 2 H), 6.20 (t, J = 2.02 Hz, 1 H), 6.54 (t, J = 5.05 Hz, 1 H), 6.86 (br.s., 2 H), 7.28 (d, J = 1.52 Hz, 1 H), 7.34 (d, J = 1.77 Hz, 1 H), 7.42 (d, J = 1.26 Hz, 1 H), 7.67 (d, J = 2.02 Hz, 1 H) | 448.2, 450.2 |
| 135 | 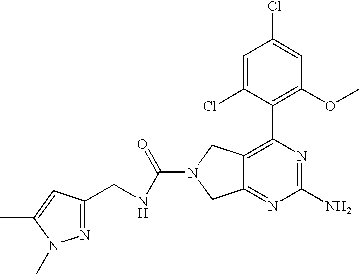 | 2-amino-4-(2,4-dichloro-6- methoxyphenyl)-N-[2-(1H-pyrazol-1- yl)ethyl]-5,7-dihydro-6H-pyrrolo[3,4- d]pyrimidine-6-carboxamide | Ex. 102 | 1H NMR (400 MHz, DMSO-d6) d ppm 2.17 (s, 3 H), 3.62 (s, 3 H), 3.77 (s, 3 H), 4.08 (d, J = 5.31 Hz, 2 H), 4.12 (s, 2 H), 4.44 (s, 2 H), 5.88 (s, 1 H), 6.73 (t, J = 5.68 Hz, 1 H), 6.84 (s, 2 H), 7.28 (d, J = 1.52 Hz, 1 H), 7.34 (d, J = 1.77 Hz, 1 H) | 462.2, 464.2 |
| 136 | 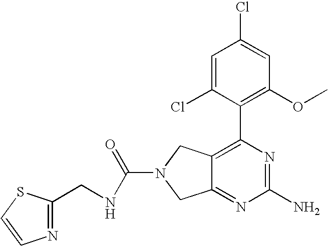 | 2-amino-4-(2,4-dichloro-6- methoxyphenyl)-N-(1,3-thiazol-2- ylmethyl)-5,7-dihydro-6H-pyrrolo[3,4- d]pyrimidine-6-carboxamide | Ex. 102 | 1H NMR (400 MHz, DMSO-d6) d ppm 3.78 (s, 3 H), 4.18 (s, 2 H), 4.43-4.56 (m, 4 H), 6.87 (s, 2 H), 7.28 (s, 1 H), 7.35 (s, 1 H), 7.36-7.42 (m, 1 H), 7.55-7.60 (m, 1 H), 7.66-7.71 (m, 1 H) | 451.0, 453.0 |
| 137 | 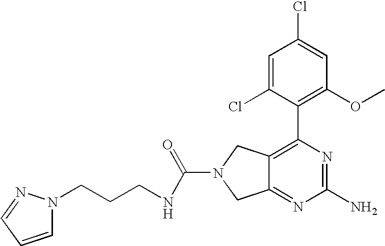 | 2-amino-4-(2,4-dichloro-6- methoxyphenyl)-N-[3-(1H-pyrazol-1- yl)propyl]5,7-dihydro-6H-pyrrolo[3,4- d]pyrimidine-6-carboxamide | Ex. 102 | 1H NMR (400 MHz, DMSO-d6) d ppm 1.89 (t, J = 6.95 Hz, 2 H), 3.02 (q, J = 6.40 Hz, 2 H), 3.77 (s, 3 H), 4.06-4.15 (m, 4 H), 4.42 (s, 2 H), 6.19 (t, J = 2.02 Hz, 1 H), 6.40 (t, J = 5.43 Hz, 1 H), 6.84 (s, 2 H), 7.28 (d, J = 2.02 Hz, 1 H), 7.34 (d, J = 1.77 Hz, 1 H), 7.39 (d, J = 1.26 Hz, 1 H), 7.72 (d, J = 2.27 Hz, 1 H) | 462.2, 464.2 |
| 138 | 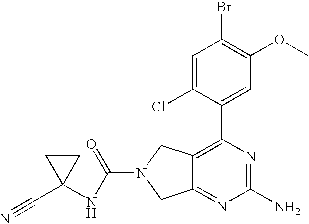 | 2-amino-4-(4-bromo-2-chloro-5- methoxyphenyl)-N-(1- cyanocyclopropyl)-5,7-dihydro-6H- pyrrolo[3,4-d]pyrimidine-6- carboxamide | Ex. 102 | 1H NMR (400 MHz, DMSO-d6) d ppm 1.06-1.17 (m, 2 H), 1.35-1.47 (m, 2 H), 3.86 (s, 3 H), 4.27 (br. s., 2 H), 4.45 (s, 2 H), 6.93 (s, 2 H), 7.15 (s, 1 H), 7.46 (br. s., 1 H), 7.87 (s, 1 H) | 463.0, 465.0 |
| 139 | 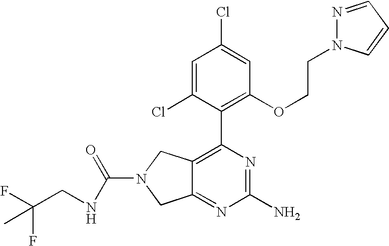 | 2-amino-4-{2,4-dichloro-6-[2-(1H- pyrazol-1-yl)ethoxy]phenyl}-N-(2,2- difluoropropyl)-5,7-dihydro-6H- pyrrolo[3,4-d]pyrimidine-6- carboxamide | Ex. 102 | 1H NMR (400 MHz, DMSO-d6) d ppm 1.54-1.66 (m, 3 H), 3.48 (td, J = 13.89, 8.08 Hz, 2 H), 3.58 (d, J = 12.88 Hz, 2 H), 3.97 (d, J = 12.88 Hz, 2 H), 4.33 (s, 4 H), 4.44 (br. s., 2 H), 6.00 (t, J = 2.02 Hz, 1 H), 6.66 (br. s., 1 H), 6.80 (s, 2 H), 7.20 (d, J = 2.02 Hz, 1 H), 7.26 (d, J = 1.52 Hz, 1 H), 7.29 (d, J = 1.52 Hz, 0 H), 7.33 (d, J = 1.52 Hz, 1 H) | 512.0, 514.0 |
| 140 | 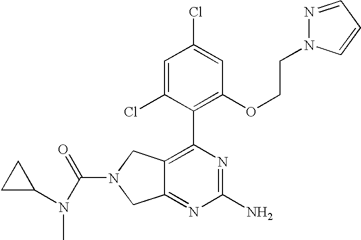 | 2-amino-N-cyclopropyl-4-{2,4- dichloro-6-[2-(1H-pyrazol-1- yl)ethoxy]phenyl}-N-methyl-5,7- dihydro-6H-pyrrolo[3,4-d]pyrimidine-6- carboxamide | Ex. 102 | 1H NMR (400 MHz, DMSO-d6) d ppm 0.50-0.63 (m, 2 H), 0.68 (dd, J = 6.57, 2.27 Hz, 2 H), 2.63 (ddd, J = 6.82, 3.28, 3.03 Hz, 1 H), 2.74 (s, 3 H), 3.82 (d, J = 13.39 Hz, 1 H), 4.18 (dd, J = 13.39, 1.52 Hz, 1 H), 4.33 (s, 4 H), 4.49-4.69 (m, 2 H), 6.06 (t, J = 2.02 Hz, 1 H), 6.76 (s, 2 H), 7.19 (d, J = 2.02 Hz, 1 H), 7.23 (d, J = 1.77 Hz, 1 H), 7.31 (d, J = 1.77 Hz, 1 H), 7.33 (d, J = 1.52 Hz, 1 H) | 489.0, 491.1 |
| 141 | 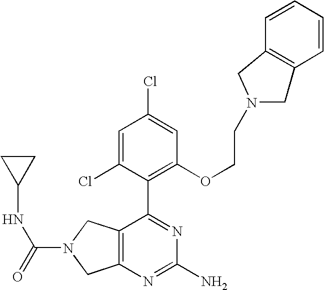 | 2-amino-N-cyclopropyl-4-{2,4- dichloro-6-[2-(1,3-dihydro-2 H-isoindol- 2-yl)ethoxy]phenyl}-5,7-dihydro-6H- pyrrolo[3,4-d]pyrimidine-6- carboxamide | Ex. 101 | 1H NMR (400 MHz, DMSO-d6) d ppm 0.32-0.41 (m, 2 H), 0.45-0.57 (m, 2 H), 1.91 (s, 1 H), 2.92 (br. s., 2 H), 3.69 (br. s., 4 H), 4.10-4.25 (m, 4 H), 4.26-4.36 (m, 1 H), 4.38-4.48 (m, 1 H), 6.45 (d, J = 2.78 Hz, 1 H), 6.90 (s, 2 H), 7.15 (s, 4 H), 7.31 (d, J = 1.77 Hz, 1 H), 7.35 (d, J = 1.52 Hz, 1 H) | 525.2, 527.2 |
| 142 | 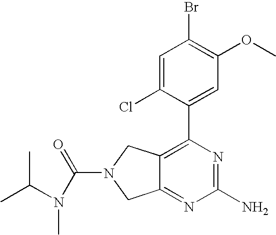 | 2-amino-4-(4-bromo-2-chloro-5- methoxyphenyl)-N-isopropyl-N-methyl- 5,7-dihydro-6H-pyrrolo[3,4- d]pyrimidine-6-carboxamide | Ex. 105 | 1H NMR (400 MHz, DMSO-d6) d ppm 1.07 (d, J = 6.57 Hz, 6 H), 2.64 (s, 3 H), 3.82-3.89 (m, 3 H), 3.94-4.08 (m, 1 H), 4.40 (s, 2 H), 4.53 (s, 2 H), 6.87 (s, 2 H), 7.16 (s, 1 H), 7.85 (s, 1 H) | 456.0, 458.0 |
| 143 | 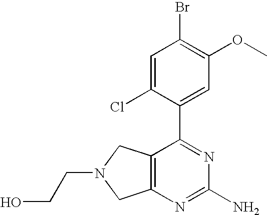 | 2-[2-amino-4-(4-bromo-2-chloro-5- methoxyphenyl)-5,7-dihydro-6H- pyrrolo[3,4-d]pyrimidin-6-yl]ethanol | Ex. 105 | 1H NMR (400 MHz, DMSO-d6) d ppm 3.44 (d, J = 10.36 Hz, 2 H), 3.71-3.79 (m, 2 H), 3.85-3.91 (m, 3 H), 4.46 (br. s., 2 H), 4.59 (s, 2 H), 5.36 (br. s., 1 H), 7.09 (s, 1 H), 7.17-7.24 (m, 2 H), 7.91 (s, 1 H) | 401.0, 403.0 |
| 144 | 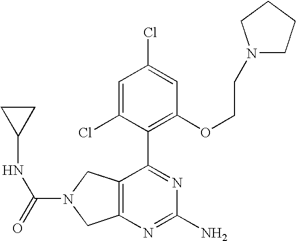 | 2-amino-N-cyclopropyl-4-[2,4-dichloro- 6-(2-pyrrolidin-1-ylethoxy)phenyl]-5,7- dihydro-6H-pyrrolo[3,4-d]pyrimidine-6- carboxamide | Ex. 101 | 1H NMR (400 MHz, DMSO-d6) d ppm 0.32-0.43 (m, 2 H), 0.46-0.58 (m, 2 H), 1.59 (br. s., 4 H), 2.25-2.45 (m, 3 H), 2.54 (s, 1 H), 2.62-2.84 (m, 2 H), 4.03-4.20 (m, 5 H), 4.29-4.48 (m, 2 H), 6.54 (s, 1 H), 6.80 (s, 2 H), 7.29 (s, 1 H), 7.34 (d, J = 1.52 Hz, 1 H) | 477.2, 479.2 |
| 145 | 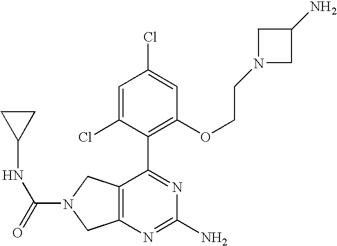 | 2-amino-4-{2-[2-(3-aminoazetidin-1- yl)ethoxy]-4,6-dichlorophenyl}-N- cyclopropyl-5,7-dihydro-6H- pyrrolo[3,4-d]pyrimidine-6- carboxamide | Ex. 101 | 1H NMR (400 MHz, DMSO-d6) d ppm 0.34-0.46 (m, 2 H), 0.47-0.60 (m, 2 H), 1.91 (s, 2 H), 2.59 (dd, J = 4.93, 3.16 Hz, 2 H), 2.62-2.75 (m, 4 H), 3.98 (t, J = 5.05 Hz, 2 H), 4.03-4.14 (m, 2 H), 4.14-4.25 (m, 2 H), 4.41 (s, 2 H), 6.46 (d, J = 2.53 Hz, 1 H), 6.77 (s, 2 H), 7.25 (d, J = 1.77 Hz, 1 H), 7.32 (d, J = 1.77 Hz, 1 H) | 478.2, 480.2 |
| 146 | 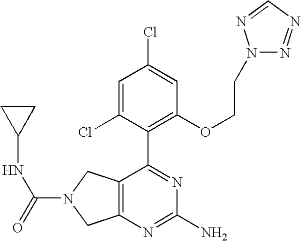 | 2-amino-N-cyclopropyl-4-{2,4- dichloro-6-[2-(2 H-tetrazol-2- yl)ethoxy]phenyl}-5,7-dihydro-6H- pyrrolo[3,4-d]pyrimidine-6- carboxamide | Ex. 101 | 1H NMR (400 MHz, DMSO-d6) d ppm 0.34-0.48 (m, 2 H), 0.47-0.59 (m, 2 H), 2.51-2.57 (m, 1 H), 3.62 (d, J = 13.14 Hz, 1 H), 3.95 (dd, J = 13.01, 1.89 Hz, 1 H), 4.28-4.45 (m, 3 H), 4.44-4.57 (m, 1 H), 4.75 (t, J = 5.05 Hz, 2 H), 6.34 (br. s., 1 H), 6.77 (br. s., 2 H), 7.33 (d, J = 1.77 Hz, 1 H), 7.36 (d, J = 1.77 Hz, 1 H), 8.82 (s, 1 H) | 476.1, 478.1 |
| 147 | 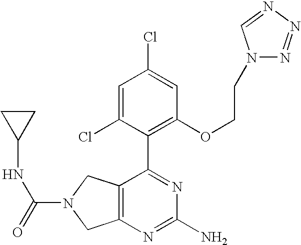 | 2-amino-N-cyclopropyl-4-{2,4- dichloro-6-[2-(1H-tetrazol-1- yl)ethoxy]phenyl}-5,7-dihydro-6H- pyrrolo[3,4-d]pyrimidine-6- carboxamide | Ex. 101 | 1H NMR (400 MHz, DMSO-d6) d ppm 0.36-0.48 (m, 2 H), 0.50-0.61 (m, 2 H), 2.53-2.58 (m, 1 H), 3.36-3.42 (m, 1 H), 3.83 (dd, J = 13.01, 2.15 Hz, 1 H), 4.25-4.47 (m, 2 H), 4.46-4.63 (m, 2 H), 4.91-5.04 (m, 2 H), 6.34 (br. s., 1 H), 6.72 (s, 2 H), 7.34 (s, 2 H), 8.61 (s, 1 H) | 476.1, 477.1 |
| 148 | 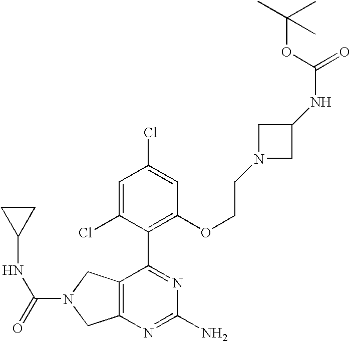 | tert-butyl {1-[2-(2-{2-amino-6- [(cyclopropylamino)carbonyl]-6,7- dihydro-5 H-pyrrolo[3,4-d]pyrimidin-4- yl}-3,5-dichlorophenoxy)ethyl]azetidin- 3-yl}carbamate | Ex. 101 | 1H NMR (400 MHz, CHLOROFORM-d) d ppm 1.44 (s, 9 H), 1.66 (br. s., 3 H), 2.70 (br. s., 4 H), 2.75-2.89 (m, 2 H), 3.37-3.47 (m, 1 H), 3.50 (s, 1 H), 3.86-4.03 (m, 2 H), 4.19 (br. s., 1 H), 4.34 (br. s., 2 H), 4.57 (s, 2 H), 4.62 (s, 1 H), 5.08 (br. s., 1 H), 5.30 (br. s., 2 H), 6.85 (d, J = 1.52 Hz, 1 H), 7.12 (d, J = 1.77 Hz, 1 H) | 578.2, 580.2 |
| 149 | 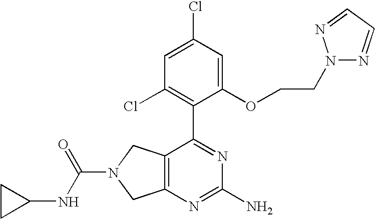 | 2-amino-N-cyclopropyl-4-{2,4- dichloro-6-[2-(2 H-1,2,3-triazol-2- yl)ethoxy]phenyl}-5,7-dihydro-6H- pyrrolo[3,4-d]pyrimidine-6- carboxamide | Ex. 101 | 1H NMR (400 MHz, DMSO-d6) d ppm 0.35-0.47 (m, 2 H), 0.56 (d, J = 7.07 Hz, 2 H), 2.52-2.60 (m, 1 H), 3.38-3.47 (m, 1 H), 3.84 (d, J = 13.14 Hz, 1 H), 4.35 (d, J = 4.80 Hz, 2 H), 4.42-4.56 (m, 2 H), 4.65 (t, J = 4.93 Hz, 2 H), 6.33 (br. s., 1 H), 6.53 (s, 1 H), 6.71 (s, 2 H), 7.26-7.37 (m, 1 H), 7.53 (s, 2 H) | 475.1, 477.1 |
| 150 | 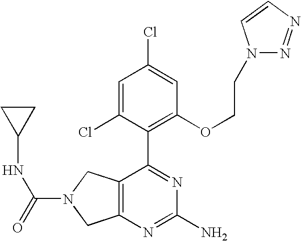 | 2-amino-N-cyclopropyl-4-{2,4- dichloro-6-[2-(1H-1,2,3-triazol-1- yl)ethoxy]phenyl}-5,7-dihydro-6H- pyrrolo[3,4-d]pyrimidine-6- carboxamide | Ex. 101 | 1H NMR (400 MHz, DMSO-d6) d ppm 0.35-0.48 (m, 2 H), 0.49-0.60 (m, 2 H), 2.52-2.58 (m, 1 H), 3.60 (d, J = 13.14 Hz, 1 H), 3.94 (dd, J = 12.88, 2.02 Hz, 1 H), 4.30-4.51 (m, 4 H), 4.59-4.71 (m, 2 H), 6.35 (br. s., 1 H), 6.79 (br. s., 2 H), 7.29 (d, J = 1.77 Hz, 1 H), 7.35 (d, J = 1.52 Hz, 1 H), 7.48 (s, 1 H), 7.56 (s, 1 H) | 475.1, 477.1 |
| 151 | 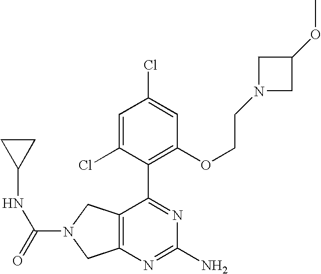 | 2-amino-N-cyclopropyl-4-{2,4- dichloro-6-[2-(3-methoxyazetidin-1- yl)ethoxy]phenyl}-5,7 dihydro-6H- pyrrolo[3,4-d]pyrimidine-6- carboxamide | Ex. 101 | 1H NMR (400 MHz, DMSO-d6) d ppm 0.34-0.46 (m, 2 H), 0.49-0.59 (m, 2 H), 2.52-2.57 (m, 1 H), 2.59-2.75 (m, 1 H), 3.07 (s, 3 H), 3.23-3.28 (m, 2 H), 3.79 (t, J = 5.68 Hz, 1 H), 3.93-4.05 (m, 2 H), 4.04-4.23 (m, 2 H), 4.29-4.48 (m, 2 H), 6.46 (d, J = 2.78 Hz, 1 H), 6.82 (s, 2 H), 7.23 (d, J = 1.52 Hz, 1 H), 7.32 (d, J = 1.77 Hz, 1 H) | 493.1, 495.1 |
| 152 | 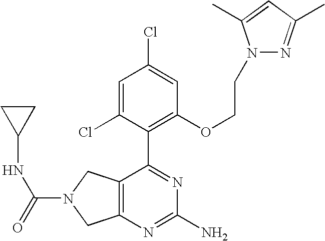 | 2-amino-N-cyclopropyl-4-{2,4- dichloro-6-[2-(3,5-dimethyl-1H-pyrazol- 1-yl)ethoxy]phenyl}-5,7-dihydro-6H- pyrrolo[3,4-d]pyrimidine-6- carboxamide | Ex. 101 | 1H NMR (400 MHz, DMSO-d6) d ppm 0.32-0.47 (m, 2 H), 0.54 (dd, J = 7.07, 2.27 Hz, 2 H), 1.66 (s, 3 H) 2.00 (s, 3 H), 2.54-2.58 (m, 1 H), 3.84 (d, J = 12.88 Hz, 1 H), 4.00-4.17 (m, 2 H), 4.16-4.27 (m, 2 H), 4.31-4.43 (m, 2 H), 5.49 (s, 1 H), 6.33 (br. s., 1 H), 6.53 (s, 1 H), 6.78 (s, 2 H), 7.25 (d, J = 1.77 Hz, 1 H), 7.31 (d, J = 1.77 Hz, 1 H) | 502.1, 504.1 |
| 153 | 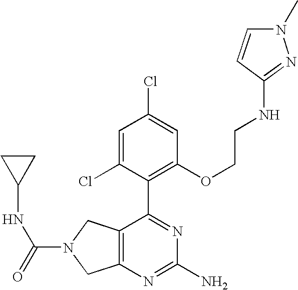 | 2-amino-N-cyclopropyl-4-(2,4-dichloro- 6-{2-[(1-methyl-1H-pyrazol-3- yl)amino]ethoxy}phenyl)-5,7-dihydro- 6H-pyrrolo[3,4-d]pyrimidine-6- carboxamide | Ex. 101 | 1H NMR (400 MHz, DMSO-d6) d ppm 0.35-0.45 (m, 2 H), 0.49-0.61 (m, 2 H), 2.53-2.58 (m, 1 H), 3.25 (t, J = 5.18 Hz, 2 H), 3.60-3.67 (m, 3 H), 4.04-4.12 (m, 2 H), 4.12-4.23 (m, 3 H), 4.40 (s, 2 H), 5.39 (d, J = 2.27 Hz, 1 H), 6.41 (br. s., 1 H), 6.62-7.04 (m, 2 H), 7.34 (d, J = 1.77 Hz, 1 H), 7.38 (d, J = 2.02 Hz, 1 H), 7.45 (d, J = 1.77 Hz, 1 H) | 503.1, 505.1 |
| 154 | 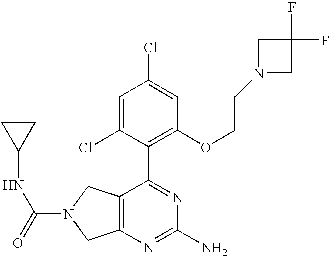 | 2-amino-N-cyclopropyl-4-{2,4- dichloro-6-[2-(3,3-difluoroazetidin-1- yl)ethoxy]phenyl}-5,7-dihydro-6H- pyrrolo[3,4-d]pyrimidine-6- carboxamide | Ex. 101 | 1H NMR (400 MHz, DMSO-d6) d ppm 0.34-0.44 (m, 2 H), 0.47-0.59 (m, 2 H), 2.74 (d, J = 3.54 Hz, 2 H), 3.22-3.35 (m, 4 H), 3.57 (s, 1 H), 3.97-4.09 (m, 2 H), 4.08-4.17 (m, 2 H), 4.26-4.51 (m, 2 H), 6.46 (d, J = 2.53 Hz, 1 H), 6.85 (s, 2 H), 7.25 (d, J = 1.77 Hz, 1 H), 7.30-7.38 (m, 1 H) | 499.1, 500.1 |
| 155 | 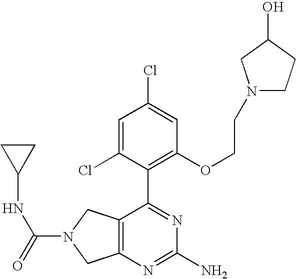 | 2-amino-N-cyclopropyl-4-{2,4- dichloro-6-[2-(3-hydroxypyrrolidin-1- yl)ethoxy]phenyl}-5,7-dihydro-6H- pyrrolo[3,4-d]pyrimidine-6- carboxamide | Ex. 101 | 1H NMR (400 MHz, DMSO-d6) d ppm 0.34-0.45 (m, 2 H), 0.47-0.58 (m, 2 H), 1.34-1.49 (m, 2 H), 1.74-1.88 (m, 2 H), 1.74-1.88 (m, 0 H), 2.17-2.34 (m, 2 H), 2.34-2.45 (m, 1 H), 2.55-2.73 (m, 3 H), 3.17 (d, J = 4.80 Hz, 2 H), 3.99-4.20 (m, 4 H), 4.38 (br. s., 2 H), 4.59 (br. s., 1 H), 6.38-6.48 (m, 1 H), 6.78 (s, 2 H), 7.27 (s, 1 H), 7.32 (d, J = 1.52 Hz, 1 H) | 493.1, 495.1 |
| 156 | 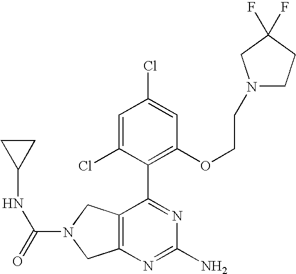 | 2-amino-N-cyclopropyl-4-{2,4- dichloro-6-[2-(3,3-difluoropyrrolidin-1- yl)ethoxy]phenyl}-5,7-dihydro-6H- pyrrolo[3,4-d]pyrimidine-6- carboxamide | Ex. 101 | 1H NMR (400 MHz, DMSO-d6) d ppm 0.33-0.43 (m, 2 H), 0.47-0.57 (m, 2 H), 2.09-2.32 (m, 2 H), 2.52-2.58 (m, 1 H), 3.00 (d, J = 3.79 Hz, 3 H), 3.13-3.21 (m, 2 H), 4.11 (s, 2 H), 4.16-4.26 (m, 2 H), 4.29-4.47 (m, 3 H), 6.44 (d, J = 2.27 Hz, 1 H), 6.82 (br. s., 2 H), 7.32 (s, 1 H), 7.37 (s, 1 H) | 513.1, 514.1 |
| 157 | 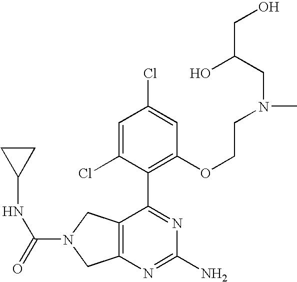 | 2-amino-N-cyclopropyl-4-(2,4-dichloro- 6-{2-[(2,3- dihydroxypropyl)(methyl)amino] ethoxy}phenyl)-5,7-dihydro-6H- pyrrolo[3,4-d]pyrimidine-6- carboxamide | Ex. 101 | 1H NMR (400 MHz, DMSO-d6) d ppm 1.90 (s, 1 H), 2.06-2.12 (m, 3 H), 2.11-2.14 (m, 2 H), 2.20 (dd, J = 12.88, 7.07 Hz, 2 H), 2.29-2.40 (m, 2 H), 2.56-2.63 (m, 2 H), 3.17-3.27 (m, 2 H), 3.39-3.49 (m, 2 H), 4.02-4.13 (m, 3 H), 4.13-4.30 (m, 2 H), 4.39 (s, 2 H), 6.43 (d, J = 2.53 Hz, 1 H), 6.77 (s, 2 H), 7.28 (d, J = 1.77 Hz, 1 H), 7.32 (d, J = 1.77 Hz, 1 H) | 512.2, 513.2 |
| 158 | 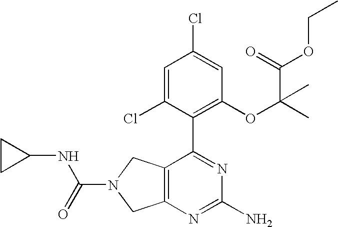 | ethyl 2-(2-{2-amino-6- [(cyclopropylamino)carbonyl]-6,7- dihydro-5 H-pyrrolo[3,4-d]pyrimidin-4- yl}-3,5-dichlorophenoxy)-2- methylpropanoate | Ex. 100 | 1H NMR (400 MHz, DMSO-d6) d ppm 0.34-0.45 (m, 2 H), 0.48-0.58 (m, 2 H), 1.17 (t, J = 7.07 Hz, 3 H), 1.24 (s, 1 H), 1.35 (s, 3 H), 1.43 (s, 3 H), 4.10-4.23 (m, 4 H), 4.40 (s, 2 H), 6.45 (d, J = 2.78 Hz, 1 H), 6.79-6.88 (m, 3 H), 7.45 (d, J = 2.02 Hz, 1 H) | 494.1, 496.1 |
| 159 | 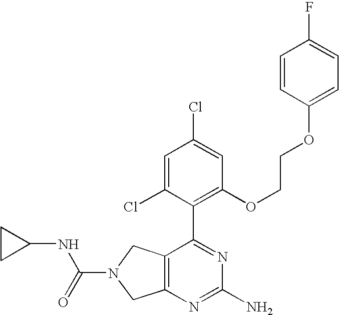 | 2-amino-N-cyclopropyl-4-{2,4- dichloro-6-[2-(4- fluorophenoxy)ethoxy]phenyl}-5,7- dihydro-6H-pyrrolo[3,4-d]pyrimidine-6- carboxamide | Ex.100 | 1H NMR (400 MHz, DMSO-d6) d ppm 0.32-0.43 (m, 2 H), 0.49-0.61 (m, 2 H), 1.23 (s, 1 H), 3.96-4.28 (m, 5 H), 4.29-4.42 (m, 3 H), 6.41 (d, J = 2.02 Hz, 1 H), 6.70-6.83 (m, 3 H), 6.95-7.06 (m, 2 H), 7.31-7.41 (m, 2 H), 7.31-7.41 (m, 2 H), 7.50-7.68 (m, 1 H) | 518.1, 520.1 |
| 160 | 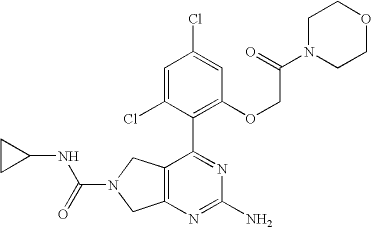 | 2-amino-N-cyclopropyl-4-[2,4-dichloro- 6-(2-morpholin-4-yl-2- oxoethoxy)phenyl]-5,7-dihydro-6H- pyrrolo[3,4-d]pyrimidine-6- carboxamide | Ex. 100 | 1H NMR (400 MHz, DMSO-d6) d ppm 0.32-0.44 (m, 2 H), 0.47-0.59 (m, 2 H), 2.54 (s, 1 H), 3.46 (s, 4 H), 3.47-3.60 (m, 4 H), 4.10 (d, J = 13.39 Hz, 1 H), 4.19-4.32 (m, 1 H), 4.39 (s, 2 H), 4.81-4.96 (m, 1 H), 4.96-5.09 (m, 1 H), 6.45 (d, J = 2.53 Hz, 1 H), 6.79 (br. s., 2 H), 7.18 (d, J = 1.26 Hz, 1 H), 7.31 (d, J = 1.52 Hz, 1 H) | 507.1, 509.1 |
| 161 | 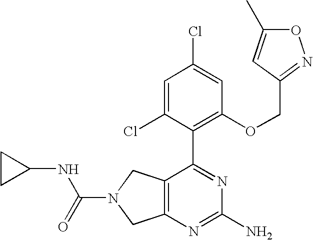 | 2-amino-N-cyclopropyl-4-{2,4- dichloro-6-[(5-methylisoxazol-3- yl)methoxy]phenyl}-5,7-dihydro-6H- pyrrolo[3,4-d]pyrimidine-6- carboxamide | Ex. 100 | 1H NMR (400 MHz, DMSO-d6) d ppm 0.34-0.45 (m, 2 H), 0.48-0.59 (m, 2 H), 2.33-2.41 (m, 3 H), 2.52-2.56 (m, 1 H), 3.96-4.16 (m, 2 H), 4.40 (s, 2 H), 5.19-5.33 (m, 2 H), 6.04 (s, 1 H), 6.44 (d, J = 2.53 Hz, 1 H), 6.84 (s, 2 H), 7.39 (d, J = 1.77 Hz, 1 H), 7.42 (d, J = 1.77 Hz, 1 H) | 475.1, 477.1 |
| 162 | 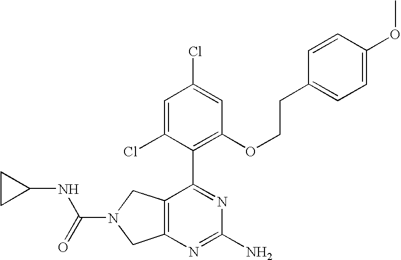 | 2-amino-N-cyclopropyl-4-{2,4- dichloro-6-[2-(4- methoxyphenyl)ethoxy]phenyl}-5,7- dihydro-6H-pyrrolo[3,4-d]pyrimidine-6- carboxamide | Ex. 100 | 1H NMR (400 MHz, DMSO-d6) d ppm 0.34-0.44 (m, 2 H), 0.47-0.58 (m, 2 H), 2.65-2.84 (m, 2 H), 3.69 (s, 3 H), 3.74 (d, J = 13.14 Hz, 1 H), 3.97 (d, J = 12.88 Hz, 1 H), 4.06-4.23 (m, 3 H), 4.41 (s, 2 H), 6.36 (br. s., 1 H), 6.69 (d, J = 8.59 Hz, 2 H), 6.83 (s, 2 H), 6.86 (s, 1 H), 6.88 (s, 1 H), 7.24 (d, J = 1.77 Hz, 1 H), 7.29 (d, J = 1.77 Hz, 1 H) | 514.1, 515.1 |
| 163 | 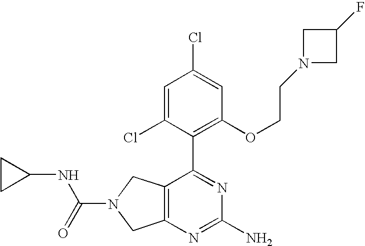 | 2-amino-N-cyclopropyl-4-{2,4- dichloro-6-[2-(3-fluoroazetidin-1- yl)ethoxy]phenyl}-5,7-dihydro-6H- pyrrolo[3,4-d]pyrimidine-6- carboxamide | Ex. 101 | 1H NMR (400 MHz, DMSO-d6) d ppm 0.32-0.44 (m, 2 H), 0.47-0.59 (m, 2 H), 2.52-2.57 (m, 1 H), 2.66 (br. s., 2 H), 2.90 (d, J = 7.83 Hz, 2 H), 4.00 (t, J = 4.93 Hz, 2 H), 4.07-4.20 (m, 4 H), 4.33-4.46 (m, 2 H), 4.84-5.11 (m, 1 H), 6.46 (d, J = 2.53 Hz, 1 H), 6.18 (s, 2 H), 7.24 (d, J = 1.52 Hz, 1 H), 7.29-7.36 (m, 1 H) | 481.1, 483.1 |
| 164 | 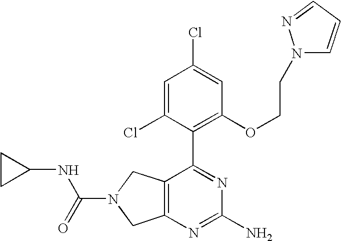 | 2-amino-N-cyclopropyl-4-{2,4- dichloro-6-[2-(1H-pyrazol-1- yl)ethoxy]phenyl}-5,7-dihydro-6H- pyrrolo[3,4-d]pyrimidine-6- carboxamide | Ex. 100 | 1H NMR (400 MHz, DMSO-d6) d ppm 0.32-0.45 (m, 2 H), 0.50-0.59 (m, 2 H), 2.52-2.57 (m, 1 H), 3.55 (d, J = 12.88 Hz, 1 H), 3.82-3.97 (m, 1 H), 4.32 (s, 4 H), 4.35-4.42 (m, 2 H), 6.00 (t, J = 2.02 Hz, 1 H), 6.33 (br. s., 1 H), 6.78 (s, 2 H), 7.18 (d, J = 2.02 Hz, 1 H), 7.25 (d, J = 1.77 Hz, 1 H), 7.29 (d, J = 1.77 Hz, 1 H), 7.32 (d, J = 1.52 Hz, 1 H) | 474.1, 476.1 |
| 165 | 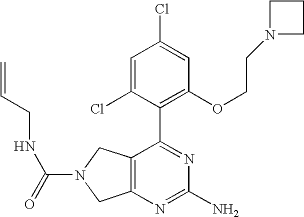 | N-allyl-2-amino-4-[2-(2-azetidin-1- ylethoxy)-4,6-dichlorophenyl]-5,7- dihydro-6H-pyrrolo[3,4-d]pyrimidine-6- carboxamide | Ex. 100 | 1H NMR (400 MHz, DMSO-d6) d ppm 1.85 (t, J = 7.07 Hz, 2 H), 2.60 (t, 2 H), 3.01 (br. s., 2 H), 3.67 (t, J = 5.31 Hz, 2 H), 3.98 (t, J = 4.93 Hz, 2 H), 4.08-4.28 (m, 4 H), 4.37-4.48 (m, 4 H), 4.37-4.48 (m, 2 H), 5.00 (dd, J = 10.23, 1.64 Hz, 1 H), 5.10 (dd, J = 17.18, 1.77 Hz, 1 H), 5.68-5.90 (m, 1 H), 6.57 (t, J = 5.68 Hz, 1 H), 6.81 (s, 2 H), 7.24 (d, J = 1.77 Hz, 1 H), 7.29-7.36 (m, 1 H) | 463.2, 464.2 |
| 166 | 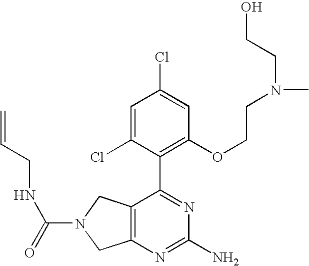 | N-allyl-2-amino-4-(2,4-dichloro-6-{2- [(2- hydroxyethyl)(methyl)amino]ethoxy} phenyl)-5,7-dihydro-6H-pyrrolo[3,4- d]pyrimidine-6-carboxamide | Ex.100 | 1H NMR (400 MHz, DMSO-d6) d ppm 2.08 (s, 3 H), 2.33 (t, J = 6.19 Hz, 2 H), 2.56-2.63 (m, 2 H), 3.67 (t, J = 5.31 Hz, 3 H), 3.97-4.17 (m, 4 H), 4.25 (d, J = 13.14 Hz, 2 H), 4.43 (br. s., 2 H), 5.00 (dd, J = 10.23, 1.64 Hz, 1 H), 5.10 (dd, J = 17.31, 1.64 Hz, 1 H), 5.73-5.88 (m, 1 H), 6.55 (t, J = 5.56 Hz, 1 H), 6.79 (s, 2 H), 7.29 (d, J = 1.77 Hz, 1 H), 7.32 (d, J = 1.77 Hz, 1 H) | 481.2, 483.2 |
| 167 | 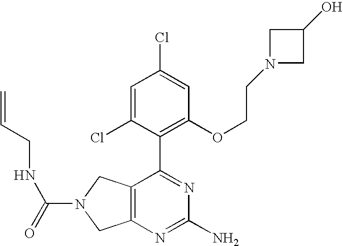 | N-allyl-2-amino-4-{2,4-dichloro-6-[2- (3-hydroxyazetidin-1- yl)ethoxy]phenyl}-5,7-dihydro-6H- pyrrolo[3,4-d]pyrimidine-6- carboxamide | Ex. 101 | 1H NMR (400 MHz, DMSO-d6) d ppm 2.56 (d, J = 6.57 Hz, 2 H), 3.63-3.72 (m, 2 H), 3.93-4.04 (m, 4 H), 4.06-4.18 (m, 2 H), 4.18-4.29 (m, 2 H), 4.39-4.49 (m, 2 H), 5.01 (dd, J = 10.23, 1.64 Hz, 1 H), 5.06-5.16 (m, 1 H), 5.20 (br. s., 1 H), 5.73-5.89 (m, 1 H), 6.51-6.61 (m, 2 H), 6,81 (s, 2 H), 7.25 (d, J = 1.77 Hz, 1 H), 7.31-7.36 (m, 1 H) | 479.2, 481.2 |
| 168 | 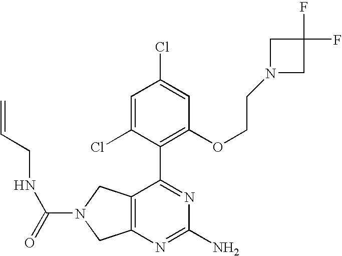 | N-allyl-2-amino-4-{2,4-dichloro-6-[2- (3,3-difluoroazetidin-1- yl)ethoxy]phenyl}-5,7-dihydro-6H- pyrrolo[3,4-d]pyrimidine-6- carboxamide | Ex. 101 | 1H NMR (400 MHz, DMSO-d6) d ppm 2.73 (d, J = 4.04 Hz, 2 H), 3.20-3.29 (m, 2 H), 3.66 (t, J = 5.31 Hz, 2 H), 3.99-4.09 (m, 2 H), 4.15 (s, 2 H), 4.29-4.51 (m, 4 H), 4.99 (dd, J = 10.23, 1.64 Hz, 1 H), 5.09 (dd, J = 17.43, 1.77 Hz, 1 H), 5.73-5.85 (m, 1 H), 6.56 (t, J = 5.56 Hz, 1 H), 6.85 (s, 2 H), 7.24 (d, J = 1.77 Hz, 1 H), 7.33 (d, J = 1.77 Hz, 1 H) | 499.0, 501.0 |
| 169 | 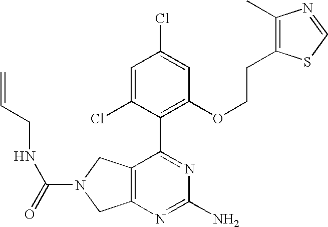 | N-allyl-2-amino-4-{2,4-dichloro-6-[2- (4-methyl-1,3-thiazol-5- yl)ethoxy]phenyl}-5,7-dihydro-6H- pyrrolo[3,4-d]pyrimidine-6- carboxamide | Ex. 100 | 1H NMR (400 MHz, DMSO-d6) d ppm 2.02 (s, 3 H), 2.99-3.12 (m, 2 H), 3.55-3.65 (m, 1 H), 3.68 (t, J = 5.18 Hz, 2 H), 3.87-4.00 (m, 1 H), 4.01-4.12 (m, 1 H), 4.23-4.34 (m, 1 H), 4.33-4.54 (m, 2 H), 5.02 (dd, J = 10.36, 1.52 Hz, 1 H), 5.13 (dd, J = 17.18, 1.52 Hz, 1 H), 5.74-5.93 (m, 1 H), 6.46 (br. s., 1 H), 6.80 (s, 2 H), 7.26 (d, J = 1.52 Hz, 1 H), 7.32 (d, J = 1.77 Hz, 1 H), 8.56 (s, 1 H) | 505.0, 507.0 |
| 170 | 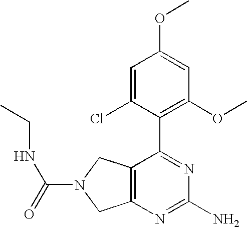 | 2-amino-4-(2-chloro-4,6- dimethoxyphenyl)-N-ethyl-5,7-dihydro- 6H-pyrrolo[3,4-d]pyrimidine-6- carboxamide | Ex. 107 | 1H NMR (400 MHz, DMSO-d6) d ppm 1.01 (t, J = 7.20 Hz, 3 H), 2.97-3.12 (m, 2 H), 3.73 (s, 3 H), 3.80-3.87 (m, 3 H), 4.05-4.17 (m, 2 H), 4.37-4.53 (m, 2 H), 6.35 (t, J = 5.43 Hz, 1 H), 6.68 (d, J = 2.27 Hz, 1 H), 6.75 (d, J = 2.27 Hz, 2 H), 7.21 (s, 1 H) | 378.1, 380.1 |
| 171 | 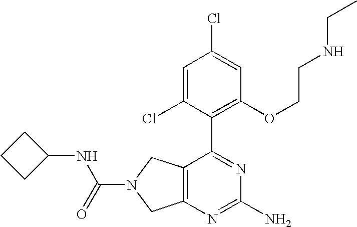 | 2-amino-N-cyclobutyl-4-{2,4-dichloro- 6-[2-(ethylamino)ethoxy]phenyl}-5,7- dihydro-6H-pyrrolo[3,4-d]pyrimidine-6- carboxamide | Ex. 100 | 1H NMR (400 MHz, DMSO-d6) d ppm 0.87 (t, J = 7.07 Hz, 3 H), 1.44-1.61 (m, 2 H), 1.83-1.96 (m, 4 H), 2.02-2.14 (m, 2 H), 2.38-2.45 (m, 2 H), 2.73 (t, J = 4.93 Hz, 2 H), 4.01-4.19 (m, 4 H), 4.40 (br. s., 2 H), 6.51 (d, J = 7.83 Hz, 1 H), 6.82 (s, 2 H), 7.31 (s, 1 H), 7.34 (s, 1 H) | 465.1, 467.1 |
| 172 | 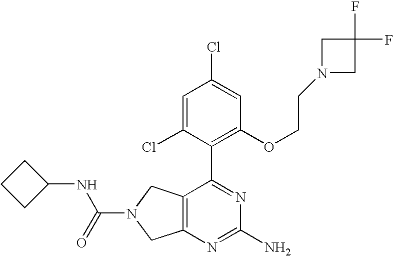 | 2-amino-N-cyclobutyl-4-{2,4-dichloro- 6-[2-(3,3-difluoroazetidin-1- yl)ethoxy]phenyl}-5,7-dihydro-6H- pyrrolo[3,4-d]pyrimidine-6- carboxamide | Ex. 100 | 1H NMR (400 MHz, DMSO-d6) d ppm 1.54 (dd, J = 9.85, 7.33 Hz, 2 H), 1.85-1.97 (m, 2 H), 2.08 (dd, J = 6.82, 3.54 Hz, 2 H), 2.74 (br. s., 2 H), 3.36-3.47 (m, 2 H), 3.98-4.08 (m, 2 H), 4.09-4.17 (m, 3 H), 4.26-4.48 (m, 2 H), 6.47-6.57 (m, 2 H), 6.47-6.57 (m, 2 H), 6.85 (s, 2 H), 7.25 (d, J = 1.77 Hz, 1 H), 7.34 (d, J = 1.52 Hz, 1 H) | 513.1, 515.1 |
| 173 | 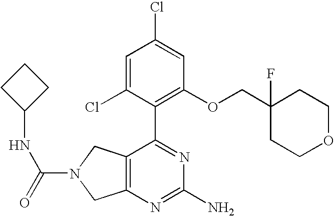 | -amino-N-cyclobutyl-4-{2,4-dichloro-6- [(4-fluorotetrahydro-2 H-pyran-4- yl)methoxy]phenyl}-5,7-dihydro-6H- pyrrolo[3,4-d]pyrimidine-6- carboxamide | Ex. 100 | 1H NMR (400 MHz, DMSO-d6) d ppm 1.46-1.60 (m, 4 H), 1.91 (br. s., 2 H), 2.09 (dd, J = 6.82, 3.28 Hz, 2 H), 3.28-3.40 (m, 4 H), 3.42-3.52 (m, 2 H), 3.56-3.68 (m, 2 H), 4.09-4.16 (m, 3 H), 4.36-4.44 (m, 2 H), 6.52-6.59 (m, 2 H), 6.82 (s, 1 H), 7.37 (dd, J = 8.08, 1.77 Hz, 2 H) | |
| 174 | 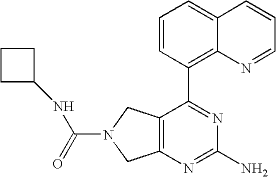 | 2-amino-N-cyclobutyl-4-quinolin-8-yl- 5,7-dihydro-6H-pyrrolo[3,4- d]pyrimidine-6-carboxamide | Ex. 18a | 1H NMR (400 MHz, DMSO-d6) d ppm 1.44-1.62 (m, 2 H), 1.81-1.94 (m, 2 H), 1.99-2.18 (m, 2 H), 3.95-4.19 (m, 3 H), 4.42 (d, J = 18.44 Hz, 2 H), 6.42 (d, J = 7.83 Hz, 1 H), 6.75 (s, 2 H), 7.59-7.66 (m, 1 H), 7.78-7.84 (m, 1 H), 8.08-8.17 (m, 1 H), 8.43-8.55 (m, 1 H), 8.93 (dd, J = 4.17, 1.64 Hz, 1 H) | 504.2, 506.2 |
| 175 | 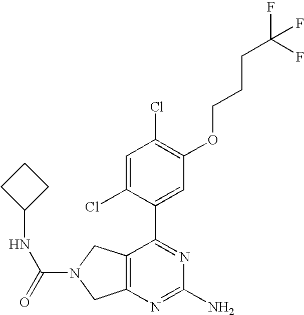 | 2-amino-N-cyclobutyl-4-[2,4-dichloro- 5-(4,4,4-trifluorobutoxy)phenyl]-5,7- dihydro-6H-pyrrolo[3,4-d]pyrimidine-6- carboxamide | Ex. 100 | 1H NMR (400 MHz, DMSO-d6) d ppm 1.45-1.65 (m, 2 H) 1.86-2.01 (m, 4 H) 2.03-2.15 (m, 2 H) 2.37-2.46 (m, 2 H) 4.06-4.21 (m, 3 H) 4.26 (s, 2 H) 4.41 (s, 2 H) 6.53 (d, J = 8.08 Hz, 1 H) 6.89 (s, 2 H) 7.23 (s, 1 H) 7.77 (s, 1 H) | 504.2, 506.2 |
| 177 | 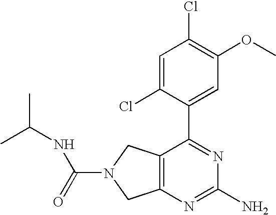 | 2-amino-4-(2,4-dichloro-5- methoxyphenyl)-N-isopropyl-5,7- dihydro-6H-pyrrolo[3,4-d]pyrimidine-6- carboxamide | Ex. 102 | 1H NMR (400 MHz, DMSO-d6) d ppm 1.05 (d, J = 6.57 Hz, 6 H) 3.77 (d, J = 7.07 Hz, 1 H) 3.87 (s, 3 H) 4.25 (s, 2 H) 4.41 (s, 2 H) 6.07 (d, J = 7.58 Hz, 1 H) 6.89 (s, 2 H) 7.20 (s, 1 H) 7.74 (s, 1 H) | 396.0, 397.0 |
| 178 | 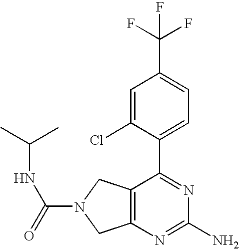 | 2-amino-4-[2-chloro-4- (trifluoromethyl)phenyl]-N-isopropyl- 5,7-dihydro-6H-pyrrolo[3,4- d]pyrimidine-6-carboxamide | Ex. 18a | 1H NMR (400 MHz, DMSO-d6) d ppm 1.05 (d, J = 6.32 Hz, 6 H) 3.77 (d, J = 6.82 Hz, 1 H) 4.25 (s, 2 H) 4.43 (s, 2 H) 6.08 (d, J = 7.83 Hz, 1 H) 6.93 (s, 2 H) 7.70 (d, J = 7.83 Hz, 1 H) 7.86 (d, J = 7.83 Hz, 1 H) 8.06 (s, 1 H) | 400.1, 402.1 |
| 179 | 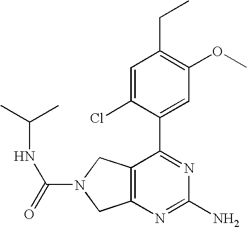 | 2-amino-4-(2-chloro-4-ethyl-5- methoxyphenyl)-N-isopropyl-5,7- dihydro-6H-pyrrolo[3,4-d]pyrimidine-6- carboxamide | Ex. 104 | 1H NMR (400 MHz, DMSO-d6) d ppm 1.00-1.11 (m, 6 H) 1.10-1.21 (m, 3 H) 2.54-2.67 (m, 2 H) 3.76-3.83 (m, 3 H) 4.27 (s, 2 H) 4.37-4.44 (m, 2 H) 6.10 (d, J = 7.83 Hz, 1 H) 6.82 (s, 2 H) 6.96 (s, 1 H) 7.27-7.35 (m, 1 H) | 390.4, 392.4 |
| 180 | 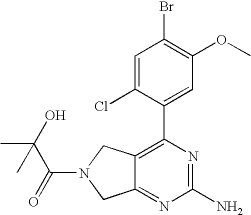 | 1-[2-amino-4-(4-bromo-2-chloro-5- methoxyphenyl)-5,7-dihydro-6H- pyrrolo[3,4-d]pyrimidin-6-yl]-2-methyl- 1-oxopropan-2-ol | Ex. 6 | 1H NMR (400 MHz, DMSO-d6) d ppm 1.31 (d, J = 13.89 Hz, 6 H) 3.85 (s, 3 H) 4.44 (d, J = 61.39 Hz, 2 H) 4.91 (d, J = 48.25 Hz, 2 H) 5.35 (d, J = 25.52 Hz, 1 H) 6.92 (s, 2 H) 7.16 (d, J = 14.65 Hz, 1 H) 7.87 (d, J = 5.05 Hz, 1 H) | |
| 181 | 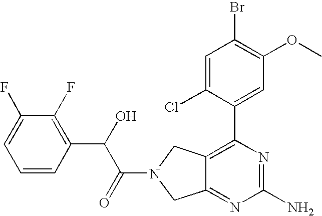 | 2-[2-amino-4-(4-bromo-2-chloro-5- methoxyphenyl)-5,7-dihydro-6H- pyrrolo[3,4-d]pyrimidin-6-yl]-1-(2,3- difluorophenyl)-2-oxoethanol | Ex. 6 | 1H NMR (400 MHz, DMSO-d6) d ppm 3.82-3.89 (m, 3 H), 4.40 (d, J = 6.06 Hz, 2 H), 4.49-4.58 (m, 2 H), 4.76-5.02 (m, 1 H), 5.60 (d, J = 24.25 Hz, 1 H), 6.86-7.05 (m, 2 H), 7.11-7.21 (m, 1 H), 7.22-7.31 (m, 2 H), 7.33-7.44 (m, 1 H), 7.86 (d, J = 1.01 Hz, 1 H) | |
| 182 | 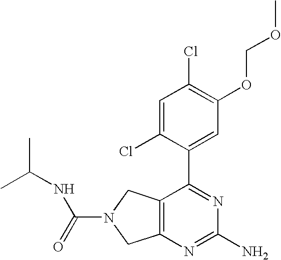 | 2-amino-4-[2,4-dichloro-5- (methoxymethoxy)phenyl]-N-isopropyl- 5,7-dihydro-6H-pyrrolo[3,4- d]pyrimidine-6-carboxamide | Ex. 15a | 1H NMR (400 MHz, DMSO-d6) d ppm 1.06 (d, J = 6.57 Hz, 6 H), 3.39-3.45 (m, 3 H), 3.78 (dd, J = 14.15, 6.57 Hz, 1 H), 4.25 (s, 2 H), 4.42 (s, 2 H), 5.33 (s, 2 H), 6.08 (d, J = 7.58 Hz, 1 H), 6.92 (s, 2 H), 7.29 (s, 1 H), 7.80 (s, 1 H) | 427.2, 429.2 |
| 183 | 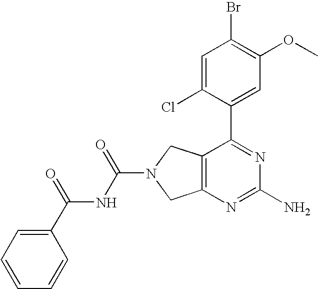 | 2-amino-N-benzoyl-4-(4-bromo-2- chloro-5-methoxyphenyl)-5,7-dihydro- 6H-pyrrolo[3,4-d]pyrimidine-6- carboxamide | Ex. 15a | 1H NMR (400 MHz, DMSO-d6) d ppm 3.85 (d, J = 10.61 Hz, 3 H), 4.45 (s, 2 H), 4.60 (s, 2 H), 4.75 (s, 1 H), 6.97 (s, 2 H), 7.16 (s, 1 H), 7.44-7.57 (m, 2 H), 7.61 (s, 1 H), 7.78-7.85 (m, 1 H), 7.85-7.93 (m, 2 H) | 505.2, 506.2 |
| 184 | 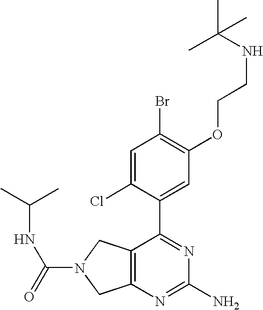 | 2-amino-4-{4-bromo-5-[2-(tert- butylamino)ethoxy]-2-chlorophenyl}-N- isopropyl-5,7-dihydro-6H-pyrrolo[3,4- d]pyrimidine-6-carboxamide | Ex. 15 | 1H NMR (400 MHz, DMSO-d6) d ppm 1.05 (t, J = 3.16 Hz, 15 H), 1.90 (s, 1 H), 2.87 (t, J = 5.43 Hz, 2 H), 3.78 (dd, J = 14.02, 6.69 Hz, 1 H), 4.09 (t, J = 5.68 Hz, 2 H), 4.25 (s, 2 H), 4.41 (s, 2 H), 6.07 (d, J = 7.58 Hz, 1 H), 6.88 (s, 2 H), 7.19 (s, 1 H), 7.86 (s, 1 H) | 527.1, 528.1 |
| 185 | 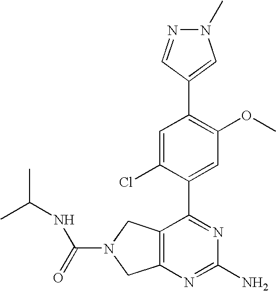 | 2-amino-4-[2-chloro-5-methoxy-4-(1- methyl-1H-pyrazol-4-yl)phenyl]-N- isopropyl-5,7-dihydro-6H-pyrrolo[3,4- d]pyrimidine-6-carboxamide | Ex. 104 | 1H NMR (400 MHz, DMSO-d6) d ppm 1.05 (t, J = 5.81 Hz, 6 H), 3.78 (s, 1 H), 3.86-3.92 (m, 6 H), 4.30 (s, 2 H), 4.43 (s, 2 H), 6.05-6.16 (m, 1 H), 6.85 (s, 2 H), 7.08 (s, 1 H), 8.30 (s, 1 H), 8.25 (s, 1 H) | 442.2, 444.2 |
| 186 | 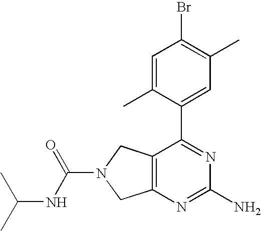 | 2-amino-4-(4-bromo-2,5- dimethylphenyl)-N-isopropyl-5,7- dihydro-6H-pyrrolo[3,4-d]pyrimidine-6- carboxamide | Ex. 15a | 1H NMR (400 MHz, DMSO-d6) d ppm 1.05 (d, J = 6.57 Hz, 6 H), 2.17 (s, 3 H), 2.33 (s, 3 H), 3.72-3.84 (m, 1 H), 4.24 (s, 2 H), 4.41 (s, 2 H), 6.06 (d, J = 7.33 Hz, 1 H), 7.28 (s, 1 H), 7.56 (s, 1 H) | 405.1, 406.1 |
| 187 | 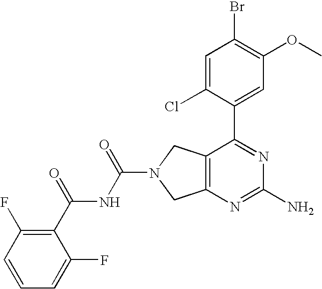 | 2-amino-4-(4-bromo-2-chloro-5- methoxyphenyl)-N-(2,6- difluorobenzoyl)-5,7-dihydro-6H- pyrrolo[3,4-d]pyrimidine-6- carboxamide | Ex. 15a | 1H NMR (400 MHz, DMSO-d6) d ppm 3.86 (d, J = 10.86 Hz, 3 H), 4.34 (s, 1 H), 4.48 (s, 1 H), 4.59 (s, 1 H), 4.74 (s, 1 H), 6.97 (s, 2 H), 7.03-7.11 (m, 1 H), 7.11-7.23 (m, 2 H), 7.46-7.59 (m, 1 H), 7.58-7.68 (m, 1 H), 7.86 (d, J = 13.64 Hz, 1 H) | 540.2, 542.2 |
| 188 | 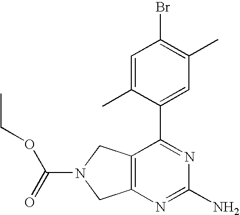 | ethyl 2-amino-4-(4-bromo-2,5- dimethylphenyl)-5,7-dihydro-6H- pyrrolo[3,4-d]pyrimidine-6-carboxylate | Ex. 1 | 1H NMR (400 MHz, DMSO-d6) d ppm 1.09-1.30 (m, 3 H), 2.18 (d, J = 7.83 Hz, 3 H), 2.33 (s, 3 H), 4.08 (dd, J = 14.02, 6.95 Hz, 2 H), 4.32 (d, J = 5.05 Hz, 2 H), 4.46 (d, J = 10.36 Hz, 2 H), 681 (s, 2 H), 7.29 (d, J = 5.05 Hz, 1 H), 7.55 (s, 1 H) | 391.1, 392.1 |
| 189 | 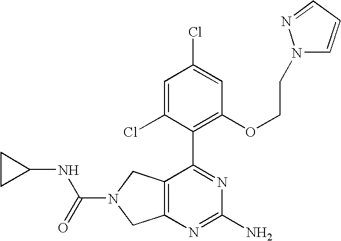 | 2-amino-N-cyclopropyl-4-{2,4- dichloro-6-[2-(1H-pyrazol-1- yl)ethoxy]phenyl}-5,7-dihydro-6H- pyrrolo[3,4-d]pyrimidine-6- carboxamide | Ex. 100 | 1H NMR (400 MHz, DMSO-d6) d ppm 0.33-0.44 (m, 2 H), 0.49-0.58 (m, 2 H), 2.51-2.56 (m, 1 H), 3.55 (d, J = 12.88 Hz, 1 H), 3.82-3.94 (m, 1 H), 4.29-4.35 (m, 4 H), 4.36-4.42 (m, 2 H), 6.00 (t, J = 2.02 Hz, 1 H), 6.32 (br. s., 1 H), 6.77 (br. s., 2 H), 7.18 (d, J = 2.27 Hz, 1 H), 7.24 (d, J = 1.77 Hz, 1 H), 7.29 (d, J = 1.52 Hz, 1 H), 7.31 (d, J = 1.52 Hz, 1 H) | 474.2, 476.2 |
| 190 | 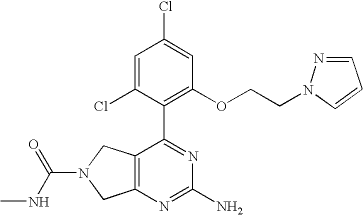 | 2-amino-4-{2,4-dichloro-6-[2-(1H- pyrazol-1-yl)ethoxy]phenyl}-N-methyl- 5,7-dihydro-6H-pyrrolo[3,4- d]pyrimidine-6-carboxamide | Ex. 106 | 1H NMR (400 MHz, DMSO-d6) d ppm 2.59 (d, J = 4.29 Hz, 3 H) 3.60 (d, J = 12.63 Hz, 1 H) 3.92 (dd, 1 H) 4.29-4.36 (m, 4 H) 4.36-4.46 (m, 2 H) 6.00 (t, J = 2.02 Hz, 1 H) 6.18 (d, J = 4.29 Hz, 1 H), 6.78 (s, 2 H) 7.19 (d, J = 2.02 Hz, 1 H) 7.25 (d, J = 1.77 Hz, 1 H) 7.29 (d, J = 1.52 Hz, 1 H) 7.32 (d, J = 1.77 Hz, 1 H) | 450.2, 448.2 |
| 191 | 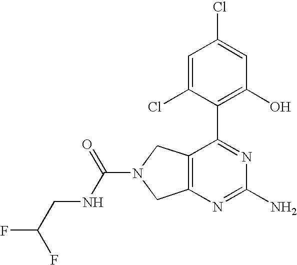 | 2-amino-4-(2,4-dichloro-6- hydroxyphenyl)-N-(2,2-difluoroethyl)- 5,7-dihydro-6H-pyrrolo[3,4- d]pyrimidine-6-carboxamide | Ex. 18 | 1H NMR (400 MHz, DMSO-d6) d ppm 3.35-3.49 (m, 2 H) 4.19 (s, 2 H) 4.45 (s, 2 H) 5.96 (tt, 1 H) 6.76-6.89 (m, 3 H) 6.96 (d, J = 1.77 Hz, 1 H) 7.15 (d, J = 2.02 Hz, 1 H) 10.66 (s, 1 H) | 406.0, 404.2 |
| 192 | 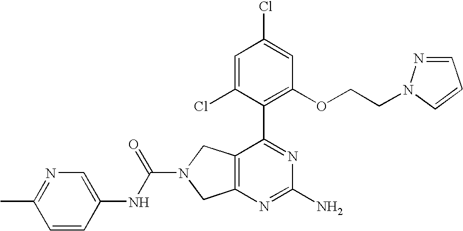 | 2-amino-4-{2,4-dichloro-6-[2-(1H- pyrazol-1-yl)ethoxy]phenyl}-N-(6- methylpyridin-3-yl)-5,7-dihydro-6H- pyrrolo[3,4-d]pyrimidine-6- carboxamide | Ex. 109 | 1H NMR (400 MHz, DMSO-d6) d ppm 2.42 (s, 3 H), 3.69 (d, J = 12.63 Hz, 1 H) 4.13 (d, J = 12.63 Hz, 1 H) 4.31-4.40 (m, 4 H) 4.49-4.63 (m, 2 H) 6.01 (t, J = 1.89 Hz, 1 H) 6.84 (s, 2 H) 7.22-7.27 (m, 3 H), 7.29 (d, J = 1.52 Hz, 1 H) 7.34 (d, J = 1.77 Hz, 1 H) 7.91 (dd, J = 8.46, 2.40 Hz, 1 H) 8.43 (s, 1 H) 8.61 (s, 1 H) | 527.2, 525.2 |
| 193 | 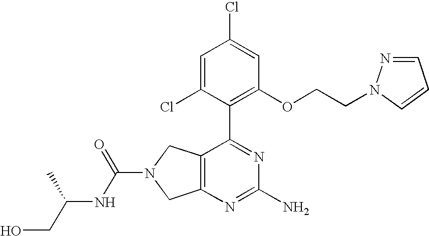 | 2-amino-4-{2,4-dichloro-6-[2-(1H- pyrazol-1-yl)ethoxy]phenyl}-N-[(1S)-2- hydroxy-1-methylethyl]-5,7-dihydro- 6H-pyrrolo[3,4-d]pyrimidine-6- carboxamide | Ex. 110 | | 494.2, 492.2 |
| 194 | 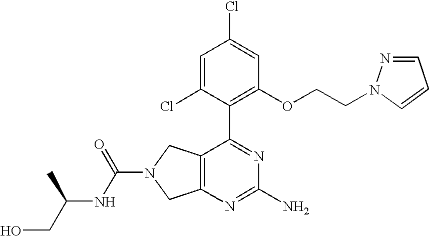 | 2-amino-4-{2,4-dichloro-6-[2-(1H- pyrazol-1-yl)ethoxy]phenyl}-N-[(1R)-2- hydroxy-1-methylethyl]-5,7-dihydro- 6H-pyrrolo[3,4-d]pyrimidine-6- carboxamide | Ex. 110 | | 494.2, 492.2 |
| 195 | 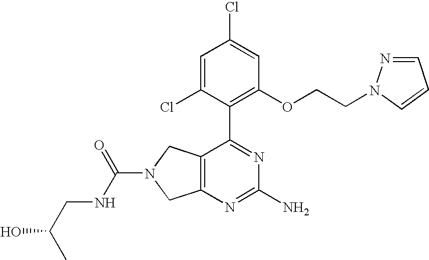 | 2-amino-4-{2,4-dichloro-6-[2-(1H- pyrazol-1-yl)ethoxy]phenyl}-N-[(2S)-2- hydroxypropyl]-5,7-dihydro-6H- pyrrolo[3,4-d]pyrimidine-6- carboxamide | Ex. 110 | 1H NMR (400 MHz, DMSO-d6) d ppm 1.01 (dd, J = 6.19, 1.39 Hz, 3 H) 2.96-3.03 (m, J = 5.56, 5.56 Hz, 2 H) 3.58-3.70 (m, 2 H) 3.95 (d, J = 12.63 Hz, 1 H) 4.30-4.37 (m, 4 H) 4.39-4.47 (m, 2 H) 6.01 (q, J = 1.94 Hz, 1 H) 6.21-6.30 (m, 1 H) 6.78 (s, 2 H) 7.19 (d, J = 2.27 Hz, 1 H) 7.25 (d, J = 1.77 Hz, 1 H) 7.30-7.32 (m, 1 H) 7.32 (d, J = 1.77 Hz, 1 H) | 494.2, 492.2 |
| 196 | 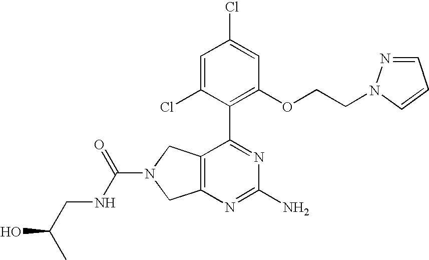 | 2-amino-4-{2,4-dichloro-6-[2-(1H- pyrazol-1-yl)ethoxy]phenyl}-N-[(2R)-2- hydroxypropyl]-5,7-dihydro-6H- pyrrolo[3,4-d]pyrimidine-6- carboxamide | Ex. 110 | 1H NMR (400 MHz, DMSO-d6) d ppm 1.01 (dd, J = 6.32, 1.26 Hz, 3 H) 2.95-3.02 (m, J = 5.56, 5.56 Hz, 2 H) 3.58-3.70 (m, 2 H) 3.95 (d, J = 12.63 Hz, 1 H) 4.30-4.38 (m, 4 H) 4.39-4.47 (m, 2 H) 6.01 (q, J = 1.85 Hz, 1 H) 6.22-6.30 (m, 1 H) 6.78 (s, 2 H) 7.19 (d, J = 2.27 Hz, 1 H) 7.25 (d, J = 1.77 Hz, 1 H) 7.29-7.31 (m, 1 H) 7.32 (d, J = 1.52 Hz, 1 H) | 494.2, 492.2 |
| 197 | 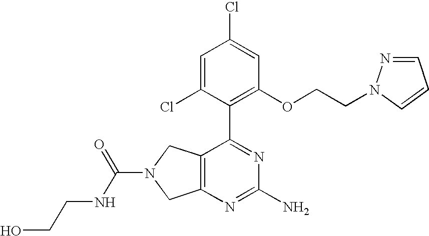 | 2-amino-4-{2,4-dichloro-6-[2-(1H- pyrazol-q-yl)ethoxy]phenyl}-N-(2- hydroxyethyl)-5,7-dihydro-6H- pyrrolo[3,4-d]pyrimidine-6- carboxamide | Ex. 110 | 1H NMR (300 MHz, DMSO-d6) d ppm 3.06-3.15 (m, 2 H) 3.37-3.43 (m, 2 H) 3.59 (d, J = 13.00 Hz, 1 H) 3.93 (d, J = 12.62 Hz, 1 H) 4.33 (s, 4 H) 4.38-4.48 (m, 2 H) 6.01 (t, J = 1.98 Hz, 1 H) 6.22-6.28 (m, 1 H) 6.79 (s, 2 H) 7.19 (d, J = 2.26 Hz, 1 H) 7.25 (d, J = 1.70 Hz, 1 H) 7.30-7.33 (m, 1.70 Hz, 2 H) | 480.2, 478.2 |
| 198 | 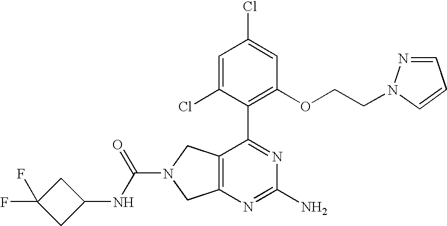 | 2-amino-4-{2,4-dichloro-6-[2-(1H- pyrazol-1-yl)ethoxy]phenyl}-N-(3,3- difluorocyclobutyl)-5,7-dihydro-6H- pyrrolo[3,4-d]pyrimidine-6- carboxamide | Ex. 110 | 1H NMR (400 MHz, DMSO-d6) d ppm 2.57-2.70 (m, 2 H) 2.77-2.90 (m, 2 H) 3.60 (d, J = 12.88 Hz, 1 H), 3.95 (d, J = 12.88 Hz, 1 H) 3.99-4.08 (m, 1 H) 4.31-4.37 (m, 4 H) 4.39-4.47 (m, 2 H) 6.00 (t, J = 2.02 Hz, 1 H) 6.60 (d, J = 6.32 Hz, 1 H) 6.77 (s, 2 H) 7.20 (d, J = 2.02 Hz, 1 H) 7.26 (d, J = 1.52 Hz, 1 H) 7.29 (d, J = 1.52 Hz, 1 H) 7.32 (d, J = 1.77 Hz, 1 H) | 526.0, 524.0 |
| 199 | 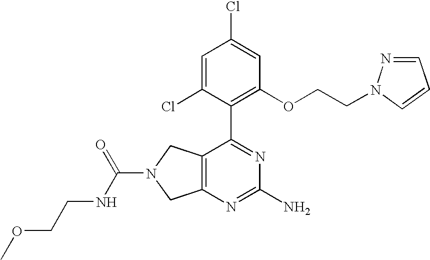 | 2-amino-4-{2,4-dichloro-6-[2-(1H- pyrazol-1-yl)ethoxy]phenyl}-N-(2- methoxyethyl)-5,7-dihydro-6H- pyrrolo[3,4-d]pyrimidine-6- carboxamide | Ex. 110 | 1H NMR (400 MHz, DMSO-d6) d ppm 3.16-3.22 (m, 2 H) 3.24 (s, 3 H) 3.31-3.37 (m, 2 H) 3.57 (d, J = 12.88 Hz, 1 H) 3.92 (d, J = 13.14 Hz, 1 H) 4.30-4.36 (m, 4 H) 4.37-4.45 (m, 2 H), 6.00 (t, J = 1.89 Hz, 1 H) 6.28-6.35 (m, 1 H) 6.79 (s, 2 H) 7.19 (d, J = 1.77 Hz, 1 H) 7.25 (d, J = 1.77 Hz, 1 H) 7.30 (d, J = 1.26 Hz, 1 H) 7.32 (d, J = 1.77 Hz, 1 H) | 494.2, 492.2 |
| 200 | 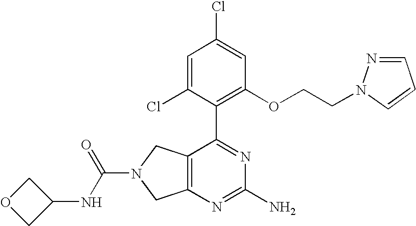 | 2-amino-4-{2,4-dichloro-6-[2-(1H- pyrazol-1-yl)ethoxy]phenyl}-N-oxetan- 3-yl-5,7-dihydro-6H-pyrrolo[3,4- d]pyrimidine-6-carboxamide | Ex. 110 | 1H NMR (300 MHz, DMSO-d6) d ppm 3.60 (d, J = 13.00 Hz, 1 H) 3.97 (d, J = 12.81 Hz, 1 H) 4.33 (s, 4 H) 4.38-4.52 (m, 4 H) 4.61-4.81 (m, 3 H) 5.97-6.02 (m, 1 H) 6.80 (s, 2 H) 6.89 (d, J = 5.65 Hz, 1 H) 7.20 (d, J = 1.88 Hz, 1 H) 7.25-7.30 (m, 2 H) 7.33 (d, J = 1.51 Hz, 1 H) | 492.2, 490.2 |
| 201 | 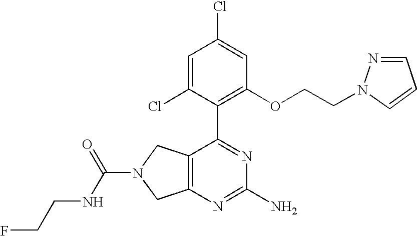 | 2-amino-4-{2,4-dichloro-6-[2-(1H- pyrazol-1-yl)ethoxy]phenyl}-N-(2- fluoroethyl)-6,7-dihydro-6H- pyrrolo[3,4-d]pyrimidine-6- carboxamide | Ex. 110 | 1H NMR (300 MHz, DMSO-d6) d ppm 3.26-3.41 (m, 2 H) 3.57 (d, J = 13.00 Hz, 1 H) 3.93 (d, J = 12.81 Hz, 1 H) 4.30-4.54 (m, 3 H) 6.00 (t, J = 1.88 Hz, 1 H) 6.50 (s, 1 H) 6.79 (s, 2 H) 7.20 (d, J = 2.07 Hz, 1 H) 7.25 (d, J = 1.70 Hz, 1 H) 7.30 (d, J = 1.51 Hz, 1 H) 7.32 (d, J = 1.51 Hz, 1 H) | 482.2, 480.2 |
| 202 | 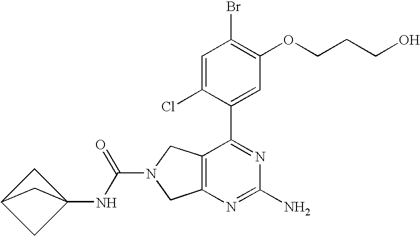 | 2-amino-N-bicyclo[1.1.1]pent-1-yl-4-[4- bromo-2-chloro-5-(3- hydroxypropoxy)phenyl]-5,7-dihydro- 6H-pyrrolo[3,4-d]pyrimidine-6- carboxamide | Ex. 111 | 1H NMR (300 MHz, DMSO-d6) d ppm 1.82-1.90 (m, 2 H) 1.93 (s, 6 H) 2.34 (s, 1 H) 3.57 (t, J = 6.22 Hz, 2 H) 4.12 (t, J = 6.12 Hz, 2 H) 4.23 (s, 2 H) 4.39 (s, 2 H) 6.90 (s, 2 H) 7.01 (s, 1 H) 7.15 (s, 1 H) 7.85 (s, 1 H) | 510.2, 508.2 |
| 203 | 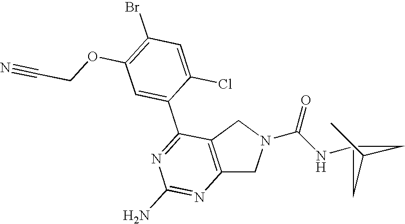 | 2-amino-N-bicyclo[1.1.1]pent-1-yl-4-[4- bromo-2-chloro-5- (cyanomethoxy)phenyl]-5,7-dihydro- 6H-pyrrolo[3,4-d]pyrimidine-6- carboxamide | Ex. 111 | 1H NMR (300 MHz, DMSO-d6) d ppm 1.93 (s, 6 H) 2.34 (s, 1 H) 4.23 (s, 2 H) 4.40 (s, 2 H) 5.31 (s, 2 H) 6.93 (s, 2 H) 6.98 (s, 1 H) 7.36 (s, 1 H) 7.98 (s, 1 H) | 491.2, 489.2 |
| 204 | 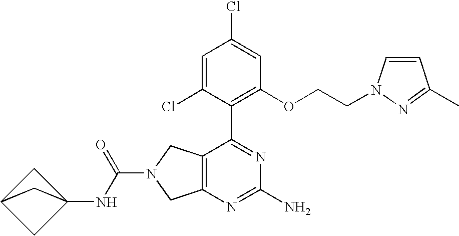 | 2-amino-N-bicyclo[1.1.1[pent-1-yl-4- {2,4-dichloro-6-[2-(3-methyl-1H- pyrazol-1-yl)ethoxy]phenyl}-5,7- dihydro-6H-pyrrolo[3,4-d]pyrimidine-6- carboxamide | Ex. 18 | 1H NMR (300 MHz, METHANOL-d4) d ppm 2.05 (s, 6 H) 2.19 (s, 3 H) 2.38 (s, 1 H) 3.65 (d, J = 12.81 Hz, 1 H) 4.06 (d, J = 12.81 Hz, 1 H) 4.32 (s, 4 H) 4.53 (s, 2 H) 5.89 (d, J = 2.07 Hz, 1 H) 7.08 (d, J = 2.26 Hz, 1 H) 7.18 (d, J = 1.70 Hz, 1 H) 7.24 (d, J = 1.70 Hz, 1 H) | 516.2, 514.2 |
| 205 | 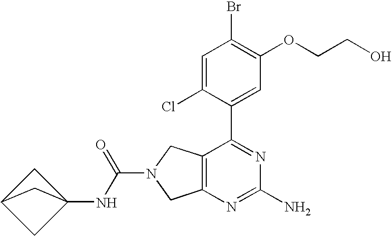 | 2-amino-N-bicyclo[1.1.1]pent-1-yl-4-[4- bromo-2-chloro-5-(2- hydroxyethoxy)phenyl]-5,7-dihydro-6H- pyrrolo[3,4-d]pyrimidine-6- carboxamide | Ex. 111 | 1H NMR (300 MHz, DMSO-d6) d ppm 1.93 (s, 6 H) 2.34 (s, 1 H) 3.72 (t, J = 4.71 Hz, 2 H) 4.09 (t, J = 4.80 Hz, 2 H) 4.23 (s, 2 H) 4.39 (s, 2 H) 6.89 (s, 2 H) 7.01 (s, 1 H) 7.18 (s, 1 H) 7.85 (s, 1 H) | 496.2, 494.2 |
| 206 | 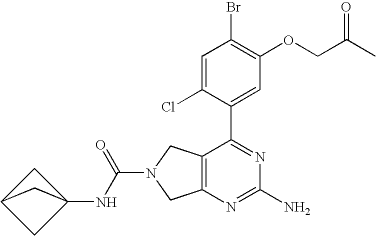 | 2-amino-N-bicyclo[1.1.1]pent-1-yl-4-[4- bromo-2-chloro-5-(2-chloro-5-(2- oxopropoxy)phenyl]phenyl]-5,7- dihydro-6H-pyrrolo[3,4-d]pyrimidine-6- carboxamide | Ex. 111 | 1H NMR (300 MHz, DMSO-d6) d ppm 1.93 (s, 6 H) 2.14 (s, 3 H) 2.34 (s, 1 H) 4.18 (s, 2 H) 4.38 (s, 2 H) 4.99 (s, 2 H) 6.88 (s, 2 H) 7.00 (s, 1 H) 7.07 (s, 1 H) 7.87 (s, 1 H) | 508.2, 506.2 |
| 207 | 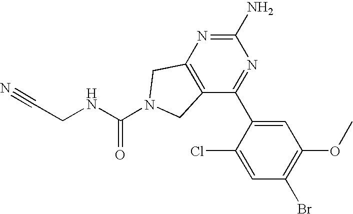 | 2-amino-4-(4-bromo-2-chloro-5- methoxyphenyl)-N-(cyanomethyl)-5,7- dihydro-6H-pyrrolo[3,4-d]pyrimidine-6- carboxamide | Ex. 2 Ex. 109 | 1H NMR (300 MHz, DMSO-d6) d ppm 3.87 (s, 3 H) 4.06 (d, J = 5.09 Hz, 2 H) 4.31 (s, 2 H) 4.47 (s, 2 H) 6.93 (s, 2 H) 7.17 (s, 1 H) 7.18 (t, J = 5.36 Hz, 1 H) 7.87 (s, 1 H) | 439.0, 437.0 |
| 208 | 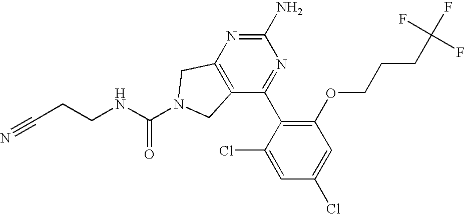 | 2-amino-N-(2-cyanoethyl)-4-[2,4- dichloro-6-(4,4,4- trifluorobutoxy)phenyl]-5,7-dihydro- 6H-pyrrolo[3,4-d]pyrimidine-6- carboxamide | Ex. 18 | 1H NMR (300 MHz, DMSO-d6) d ppm 1.70-1.82 (m, 2 H) 2.02-2.20 (m, 2 H) 2.62 (t, J = 6.31 Hz, 2 H) 3.25 (q, J = 6.09 Hz, 2 H) 4.04-4.20 (m, 4 H) 4.35-4.50 (m, 2 H) 6.81 (t, J = 5.46 Hz, 1 H) 6.85 (s, 2 H) 7.31 (d, J = 1.51 Hz, 1 H) 7.36 (d, J = 1.32 Hz, 1 H) | 505.2, 503.2 |
| 209 | 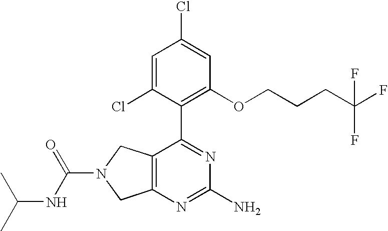 | 2-amino-4-[2,4-dichloro-6-(4,4,4- trifluorobutoxy)phenyl]-N-isopropyl- 5,7-dihydro-6H-pyrrolo[3,4- d]pyrimidine-6-carboxamide | Ex. 8a Ex 18 | 1H NMR (300 MHz, DMSO-d6) d ppm 1.05 (d, J = 5.27 Hz, 6 H) 1.69-1.83 (m, 2 H) 2.01-2.20 (m, 2 H) 3.70-3.85 (m, 1 H) 4.04-4.18 (m, J = 7.54 Hz, 4 H) 4.37-4.44 (m, 2 H) 6.04 (d, J = 7.72 Hz, 1 H) 6,81 (tr. s., 2 H) 7.31 (d, J = 1.32 Hz, 1 H) 7.36 (d, J = 1.51 Hz, 1 H) | 494.2, 492.2 |
| 210 | 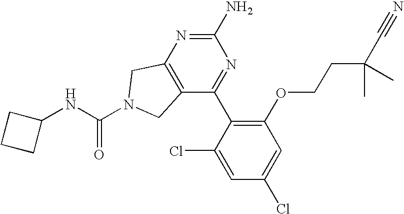 | 2-amino-N-cyclobutyl-4-[2,4-dichloro- 6-(3-cyano-3-methylbutoxy)phenyl]- 5,7-dihydro-6H-pyrrolo[3,4- d]pyrimidine-6-carboxamide | Ex. 18 | 1H NMR (300 MHz, DMSO-d6) d ppm 1.19 (d, J = 3.01 Hz, 6 H) 1.46-1.60 (m, 2 H) 1.81-1.99 (m, 4 H) 2.02-2.15 (m, 2 H) 4.07-4.23 (m, 5 H) 4.30-4.45 (m, 2 H) 6.51 (d, J = 7.72 Hz, 1 H) 6.80 (s, 2 H) 7.36-7.39 (m, J = 3.20 Hz, 2 H) | 491.2, 489.2 |
| 211 | 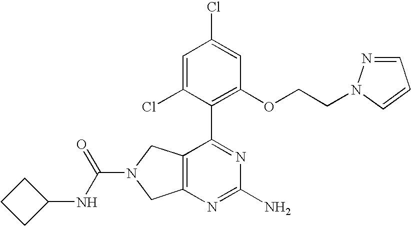 | 2-amino-N-cyclobutyl-4-{2,4-dichloro- 6-[2-(1H-pyrazol-1-yl)ethoxy]phenyl}- 5,7-dihydro-6H-pyrrolo[3,4- d]pyrimidine-6-carboxamide | Ex. 18 | 1H NMR (300 MHz, DMSO-d6) d ppm 1.48-1.62 (m, 2 H) 1.85-2.01 (m, 2 H) 2.04-2.18 (m, 2 H) 3.57 (d, J = 13.00 Hz, 1 H) 3.93 (d, J = 12.24 Hz, 1 H) 4.08-4.20 (m, 1 H) 4.29-4.46 (m, 6 H) 5.98-6.00 (m, 1 H) 6.40 (d, J = 7.72 Hz, 1 H) 6.79 (s, 2 H) 7.19 (d, J = 2.26 Hz, 1 H) 7.26 (d, J = 1.32 Hz, 1 H) 7.28 (d, J = 1.51 Hz, 1 H) 7.33 (d, J = 1.32 Hz, 1 H) | 490.2, 488.2 |
| 212 | 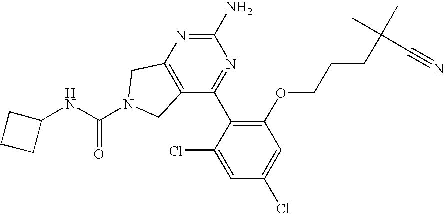 | 2-amino-N-cyclobutyl-4-{2,4-dichloro- 6-[(4-cyano-4- methylpentyl)oxy]phenyl}-5,7-dihydro- 6H-pyrrolo[3,4-d]pyrimidine-6- carboxamide | Ex. 18 | 1H NMR (300 MHz, DMSO-d6) d ppm 1.17 (s, 6 H) 1.32-1.42 (m, 2 H) 1.48-1.72 (m, 4 H) 1.83-1.99 (m, 2 H) 2.02-2.15 (m, 2 H) 4.02-4.20 (m, 5 H) 4.33-4.47 (m, 2 H) 6.52 (d, J = 7.72 Hz, 1 H) 6.83 (s, 2 H) 7.29 (d, J = 1.32 Hz, 1 H) 7.34 (d, J = 1.70 Hz, 1 H) | 505.2, 503.2 |
| 213 | 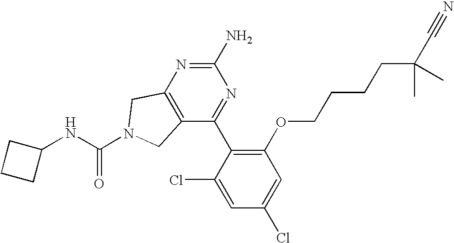 | 2-amino-N-cyclobutyl-4-{2,4-dichloro- 6-[(5-cyano-5- methylhexyl)oxy]phenyl}-5,7-dihydro- 6H-pyrrolo[3,4-d]pyrimidine-6- carboxamide | Ex. 18 | 1H NMR (300 MHz, DMSO-d6) d ppm 1.18 (d, J = 2.07 Hz, 6 H) 1.25-1.64 (m, 8 H) 1.83-1.99 (m, 2 H) 2.02-2.15 (m, 2 H) 4.00-4.18 (m, 5 H) 4.40 (s, 2 H) 6.50 (d, J = 7.72 Hz, 1 H) 6.81 (s, 2 H) 7.29 (d, J = 1.51 Hz, 1 H) 7.33 (d, J = 1.70 Hz, 1 H) | 519.2, 517.2 |
| 214 | 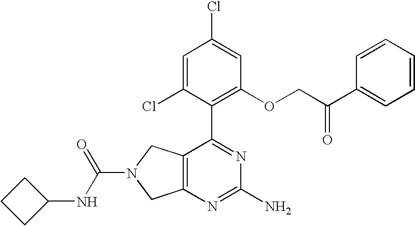 | 2-amino-N-cyclobutyl-4-[2,4-dichloro- 6-(2-oxo-2-phenylethoxy)phenyl]-5,7- dihydro-6H-pyrrolo[3,4-d]pyrimidine-6- carboxamide | Ex. 18 | 1H NMR (400 MHz, DMSO-d6) d ppm 1.47-1.61 (m, 2 H) 1.87-1.99 (m, 2 H) 2.05-2.14 (m, 2 H) 4.07-4.21 (m, 2 H) 4.29 (d, J = 13.14 Hz, 1 H) 4.41 (s, 2 H) 5.66 (d, J = 17.94 Hz, 1 H) 5.79 (d, J = 17.68 Hz, 1 H) 6.54 (d, J = 7.83 Hz, 1 H) 6.83 (s, 2 H) 7.29 (d, J = 1.77 Hz, 1 H) 7.34 (d, J = 1.77 Hz, 1 H) 7.54 (t, J = 7.71 Hz, 2 H) 7.67 (t, J = 7.45 Hz, 1 H) 7.93 (d, J = 7.33 Hz, 2 H) | 514.2, 512.2 |
| 215 | 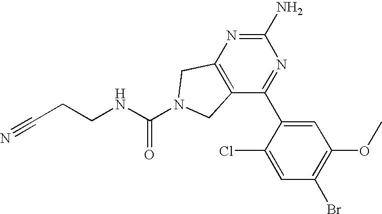 | 2-amino-4-(4-bromo-2-chloro-5- methoxyphenyl)-N-(2-cyanoethyl)-5,7- dihydro-6H-pyrrolo[3,4-d]pyrimidine-6- carboxamide | Ex. 2, Ex. 109 | 1H NMR (300 MHz, DMSO-d6) d ppm 2.63 (t, J = 6.40 Hz, 2 H) 3.21-3.30 (m, 2 H) 3.86 (s, 3 H) 4.28 (s, 2 H) 4.44 (s, 2 H) 6.38 (t, J = 4.52 Hz, 1 H) 6.93 (s, 2 H) 7.18 (s, 1 H) 7.87 (s, 1 H) | 453.0, 451.0 |
| 216 | 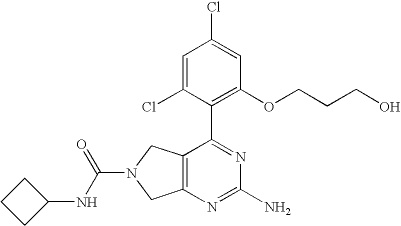 | 2-amino-N-cyclobutyl-4-[2,4-dichloro- 6-(3-hydroxypropoxy)phenyl]-5,7- dihydro-6H-pyrrolo[3,4-d]pyrimidine-6- carboxamide | Ex. 18 | 1H NMR (300 MHz, DMSO-d6) d ppm 1.47-1.60 (m, 2 H) 1.62-1.73 (m, 2 H) 1.84-1.99 (m, 2 H) 2.02-2.15 (m, 2 H) 3.31 (t, J = 5.56 Hz, 2 H) 4.04-4.18 (m, 5 H) 4.41 (s, 2 H) 6.51 (d, J = 7.54 Hz, 1 H) 6.82 (s, 2 H) 7.28 (s, 1 H) 7.32 (s, 1 H) | 454.2, 452.2 |
| 217 | 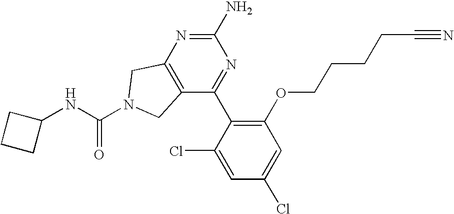 | 2-amino-N-cyclobutyl-4-[2,4-dichloro- 6-(4-cyanobutoxy)phenyl]-5,7-dihydro- 6H-pyrrolo[3,4-d]pyrimidine-6- carboxamide | Ex. 18 | 1H NMR (300 MHz, DMSO-d6) d ppm 1.43-1.72 (m, 6 H) 1.84-2.00 (m, 2 H) 2.03-2.17 (m, 2 H) 2.40 (t, J = 7.06 Hz, 2 H) 4.02-4.21 (m, 5 H) 4.41 (s, 2 H) 6.50 (d, J = 7.91 Hz, 1 H) 6.81 (s, 2 H) 7.29 (d, J = 1.70 Hz, 1 H) 7.34 (d, J = 1.51 Hz, 1 H) | 477.2, 475.2 |
| 218 | 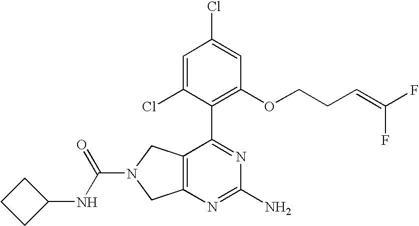 | 2-amino-N-cyclobutyl-4-{2,4-dichloro- 6-[(4,4-difluorobut-3-en-1- yl)oxy]phenyl}-5,7-dihydro-6H- pyrrolo[3,4-d]pyrimidine-6- carboxamide | Ex. 18 | 1H NMR (300 MHz, DMSO-d6) d ppm 1.46-1.61 (m, 2 H) 1.82-1.99 (m, 2 H) 2.02-2.14 (m, 2 H) 2.15-2.28 (m, 2 H) 3.94-4.46 (m, 8 H) 6.48 (d, J = 8.48 Hz, 1 H) 6.79 (s, 2 H) 7.28 (d, J = 1.32 Hz, 1 H) 7.34 (d, 1 H) | 486.2, 484.2 |
| 219 | 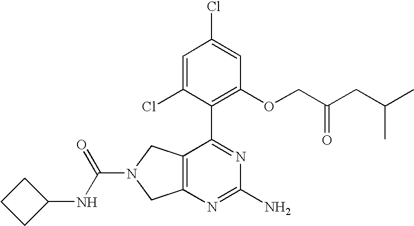 | 2-amino-N-cyclobutyl-4-{2,4-dichloro- 6-[(4-methyl-2-oxopentyl)oxy]phenyl}- 5,7-dihydro-6H-pyrrolo[3,4- d]pyrimidine-6-carboxamide | Ex. 18 | 1H NMR (300 MHz, DMSO-d6) d ppm 0.80 (dd, J = 6.59, 2.45 Hz, 6 H) 1.48-1.60 (m, 2 H) 1.86-2.15 (m, 5 H) 2.24 (dd, J = 6.78, 2.07 Hz, 2 H) 4.06-4.29 (m, 3 H) 4.41 (s, 2 H), 4.81 (d, J = 17.52 Hz, 1 H) 4.93 (d, J = 17.33 Hz, 1 H) 6.52 (d, J = 7.72 Hz, 1 H) 6.83 (s, 2 H) 7.13 (d, J = 1.32 Hz, 1 H) 7.35 (d, J = 1.70 Hz, 1 H) | 494.0, 492.0 |
| 220 | 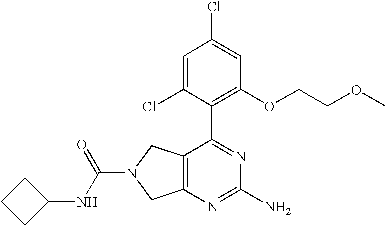 | 2-amino-N-cyclobutyl-4-[2,4-dichloro- 6-(2-methoxyethoxy)phenyl]-5,7- dihydro-6H-pyrrolo[3,4-d]pyrimidine-6- carboxamide | Ex. 18 | 1H NMR (300 MHz, DMSO-d6) d ppm 1.48-1.60 (m, 2 H) 1.83-1.99 (m, 2 H) 2.02-2.14 (m, 2 H) 3.12 (s, 3 H) 3.45-3.52 (m, 2 H) 3.45-3.52 (m, 2 H) 4.06-4.22 (m, 5 H) 4.40 (br. s., 2 H) 6.49 (d, J = 8.10 Hz, 1 H) 6.81 (br. s., 2 H) 7.31 (d, J = 1.51 Hz, 1 H) 7.34 (d, J = 1.70 Hz, 1 H) | 454.0, 452.0 |
| 221 | 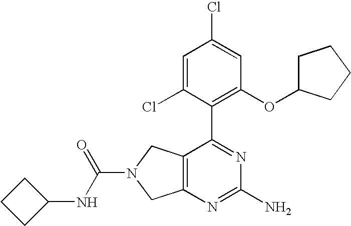 | 2-amino-N-cyclobutyl-4-[2,4-dichloro- 6-(cyclopentyloxy)phenyl]-5,7-dihydro- 6H-pyrrolo[3,4-d]pyrimidine-6- carboxamide | Ex. 18 | 1H NMR (300 MHz, DMSO-d6) d ppm 1.39-1.98 (m, 12 H) 2.02-2.15 (m, 2 H) 4.03-4.18 (m, 3 H) 4.40 (s, 2 H) 4.89-4.97 (m, 1 H) 6.52 (d, J = 7.91 Hz, 1 H) 6.80 (s, 2 H) 7.26 (d, J = 1.70 Hz, 1 H) 7.30 (d, J = 1.70 Hz, 1 H) | 464.0, 462.0 |
| 222 | 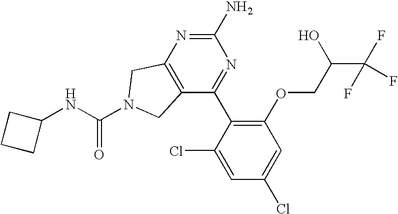 | 2-amino-N-cyclobutyl-4-[2,4-dichloro- 6-(3,3,3-trifluoro-2- hydroxypropoxy)phenyl]-5,7-dihydro- 6H-pyrrolo[3,4-d]pyrimidine-6- carboxamide | Ex. 18 | 1H NMR (300 MHz, DMSO-d6) d ppm 1.48-1.59 (m, 2 H) 1.84-1.99 (m, 2 H) 2.03-2.14 (m, 2 H) 4.04-4.25 (m, 6 H) 4.39 (s, 2 H) 6.48 (d, J = 7.35 Hz, 1 H) 6.63 (br. s., 1 H) 6.78 (s, 2 H) 7.38 (s, 1 H) 7.41 (s, 1 H) | 508.0, 506.0 |
| 223 | 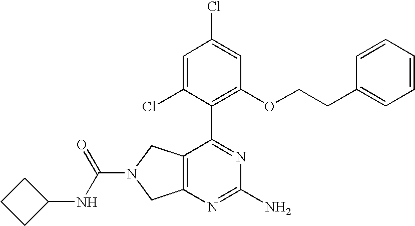 | 2-amino-N-cyclobutyl-4-[2,4-dichloro- 6-(2-phenylethoxy)phenyl]-5,7-dihydro- 6H-pyrrolo[3,4-d]pyrimidine-6- carboxamide | Ex. 18 | 1H NMR (300 MHz, DMSO-d6) d ppm 1.48-1.62 (m, 2 H) 1.84-2.01 (m, 2 H) 2.04-2.19 (m, 2 H) 2.70-2.92 (m, 2 H) 3.60 (d, J = 12.62 Hz, 1 H) 3.94 (d, J = 13.19 Hz, 1 H) 4.07-4.46 (m, 5 H) 6.39 (d, J = 7.72 Hz, 1 H) 6.83 (br. s., 2 H) 6.98 (d, J = 6.41 Hz, 2 H) 7.04-7.19 (m, 3 H) 7.26 (s, 1 H) 7.30 (s, 1 H) | 500.0, 498.0 |
| 224 | 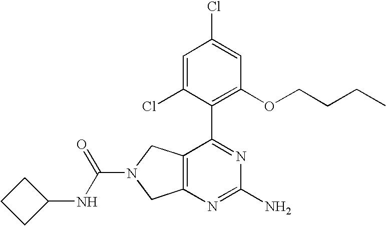 | 2-amino-4-(2-butoxy-4,6- dichlorophenyl)-N-cyclobutyl-5,7- dihydro-6H-pyrrolo[3,4-d]pyrimidine-6- carboxamide | Ex. 18 | 1H NMR (400 MHz, DMSO-d6) d ppm 0.78 (t, J = 7.45 Hz, 3 H) 1.14-1.25 (m, 2 H) 1.46-1.58 (m, 4 H) 1.86-1.97 (m, 2 H) 2.04-2.13 (m, 2 H) 3.98-4.05 (m, 2 H) 4.08-4.16 (m, 3 H) 4.40 (s, 2 H) 6.52 (d, J = 7.83 Hz, 1 H) 6.82 (s, 2 H) 7.28 (d, J = 1.77 Hz, 1 H) 7.32 (d, J = 1.77 Hz, 1 H) | 452.0, 450.0 |
| 225 | 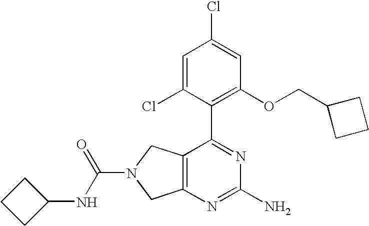 | 2-amino-N-cyclobutyl-4-[2,4-dichloro- 6-(cyclobutylmethoxy)phenyl]-5,7- dihydro-6H-pyrrolo[3,4-d]pyrimidine-6- carboxamide | Ex. 18 | 1H NMR (300 MHz, DMSO-d6) d ppm 1.45-2.00 (m, 11 H) 2.02-2.16 (m, 2 H) 3.98 (d, J = 5.84 Hz, 2 H) 4.08-4.20 (m, 3 H) 4.36-4.48 (m, 2 H) 6.51 (d, J = 7.91 Hz, 1 H) 6.81 (s, 2 H) 7.28 (s, 1 H) 7.33 (s, 1 H) | 464.2, 462.2 |
| 226 | 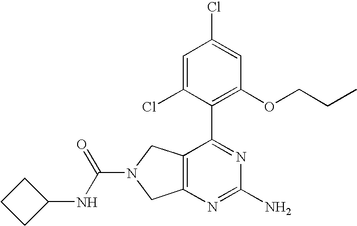 | 2-amino-N-cyclobutyl-4-(2,4-dichloro- 6-propoxyphenyl)-5,7-dihydro-6H- pyrrolo[3,4-d]pyrimidine-6- carboxamide | Ex. 18 | 1H NMR (300 MHz, DMSO-d6) d ppm 0.76 (t, J = 7.35 Hz, 3 H) 1.47-1.62 (m, 4 H) 1.83-2.01 (m, 2 H) 2.02-2.15 (m, 2 H) 3.93-4.05 (m, 2 H) 4.07-4.17 (m, 3 H) 4.40 (s, 2 H) 6.51 (d, J = 8.29 Hz, 1 H) 6.82 (br. s., 2 H) 7.27 (d, J = 1.32 Hz, 1 H) 7.32 (d, J = 1.32 Hz, 1 H) | 438.2, 436.2 |
| 227 | 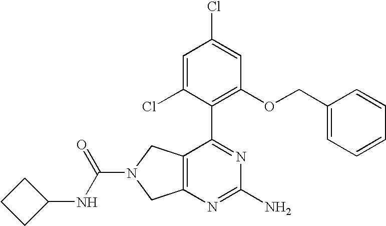 | 2-amino-4-[2-(benzyloxy)-4,6- dichlorophenyl]-N-cyclobutyl-5,7- dihydro-6H-pyrrolo[3,4-d]pyrimidine-6- carboxamide | Ex. 18 | 1H NMR (300 MHz, DMSO-d6) d ppm 1.48-1.60 (m, 2 H) 1.84-2.01 (m, 2 H) 2.03-2.15 (m, 2 H) 4.07-4.20 (m, 3 H) 4.40 (s, 2 H) 5.20 (s, 2 H) 6.51 (d, J = 7.91 Hz, 1 H) 6.85 (br. s., 2 H) 7.19-7.25 (m, 2 H) 7.26-7.34 (m, 3 H) 7.36 (s, 2 H) | 486.2, 484.2 |
| 228 | 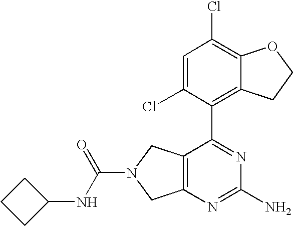 | 2-amino-N-cyclobutyl-4-(5,7-dichloro- 2,3-dihydro-1-benzofuran-4-yl)-5,7- dihydro-6H-pyrrolo[3,4-d]pyrimidine-6- carboxamide | Ex. 7 | 1H NMR (400 MHz, DMSO-d6) d ppm 1.47-1.61 (m, 2 H) 1.87-1.99 (m, 2 H) 2.05-2.13 (m, 2 H) 2.99-3.21 (m, 2 H) 4.07-4.21 (m, 2 H) 4.27 (d, J = 13.14 Hz, 1 H) 4.36-4.48 (m, 2 H) 4.61-4.74 (m, 2 H) 6.54 (d, J = 7.83 Hz, 1 H) 6.90 (br. s., 2 H) 7.53 (s, 1 H) | 422.2, 420.2 |
| 229 | 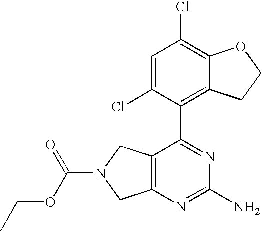 | ethyl 2-amino-4-(5,7-dichloro-2,3- dihydro-1-benzofuran-4-yl)-5,7- dihydro-6H-pyrrolo[3,4-d]pyrimidine-6- carboxylate | Ex. 1 | 1H NMR (300 MHz, DMSO-d6) d ppm 1.13-1.27 (m, 3 H) 2.95-3.26 (m, 2 H) 3.98-4.16 (m, 2 H) 4.22-4.39 (m, 2 H) 4.41-4.58 (m, 2 H) 4.61-4.76 (m, 2 H) 6.94 (br. s., 2 H) 7.51 (s, 1 H) | 397.2, 395.2 |
| 230 | 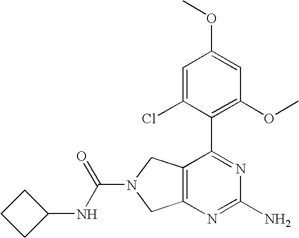 | 2-amino-4-(2-chloro-4,6- dimethoxyphenyl)-N-cyclobutyl-5,7- dihydro-6H-pyrrolo[3,4-d]pyrimidine-6- carboxamide | Ex. 117 | 1H NMR (300 MHz, DMSO-d6) d ppm 1.48-1.60 (m, 2 H) 1.84-2.00 (m, 2 H) 2.03-2.15 (m, 2 H) 3.72 (s, 3 H) 3.83 (s, 3 H) 4.04-4.19 (m, 3 H) 4.40 (s, 2 H) 6.51 (d, J = 7.91 Hz, 1 H) 6.67 (d, J = 2.07 Hz, 1 H) 6.71-6.77 (m, 3 H) | 406.0, 404.0 |
| 231 | 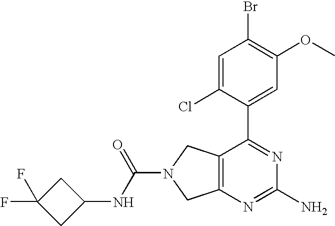 | 2-amino-4-(4-bromo-2-chloro-5- methoxyphenyl)-N-(3,3- difluorocyclobutyl)-5,7-dihydro-6H- pyrrolo[3,4-d]pyrimidine-6- carboxamide | Ex. 2, Ex. 109 | 1H NMR (300 MHz, DMSO-d6) d ppm 2.53-2.89 (m, 4 H) 3.87 (s, 3 H) 3.95-4.08 (m, 1 H) 4.28 (s, 2 H) 4.44 (s, 2 H) 6.75 (d, J = 6.78 Hz, 1 H) 6.91 (br. s., 2 H) 7.17 (s, 1 H) 7.87 (s, 1 H) | 489.8, 487.8 |
| 232 | 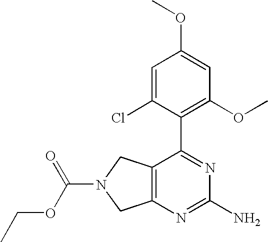 | ethyl 2-amino-4-(2-chloro-4,6- dimethoxyphenyl)-5,7-dihydro-6H- pyrrolo[3,4-d]pyrimidine-6-carboxylate | Ex. 117a | 1H NMR (300 MHz, DMSO-d6) d ppm 1.13-1.24 (m, 3 H) 3.72 (s, 3 H) 3.83 (s, 3 H) 4.00-4.19 (m, 4 H) 4.47 (d, J = 8.48 Hz, 2 H) 6.66 (d, J = 2.26 Hz, 1 H) 6.74 (d, J = 2.07 Hz, 1 H) 6.79 (br. s., 2 H) | 379.2 |
| 233 | 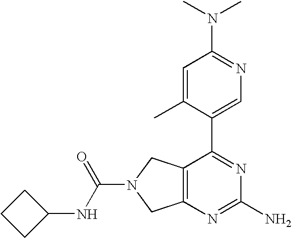 | 2-amino-N-cyclobutyl-4-[6- (dimethylamino)-4-methylpyridin-3-yl]- 5,7-dihydro-6H-pyrrolo[3,4- d]pyrimidine-6-carboxamide | Ex. 117 | 1H NMR (300 MHz, DMSO-d6) d ppm 1.48-1.63 (m, 2 H) 1.87-2.02 (m, 2 H) 2.04-2.17 (m, 2 H) 2.28 (s, 3 H) 3.06 (s, 6 H) 4.06-4.22 (m, 1 H) 4.34-4.48 (m, 4 H) 6.52-6.69 (m, 4 H) 8.05 (s, 1 H) | 343.4 |
| 234 | 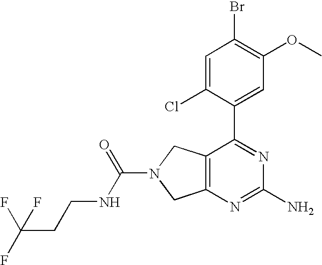 | 2-amino-4-(4-bromo-2-chloro-5- methoxyphenyl)-N-(3,3,3- trifluoropropyl)-5,7-dihydro-6H- pyrrolo[3,4-d]pyrimidine-6- carboxamide | Ex. 2, Ex. 109 | 1H NMR (300 MHz, DMSO-d6) d ppm 2.32-2.47 (m, 2 H) 3.22-3.30 (m, 2 H) 3.86 (s, 3 H) 4.27 (s, 2 H) 4.42 (s, 2 H) 6.63 (t, J = 5.27 Hz, 1 H) 6.90 (br. s., 2 H) 7.17 (s, 1 H) 7.86 (s, 1 H) | 496.0, 493.9 |
| 235 | 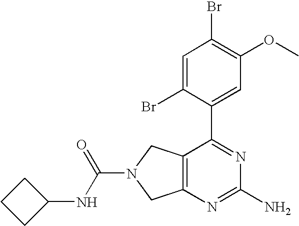 | 2-amino-N-cyclobutyl-4-(2,4-dibromo- 5-methoxyphenyl)-5,7-dihydro-6H- pyrrolo[3,4-d]pyrimidine-6- carboxamide | Ex. 117 | 1H NMR (300 MHz, DMSO-d6) d ppm 1.47-1.63 (m, 2 H) 1.84-2.01 (m, 2 H) 2.02-2.16 (m, 2 H) 3.85 (s, 3 H) 4.05-4.20 (m, 1 H) 4.24 (s, 2 H) 4.41 (s, 2 H) 6.52 (d, J = 7.91 Hz, 1 H) 6.88 (br. s., 2 H) 7.15 (s, 1 H) 7.96 (s, 1 H) | 498.2 |
| 236 | 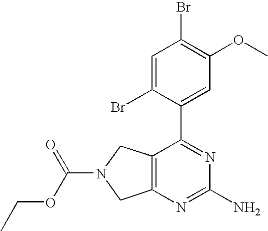 | ethyl 2-amino-4-(2,4-dibromo-5- methoxyphenyl)-5,7-dihydro-6H- pyrrolo[3,4-d]pyrimidine-6-carboxylate | Ex. 117a | 1H NMR (300 MHz, DMSO-d6) d ppm 1.13-1.27 (m, 3 H) 3.85 (s, 3 H) 4.02-4.16 (m, 2 H) 4.29-4.35 (m, 2 H) 4.45-4.52 (m, 2 H) 6.94 (br. s., 2 H) 7.12 (s, 1 H) 7.96 (s, 1 H) | 473.2 |
| 237 | 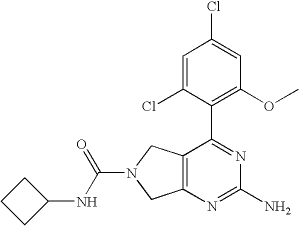 | 2-amino-N-cyclobutyl-4-(2,4-dichloro- 6-methoxyphenyl)-5,7-dihydro-6H- pyrrolo[3,4-d]pyrimidine-6- carboxamide | Ex. 117 | 1H NMR (300 MHz, DMSO-d6) d ppm 1.48-1.61 (m, 2 H) 1.85-2.00 (m, 2 H) 2.03-2.15 (m, 2 H) 3.77 (s, 3 H) 4.41 (s, 2 H) 6.50 (d, J = 7.91 Hz, 1 H) 6.82 (br. s., 2 H) 7.28 (d, J = 1.70 Hz, 1 H) 7.34 (d, J = 1.70 Hz, 1 H) | 408.2 |
| 238 | 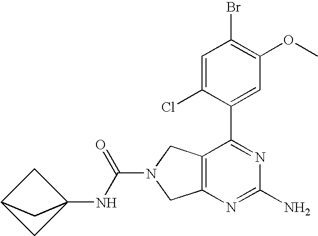 | 2-amino-N-bicyclo[1.1.1]pent-1-yl-4-(4- bromo-2-chloro-5-methoxyphenyl)-5,7- dihydro-6H-pyrrolo[3,4-d]pyrimidine-6- carboxamide | Ex. 2, Ex. 109 | 1H NMR (300 MHz, DMSO-d6) d ppm 1.93 (s, 6 H) 2.34 (s, 1 H) 3.86 (s, 3 H) 4.23 (s, 2 H) 4.39 (s, 2 H) 6.87 (br. s., 2 H) 6.99 (s, 1 H) 7.15 (s, 1 H) 7.85 (s, 1 H) | 466.0, 464.0 |
| 239 | 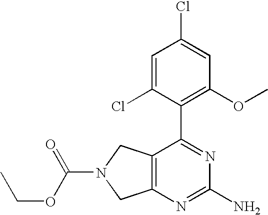 | ethyl 2-amino-4-(2,4-dichloro-6- methoxyphenyl)-5,7-dihydro-6H- pyrrolo[3,4-d]pyrimidine-6-carboxylate | Ex. 117a | 1H NMR (300 MHz, DMSO-d6) d ppm 1.13-1.24 (m, 3 H) 3.77 (s, 3 H) 4.00-4.12 (m, 2 H) 4.14 (s, 1 H) 4.17 (s, 1 H) 4.47 (s, 1 H) 4.49 (s, 1 H) 6.87 (br. s., 2 H) 7.25 (d, J = 1.51 Hz, 1 H) 7.32 (d, J = 1.51 Hz, 1 H) | 385.2, 383.2 |
| 240 | 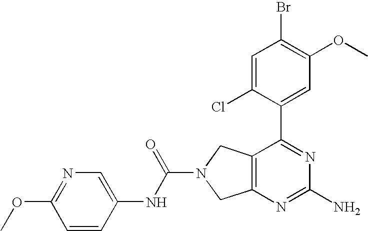 | 2-amino-4-(4-bromo-2-chloro-5- methoxyphenyl)-N-(6-methoxypyridin- 3-yl)-5,7-dihydro-6H-pyrrolo[3,4- d]pyrimidine-6-carboxamide | Ex. 118 | 1H NMR (300 MHz, DMSO-d6) d ppm 3.79 (s, 3 H) 3.87 (s, 3 H) 4.46 (s, 2 H) 4.58 (s, 2 H) 6.73 (d, J = 8.85 Hz, 1 H) 6.94 (s, 2 H) 7.19 (s, 1 H) 7.79 (dd, J = 8.95, 2.54 Hz, 1 H) 7.88 (s, 1 H) 8.21 (d, J = 2.64 Hz, 1 H) 8.37 (s, 1 H) | 507.2, 505.2 |
| 241 | 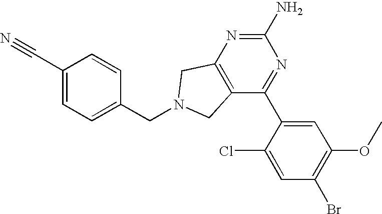 | 4-{[2-amino-4-(4-bromo-2-chloro-5- methoxyphenyl)-5,7-dihydro-6H- pyrrolo[3,4-d]pyrimidin-6- yl]methyl}benzonitrile | Ex. 120 | 1H NMR (300 MHz, DMSO-d6) d ppm 3.63 (s, 2 H) 3.78 (s, 2 H) 3.84 (s, 3 H) 3.94 (s, 2 H) 6.76 (br. s., 2 H) 7.09 (s, 1 H) 7.54 (d, J = 8.10 Hz, 2 H) 7.76-7.83 (m, 3 H) | 472.2, 470.2 |
| 242 | 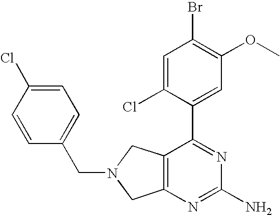 | 4-(4-bromo-2-chloro-5-methoxyphenyl)- 6-(4-chlorobenzyl)-6,7-dihydro-5 H- pyrrolo[3,4-d]pyrimidin-2-amine | Ex. 120 | 1H NMR (300 MHz, DMSO-d6) d ppm 3.59 (s, 2 H) 3.57 (s, 2 H) 3.82-3.86 (m, 5 H) 6.77 (br. s., 2 H) 7.09 (s, 1 H) 7.35-7.39 (m, 4 H) 7.79 (s, 1 H) | 481.0, 479.0 |
| 243 | 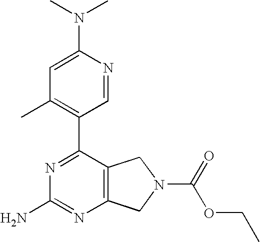 | ethyl 2-amino-4-[6-(dimethylamino)-4- methylpyridin-3-yl]-5,7-dihydro-6H- pyrrolo[3,4-d]pyrimidine-6-carboxylate | Ex. 117a | 1H NMR (300 MHz, DMSO-d6) d ppm 1.15-1.26 (m, 3 H) 2.28 (s, 3 H) 3.06 (s, 6 H) 4.02-4.15 (m, 2 H) 4.40-4.50 (m, 4 H) 6.56 (s, 1 H) 6.67 (s, 2 H) 8.06 (d, J = 3.58 Hz, 1 H) | 343.4 |
| 244 | 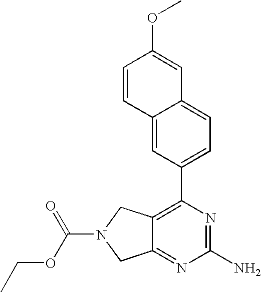 | ethyl 2-amino-4-(6-methoxy-2- naphthyl)-5,7-dihydro-6H-pyrrolo[3,4- d]pyrimidine-6-carboxylate | Ex. 1 | 1H NMR (300 MHz, DMSO-d6) d ppm 1.24 (t, J = 7.06 Hz, 3 H) 3.91 (s, 3 H) 4.14 (q, J = 6.78 Hz, 2 H) 4.44-4.51 (m, 2 H) 4.86 (s, 2 H) 7.24 (dd, J = 8.95, 2.35 Hz, 1 H) 7.40 (d, J = 1.88 Hz, 1 H) 7.93-8.04 (m, 3 H) 8.32 (d, J = 3.20 Hz, 1 H) | 365.4 |
| 245 | 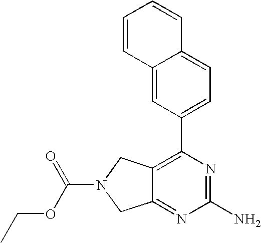 | ethyl 2-amino-4-(2-naphthyl)-5,7- dihydro-6H-pyrrolo[3,4-d]pyrimidine-6- carboxylate | Ex. 1 | 1H NMR (300 MHz, DMSO-d6) d ppm 1.20-1.28 (m, 3 H) 4.14 (q, J = 7.16 Hz, 2 H) 4.45-4.51 (m, 2 H) 4.87 (s, 2 H) 6.86 (br. s., 2 H) 7.57-7.67 (m, 2 H) 7.95-8.14 (m, 4 H) 8.38 (d, J = 3.58 Hz, 1 H) | 335.2 |
| 246 | 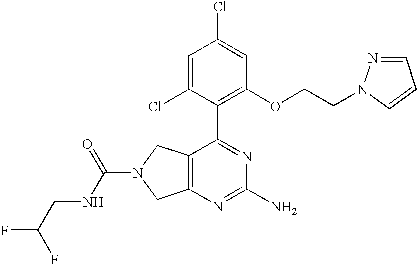 | 2-amino-4-{2,4-dichloro-6-[2-(1H- pyrazol-1-yl)ethoxy]phenyl}-N-(2,2- difluoroethyl)-5,7-dihydro-6H- pyrrolo[3,4-d]pyrimidine-6- carboxamide | Ex. 110 | 1H NMR (300 MHz, DMSO-d6) d ppm 3.33-3.51 (m, 2 H) 3.56 (d, J = 12.81 Hz, 1 H) 3.94 (d, J = 12.62 Hz, 1 H) 4.29-4.38 (m, 4 H) 4.40-4.50 (m, 2 H) 5.77-6.20 (m, 2 H) 5.77-6.20 (m, 2 H) 6.70 (br. s., 1 H) 6.80 (br. s., 1 H) 7.20 (d, J = 2.07 Hz, 1 H) 7.26 (d, J = 1.51 Hz, 1 H) 7.30 (d, J = 1.51 Hz, 1 H) 7.32 (d, J = 1.70 Hz, 1 H) | 500.2, 498.2 |
| 247 | 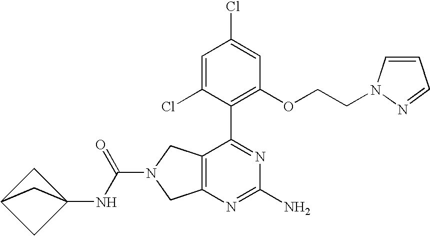 | 2-amino-N-bicyclo[1.1.1]pent-1-yl-4- {2,4-dichloro-6-[2-(1H-pyrazol-1- yl)ethoxy]phenyl}-5,7-dihydro-6H- pyrrolo[3,4-d]pyrimidine-6- carboxamide | Ex. 110 | 1H NMR (300 MHz, DMSO-d6) d ppm 1.95 (s, 6 H) 2.36 (s, 1 H) 3.53 (d, J = 13.19 Hz, 1 H) 3.89 (d, J = 13.00 Hz, 1 H) 4.33 (s, 6 H) 5.99-6.02 (m, 1 H) 6.77 (br. s., 2 H) 6.85 (br. s., 1 H) 7.19 (d, J = 2.07 Hz, 1 H) 7.25 (d, J = 1.51 Hz, 1 H) 7.30 (d, J = 1.70 Hz, 1 H) 7.32 (d, J = 1.70 Hz, 1 H) | 502.2, 500.2 |
| 248 | 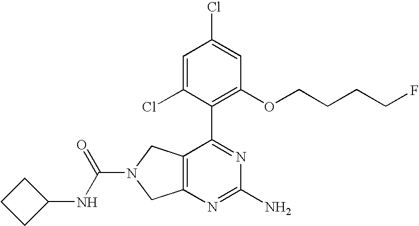 | 2-amino-N-cyclobutyl-4-[2,4-dichloro- 6-(4-fluorobutoxy)phenyl]-5,7-dihydro- 6H-pyrrolo[3,4-d]pyrimidine-6- carboxamide | Ex. 18 | 1H NMR (300 MHz, DMSO-d6) d ppm 1.61 (d, J = 5.27 Hz, 6 H) 1.84-1.98 (m, 2 H) 2.03-2.15 (m, 2 H) 4.03-4.28 (m, 6 H) 4.37-4.43 (m, 3 H) 6.51 (d, J = 7.19 Hz, 1 H) 6.82 (br. s., 2 H) 7.29 (d, J = 1.32 Hz, 1 H) 7.34 (d, J = 1.13 Hz, 1 H) | 470.2, 468.2 |
| 249 | 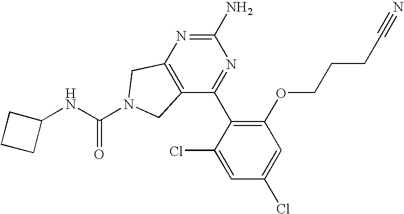 | 2-amino-N-cyclobutyl-4-[2,4-dichloro- 6-(3-cyanopropoxy)phenyl]-5,7- dihydro-6H-pyrrolo[3,4-d]pyrimidine-6- carboxamide | Ex. 18 | 1H NMR (400 MHz, DMSO-d6) d ppm 1.47-1.59 (m, 2 H) 1.82-1.98 (m, 4 H) 2.04-2.13 (m, 2 H) (t, J = 7.20 Hz, 2 H) 4.06-4.18 (m, 5 H) 4.40 (br. s., 2 H) 6.50 (d, J = 7.83 Hz, 1 H) 6.82 (br. s., 2 H) 7.33 (d, J = 1.77 Hz, 1 H) 7.37 (d, J = 1.77 Hz, 1 H) | 463.2, 461.2 |
| 250 | 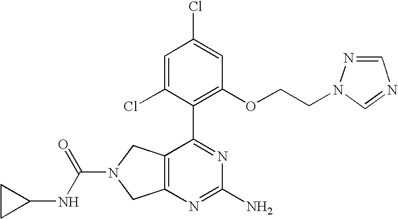 | 2-amino-N-cyclobutyl-4-{2,4-dichloro- 6-[2-(1 H,1,2,4-triazol-1- yl)ethoxy]phenyl}-5,7-dihydro-6H- pyrrolo[3,4-d]pyrimidine-6- carboxamide | Ex. 15 | 1H NMR (400 MHz, DMSO-d6) d ppm: 7.96 (s, 1 H), 7.73 (s, 1 H), 7.33 (d, J = 1.77 Hz, 1 H), 7.29 (d, J = 1.77 Hz, 1 H), 6.75 (s, 2 H), 6.35 (br. s., 1 H), 4.37-4.51 (m, 4 H), 4.25-4.36 (m, 2 H), 3.87 (dd, J = 13.01, 2.15 Hz, 1 H), 3.44-3.52 (m, 1 H), 2.52-2.57 (m, 1 H), 0.50-0.58 (m, 2 H), 0.42 (d, J = 3.28 Hz, 2 H). | 475.0, 477.0 |
| 251 | 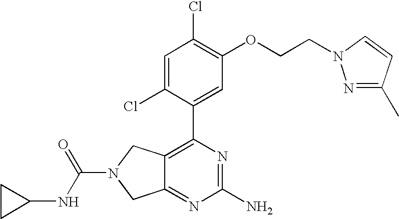 | 2-amino-N-cyclopropyl-4-{2,4- dichloro-5-[2-(3-methyl-1H-pyrazol-1- yl)ethoxy]phenyl}-5,7-dihydro-6H- pyrrolo[3,4-d]pyrimidine-6- carboxamide | Ex. 127 | 1H-NMR (CDCl3, 300 MHz): d 7.46 (s, 2H), 6.72 (s, 1H), 5.97 (s, 1H), 5.19 (s, 2H), 4.52 (s, 2H), 4.51 (s, 1H), 4.46 (t, 2H, J = 5 Hz), 4.38 (s, 2H), 4.31 (t, 2H, J = 5 Hz), 2.67 (bs, 1H), 2.20 (s, 3H), 0.70 (q, 2H, J = 5 Hz), 0.51 (m, 2H). | 488.0, 490.0 |
| 252 | 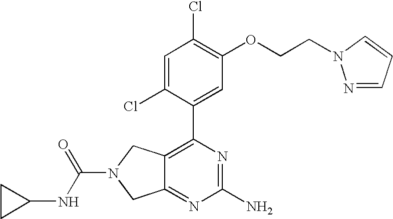 | 2-amino-N-cyclopropyl-4-{2,4- dichloro-5-[2-(1H-pyrazol-1- yl)ethoxy]phenyl}-5,7-dihydro-6H- pyrrolo[3,4-d]pyrimidine-6- carboxamide | Ex. 127 | 1H-NMR (CDCl3, 300 MHz): d 7.60 (s, 1H), 7.49 (s, 1H), 7.44 (s, 1H), 6.72 (s, 1H), 6.24 (s, 1H), 5,18 (s, 2H), 6.24 (s, 1H), 5.18 (s, 2H), 4.54 (m, 5H), 4.38 (s, 2H), 4.32 (t, 2H, J = 5 Hz), 2.66 (bs, 1H), 0.73 (q, 2H, J = 6 Hz), 0.51 (m, 2H). | 474.0, 476.0 |
| 253 | 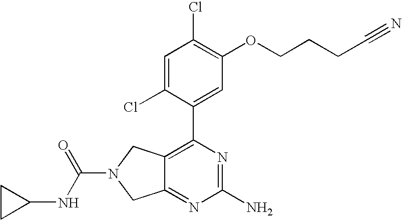 | 2-amino-N-cyclopropyl-4-[2,4-dichloro- 5-(3-cyanopropoxy)phenyl]-5,7- dihydro-6H-pyrrolo[3,4-d]pyrimidine-6- carboxamide | Ex. 127 | 1H-NMR (CDCl3, 300 MHz): d 7.48 (s, 1H), 6.87 (s, 1H), 5.18 (s, 2H), 4.56 (s, 3H), 4.48 (s, 2H), 4.12 (t, 2H, J = 6 Hz), 2.68 (bs, 2H), 2.65 (t, 2H, J = 6 Hz), 2.19 (q, 2H, J = 6 Hz), 0.72 (m, 2H), 0.50 (m, 2H). | 446.0, 448.0 |
| 254 | 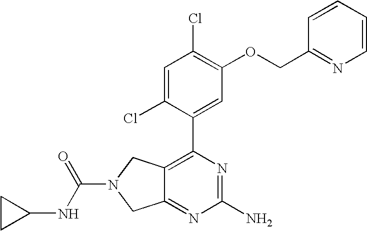 | 2-amino-N-cyclopropyl-4-[2,4-dichloro- 5-(pyridin-2-ylmethoxy)phenyl]-5,7- dihydro-6H-pyrrolo[3,4-d]pyrimidine-6- carboxamide | Ex. 127 | 1H-NMR (CDCl3, 300 MHz): d 8.55 (d, 1H, J = 6 Hz), 7.72 (t, 1H, J = 6 Hz), 7.60 (d, 1H, J = 6 Hz), 7.51 (s, 1H), 7.25 (m, 1H), 6.95 (s, 1H), 5.24 (s, 2H), 5.14 (s, 2H), 4.55 (s, 2H), 4.49 (s, 1H), 4.32 (s, 2H), 2.68 (bs, 1H), 0.73 (q, 2H, J = 6 Hz), 0.50 (m, 2H). | 471.0, 473.0 |
| 255 | 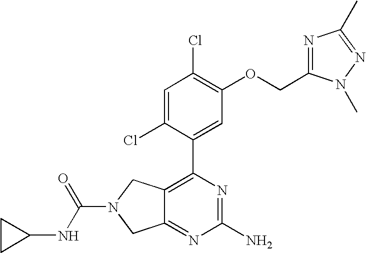 | 2-amino-N-cyclopropyl-4-{2,4- dichloro-5-[(1,3-dimethyl-1H-1,2,4- triazol-5-yl)methoxy]phenyl}-5,7- dihydro-6H-pyrrolo[3,4-d]pyrimidine-6- carboxamide | Ex 127 | 1H-NMR (CDCl3, 300 MHz): d 7.49 (s, 1H), 7.16 (s, 1H), 5.23 (s, 2H), 5.20 (s, 2H), 4.57 (s, 1H), 4.56 (s, 2H), 4.38 (s, 2H), 3.93 (s, 3H), 2.69 (bs, 1H), 2.33 (s, 3H), 0.74 (q, 2H, J = 6 Hz), 0.51 (m, 2H). | 489.0, 491.0 |
| 256 | 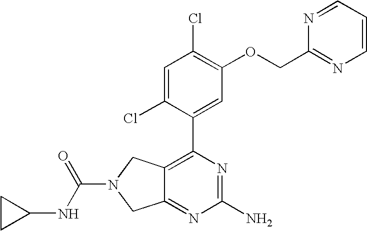 | 2-amino-N-cyclopropyl-4-[2,4-dichloro- 5-(pyrimidin-2-ylmethoxy)phenyl]-5,7- dihydro-6H-pyrrolo[3,4-d]pyrimidine-6- carboxamide | Ex. 127 | 1H-NMR (CDCl3, 300 MHz): d 8.79 (d, 2H, J = 6 Hz), 7.51 (s, 1H), 7.27 (t, 1H), 6.97 (s, 1H, J = 6 Hz), 5.37 (s, 2H), 5.17 (s, 2H), 4.52 (s, 2H), 5.41 (s, 1H)., 4.32 (s, 2H), 2.68 (bs, 1H), 0.73 (m, 2H), 0.50 (s, 2H). | 473.0, 475.0 |
| 257 | 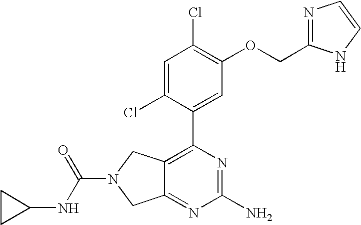 | 2-amino-N-cyclopropyl-4-[2,4-dichloro- 5-(1H-imidazol-2-ylmethoxy)phenyl]- 5,7-dihydro-6H-pyrrolo[3,4- d]pyrimidine-6-carboxamide | Ex. 127 | 1H-NMR (DMSO-d6, 300 MHz): d 7.59 (s, 1H), 7.46 (s, 1H), 6.85 (bs, 2H), 6.77 (s, 1H), 6.44 (s, 1H), 4.50 (d, 1H, J = 3 Hz), 4.39 (m, 4H), 4.14 (s, 1H), 2.06 (2H), 0.50 (m, 2H), 0.39 (m, 2H). | 460.0, 462.0 |
| 258 | 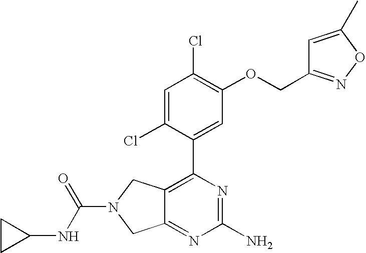 | 2-amino-N-cyclopropyl-4-{2,4- dichloro-5-[(5-methylisoxazol-3- yl)methoxy]phenyl}-5,7-dihydro-6H- pyrrolo[3,4-d]pyrimidine-6- carboxamide | Ex. 127 | 1H-NMR (CDCl3, 300 MHz): d 7.50 (s, 1H), 7.01 (s, 1H), 6.16 (s, 1H), 5.21 (s, 2H), 5.16 (s, 2H), 4.66 (s, 1H), 4.59 (s, 2H), 4.42 (s, 2H), 2.70 (bs, 1H), 2.44 (s, 3H), 0.76 (m, 2H), 0.54 (m, 2H). | 477.0, 479.0 |
| 259 | 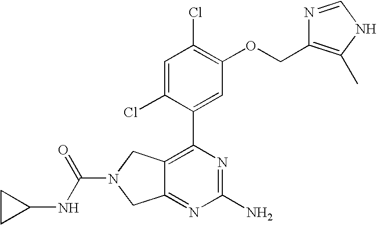 | 2-amino-N-cyclopropyl-4-{2,4- dichloro-5-[(5-methyl-1H-imidazol-4- yl)methoxy]phenyl}-5,7-dihydro-6H- pyrrolo[3,4-d]pyrimidine-6- carboxamide | Ex. 127 | 1H-NMR (DMSO-d6, 300 MHz): d 7.42 (s, 1H), 7.36 (s, 1H), 6.77 (s, 1H), 6.42 (s, 1H), 4.68 (s, 2H), 4.30 (d, 2H, J = 3 Hz), 4.21 (s, 2H), 2.10 (bs, 2H) 2.06 (s, 3H) 0.51 (m, 2H), 0.39 (m, 2H). | 476.0, 478.0 |
| 260 | 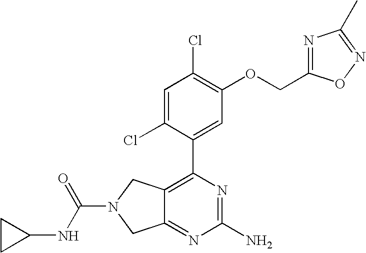 | 2-amino-N-cyclopropyl-4-{2,4- dichloro-5-[(3-methyl-1,2,4-oxadiazol- 6-yl)methoxy]phenyl}-5,7-dihydro-6H- pyrrolo[3,4-d]pyrimidine-6- carboxamide | Ex. 127 | 1H-NMR (CDCl3, 300 MHz): d 7.56 (s, 1H), 7.01 (s, 1H), 5.34 (s, 2H), 5.16 (s, 1H), 4.59 (s, 2H), 4.42 (s, 2H), 2.65 (bs, 1H), 2.44 (s, 3H), 0.74 (m, 2H), 0.52 (m, 2H). | 475.0, 477.0 |
| 261 | 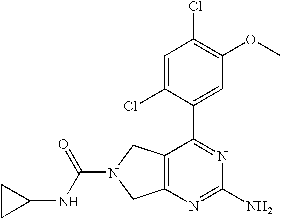 | 2-amino-N-cyclopropyl-4-(2,4-dichloro- 5-methoxyphenyl)-5,7-dihydro-6H- pyrrolo[3,4-d]pyrimidine-6- carboxamide | Ex. 14 | 1H NMR (400 MHz, DMSO-d6) d ppm: 7.74 (s, 1 H), 7.20 (s, 1 H), 6.89 (s, 2 H), 6.47 (d, J = 2.78 Hz, 1 H), 4.41 (s, 2 H), 4.24 (s, 2 H), 3.87 (s, 3 H), 2.52-2.55 (m, 1 H), 0.50-0.57 (m, 1 H), 0.35-0.42 (m, 2 H). | 394.1, 396.1 |
| 262 | 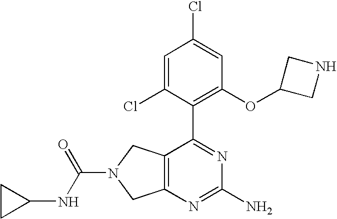 | 2-amino-4-[2-(azetidin-3-yloxy)-4,6- dichlorophenyl]-N-cyclopropyl-5,7- dihydro-6H-pyrrolo[3,4-d]pyrimidine-6- carboxamide | Ex. 129 | 1H NMR (400 MHz, DMSO-d6) d ppm: 7.39 (d, J = 1.77 Hz, 1 H), 7.01 (d, J = 1.77 Hz, 1 H), 6.84 (s, 2 H), 6.45 (d, J = 2.53 Hz, 1 H), 5.02-5.13 (m, 1 H), 4.42 (s, 2 H), 4.05-4.19 (m, 2 H), 3.89-4.04 (m, 2 H), 3.45-3.55 (m, 2 H), 3.17-3.45 (m, 2 H), 0.47-0.58 (m, 2 H), 0.33-0.44 (m, 2 H). | 435.2, 437.2 |
| 263 | 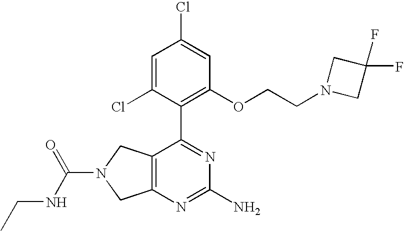 | 2-amino-4-{2,4-dichloro-6-[2-(3,3- difluoroazetidin-1-yl)ethoxy]phenyl}-N- ethyl-5,7-dihydro-6H-pyrrolo[3,4- d]pyrimidine-6-carboxamide | Ex. 15 | 1H NMR (400 MHz, DMSO-d6) d ppm: 7.34 (d, J = 1.77 Hz, 1 H), 7.25 (d, J = 1.77 Hz, 1 H), 6.85 (s, 2 H), 6.35 (t, J = 5.31 Hz, 1 H), 4.30-4.47 (m, 2 H), 4.13 (s, 2 H), 4.00-4.08 (m, 2 H), 3.21-3.29 (m, 2 H), 3.21-3.29 (m, 2 H), 3.01-3.10 (m, 2 H), 2.70-2.77 (m, 2 H), 1.01 (t, J = 7.20 Hz, 3 H). | 487.0, 489.0 |
| 264 | 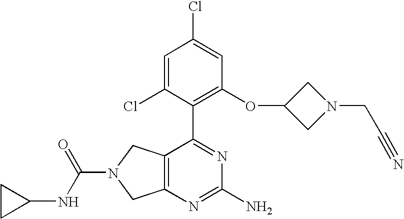 | 2-amino-N-cyclopropyl-4-(2,4-dichloro- 6-{[1-(cyanomethyl)azetidin-3- yl]oxy}phenyl)-5,7-dihydro-6H- pyrrolo[3,4-d]pyrimidine-6- carboxamide | Ex. 130 | 1H NMR (400 MHz, CHLOROFORM-d) d ppm: 7.16 (d, J = 1.77 Hz, 1 H), 6.59 (d, J = 1.77 Hz, 1 H), 5.24 (s, 2 H), 2.75-4.84 (m, 1 H), 4.53-4.66 (m, 3 H), 4.33-4.43 (m, 2 H), 3.76-3.90 (m, 2 H), 3.51 (s, 2 H), 3.27-3.38 (m, 2 H), 2.64-2.75 (m, 1 H), 0.67-0.80 (m, 2 H), 0.46-0.56 (m, 2 H). | 474.0, 476.0 |
| 265 | 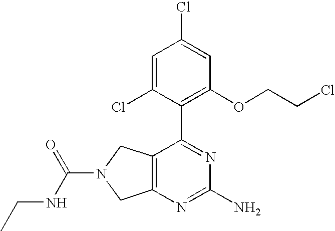 | 2-amino-4-[2,4-dichloro-6-(2- chloroethoxy)phenyl]-N-ethyl-5,7- dihydro-6H-pyrrolo[3,4-d]pyrimidine-6- carboxamide | Ex. 14 | 1H NMR (400 MHz, CHLOROFORM-d) d ppm: 7.17 (d, J = 1.77 Hz, 1 H), 6.89 (d, J = 1.77 Hz, 1 H), 5.18 (s, 2 H), 4.61 (s, 2 H), 4.31-4.43 (m, 2 H), 4.31-4.43 (m, 2 H), 4.16-4.26 (m, 3 H), 3.62-3.69 (m, 3 H), 3.62-3.69 (m, 2 H), 3.27-3.37 (m, 2 H), 1.17 (t, J = 7.20 Hz, 3 H). | 430.0, 432.0 |
| 266 | 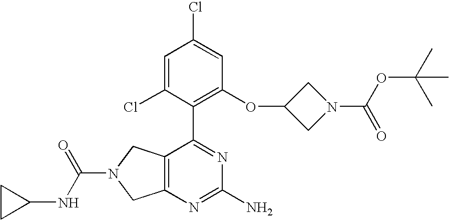 | tert-butyl 3-(2-{2-amino-6- [(cyclopropylamino)carbonyl]-6,7- dihydro-5 H-pyrrolo[3,4-d]pyrimidin-4- yl}-3,5-dichlorophenoxy)azetidine-1- carboxylate | Ex. 128 | 1H NMR (400 MHz, CHLOROFORM-d) d ppm: 7.15 (d, J = 1.26 Hz, 1 H), 6.52 (s, 1 H), 5.24-5.38 (m, 2 H), 4.78-4.90 (m, 1 H), 4.65 (br. s., 1 H), 4.56 (br.s., 2 H), 4.32-4.43 (m, 1 H), 4.19-4.32 (m, 3 H), 3.76-3.93 (m, 2 H), 2.60-2.75 (m, 1 H), 1.42 (s, 9 H), 0.74 (t, J = 5.94 Hz, 2 H), 0.46-0.59 (m, 2 H). | 534.1, 536.1 |
| 267 | 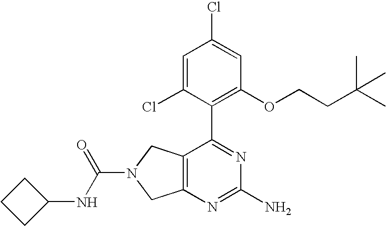 | 2-amino-N-cyclobutyl-4-[2,4-dichloro- 6-(3,3-dimethylbutoxy)phenyl]-5,7- dihydro-6H-pyrrolo[3,4-d]pyrimidine-6- carboxamide | Ex. 127 | 1H NMR (400 MHz, DMSO-d6) d ppm: 7.32 (s, 2 H), 6.80 (br. s., 2 H), 6.52 (d, J = 7.83 Hz, 1 H), 4.39 (d, J = 4.04 Hz, 2 H), 4.09-4.17 (m, 3 H), 4.00-4.08 (m, 2 H), 2.03-2.15 (m, 2 H), 1.84-1.99 (m, 2 H), 1.49-1.62 (m, 2 H), 1.49-1.62 (m, 2 H), 1.45 (t, J = 6.69 Hz, 2 H), 0.80 (s, 9 H). | 478.0, 480.0 |
| 268 | 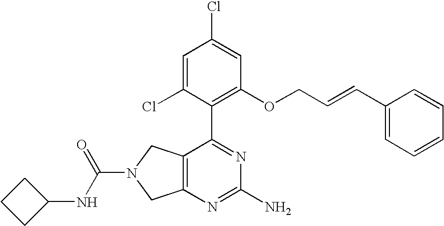 | 2-amino-N-cyclobutyl-4-(2,4-dichloro- 6-{[(2E)-3-phenylprop-2-en-1- yl]oxy}phenyl)-5,7-dihydro-6H- pyrrolo[3,4-d]pyrimidine-6- carboxamide | Ex. 127 | 1H NMR (400 MHz, DMSO-d6) d ppm: 7.22-7.42 (m, 7 H), 6.87 (s, 2 H), 6.46-6.57 (m, 2 H), 6.28-6.40 (m, 1 H), 4.81 (d, J = 4.80 Hz, 2 H), 4.42 (s, 2 H), 4.17 (s, 2 H), 4.06-4.15 (m, 1 H), 2.02-2.14 (m, 2 H), 1.82-1.98 (m, 2 H), 1.46-1.60 (m, 2 H). | 510.0, 512.0 |
| 269 | 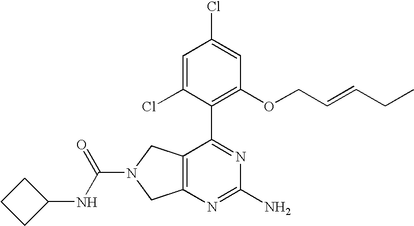 | 2-amino-N-cyclobutyl-4-(2,4-dichloro- 6-[(2E)-pent-2-en-1-yloxy]phenyl}-5,7- dihydro-6H-pyrrolo[3,4-d]pyrimidine-6- carboxamide | Ex. 127 | 1H NMR (400 MHz, DMSO-d6) d ppm: 7.33 (d, J = 1.77 Hz, 1 H), 7.28 (d, J = 1.52 Hz, 1 H), 6.83 (br. s., 2 H), 6.48-6.55 (m, 1 H), 5.64-5.76 (m, 1 H), 5.64-5.76 (m, 1 H), 5.42-5.53 (m, 1 H), 4.57 (d, J = 5.56 Hz, 2 H), 4.40 (s, 2 H), 4.07-4.17 (m, 3 H), 2.04-2.13 (m, 2 H), 1.85-2.02 (m, 4 H), 1.46-1.59 (m, 2 H), 0.88 (t, J = 7.45 Hz, 3 H). | 462.0, 464.0 |
| 270 | 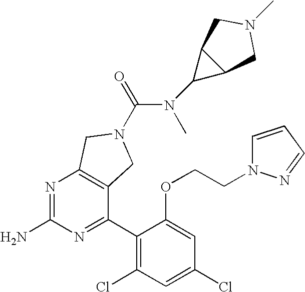 | 2-amino-4-{2,4-dichloro-6-[2-(1H- pyrazol-1-yl)ethoxy]phenyl}-N-methyl- N-[(1R,5S)-3-methyl-3- azabicyclo[3.1.0]hex-6-yl]-5,7-dihydro- 6H-pyrrolo[3,4-d]pyrimidine-6- carboxamide | Ex. 102 | 1H NMR (400 MHz, DMSO-d6) d ppm 1.66 (s, 1 H) 1.85 (s, 1 H) 2.19 (s, 3 H) 2.26-2.35 (m, 2 H) 2.85-2.91 (m, 1 H) 2.97-3.12 (m, 2 H) 3.56-3.65 (m, 2 H) 4.04-4.11 (m, 2 H) 4.28-4.42 (m, 4 H) 5.86-5.91 (m, 1 H) 7.17 (d, J = 2.02 Hz, 1 H) 7.19 (d, J = 1.52 Hz, 1 H) 7.29 (s, 1 H) 7.35 (s, 1 H) | 543.2, 544.2 |
| 271 | 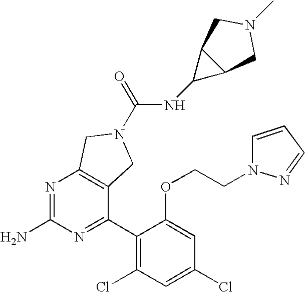 | 2-amino-4-{2,4-dichloro-6-[2-(1H- pyrazol-1-yl)ethoxy]phenyl}-N- [(1R,5S)-3-methyl-3- azabicyclo[3.1.0]hex-6-yl]-5,7-dihydro- 6H-pyrrolo[3,4-d]pyrimidine-6- carboxamide | Ex. 102 | 1H NMR (400 MHz, DMSO-d6) d ppm 1.81-1.97 (m, 2 H) 2.54 (s, 3 H) 2.60-2.72 (m, J = 1.52 Hz, 2 H) 3.03 (s, 2 H) 3.11-3.20 (m, 1 H) 3.60-3.70 (m, J = 19.45 Hz, 1 H) 3.85 (d, J = 12.63 Hz, 2 H) 4.29-4.33 (m, 4 H) 4.37 (d, J = 7.58 Hz, 2 H) 5.94-6.02 (m, 1 H) 7.17 (d, J = 1.77 Hz, 1 H) 7.23 (d, J = 1.52 Hz, 1 H) 7.29 (d, J = 1.52 Hz, 1 H) 7.30 (d, J = 1.52 Hz, 1 H) | 464.0, 466.0 |
| 272 | 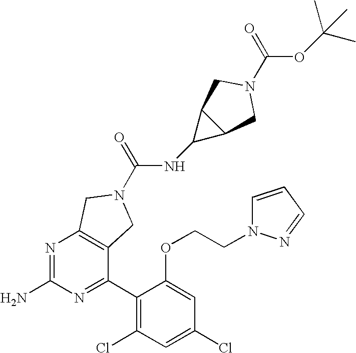 | tert-butyl (1R,5S)-6-{[(2-amino-4-{2,4- dichloro-6-[2-(1H-pyrazol-1- yl)ethoxy]phenyl}-5,7-dihydro-6H- pyrrolo[3,4-d]pyrimidin-6- yl)carbonyl]amino}-3- azabicyclo[3.1.0]hexane-3-carboxylate | Ex. 102 | 1H NMR (400 MHz, DMSO-d6) d ppm 1.20-1.26 (m, 2 H) 1.54-1.67 (m, J = 23.49 Hz, 2 H) 2.13-2.23 (m, J = 2.02 Hz, 1 H) 2.82-2.94 (m, 2 H) 3.30-3.36 (m, 1 H) 3.45 (dd, J = 10.74, 4.17 Hz, 2 H) 3.52-3.59 (m, 1 H) 3.84-3.96 (m, 1 H) 4.32 (s, 2 H) 4.38 (d, J = 6.82 Hz, 1 H) 6.00 (t, J = 1.89 Hz, 1 H) 6.42 (s, 1 H) 6.79 (s, 1 H) 7.22 (dd, J = 26.78, 1.77 Hz, 1 H) 7.31 (dd, J = 8.34, 1.52 Hz, 1 H) | 615.2, 617.2 |
| 273 | 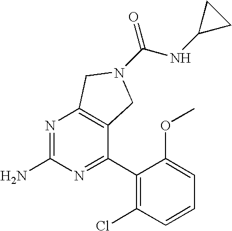 | 2-amino-4-(2-chloro-6-methoxyphenyl)- N-cyclopropyl-5,7-dihydro-6H- pyrrolo[3,4-d]pyrimidine-6- carboxamide | Ex. 18 | 1H NMR (400 MHz, DMSO-d6) d ppm 0.33-0.43 (m, 2 H) 0.47-0.60 (m, 2 H) 2.49-2.58 (m, 1 H) 3.73 (s, 3 H) 4.08 (s, 2 H) 4.40 (s, 2 H) 6.46 (d, J = 2.78 Hz, 1 H) 6.78 (s, 2 H) 7.14 (dd, J = 8.08, 5.56 Hz, 2 H) 7.44 (t, J = 8.21 Hz, 1 H) | 360.2, 362.2 |
| 274 | 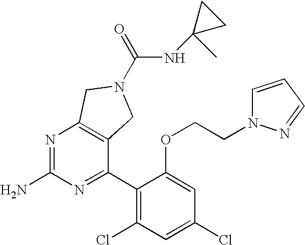 | 2-amino-4-{2,4-dichloro-6-[2-(1H- pyrazol-1-yl)ethoxy]phenyl}-N-(1- methylcyclopropyl)-5,7-dihydro-6H- pyrrolo[3,4-d]pyrimidine-6- carboxamide | Ex. 102 | 1H NMR (400 MHz, DMSO-d6) d ppm 0.44-0.48 (m, 2 H) 0.55-0.68 (m, 2 H) 1.29 (s, 3 H) 3.50 (d, J = 13.39 Hz, 1 H) 3.86 (dd, J = 13.01, 1.89 Hz, 1 H) 4.26-4.34 (m, 4 H) 4.34-4.44 (m, 2 H) 5.99 (t, J = 2.02 Hz, 1 H) 6.46-6.55 (m, 1 H) 6.77 (s, 2 H) 7.18 (d, J = 2.02 Hz, 1 H) 7.25 (d, J = 1.77 Hz, 1 H) 7.28 (d, J = 1.26 Hz, 1 H) 7.32 (d, J = 1.77 Hz, 1 H) | 488.2, 490.2 |
| 275 | 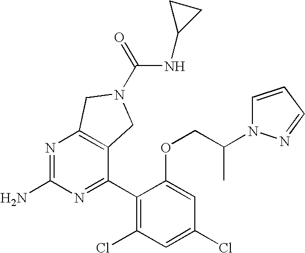 | 2-amino-N-cyclopropyl-4-{2,4- dichloro-6-[2-(1H-pyrazol-1- yl)propoxy]phenyl}-5,7-dihydro-6H- pyrrolo[3,4-d]pyrimidine-6- carboxamide | Ex. 18 | 1H NMR (400 MHz, DMSO-d6) d ppm 0.35-0.45 (m, 2 H) 0.49-0.64 (m, 2 H) 0.49-0.64 (m, 2 H) 1.34 (d, J = 2.02 Hz, 3 H) 2.52-2.58 (m, 1 H) 3.35-3.40 (m, 1 H) 3.75-3.88 (m, 1 H) 4.14-4.44 (m, 4 H) 4.51-4.65 (m, 1 H) 6.01 (t, J = 2.02 Hz, 1 H) 6.27-6.44 (m, 1 H) 6.74-6.79 (m, 2 H) 7.21-7.27 (m, 2 H) 7.29-7.36 (m, 2 H) | 488.1, 490.1 |
| 276 | 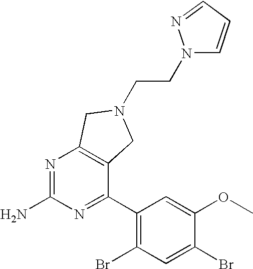 | 4-(2,4-dibromo-5-methoxyphenyl)-6-[2- (1H-pyrazol-1-yl)ethyl]-6,7-dihydro- 5 H-pyrrolo[3,4-d]pyrimidin-2-amine | Ex. 105 | 1H NMR (400 MHz, DMSO-d6) d ppm 3.80-3.91 (m, J = 3.79 Hz, 3 H) 4.25 (s, 1 H) 4.27-4.41 (m, 6 H) 4.45 (s, 1 H) 6.24 (d, J = 2.02 Hz, 1 H) 6.94 (s, 2 H) 7.13 (d, J = 2.78 Hz, 1 H) 7.45 (s, 1 H) 7.79 (d, J = 1.77 Hz, 1 H) 7.86 (d, J = 8.84 Hz, 1 H) | 494.0, 496.1 |
| 277 | 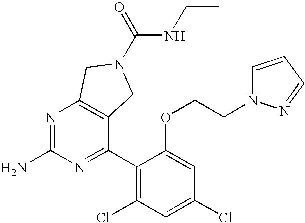 | 2-amino-4-{2,4-dichloro-6-[2-(1H- pyrazol-1-yl)ethoxy]phenyl}-N-ethyl- 5,7-dihydro-6H-pyrrolo[3,4- d]pyrimidine-6-carboxamide | Ex. 18 | 1H NMR (400 MHz, DMSO-d6) d ppm 1.02 (t, J = 7.20 Hz, 3 H) 2.98-3.14 (m, 2 H) 3.59 (d, J = 12.88 Hz, 1 H) 3.87-3.97 (m, 1 H) 4.33 (s, 4 H) 4.36-4.47 (m, 2 H) 6.00 (t, J = 2.02 Hz, 1 H) 6.23 (t, J = 4.80 Hz, 1 H) 6.78 (s, 2 H) 7.19 (d, J = 1.77 Hz, 1 H) 7.25 (d, J = 1.77 Hz, 1 H) 7.29 (d, J = 1.26 Hz, 1 H) 7.32 (d, J = 1.77 Hz, 1 H) | 462.1, 464.1 |
| 278 | 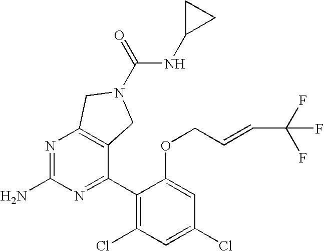 | 2-amino-N-cyclopropyl-4-(2,4-dichloro- 6-{[(2E)-4,4,4-trifluorobut-2-en-1- yl]oxy}phenyl)-5,7-dihydro-6H- pyrrolo[3,4-d]pyrimidine-6- carboxamide | Ex. 18 | 1H NMR (400 MHz, DMSO-d6) d ppm 0.33-0.43 (m, 2 H) 0.47-0.59 (m, 2 H) 4.02-4.18 (m, 2 H) 4.39 (d, J = 7.33 Hz, 2 H) 4.74-4.91 (m, 2 H) 5.61-5.74 (m, 1 H) 6.43 (d, J = 2.53 Hz, 1 H) 6.43 (d, J = 2.53 Hz, 1 H) 6.57-6.67 (m, 1 H) 7.32 (d, J = 1.77 Hz, 1 H) 7.40 (d, J = 1.77 Hz, 1 H) | 488.0, 490.1 |
| 279 | 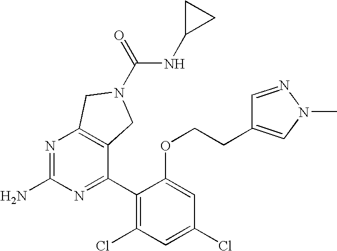 | 2-amino-N-cyclopropyl-4-{2,4- dichloro-6-[2-(1-methyl-1H-pyrazol-4- yl)ethoxy]phenyl}-5,7-dihydro-6H- pyrrolo[3,4-d]pyrimidine-6- carboxamide | Ex. 18 | 1H NMR (400 MHz, DMSO-d6) d ppm 0.34-0.44 (m, 2 H) 0.53 (dd, J = 6.82, 2.27 Hz, 2 H) 2.53-2.56 (m, 1 H) 2.61-2.70 (m, 2 H) 3.70 (s, 3 H) 3.85 (d, J = 13.14 Hz, 1 H) 3.96-4.05 (m, 1 H) 4.06-4.16 (m, 2 H) 4.35-4.50 (m, 2 H) 6.41 (d, J = 2.02 Hz, 1 H) 6.85 (s, 2 H) 7.00 (s, 1 H) 7.17 (s, 1 H) 7.27 (d, J = 1.77 Hz, 1 H) 7.31 (d, J = 1.77 Hz, 1 H) | 488.1, 490.1 |
| 280 | 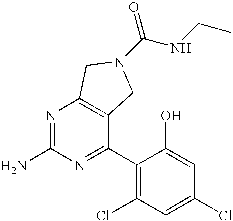 | 2-amino-4-(2,4-dichloro-6- hydroxyphenyl)-N-ethyl-5,7-dihydro- 6H-pyrrolo[3,4-d]pyrimidine-6- carboxamide | Ex. 18 | 1H NMR (400 MHz, DMSO-d6) d ppm 1.00 (t, J = 7.20 Hz, 3 H) 2.97-3.13 (m, 2 H) 4.14 (s, 2 H) 4.38-4.48 (m, 2 H) 6.35 (t, J = 5.43 Hz, 1 H) 6.76-6.83 (m, 2 H) 6.95 (d, J = 1.77 Hz, 1 H) 7.14 (d, J = 1.77 Hz, 1 H) 10.68 (s, 1 H) | 368.1, 370.0 |
| 281 | 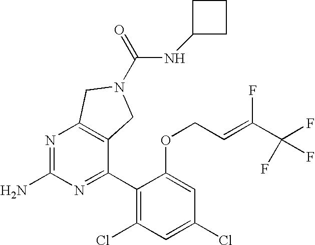 | 2-amino-N-cyclobutyl-4-(2,4-dichloro- 6-{[(2Z)-3,4,4,4-tetrafluorobut-2-en-1- yl]oxy}phenyl)-5,7-dihydro-6H- pyrrolo[3,4-d]pyrimidine-6- carboxamide | Ex. 18 | 1H NMR (400 MHz, DMSO-d6) d ppm 1.46-1.65 (m, 2 H) 1.85-2.00 (m, 2 H) 2.02-2.14 (m, 2 H) 4.01-4.24 (m, 3 H) 4.35-4.44 (m, 2 H) 5.63-5.84 (m, 1 H) 6.49 (d, J = 7.83 Hz, 1 H) 6.86 (d, J = 2.53 Hz, 2 H) 7.42-7.49 (m, 1 H) 7.53-7.69 (m, 1 H) | 520.0, 522.0 |
| 282 | 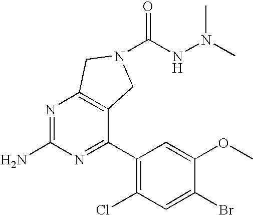 | 2-amino-4-(4-bromo-2-chloro-5- methoxyphenyl)-N′,N′-dimethyl-5,7- dihydro-6H-pyrrolo[3,4-d]pyrimidine-6- carbohydrazide | Ex. 102 | 1H NMR (400 MHz, DMSO-d6) d ppm 2.46-2.57 (m, 6 H) 3.86 (s, 3 H) 4.20-4.34 (m, 2 H) 4.37-4.57 (m, 1 H) 6.91 (s, 2 H) 7.16 (s, 1 H) 7.56 (s, 1 H) 7.87 (s, 1 H) | 441.9, 442.9 |
| 283 | 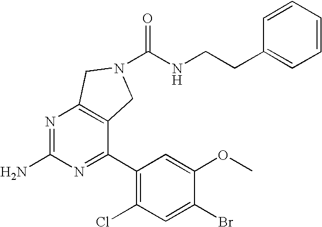 | 2-amino-4-(4-bromo-2-chloro-5- methoxyphenyl)-N-(2-phenylethyl)-5,7- dihydro-6H-pyrrolo[3,4-d]pyrimidine-6- carboxamide | Ex. 102 | 1H NMR (400 MHz, DMSO-d6) d ppm 2.65-2.82 (m, 2 H) 3.16-3.30 (m, 2 H) 3.87 (s, 3 H) 4.27 (s, 2 H) 4.43 (s, 2 H) 6.53 (t, J = 5.43 Hz, 1 H) 6.92 (s, 2 H) 7.10-7.22 (m, 4 H) 7.24-7.37 (m, 2 H) 7.88 (s, 2 H) | 502.9, 503.9 |
| 284 | 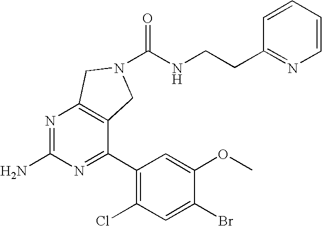 | 2-amino-4-(4-bromo-2-chloro-5- methoxyphenyl)-N-(2-pyridin-2- ylethyl)-5,7-dihydro-6H-pyrrolo[3,4- d]pyrimidine-6-carboxamide | Ex. 102 | 1H NMR (400 MHz, DMSO-d6) d ppm 2.81-2.93 (m, 2 H) 3.35-3.45 (m, 2 H) 4.19-4.29 (m, 2 H) 4.36-4.50 (m, 2 H) 6.54 (t, J = 5.68 Hz, 1 H) 6.19 (s, 2 H) 7.12-7.27 (m, 3 H) 7.61-7.74 (m, 1 H) 7.87 (s, 1 H) 8.47 (d, J = 1.01 Hz, 1 H) | 503.8, 504.9 |
| 285 | 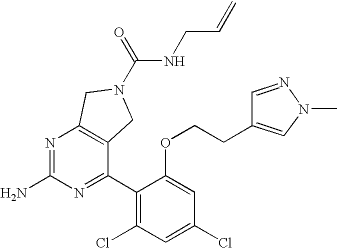 | N-allyl-2-amino-4-{2,4-dichloro-6-[2- (1-methyl-1H-pyrazol-4- yl)ethoxy]phenyl}-5,7-dihydro-6H- pyrrolo[3,4-d]pyrimidine-6- carboxamide | Ex. 18 | 1H NMR (400 MHz, MeOD) d ppm 2.77 (t, J = 5.43 Hz, 2 H) 3.77 (s, 3 H) 3.79 (d, J = 5.31 Hz, 2 H) 3.96-4.12 (m, 2 H) 4.14-4.25 (m, 2 H) 4.49-4.69 (m, 2 H) 4.49-4.69 (m, 2 H) 5.06 (dd, J = 10.11, 1.26 Hz, 1 H) 5.16 (dd, J = 17.18, 1.52 Hz, 1 H) 5.81-5.96 (m, 1 H) 7.04 (s, 1 H) 7.12-7.17 (m, 2 H) 7.19 (d, J = 1.77 Hz, 1 H) | 488.1, 489.1 |
| 286 | 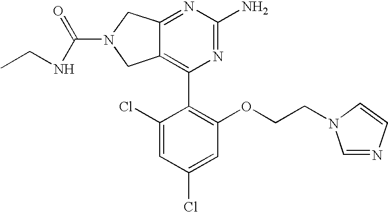 | 2-amino-4-{2,4-dichloro-6-[2-(1H- imidazol-1-yl)ethoxy]phenyl}-N-ethyl- 5,7-dihydro-6H-pyrrolo[3,4- d]pyrimidine-6-carboxamide | Ex. 111 | 1H NMR (300 MHz, DMSO-d6) d ppm 1.04 (t, J = 7.1 Hz, 3H), 3.04-3.13 (m, 2H), 3.75 (d, J = 13.2 Hz, 1H), 4.01 (d, J = 13.2 Hz, 1H), 4.19-4.56 (m, 6H), 6.27 (br s, 1H), 6.71 (s, 1H), 6.75-6.86 (m, 3H), 7.28 (s, 2H), 7.37 (s, 1H). | 462.2, 464.2 |
| 287 | 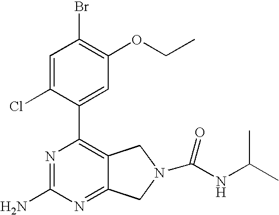 | 2-amino-4-(4-bromo-2-chloro-5- ethoxyphenyl)-N-isopropyl-5,7-dihydro- 6H-pyrrolo[3,4-d]pyrimidine-6- carboxamide | Ex. 111 | 1H NMR (400 MHz, DMSO-d6) d ppm 1.06 (d, J = 6.5 Hz, 6H), 1.35 (t, J = 6.9 Hz, 3H), 3.75 (m, 1H), 4.09-4.17 (m, 2H), 4.25 (s, 2H), 4.42 (s, 2H), 6.07 (d, J = 8.6 Hz, 1H), 6.88 (s, 2H), 7.15 (s, 1H), 7.86 (s, 1H). | 454.2, 456.2 |
| 288 | 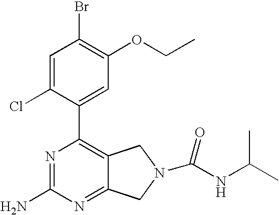 | 2-amino-4-(4-bromo-2-chloro-5- ethoxyphenyl)-N-ethyl-5,7-dihydro-6H- pyrrolo[3,4-d]pyrimidine-6- carboxamide | Ex. 111 | 1H NMR (300 MHz, DMSO-d6) d ppm 0.98-1.07 (m, 3H), 1.30-1.41 (m, 3H), 3.01-3.14 (m, 2H), 4.08-4.20 (m, 2H), 4.26 (s, 2H), 4.43 (s, 2H), 6.39 (br s, 1H), 6.92 (br s, 2H), 7.17 (s, 1H), 7.87 (s, 1H) | 440.2, 442.2 |
| 289 | 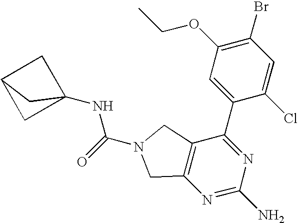 | 2-amino-N-bicyclo[1.1.1]pent-1-yl-4-(4- bromo-2-chloro-5-ethoxyphenyl)-5,7- dihydro-6H-pyrrolo[3,4-d]pyrimidine-6- carboxamide | Ex. 111 | 1H NMR (300 MHz, DMSO-d6) d ppm 1.36 (t, J = 7.2 Hz, 3H), 1.95 (s, 6H), 2.36 (s, 1H), 4.10-4.18 (m, 2H, partially obscured), 4.25 (s, 2H), 4.40 (s, 2H), 6.90 (br s, 2H), 7.02 9br s, 1H), 7.16 (s, 1H), 7.87 (s, 1H) | 478.2, 480.2 |
| 290 | 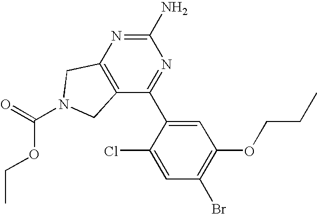 | ethyl 2-amino-4-(4-bromo-2-chloro-5- propoxyphenyl)-5,7-dihydro-6H- pyrrolo[3,4-d]pyrimidine-6-carboxylate | Ex. 108 | 1H NMR (300 MHz, DMSO-d6) d ppm 0.96-1.04 (m, 3H), 1.14-1.29 (m, 3H), 1.69-1.82 (m, 2H), 3.98-4.16 (m, 4H), 4.32-4.39 (m, 2H), 4.46-4.56 (m, 2H), 6.97 (br s, 2H), 7.15 (s, 1H), 7.86 (s, 1H) | 455.0, 457.0 |
| 291 | 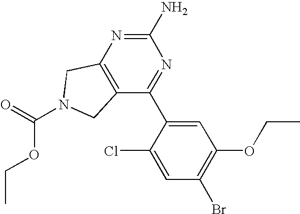 | ethyl 2-amino-4-(4-bromo-2-chloro-5- ethoxyphenyl)-5,7-dihydro-6H- pyrrolo[3,4-d]pyrimidine-6-carboxylate | Ex. 108 | 1H NMR (300 MHz, DMSO-d6) d ppm 1.13-1.26 (m, 3H), 1.31-1.40 (m, 3H), 4.04-4.17 9m, 4H), 4.31-4.38 (m, 2H), 4.47-4.54 (m, 2H), 6.96 (br s, 2H), 7.15 | 441.0, 443.0 |
| 292 | 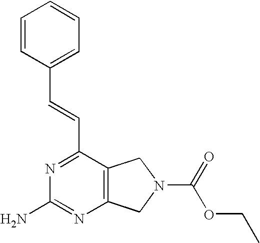 | ethyl 2-amino-4-[(E)-2-phenylvinyl]- 5,7-dihydro-6H-pyrrolo[3,4- d]pyrimidine-6-carboxylate | Ex. 1 | 1H NMR (300 MHz, DMSO-d6) d ppm 1.22-1.30 (m, 3H), 4.09-4.16 (m, 2H), 4.42 (d, J = 7.6 Hz, 2H), 4.67 (d, J = 7.5 Hz, 2H), 6.62 (s, 2H), 7.11 (dd, J = 6.9, 15.8 Hz, 1H), 7.36-7.48 (m, 3H), 7.71-7.79 (m, 3H). | 311.4 |

### HSP-90 Biochemical Assay

Compounds of the present invention were evaluated for potency against HSP-90 using a SPA (scintillation proximity assay) competition binding assay. Briefly, either full length or N-terminal HSP-90 that contains a 6-His tag on its C-terminus binds to copper on Yttrium-silicate scintillant beads via the His-tag. Tritiated propyl-Geldanamycin (pGA), whose structure is shown below, is an analog of a natural inhibitor of HSP-90 called Geldanamycin. Tritiated pGA, which contains a tritiated propyl-amine group added at the #17 position, binds HSP-90 and brings the isotope into proximity with the beads. 17-n-propylamino-Geldanamycin can be prepared as described in U.S. Pat. No. 4,261,989, which is incorporated herein by reference. A second tritiated compound that can also be used in this assay is shown below and is designated as Compound A.

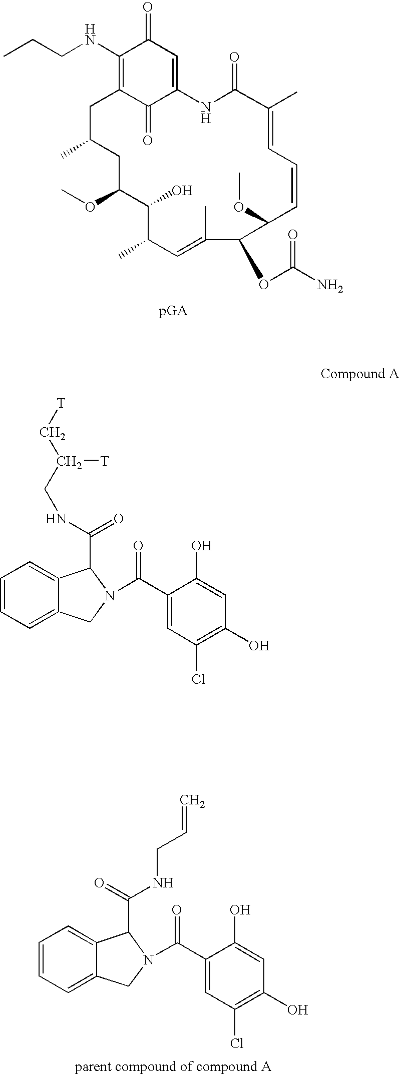

The “T” in the structure of Compound A above indicates the position of the labeled tritiated hydrogen atoms. This compound has a K_{d} of 40 nM and can be prepared as follows. Compound A can be prepare from the parent compound of Compound A, (N-allyl-2-(5-chloro-2,4-dihydroxybenzoyl)isoindoline-1-carboxamide) as described in the following. Allylamine (2.5 mL, 5 mmol, 2M in THF) was added to a solution of Boc(R,S)-1,3-dihydro-2H-isoindole carboxylic acid (263 mg, 1 mmole), diisopropylethyl amine (0.9 mL, 5 mmol), and O-(7-azabenzotriazol-1-yl)-N,N,N′,N′-tetramethyluronium phosphorus pentafloride (HATU) (420 mg, 1.1 mmol) in 5 mL of DMF under a nitrogen atmosphere. The reaction was allowed to stir at room temperature for 12 hours. Saturated NaHCO₃ (30 mL) was added to the reaction mixture to quench the reaction. EtOAc (2×50 mL) was then added to extract the aqueous solution. Dry EtOAc layer over Na₂SO₄. The Na₂SO₄ was filtered off and the filtrate was evaporated to give a brown oil residue. The residue was purified by silica gel chromatography (gradient elution 40→50% EtOAc in hexanes) to give the desired intermediate product (321 mg, quantitative yield) tert-butyl 1-[(allylamino)carbonyl]-1,3-dihydro-2H-isoindole-2-carboxylate.

Hydrogen chloride (3 mL, 12 mmol; 4 M in dioxane) was added to a solution of tert-butyl 1-[(allylamino)carbonyl]-1,3-dihydro-2H-isoindole-2-carboxylate (1 mmol) in DCM (5 mL) at room temperature. The reaction was heated and stirred at room temperature for 12 hours. The reaction mixture was evaporated to give an oil residue. The residue (N-allylisoindoline-1-carboxamide) was used for the next step reaction without further purification.

N-allylisoindoline-1-carboxamide (1 mmol) was then added to a solution of 5-chloro-2,4-bis(methoxymethoxy)benzoic acid (which can be prepared as shown in WO 2006/117669) (340 mg, 1.2 mmol), 4-methylmorpholine (2.2 mL, 20 mmol), N-(3-dimethylaminopropyl)-N′-ethylcarbodiimide hydrochloride (460 mg, 2.4 mmol), and 1-hydroxy benzotriazole (330 mg, 2.4 mmol) in 12 mL of DMF under a nitrogen atmosphere. The reaction was allowed to stir at room temperature for 12 hours. H₂O (50 mL) was added to the reaction mixture to quench the reaction. EtOAc (2×100 mL) was then added to extract the aqueous solution. Dry EtOAc layer over Na₂SO₄. The Na₂SO₄ was filtered off and the filtrate was evaporated to give a brown oil residue. The residue was purified by silica gel chromatography (gradient elution 50→60% EtOAc in hexanes) to give the desired intermediate product (423 mg, 91.8% yield) N-allyl-2-[5-chloro-2,4-bis(methoxymethoxy)benzoyl]isoindoline-1-carboxamide.

Hydrogen chloride (4 mL, 16 mmol; 4 M in dioxane) was added to a solution of N-allyl-2-[5-chloro-2,4-bis(methoxymethoxy)benzoyl]isoindoline-1-carboxamide (392 mg, 0.85 mmol) in DCM (5 mL). The reaction was stirred at room temperature for 12 hours. The reaction mixture was neutralized with saturated NaHCO₃ (aq) and then extracted with EtOAc (2×50 mL). The combined organic layers were dried, filtered, and evaporated to give the desired final product as the parent compound (N-allyl-2-(5-chloro-2,4-dihydroxybenzoyl)isoindoline-1-carboxamide) as a white solid (221 mg, 69.7% yield). ¹H NMR (400 MHz, DMSO-D6) δ ppm 3.57 (d, J=79.33 Hz, 2H) 4.65-4.93 (m, 1H) 4.97-5.19 (m, 1H) 5.42-5.70 (m, 1H) 5.68-5.95 (m, 1H) 6.40-6.71 (m, 1H) 6.92 (s, 1H) 7.15-7.67 (m, 4H) 8.28 (s, 1H) 10.06 (s, 1H) 10.40 (s, 1H). Anal. Calcd for C₁₉H₁₇ClN₂O₄: C, 61.21; H, 4.60; N, 7.51. Found: C, 61.02; H, 4.63; N, 7.36.

Once the parent compound was made, Compound A was prepared using standard hydrogenation methods using tritium gas.

The beta signal emitted from the isotope excites the scintillant, which creates a measurable signal. As competitive compounds are added to the assay mixture, they compete with bound tritiated pGA or Compound A at the ATP-binding site on the N-terminal of HSP-90. When a compound displaces the labeled pGA or Compound A, the signal is reduced (the beta-particles are no longer in proximity with the bead). This reduction in signal is used to quantify the extent to which the inhibitor/compound is competitive with pGA or Compound A.

The SPA assay for ³H-pGA (designated G1) and Compound A (designated G2) binding to HSP-90 was performed in 96-well flat bottom white plates (Corning #3604). For G1, typical reaction solutions contained 30 nM HSP-90 and 200 nM ³H-pGA in binding buffer (100 mM Hepes, pH 7.5 and 150 mM KCl). For G2, typical reaction solutions contained 5 nM HSP-90 and 50 nM of Compound A. For G1, the ³H-pGA was first diluted to 33% label with unlabeled pGA that was synthesized and purified to give a final concentration of 200 nM. For G2, labeled Compound A was diluted with unlabeled Compound A to provide a ratio of labeled:unlabeled of 1:2 for a final concentration of 50 nM. Inhibitors were added to the HSP-90/³H-pGA (or HSP-90/Compound A) solutions at eleven different concentrations for Kᵢ determinations. The range of inhibitor concentrations were 100 μM, or an appropriate range, for solid samples and 10 μM for targeted library compounds and 4 mM liquid stocks. To determine percent inhibition, the compound was tested at 1 and 10 μM. The final DMSO in the samples was 4%. Copper-Ysi beads (Amersham, #RPNQ0096) that have been diluted in binding buffer were added to each well to give a final concentration of 100 μg/well. The plates were sealed, covered with a foil-covered lid and shaken for 30 minutes at room temperature. The beads were allowed to settle for 30 minutes after which the plates were counted using a Packard TopCount NXT instrument. This procedure has also been adapted for medium throughput using a Beckman Biomek FX. Samples were run in duplicate and on two separate days to assure an accurate value of Kᵢ.

For Kᵢ determinations, the corrected cpm's (actual cpm's minus background) were plotted vs. inhibitor concentration using GraphPad. Prism software. The data were fit to a generic IC₅₀ equation, Y=YI/(1+[X]/IC₅₀), where YI=Y-intercept and [X] is the competing ligand/inhibitor. The IC₅₀ was then used to calculate the Ki by using the Cheng-Prusoff equation:

$$Ki \{ cl \} = \frac{{IC}_{50} \{ cl \}}{1 + ( [ hl ] / Kd \{ hl \} )}$$

Where cl=cold ligand concentration (varies), [hl]=concentration of hot ligand (200 nM or 50 nM) and Kd{hl}=240 nM (for ³H-pGA) or 40 nM (for Compound A). Error was calculated as follows: IC₅₀ error/IC₅₀ value=fractional error and fractional error*Kᵢ value=Kᵢ error.

In the cases in which inhibitor binds to HSP-90 so tightly that the population of free inhibitor molecules is significantly depleted by formation of the enzyme-inhibitor complex, the above equation is no longer valid. This is normally true when the observed IC₅₀ is about the same as the HSP-90 concentration. For a tight binding inhibitor, the following equation can be applied:

$$\frac{EL}{{EL}_{o}} = \frac{- ( {K}_{I}^{app} + {I}_{o} - {E}_{o} ) + \sqrt{{( {K}_{I}^{app} + {I}_{o} - {E}_{o} )}^{2} + 4 \times {E}_{o} \times {K}_{I}^{app}}}{2 \times {E}_{o}}$$

$$Where {K}_{I}^{app} = {K}_{I} \times ( 1 + \frac{{L}_{o}}{{K}_{L}} )$$

EL and ELₒ are the radioligand-HSP-90 complexes in the presence and absence of inhibitor, respectively. EL/ELₒ represents the fractional signal in the presence of inhibitor. Io, Eₒ, and Lₒ are the inhibitor, HSP-90, and radioligand concentrations, respectively. K_{I} is the inhibition constant for the ligand, while K_{L} is the binding affinity constant between the enzyme (HSP-90) and the ligand.

The Ki assay data of the compound of Examples 1-292 are listed in the following Table 3.

TABLE 3

| Example No. | (G1): Ki (μM) | (G2): Ki (μM) |
|---|---|---|
| 1 | 0.54 | 0.112 |
| 2 | 2.77 | |
| 3 | 0.052 | |
| 4 | 1.7 | 0.475 |
| 5 | 0.244 | |
| 6 | 4.6 | |
| 7 | 0.952 | 0.12 |
| 8 | 0.369 | |
| 9 | 4 | |
| 10 | | 19.2 |
| 11 | | 2.4 |
| 12 | | 16 |
| 13 | 0.388 | |
| 14 | 1.74 | |
| 15 | 0.57 | |
| 17 | | 17.5 |
| 18 | 0.008 | |
| 20 | | 27 |
| 22 | | 21.5 |
| 23 | | 0% inhibition |
| | | at 10 μM |
| 24 | | 38 |
| 25 | | 0% inhibition |
| | | at 10 μM |
| 26 | 44 | |
| 27 | | 0% inhibition |
| | | at 10 μM |
| 29 | | 0.304 |
| 30 | | 0.56 |
| 31 | | 0.36 |
| 32 | 1.9 | 0.499 |
| 33 | 1.3 | 0.268 |
| 34 | 2.6 | 0.74 |
| 35 | 1.5 | 0.437 |
| 36 | 7.3 | 2.86 |
| 37 | 4 | 1.54 |
| 38 | 2.5 | 0.822 |
| 39 | 1.66 | 0.300 |
| 40 | 4 | 0.831 |
| 41 | 2.5 | 0.56 |
| 42 | 0.56 | 0.082 |
| 43 | 0.47 | 0.105 |
| 44 | 0.72 | 0.17 |
| 45 | 0.78 | 0.167 |
| 46 | | 0% inhibition |
| | | at 10 μM |
| 47 | | 32 |
| 48 | | 20 |
| 49 | | 3.4 |
| 50 | | 11.4 |
| 51 | 2.71 | |
| 52 | 0.626 | |
| 53 | 0.289 | |
| 54 | 0.4 | |
| 55 | 0.143 | |
| 56 | 0.104 | |
| 57 | 0.304 | |
| 58 | 0.648 | |
| 59 | 9.78 | |
| 60 | 0.343 | |
| 61 | 0.154 | |
| 62 | 0.058 | |
| 63 | 3.32 | |
| 64 | 0.134 | |
| 65 | 0.409 | |
| 66 | 0.3 | |
| 67 | 0.169 | |
| 68 | 0.249 | |
| 69 | 0.089 | |
| 70 | 0.209 | |
| 71 | 0.209 | |
| 72 | 0.535 | |
| 73 | 0.314 | |
| 74 | 0.631 | |
| 75 | 1.59 | |
| 76 | 0.431 | |
| 77 | 0.155 | |
| 78 | 0.157 | |
| 79 | 0.111 | |
| 80 | 0.081 | |
| 81 | 36 | |
| 82 | 2.4 | |
| 83 | 2.95 | |
| 84 | 4.34 | |
| 85 | 24 | |
| 86 | 1.65 | |
| 87 | 1.95 | |
| 88 | 3.78 | |
| 89 | 2.38 | |
| 90 | | 1.93 |
| 91 | | 0.745 |
| 92 | | 0.619 |
| 93 | | 0.349 |
| 94 | 0.82 | 0.216 |
| 95 | 12 | 3.71 |
| 96 | 1.66 | 0.3 |
| 97 | | 1.5 |
| 98 | 1.5 | 0.245 |
| 100 | | 0.014 |
| 101 | | 0.115 |
| 101a | | 0.149 |
| 102 | | 0.00918 |
| 102b | | 12 |
| 103 | | 0.0265 |
| 104 | | 0.765 |
| 105 | | 1.22 |
| 106 | | 0.319 |
| 107 | | 0.99 |
| 108 | | 0.0202 |
| 108a | | 1.9 |
| 109 | | 0.00825 |
| 110 | | 0.0101 |
| 111 | | 0.064 |
| 112 | | 0.17 |
| 113 | | 0.0245 |
| 114 | | 0.0165 |
| 115 | | 0.0665 |
| 116 | | 0.0274 |
| 117 | | 0.547 |
| 118 | | 0.19 |
| 119 | | 8.77 |
| 120 | | 0.324 |
| 121 | | 0.354 |
| 122 | | 1.05 |
| 123 | | 36 |
| 124 | | 0.844 |
| 125 | | 9.38 |
| 126 | | 2.35 |
| 127 | | 0.0688 |
| 127a | | 0.0628 |
| 127b | | 0.0839 |
| 128 | | 0.648 |
| 129 | | 0.449 |
| 130 | | 0.154 |
| 131 | | 0.775 |
| 132 | | 0.268 |
| 133 | | 0.273 |
| 134 | | 1.63 |
| 135 | | 1.14 |
| 136 | | 0.267 |
| 137 | | 0.332 |
| 138 | | 0.132 |
| 139 | | 0.0128 |
| 140 | | 0.132 |
| 141 | | 0.154 |
| 142 | | 0.303 |
| 143 | | 1.3 |
| 144 | | 0.473 |
| 145 | | 0.434 |
| 146 | | 0.00792 |
| 147 | | 0.0517 |
| 148 | | 2.26 |
| 149 | | 0.329 |
| 150 | | 0.0528 |
| 151 | | 0.463 |
| 152 | | 0.332 |
| 153 | | 0.811 |
| 154 | | 0.029 |
| 155 | | 0.546 |
| 156 | | 0.0267 |
| 157 | | 1.2 |
| 158 | | 4.72 |
| 159 | | 0.062 |
| 160 | | 0.685 |
| 161 | | 0.592 |
| 162 | | 0.03 |
| 163 | | 0.104 |
| 164 | | 0.00838 |
| 165 | | 0.392 |
| 166 | | 2.99 |
| 167 | | 2.81 |
| 168 | | 0.067 |
| 169 | | 0.14 |
| 170 | | 1.3 |
| 171 | | 0.247 |
| 172 | | 0.0162 |
| 173 | | 1.02 |
| 174 | | 17.1 |
| 175 | | 0.378 |
| 176 | | 0.319 |
| 177 | | 0.235 |
| 178 | | 2.14 |
| 179 | | 1.15 |
| 180 | | 2.28 |
| 181 | | 0.735 |
| 182 | | 0.24 |
| 183 | | 0.504 |
| 184 | | 0.658 |
| 185 | | 2.88 |
| 186 | | 0.802 |
| 187 | | 0.577 |
| 188 | | 1.77 |
| 189 | | 0.0116 |
| 190 | | 0.0201 |
| 191 | | 0.396 |
| 192 | | 0.014 |
| 193 | | 0.0296 |
| 194 | | 0.0466 |
| 195 | | 0.0467 |
| 196 | | 0.0355 |
| 197 | | 0.0139 |
| 198 | | 0.0091 |
| 199 | | 0.0427 |
| 200 | | 0.0144 |
| 201 | | 0.0117 |
| 202 | | 0.0631 |
| 203 | | 0.02 |
| 204 | | 0.00862 |
| 205 | | 0.0487 |
| 206 | | 0.0353 |
| 207 | | 0.07 |
| 208 | | 0.0379 |
| 209 | | 0.0214 |
| 210 | | 0.306 |
| 211 | | 0.00665 |
| 212 | | 0.224 |
| 213 | | 0.03 |
| 214 | | 0.255 |
| 215 | | 1.05 |
| 216 | | 0.185 |
| 217 | | 0.0135 |
| 218 | | 0.00593 |
| 219 | | 0.0676 |
| 220 | | 0.121 |
| 221 | | 0.243 |
| 222 | | 0.0568 |
| 223 | | 0.0073 |
| 224 | | 0.0549 |
| 225 | | 0.0794 |
| 226 | | 0.105 |
| 227 | | 0.48 |
| 228 | | 0.179 |
| 229 | | 1.25 |
| 230 | | 0.225 |
| 231 | | 0.069 |
| 232 | | 3.18 |
| 233 | | 2.26 |
| 234 | | 0.184 |
| 235 | | 0.0318 |
| 236 | | 0.21 |
| 237 | | 0.205 |
| 238 | | 0.0455 |
| 239 | | 0.699 |
| 240 | | 0.13 |
| 241 | | 0.472 |
| 242 | | 1.08 |
| 243 | | 3.82 |
| 244 | | 39 |
| 245 | | 14.9 |
| 246 | | 0.00853 |
| 247 | | 0.00661 |
| 248 | | 0.016 |
| 249 | | 0.039 |
| 250 | | 0.0092 |
| 251 | | 0.144 |
| 252 | | 0.104 |
| 253 | | 0.0255 |
| 254 | | 0.145 |
| 255 | | 0.275 |
| 256 | | 0.164 |
| 257 | | 6.29 |
| 258 | | 0.114 |
| 259 | | 5.85 |
| 260 | | 0.213 |
| 261 | | 0.0831 |
| 262 | | 0.267 |
| 263 | | 0.0355 |
| 264 | | 0.195 |
| 265 | | 0.098 |
| 266 | | 0.504 |
| 267 | | 0.0642 |
| 268 | | 0.46 |
| 269 | | 0.0911 |
| 270 | | 0.00352 |
| 271 | | 0.0148 |
| 272 | | 0.0649 |
| 273 | | 1.32 |
| 274 | | 0.0149 |
| 275 | | 0.0141 |
| 276 | | 0.775 |
| 277 | | 0.0125 |
| 278 | | 0.0352 |
| 279 | | 0.0108 |
| 280 | | 0.565 |
| 281 | | 0.098 |
| 282 | | 0.22 |
| 283 | | 0.53 |
| 284 | | 0.635 |
| 285 | | 0.023 |
| 286 | | 0.00693 |
| 287 | | 0.155 |
| 288 | | 0.0814 |
| 289 | | 0.0557 |
| 290 | | 0.807 |
| 291 | | 0.224 |
| 292 | | 2.51 |

1. A compound of formula (I) 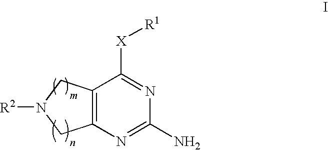
wherein:
m is 1 or 2, n is 1 or 2, when m is 2, n is 1;
X is a bond or a diradical selected from the group consisting of —O—, —S—, —(C₁-C₃ alkylene)-, —O—(C₁-C₃ alkylene)-, —NH—(C₁-C₃ alkylene)-, —S—(C₁-C₃ alkylene)-, —C(O)—, —C(O)—O—, —C(O)—NH—, —OC(O)—NH—, —NH—C(O)—NH—, —S(O)—, —S(O)₂—, —S(O)₂—O— and —S(O)₂—NH—, wherein each end of the diradical may be connected to R¹ or to the aminopyrimidine ring of formula I;
where permissible, each nitrogen or carbon atom of X is optionally further substituted by one group selected from —(C₁-C₃ alkylene)ₜ-CN, —(C₁-C₃ alkylene)ₜ-F, —(C₁-C₃ alkylene)ₜ-(C₁-C₃ perfluoroalkyl), —(C₁-C₃ alkylene)ₜ-O—(C₁-C₆ alkyl), —(C₁-C₃ alkylene)ₜ-OH, —(C₁-C₃ alkylene)ₜ-NH₂, —(C₁-C₃ alkylene)ₜ-NH(C₁-C₃ alkyl) and —(C₁-C₃ alkylene)ₜ-N(C₁-C₃ alkyl)(C₁-C₃ alkyl), and t is 0 or 1;
R¹ is selected from the group consisting of C₆-C₁₂ aryl, 5 to 12 member heteroaryl, C₃-C₁₂ cycloalkyl, 3-12 member heterocyclyl and C₅-C₁₂ unsaturated nonaromatic carbocyclyl, and each R¹ is optionally further substituted with 1-5 R, provided that when R¹ is phenyl, then R¹ is further substituted with at least two R and at least one of the R is not a halogen;
R is selected from the group consisting of R^{x}, —(C₁-C₆ alkylene)ₚ-O—(C₁-C₆ alkylene)ₚ-(C₆-C₁₀ aryl), —(C₁-C₆ alkylene)ₚ-O—(C₁-C₆ alkylene)ₚ-(C₇-C₁₀ cycloalkyl), —(C₁-C₆ alkylene)ₚ-O—(C₁-C₆ alkylene)ₚ-(7-10 member heteroaryl), —(C₁-C₆ alkylene)ₚ—O—(C₁-C₆ alkylene)ₚ-(7-10 member heterocyclyl), —(C₁-C₆ alkylene)ₚ-O—(C₂-C₆ alkenyl), —(C₁-C₆ alkylene)ₚ-O—(C₂-C₆ alkenylene)ₚ-(C₆-C₁₀ aryl), —(C₁-C₆ alkylene)ₚ-O—(C₂-C₆ alkenylene)ₚ-(C₃-C₁₀ cycloalkyl), —(C₁-C₆ alkylene)ₚ-O—(C₂-C₆ alkenylene)ₚ-(5-10 member heteroaryl), —(C₁-C₆ alkylene)ₚ-O—(C₂-C₆ alkenylene)ₚ-(3-10 member heterocyclyl), —(C₁-C₆ alkylene)ₚ-O—(C₂-C₆ alkynyl), —(C₁-C₆ alkylene)ₚ-O—(C₂-C₆ alkynylene)ₚ-(C₆-C₁₀ aryl), —(C₁-C₆ alkylene)ₚ-O—(C₂-C₆ alkynylene)ₚ-(C₃-C₁₀ cycloalkyl), —(C₁-C₆ alkylene)ₚ-O—(C₂-C₆ alkynylene)ₚ-(5-10 member heteroaryl) and —(C₁-C₆ alkylene)ₚ-O—(C₂-C₆ alkynylene)ₚ-(3-10 member heterocyclyl);
R² is selected from the group consisting of —(C₁-C₆ alkylene)ₚ-C(O)—R^{b}, —(C₁-C₆ alkylene)ₚ-C(O)—O—R^{a}, —(C₁-C₆ alkylene)ₚ-C(O)—N(R^{a})₂, —(C₁-C₆ alkylene)ₚ-S(O)—R^{a}, —(C₁-C₆ alkylene)ₚ-S(O)₂—R^{a}, —(C₁-C₆ alkylene)ₚ-S(O)₂—N(R^{a})₂, —(C₁-C₆ alkylene)ₚ-S(O)₂—O—R^{a}, and R³, wherein R³ is selected from —(C₁-C₆ alkylene)-(C₁-C₃ perfluoroalkyl), C₂-C₈ alkenyl, C₂-C₈ alkynyl, —(C₁-C₆ alkylene)ₚ-(C₃-C₁₂ cycloalkyl), —(C₁-C₆ alkylene)ₚ-(3-12 member heterocyclyl), —(C₁-C₆ alkylene)ₚ-(5-12 member heteroaryl) and —(C₁-C₆ alkylene)ₚ-(C₅-C₁₂ unsaturated nonaromatic carbocyclyl);
each R^{a} is independently selected from the group consisting of H, C₁-C₈ alkyl, C₂-C₈ alkenyl, C₂-C₈ alkynyl, C₁-C₈ perfluoroalkyl, —(C₁-C₆ alkylene)ₚ-(C₆-C₁₂ aryl), —(C₁-C₆ alkylene)ₚ-(5 to 12 member heteroaryl), —(C₁-C₆ alkylene)ₚ-(C₃-C₁₂ cycloalkyl), —(C₁-C₆ alkylene)ₚ-(3-12 member heterocyclyl), —(C₁-C₆ alkylene)ₚ-(C₅-C₁₂ unsaturated nonaromatic carbocyclyl);
two R^{a} attached to the same nitrogen atom, together with the nitrogen atom, may optionally form a 3-12 member heterocyclyl or a 5-12 member heteroaryl; the said 3-12 member heterocyclyl and the said 5-12 member heteroaryl is optionally further substituted by 1-5 R^{x};
R^{b} is selected from the group consisting of —NR^{a}N(R^{a})₂, —NR^{a}OR^{a}, C₂-C₈ alkenyl, C₂-C₈ alkynyl, C₁-C₈ perfluoroalkyl, —(C₃-C₆ alkylene)-(C₁-C₃ perfluoroalkyl), —(C₁-C₆ alkylene)ₚ-(C₆-C₁₂ aryl), —(C₁-C₆ alkylene)ₚ-(C₃-C₁₂ cycloalkyl), —(C₁-C₆ alkylene)ₚ-(3-12 member heterocyclyl), —(C₁-C₆ alkylene)ₚ-(5-12 member heteroaryl), —(C₁-C₆ alkylene)ₚ-(C₅-C₁₂ unsaturated nonaromatic carbocyclyl);
p is 0 or 1;
each R, R^{a}, R^{b} and R³ is optionally further substituted with 1-5 R^{x};
each R^{x} is independently selected from the group consisting of -oxo-, —(C₁-C₄ alkylene)-, halogen, —CN, —OH, C₁-C₆ alkyl, C₂-C₆ alkenyl, C₂-C₆ alkynyl, C₁-C₆ perfluoroalkyl, —(C₁-C₆ alkylene)-halogen, —(C₁-C₆ alkylene)-OH, —(C₁-C₆ alkylene)-CN, —(C₁-C₆ alkylene)_{q}-(C₃-C₆ cycloalkyl), —(C₁-C₆ alkylene)_{q}-(3-6 member heterocyclyl), —(C₁-C₆ alkylene)_{q}-(5-6 member heteroaryl), —(C₁-C₆ alkylene)_{q}-C(O)—(C₁-C₆ alkyl), —(C₁-C₆ alkylene)_{q}-C(O)—(C₃-C₆ cycloalkyl), —(C₁-C₆ alkylene)_{q}-C(O)—(C₁-C₆ alkylene)-(C₃-C₆ cycloalkyl), —(C₁-C₆ alkylene)_{q}-C(O)—O—(C₁-C₆ alkyl), —(C₁-C₆ alkylene)_{q}-C(O)—NH—(C₁-C₆ alkyl), —(C₁-C₆ alkylene)_{q}-C(O)—N(C₁-C₆ alkyl)(C₁-C₆ alkyl), —(C₁-C₆ alkylene)_{q}-O—(C₁-C₆ alkyl), —(C₁-C₆ alkylene)_{q}-O—(C₁-C₆ alkylene)-halogen, —(C₁-C₆ alkylene)_{q}-O—(C₁-C₆ alkylene)-(C₁-C₃ perfluoroalkyl), —(C₁-C₆ alkylene)_{q}-O—(C₁-C₆ alkylene)_{q}-(C₃-C₆ cycloalkyl), —(C₁-C₆ alkylene)_{q}-O—(C₁-C₆ alkylene)_{q}-(3-6 member heterocyclyl), —(C₁-C₆ alkylene)_{q}-O—(C₁-C₆ alkylene)_{q}-(5-6 member heteroaryl), —(C₁-C₆ alkylene)_{q}-O—(C₁-C₆ alkylene)-NH₂, —(C₁-C₆ alkylene)_{q}-O—(C₁-C₆ alkylene)-NH—(C₁-C₆ alkyl), —(C₁-C₆ alkylene)_{q}-O—(C₁-C₆ alkylene)-NH—(C₃-C₆ cycloalkyl), —(C₁-C₆ alkylene)_{q}-O—(C₁-C₆ alkylene)-N(C₁-C₆ alkyl)₂, —(C₁-C₆ alkylene)_{q}-NH₂, —(C₁-C₆ alkylene)_{q}-NH—(C₁-C₆ alkyl), —(C₁-C₆ alkylene)_{q}-NH—(C₃-C₆ cycloalkyl), —(C₁-C₆ alkylene)_{q}-N(C₁-C₆ alkyl)(C₁-C₆ alkyl), —(C₁-C₆ alkylene)_{q}-NHC(O)—(C₁-C₆ alkyl), —(C₁-C₆ alkylene)_{q}-NH—SO₂—(C₁-C₆ alkyl), —(C₁-C₆ alkylene)_{q}-SO₂—(C₁-C₆ alkyl), —(C₁-C₆ alkylene)_{q}-SO₂—(C₁-C₃ alkylene)_{q}-(C₃-C₆ cycloalkyl), —(C₁-C₆ alkylene)_{q}-SO₂—NH₂, —(C₁-C₆ alkylene)_{q}-SO₂—NH(C₁-C₃ alkyl), —(C₁-C₆ alkylene)_{q}-SO₂—NH—(C₁-C₃ alkylene)_{q}-(C₃-C₆ cycloalkyl) and —(C₁-C₆ alkylene)_{q}-SO₂—N(C₁-C₃ alkyl)₂; each q is independently 0 or 1; where permissible, each carbon atom of R^{x} is optionally further substituted by 1-3 fluorine;
or a pharmaceutically acceptable salt thereof.

2. The compound of claim 1, or a pharmaceutically acceptable salt thereof, wherein m is 1 and n is 1, and the compound is of formula II, 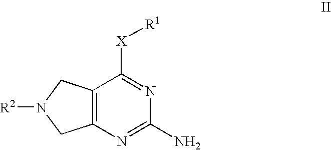
or a pharmaceutically acceptable salt thereof.

3. The compound of claim 2, or a pharmaceutically acceptable salt thereof, wherein X is a bond or —O—, R¹ is C₆-C₁₂ aryl, 5 to 12 member heteroaryl, or 3-12 member heterocyclyl, and R¹ is further substituted with 2-5 R.

4. The compound of claim 3, or a pharmaceutically acceptable salt thereof, wherein X is a bond and R¹ is a C₆-C₁₂ aryl further substituted with 2-5 R.

5. The compound of claim 4, or a pharmaceutically acceptable salt thereof, wherein R¹ is phenyl further substituted with 2-5 R and at least one of the R is not a halogen.

6. The compound of claim 5, or a pharmaceutically acceptable salt thereof, wherein R is selected from the group consisting of F, Cl, Br, —OH, —CN, C₁-C₃ alkyl, C₁-C₃ perfluoroalkyl, —(C₁-C₆ alkylene)-OH, —O—(C₁-C₆ alkyl), —(C₁-C₆ alkylene)-O—(C₁-C₆ alkyl), —(C₁-C₆ alkylene)ₚ-O—(C₁-C₆ alkylene)ₚ-(C₆-C₁₀ aryl), —(C₁-C₆ alkylene)ₚ-O—(C₁-C₆ alkylene)ₚ-(C₃-C₁₀ cycloalkyl), —(C₁-C₆ alkylene)ₚ-O—(C₁-C₆ alkylene)ₚ-(5-10 member heteroaryl), —(C₁-C₆ alkylene)ₚ—O—(C₁-C₆ alkylene)ₚ-(3-10 member heterocyclyl), —(C₁-C₆ alkylene)ₚ-O—(C₂-C₆ alkenyl), —(C₁-C₆ alkylene)ₚ-O—(C₂-C₆ alkenylene)ₚ-(C₆-C₁₀ aryl), —(C₁-C₆ alkylene)ₚ-O—(C₂-C₆ alkenylene)ₚ-(C₃-C₁₀ cycloalkyl), —(C₁-C₆ alkylene)ₚ-O—(C₂-C₆ alkenylene)ₚ-(5-10 member heteroaryl), —(C₁-C₆ alkylene)ₚ-O—(C₂-C₆ alkenylene)ₚ-(3-10 member heterocyclyl), —(C₁-C₆ alkylene)ₚ-O—(C₂-C₆ alkynyl), —(C₁-C₆ alkylene)ₚ-O—(C₂-C₆ alkynylene)ₚ-(C₆-C₁₀ aryl), —(C₁-C₆ alkylene)ₚ-O—(C₂-C₆ alkynylene)ₚ-(C₃-C₁₀ cycloalkyl), —(C₁-C₆ alkylene)ₚ-O—(C₂-C₆ alkynylene)ₚ-(5-10 member heteroaryl) and —(C₁-C₆ alkylene)ₚ-O—(C₂-C₆ alkynylene)ₚ-(3-10 member heterocyclyl); wherein each R is optionally further substituted by 1-5 R^{x}.

7. The compound of claim 2, of formula V, 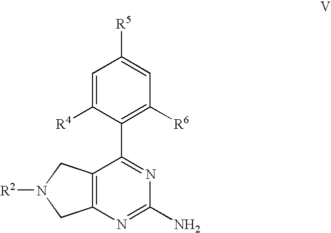
wherein
R⁴ and R⁵ are independently F, Cl, Br, —OH, —CN, unsubstituted C₁-C₃ alkyl, C₁-C₃ perfluoroalkyl, unsubstituted —(C₁-C₆ alkylene)-OH or unsubstituted —O—(C₁-C₆ alkyl);
R⁶ is selected from the group consisting of —(C₁-C₆ alkylene)-OH, —O—(C₁-C₆ alkyl), —(C₁-C₆ alkylene)-O—(C₁-C₆ alkyl), —(C₁-C₆ alkylene)ₚ-O—(C₁-C₆ alkylene)ₚ-(C₆-C₁₀ aryl), —(C₁-C₆ alkylene)ₚ-O—(C₁-C₆ alkylene)ₚ-(C₃-C₁₀ cycloalkyl), —(C₁-C₆ alkylene)ₚ-O—(C₁-C₆ alkylene)ₚ-(5-10 member heteroaryl), —(C₁-C₆ alkylene)ₚ-O—(C₁-C₆ alkylene)ₚ-(3-10 member heterocyclyl), —(C₁-C₆ alkylene)ₚ-O—(C₂-C₆ alkenyl), —(C₁-C₆ alkylene)ₚ-O—(C₂-C₆ alkenylene)ₚ-(C₆-C₁₀ aryl), —(C₁-C₆ alkylene)ₚ-O—(C₂-C₆ alkenylene)ₚ-(C₃-C₁₀ cycloalkyl), —(C₁-C₆ alkylene)ₚ-O—(C₂-C₆ alkenylene)ₚ-(5-10 member heteroaryl), —(C₁-C₆ alkylene)ₚ-O—(C₂-C₆ alkenylene)ₚ-(3-10 member heterocyclyl), —(C₁-C₆ alkylene)ₚ-O—(C₂-C₆ alkynyl), —(C₁-C₆ alkylene)ₚ-O—(C₂-C₆ alkynylene)ₚ-(C₆-C₁₀ aryl), —(C₁-C₆ alkylene)ₚ-O—(C₂-C₆ alkynylene)ₚ-(C₃-C₁₀ cycloalkyl), —(C₁-C₆ alkylene)ₚ-O—(C₂-C₆ alkynylene)ₚ-(5-10 member heteroaryl) and —(C₁-C₆ alkylene)ₚ-O—(C₂-C₆ alkynylene)ₚ-(3-10 member heterocyclyl); and R⁶ is optionally further substituted with 1-5 R^{x};
or a pharmaceutically acceptable salt thereof.

8. The compound of claim 2, of formula VI 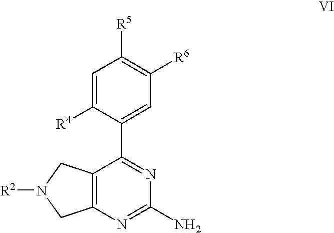
wherein
R⁴ and R⁵ are independently F, Cl, Br, —OH, —CN, unsubstituted C₁-C₃ alkyl, C₁-C₃ perfluoroalkyl, unsubstituted —(C₁-C₆ alkylene)-OH or unsubstituted —O—(C₁-C₆ alkyl);
R⁶ is selected from the group consisting of —(C₁-C₆ alkylene)-OH, —O—(C₁-C₆ alkyl), —(C₁-C₆ alkylene)-O—(C₁-C₆ alkyl), —(C₁-C₆ alkylene)ₚ-O—(C₁-C₆ alkylene)ₚ-(C₆-C₁₀ aryl), —(C₁-C₆ alkylene)ₚ-O—(C₁-C₆ alkylene)ₚ-(C₃-C₁₀ cycloalkyl), —(C₁-C₆ alkylene)ₚ-O—(C₁-C₆ alkylene)ₚ-(5-10 member heteroaryl), —(C₁-C₆ alkylene)ₚ-O—(C₁-C₆ alkylene)ₚ-(3-10 member heterocyclyl), —(C₁-C₆ alkylene)ₚ-O—(C₂-C₆ alkenyl), —(C₁-C₆ alkylene)ₚ-O—(C₂-C₆ alkenylene)ₚ-(C₆-C₁₀ aryl), —(C₁-C₆ alkylene)ₚ-O—(C₂-C₆ alkenylene)ₚ-(C₃-C₁₀ cycloalkyl), —(C₁-C₆ alkylene)ₚ-O—(C₂-C₆ alkenylene)ₚ-(5-10 member heteroaryl), —(C₁-C₆ alkylene)ₚ-O—(C₂-C₆ alkenylene)ₚ-(3-10 member heterocyclyl), —(C₁-C₆ alkylene)ₚ-O—(C₂-C₆ alkynyl), —(C₁-C₆ alkylene)ₚ-O—(C₂-C₆ alkynylene)ₚ-(C₆-C₁₀ aryl), —(C₁-C₆ alkylene)ₚ-O—(C₂-C₆ alkynylene)ₚ-(C₃-C₁₀ cycloalkyl), —(C₁-C₆ alkylene)ₚ-O—(C₂-C₆ alkynylene)ₚ-(5-10 member heteroaryl) and —(C₁-C₆ alkylene)ₚ-O—(C₂-C₆ alkynylene)ₚ-(3-10 member heterocyclyl); and R⁶ is optionally further substituted with 1-5 R^{x};
or a pharmaceutically acceptable salt thereof.

9. The compound of claim 7, or a pharmaceutically acceptable salt thereof, wherein R⁶ is —O—(C₁-C₆ alkylene)-(5-10 member heteroaryl), and R⁶ is optionally further substituted with 1-5 R^{x}.

10. The compound of claim 7, or a pharmaceutically acceptable salt thereof, wherein R⁶ is —O—(C₁-C₆ alkylene)-(5 member heteroaryl), and R⁶ is optionally further substituted with 1-5 R^{x}.

11. The compound of claim 7, or a pharmaceutically acceptable salt thereof, wherein R⁶ is —O—(C₁-C₆ alkylene)-(3-10 member heterocyclyl), and R⁶ is optionally further substituted with 1-5 R^{x}.

12. The compound of claim 7, or a pharmaceutically acceptable salt thereof, wherein R⁶ is —O—(C₁-C₆ alkyl) or —O—(C₂-C₆ alkenyl), and R⁶ is optionally further substituted with 1-5 R^{x}.

13. The compound of claim 7, or a pharmaceutically acceptable salt thereof, wherein R² is —C(O)—N(R)₂.

14. The compound of claim 7, or a pharmaceutically acceptable salt thereof, wherein R² is —C(O)—OR^{a}.

15. A pharmaceutical composition comprising a compound of claim 1, or a pharmaceutically acceptable salt thereof, and a pharmaceutical acceptable carrier.

16. The use of the compound of claim 1, or a pharmaceutically acceptable salt thereof, in the preparation of a medicament for the treatment of cancer.

17. A method of modulating the activity of HSP-90, comprising contacting a cell with a compound of claim 1, or a pharmaceutically acceptable salt thereof.
